(19) United States
(12) Patent Application Publication
(10) Pub. No.: US 2024/0150348 A1
(43) Pub. Date: May 9, 2024

(54) BRD9 DEGRADERS AND USES THEREOF

(71) Applicant: FOGHORN THERAPEUTICS INC., Cambridge, MA (US)

(72) Inventors: Sabine K. RUPPEL, Cambridge, MA (US); Zhaoxia YANG, Belmont, MA (US); Jason T. LOWE, East Bridgewater, MA (US); Johannes H. VOIGT, Cambridge, MA (US); Matthew NETHERTON, Cambridge, MA (US); Francois BRUCELLE, Belmont, MA (US)

(21) Appl. No.: 18/456,301

(22) Filed: Aug. 25, 2023

Related U.S. Application Data
(63) Continuation of application No. 17/382,543, filed on Jul. 22, 2021, now Pat. No. 11,787,800.
(60) Provisional application No. 63/058,249, filed on Jul. 29, 2020.

(51) Int. Cl. C07D 471/04; A61P 35/00

(52) U.S. Cl. CPC C07D 471/04; A61P 35/00

(57) ABSTRACT

The present invention relates to methods and compositions for the treatment of BAF-related disorders such as cancers and viral infections.

### SEQUENCE LISTING

The instant application contains a Sequence Listing which has been submitted electronically in XML format and is hereby incorporated by reference in its entirety. Said XML copy, created on Aug. 25, 2023, is named “51121-053003_Sequence_Listing_8_25_23” and is 456,626 bytes in size.

### BACKGROUND

Disorders can be affected by the BAF complex. BRD9 is a component of the BAF complex. The present invention relates to useful compositions and methods for the treatment of BAF complex-related disorders, such as cancer and infection.

### SUMMARY

Bromodomain-containing protein 9 (BRD9) is a protein encoded by the BRD9 gene on chromosome 5. BRD9 is a component of the BAF (BRG1- or BRM-associated factors) complex, a SWI/SNF ATPase chromatin remodeling complex, and belongs to family IV of the bromodomain-containing proteins. BRD9 is present in several SWI/SNF ATPase chromatin remodeling complexes and is upregulated in multiple cancer cell lines. Accordingly, agents that reduce the levels and/or activity of BRD9 may provide new methods for the treatment of disease and disorders, such as cancer and infection. The inventors have found that depleting BRD9 in cells results in the depletion of the SS18-SSX fusion protein in those cells. The SS18-SSX fusion protein has been detected in more than 95% of synovial sarcoma tumors and is often the only cytogenetic abnormality in synovial sarcoma. Additionally, evidence suggests that the BAF complex is involved in cellular antiviral activities. Thus, agents that degrade BRD9 (e.g., compounds) are useful in the treatment of disorders (e.g., cancers or infections) related to BAF, BRD9, and/or SS18-SSX.

The present disclosure features compounds and methods useful for treating BAF-related disorders (e.g., cancer or infection).

In an aspect, the disclosure features a compound having the structure of Formula I:

A-L-B Formula I,
where
L has the structure of Formula II:

A′-E′-F-E²-A², Formula II
A¹ is a bond between the linker and A;
A² is a bond between B and the linker;
each of E¹ and E² is, independently, absent, CH₂, O, or NCH₃; and
F is optionally substituted C₃-C₁₀ carbocyclylene or optionally substituted C₂₋₁₀ heterocyclylene;
B is a degradation moiety; and
A has the structure of Formula III:

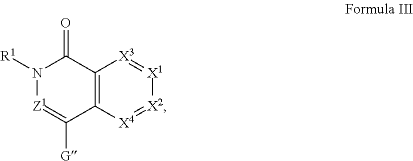

where
R¹ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
Z¹ is CR² or N;
R² is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, or optionally substituted C₂-C₉ heteroaryl;
X¹ is N or CH, and X² is C—R⁷″; or X¹ is C—R⁷″, and X² is N or CH;
R⁷″ is

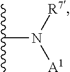

optionally substituted C₁-C₆ alkyl, optionally substituted C₁-C₆ heteroalkyl, optionally substituted C₁-C₆ alkoxy, optionally substituted amino, optionally substituted sulfone, optionally substituted sulfonamide, optionally substituted carbocyclyl having 3 to 6 atoms, or optionally substituted heterocyclyl having 3 to 6 atoms;
R⁷′ is H, optionally substituted C₁-C₆ alkyl, optionally substituted C₁-C₆ heteroalkyl, or optionally substituted C₃-C₁₀ carbocyclyl;
X³ is N or CH;
X⁴ is N or CH;
G″ is

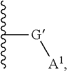

optionally substituted C₃-C₁₀ carbocyclyl, C₂-C₉ heterocyclyl, optionally substituted C₆-C₁₀ aryl, or optionally substituted C₂-C₉ heteroaryl;
G′ is optionally substituted C₃-C₁₀ carbocyclylene, C₂-C₉ heterocyclylene, optionally substituted C₆-C₁₀ arylene, or optionally substituted C₂-C₉ heteroarylene; and
A¹ is a bond between A and the linker,
where G″ is

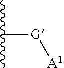

or R⁷″ is

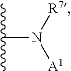

or a pharmaceutically acceptable salt thereof.

In some embodiments, R¹ is H, optionally substituted C₁-C₆ alkyl, optionally substituted C₁-C₆ heteroalkyl, or optionally substituted C₃-C₁₀ carbocyclyl. In some embodiments, R¹ is H, optionally substituted C₁-C₆ alkyl, optionally substituted C₂-C₆ alkenyl, or optionally substituted C₃-C₁₀ carbocyclyl. In some embodiments, R¹ is H, optionally substituted C₁-C₆ alkyl, or optionally substituted C₃-C₁₀ carbocyclyl.

In some embodiments, R¹ is H. In some embodiments, R¹ is optionally substituted C₁-C₆ alkyl. In some embodiments, R¹ is optionally substituted C₂-C₆ alkenyl. In some embodiments, R¹ is optionally substituted C₃-C₁₀ carbocyclyl.

In some embodiments, optionally substituted C₁-C₆ alkyl is C₁-C₆ perfluoroalkyl.

In some embodiments, R¹ is

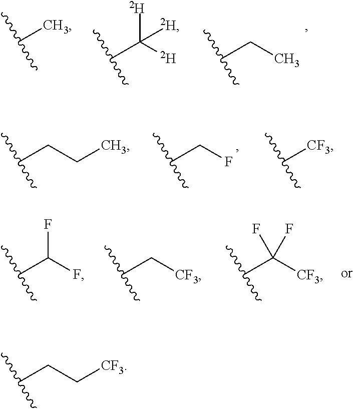

In some embodiments, R¹ is

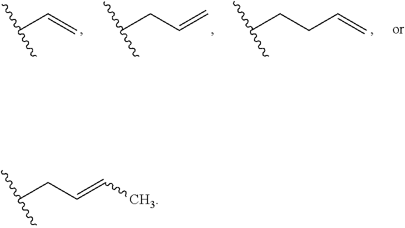

In some embodiments, R¹ is

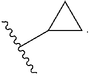 [SMILES: *C1CC1]

In some embodiments, R¹ is H,

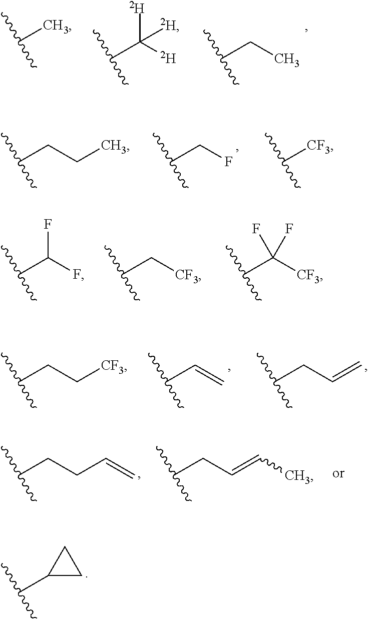

In some embodiments, R¹ is

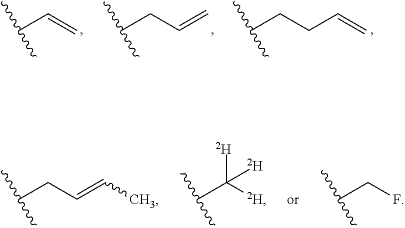

In some embodiments, R¹ is H,

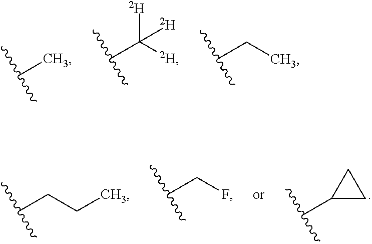

In some embodiments, R¹ is H,

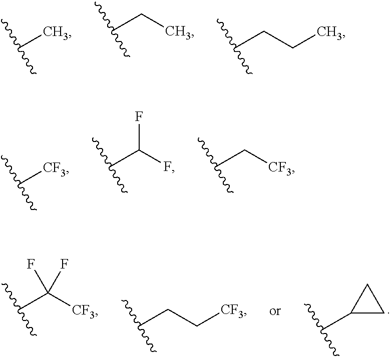

In some embodiments, R¹ is H,

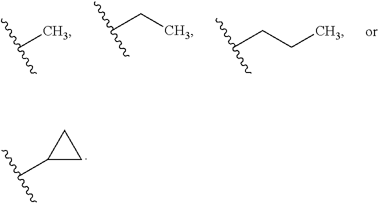

In some embodiments, R¹ is H or

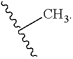

In some embodiments, R¹ is H. In some embodiments, R¹ is

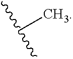

In some embodiments, Z¹ is CR². In some embodiments, Z¹ is N.

In some embodiments, R² is H, halogen, optionally substituted C₁-C₆ alkyl, optionally substituted C₃-C₁₀ carbocyclyl, or optionally substituted C₆-C₁₀ aryl.

In some embodiments, R² is H, halogen, or optionally substituted C₁-C₆ alkyl.

In some embodiments, R² is H, F, or

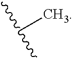

In some embodiments, R² is H. In some embodiments, R² is F. In some embodiments, R² is

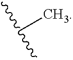

In some embodiments, R⁷″ is optionally substituted C₁-C₆ alkyl, optionally substituted C₁-C₆ heteroalkyl, optionally substituted C₁-C₆ alkoxy, optionally substituted carbocyclyl having 3 to 6 atoms, or optionally substituted heterocyclyl having 3 to 6 atoms. In some embodiments, R⁷″ is optionally substituted C₁-C₆ alkyl, optionally substituted C₁-C₆ heteroalkyl, optionally substituted carbocyclyl having 3 to 6 atoms, or optionally substituted heterocyclyl having 3 to 6 atoms. In some embodiments, R⁷″ is optionally substituted C₁-C₆ alkoxy or optionally substituted amino. In some embodiments, R⁷″ is optionally substituted sulfone or optionally substituted sulfonamide.

In some embodiments, R⁷″ is optionally substituted C₁-C₆ alkyl or optionally substituted carbocyclyl having 3 to 6 atoms. In some embodiments, R⁷″ is optionally substituted C₁-C₆ heteroalkyl or optionally substituted heterocyclyl having 3 to 6 atoms. In some embodiments, R⁷″ is optionally substituted C₁-C₆ alkyl or optionally substituted C₁-C₆ heteroalkyl.

In some embodiments, R⁷″ is optionally substituted C₁-C₆ alkyl. In some embodiments, R⁷″ is optionally substituted C₁-C₆ heteroalkyl. In some embodiments, R⁷″ is optionally substituted C₁-C₆ alkoxy. In some embodiments, R⁷″ is optionally substituted amino. In some embodiments, R⁷″ is optionally substituted carbocyclyl having 3 to 6 atoms. In some embodiments, R⁷″ is optionally substituted heterocyclyl having 3 to 6 atoms. In some embodiments, R⁷″ is optionally substituted sulfone. In some embodiments, R⁷″ is optionally substituted sulfonamide.

In some embodiments, R⁷″ is optionally substituted C₁-C₃ alkyl. In some embodiments, R⁷″ is optionally substituted C₁-C₃ heteroalkyl.

In some embodiments, R⁷″ is

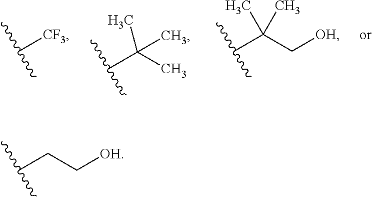

In some embodiments, R⁷″ is —NR³R⁴ or —OR⁴, where R³ is H or optionally substituted C₁-C₆ alkyl, and R⁴ is optionally substituted C₁-C₆ alkyl.

In some embodiments, R⁷″ is —NR³R⁴. In some embodiments, R⁷″ is —OR⁴.

In some embodiments, R³ is H. In some embodiments, R³ is optionally substituted C₁-C₆ alkyl.

In some embodiments, R³ is H and R⁴ is methyl. In some embodiments, R³ is methyl and R⁴ is methyl.

In some embodiments, R⁷″ is

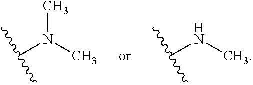

In some embodiments, R⁷″ is

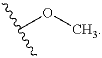 [SMILES: *OC]

In some embodiments, R⁷″ is optionally substituted carbocyclyl having 3 to 6 atoms or optionally substituted heterocyclyl having 3 to 6 atoms. In some embodiments, R⁷″ is optionally substituted carbocyclyl having 3 to 6 atoms. In some embodiments, R⁷″ is optionally substituted heterocyclyl having 3 to 6 atoms.

In some embodiments, R⁷″ is carbocyclyl having 3 to 6 atoms or heterocyclyl having 3 to 6 atoms. In some embodiments, R⁷″ is carbocyclyl having 3 to 6 atoms. In some embodiments, R⁷″ is heterocyclyl having 3 to 6 atoms.

In some embodiments, R⁷″ is

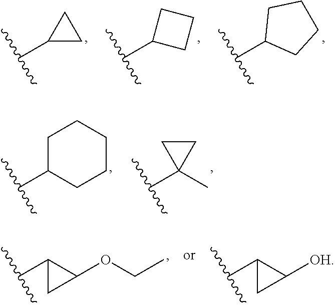

In some embodiments, R⁷″ is

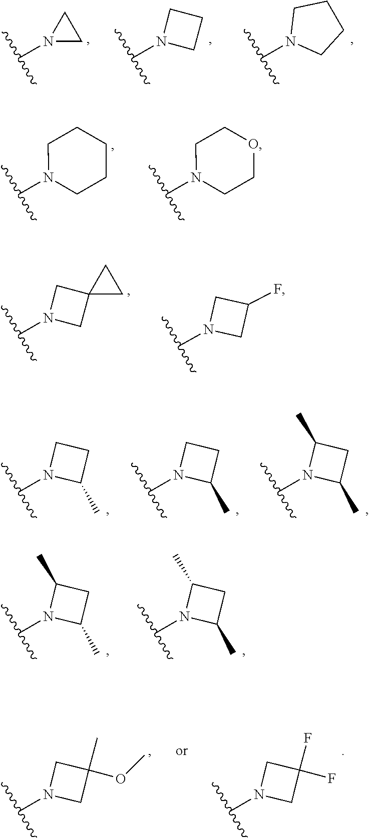

In some embodiments, R⁷″ is

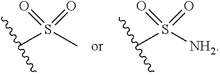

In some embodiments, R⁷″ is

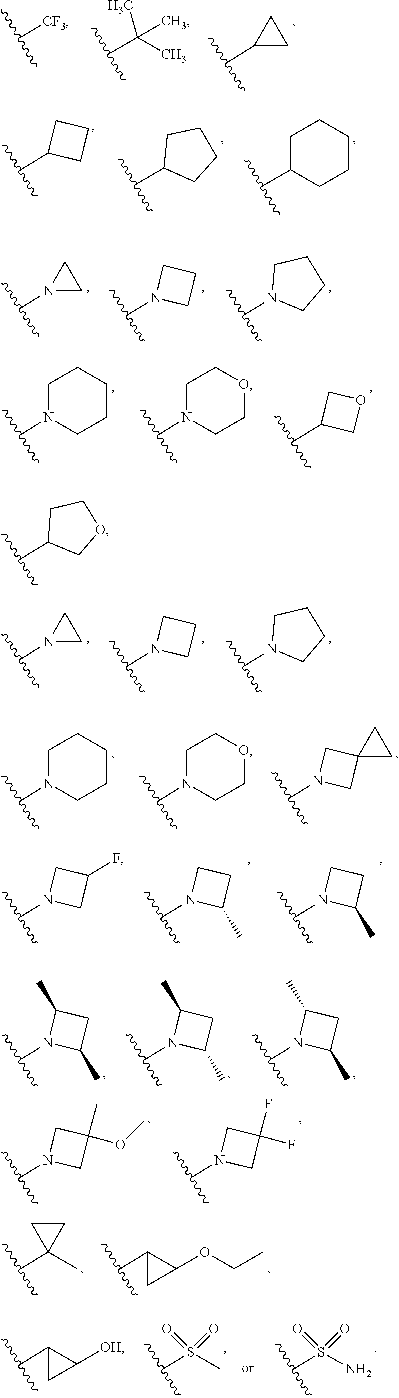

In some embodiments, R⁷″ is

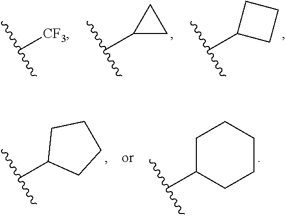

In some embodiments, R⁷″ is

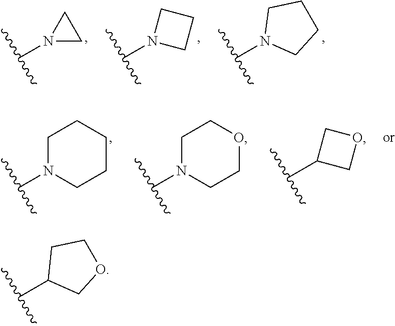

In some embodiments, R⁷″ is

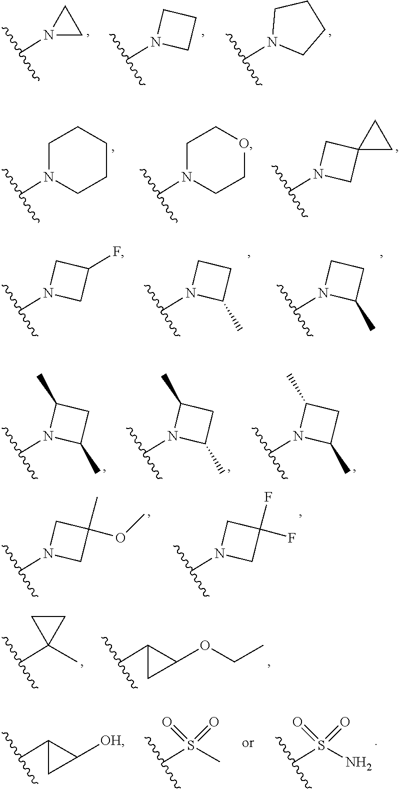

In some embodiments, R⁷″ is

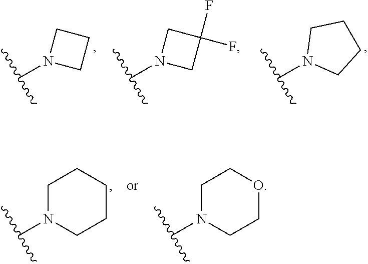 [SMILES: *N1CCC1 ; *N1CC(F)(F)C1 ; *N1CCCC1 ; *N1CCCCC1 ; *N1CCOCC1]

In some embodiments, R⁷″ is

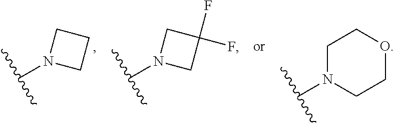 [SMILES: *N1CCC1 ; *N1CC(F)(F)C1 ; "or" ; *N1CCOCC1]

In some embodiments, X¹ is N and X² is C—R⁷″. In some embodiments, X¹ is OH and X² is C—R⁷″. In some embodiments, X¹ is C—R⁷″ and X² is N. In some embodiments, X¹ is C—R⁷″ and X² is OH.

In some embodiments, X¹ is N or CH, and X² is C—NR³R⁴, C—OR⁴,

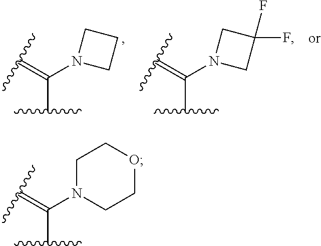

or X¹ is C—NR³R⁴, C—OR⁴,

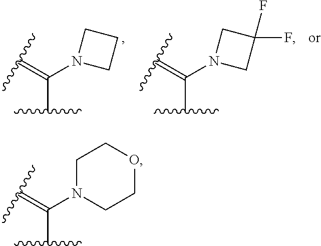

and X² is N or CH. In some embodiments, X¹ is N or CH, and X² is C—NR³R⁴,

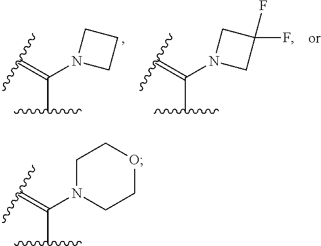

or X¹ is C—NR³R⁴,

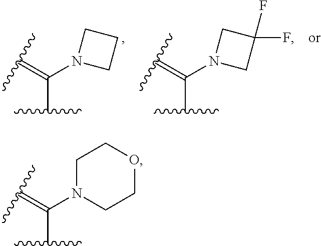

and X² is N or CH. In some embodiments, X¹ is N or CH, and X² is C—NR³R⁴ or

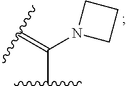 [SMILES: *C(=*)N1CCC1]

or X¹ is C—NR³R⁴ or

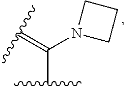 [SMILES: *C(=*)N1CCC1]

and X² is N or CH. In some embodiments, X¹ is N or CH, and X² is C—NR³R⁴ or

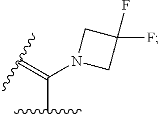 [SMILES: *C(=*)N1CC(F)(F)C1]

or X¹ is C—NR³R⁴ or

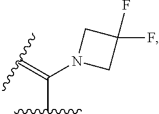 [SMILES: *C(=*)N1CC(F)(F)C1]

and X² is N or CH. In some embodiments, X¹ is N or CH, and X² is C—NR³R⁴ or

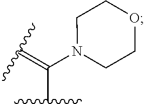 [SMILES: *C(=*)N1CCOCC1]

or X¹ is C—NR³R⁴ or

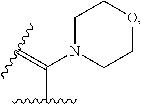 [SMILES: *C(=*)N1CCOCC1]

### and X² is N or CH.

In some embodiments, R⁷″ is —NR³R⁴, —OR⁴, or optionally substituted heterocyclyl having 3 to 6 atoms.

In some embodiments, X¹ is N and X² is C—NR³R⁴. In some embodiments, X¹ is C—NR³R⁴ and X² is N.

In some embodiments, R³ is H. In some embodiments, R³ is optionally substituted C₁-C₆ alkyl.

In some embodiments, R³ is

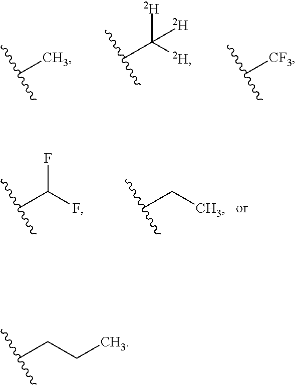

In some embodiments, R³ is

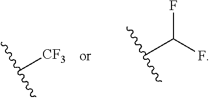

In some embodiments, R³ is

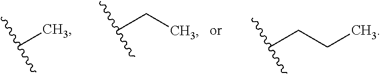

In some embodiments, R³ is methyl, ethyl

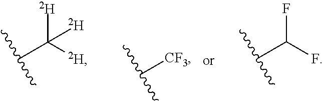

In some embodiments, R⁴ is

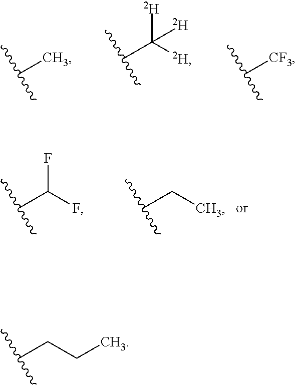

In some embodiments, R⁴ is

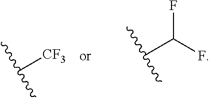

In some embodiments, R⁴ is

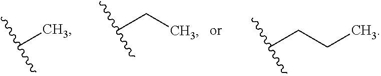

In some embodiments, R⁴ is methyl, ethyl,

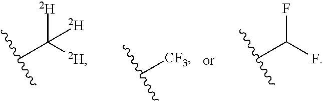

In some embodiments, X³ is N. In some embodiments, X³ is CH.

In some embodiments, X⁴ is N. In some embodiments, X⁴ is CH.

In some embodiments, X³ is N and X⁴ is N.

In some embodiments, X³ is N and X⁴ is CH.

In some embodiments, X³ is CH and X⁴ is N.

In some embodiments, X³ is CH and X⁴ is CH.

In some embodiments, G″ is

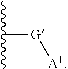

In some embodiments, G′ is optionally substituted C₃-C₁₀ carbocyclylene or optionally substituted C₂-C₉ heterocyclylene. In some embodiments, G′ is optionally substituted C₆-C₁₀ arylene or optionally substituted C₂-C₉ heteroarylene.

In some embodiments, G′ is optionally substituted C₃-C₁₀ carbocyclylene. In some embodiments, G′ is optionally substituted C₆-C₁₀ arylene. In some embodiments, G′ is optionally substituted C₂-C₉ heterocyclylene. In some embodiments, G′ is optionally substituted C₂-C₉ heteroarylene.

In some embodiments, G′ is

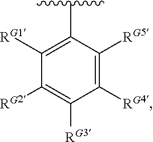

where
each of R^{G1}′, R^{G2}′, R^{G3}′, R^{G4}′, and R^{G5}′ is, independently, H, A¹, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G1}′ and R^{G2}′, R^{G2}′ and R^{G3}′, R^{G3}′ and R^{G4}′, and/or R^{G4}′ and R^{G5}′, together with the carbon atoms to which each is attached, combine to form

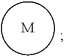

and

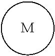

is optionally substituted C₆-C₁₀ aryl, optionally substituted C₃-C₁₀ carbocyclyl, optionally substituted C₂-C₉ heteroaryl, or optionally substituted C₂-C₉ heterocyclyl, any of which is optionally substituted with A¹, where one of R^{G1}′, R^{G2}′, R^{G3}′, R^{G4}′, and R^{G5}′ is A¹, or

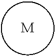

is substituted with A¹.

In some embodiments, each of R^{G1}′, R^{G2}′, R^{G3}′, R^{G4}′, and R^{G5}′ is, independently, H, A¹, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G1}′ and R^{G2}′ R^{G2}′ and R^{G3}′, R^{G3}′ and R^{G4}′, and/or R^{G4}′ and R^{G5}′, together with the carbon atoms to which each is attached, combine to form

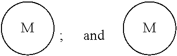

is optionally substituted C₆-C₁₀ aryl, optionally substituted C₃-C₁₀ carbocyclyl, optionally substituted C₂-C₉ heteroaryl, or optionally substituted C₂-C₉ heterocyclyl, any of which is optionally substituted with A¹, where one of R^{G1}′, R^{G2}′, R^{G3}′, R^{G4}′, and R^{G5}′ is A¹, or

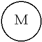

is substituted with A¹.

In some embodiments, each of R^{G1}′, R^{G2}′, R^{G3}′, R^{G4}′, and R^{G5}′ is, independently, H, A¹, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl; or R^{G1}′ and R^{G2}′, R^{G2}′ and R^{G3}′, R^{G3}′ and R^{G4}′, and/or R^{G4}′ and R^{G5}′, together with the carbon atoms to which each is attached, combine to form

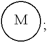

and

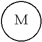

is optionally substituted C₂-C₉ heteroaryl or optionally substituted C₂-C₉ heterocyclyl, any of which is optionally substituted with A¹, where one of R^{G1}′, R^{G2}′, R^{G3}′, R^{G4}′ and R^{G5}′ is A¹, or

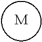

is substituted with A¹.

In some embodiments, each of R^{G1}′, R^{G2}′, R^{G3}′, R^{G4}′, and R^{G5}′ is, independently, H, A¹, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl.

In some embodiments, each of R^{G1}′, R^{G2}′, R^{G3}′, R^{G4}′, and R^{G5}′ is, independently, H, A¹, F, Cl,

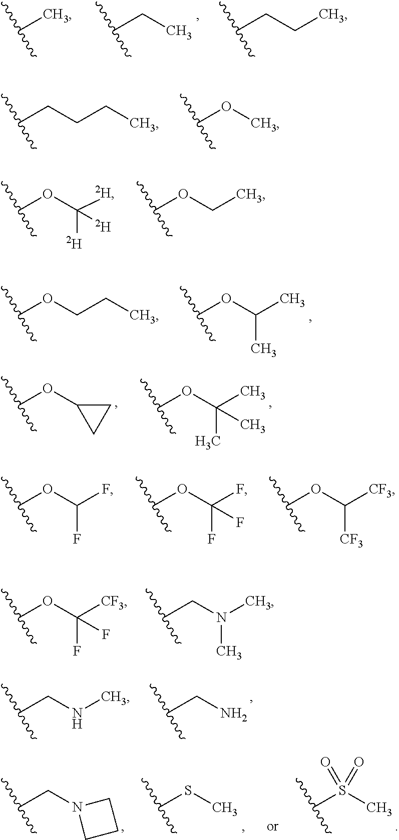

In some embodiments, each of R^{G1}′, R^{G2}′, R^{G3}′, R^{G4}′, and R^{G5}′ is, independently, H, A¹, F,

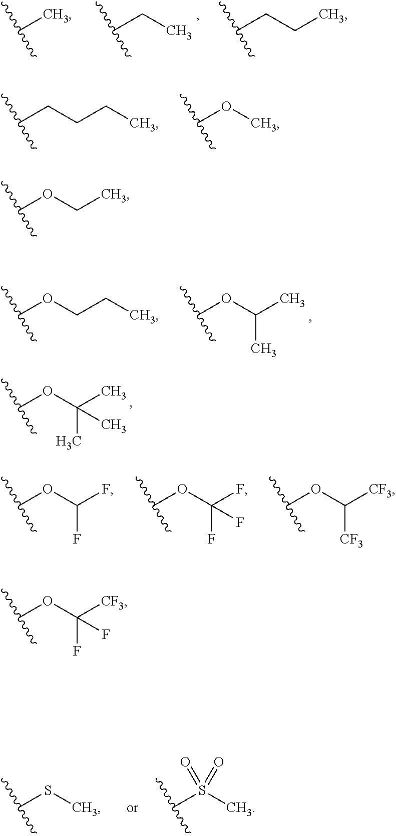

In some embodiments, each of R^{G1}′, R^{G2}′, R^{G3}′, R^{G4}′, and R^{G5}′ is, independently, H, A¹, F, Cl,

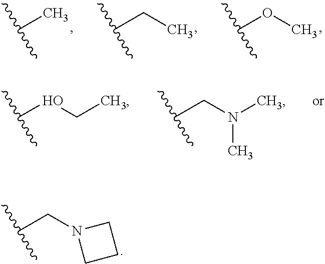

In some embodiments, R^{G3}′ is A¹.

In some embodiments, R^{G1}′ is H; R^{G2}′ is

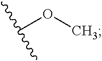 [SMILES: *OC]

R^{G3}′ is A; R^{G4}′ is

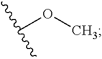 [SMILES: *OC]

and R^{G5}′ is H. In some embodiments, R^{G1}′ is H; R^{G2}′ is

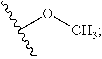 [SMILES: *OC]

R^{G3}′ is A¹; R^{G4}′ is H; and R^{G5}′ is

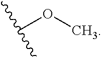 [SMILES: *OC]

In some embodiments, R^{G1}′ is H; R^{G2}′ is

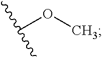 [SMILES: *OC]

R^{G3}′ is A¹; R^{G4}′ is Cl or F; and R^{G5}′ is H. In some embodiments, R^{G1}′ is H; R^{G2}′ is

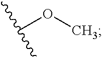 [SMILES: *OC]

R^{G3}′ is A¹; R^{G4}′ is H; and R^{G5}′ is H. In some embodiments, R^{G1}′ is H; R^{G2}′ is

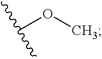 [SMILES: *OC]

R^{G3}′ is A¹; R^{G4}′ is

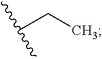 [SMILES: *CC]

### and R^{G5}′ is H.

In some embodiments, R^{G1}′ and R^{G2}′, R^{G2}′ and R^{G3}′, R^{G3}′ and R^{G4}′, and/or R^{G4}′ and R^{G5}′, together with the carbon atoms to which each is attached, combine to form

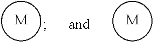

is optionally substituted C₂-C₉ heterocyclyl, which is optionally substituted with A¹, where one of R^{G1}′, R^{G2}′, R^{G3}′, R^{G4}′, and R^{G5}′ is A¹, or

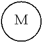

is substituted with A¹. In some embodiments, R^{G1}′ and R^{G2}′, R^{G2}′ and R^{G3}, R^{G3}′ and R^{G4}, and/or R^{G4}′ and R^{G5}′, together with the carbon atoms to which each is attached, combine to form

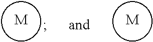

and optionally substituted C₂-C₉ heteroaryl, which is optionally substituted with A¹, where one of R^{G1}′, R^{G2}′, R^{G3}′, R^{G4}, and R^{G5}′ is A¹, or

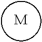

is substituted with A¹.

In some embodiments, G′ is

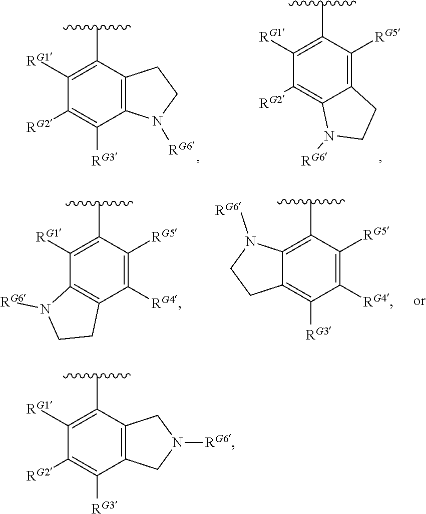

where R^{G6}′ is H, A¹, or optionally substituted C₁-C₆ alkyl. In some embodiments, G′ is

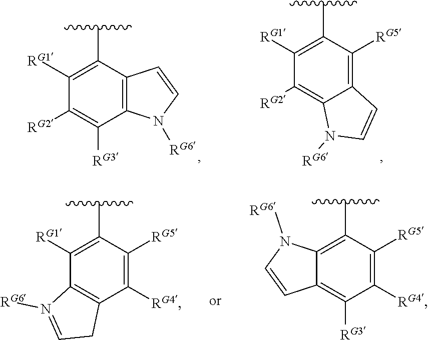

where R^{G6}′ is H, A¹, or optionally substituted C₁-C₆ alkyl.

In some embodiments, R^{G1}′ and R^{G2}′, R^{G2}′ and R^{G3}′, R^{G3}′ and R^{G4}′ and/or R^{G4}′ and R^{G5}, together with the carbon atoms to which each is attached, combine to form

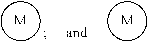

and is optionally substituted C₂-C₉ heterocyclyl or optionally substituted C₂-C₉ heteroaryl, any of which is optionally substituted with A¹, where one of R^{G1}′, R^{G2}′, R^{G3}′, R^{G4}′, and R^{G5}′ is A¹, or

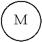

is substituted with A¹.

In some embodiments, G′ is

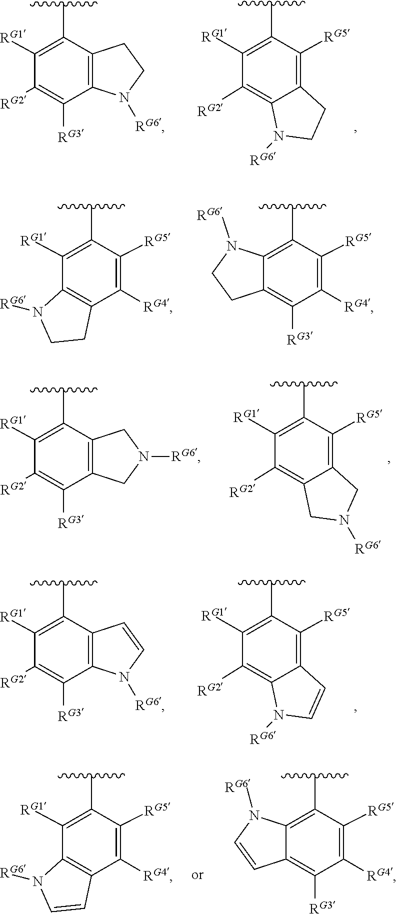

where R^{G6}′ is H, A¹, or optionally substituted C₁-C₆ alkyl.

In some embodiments, R^{G6}′ is H, A¹,

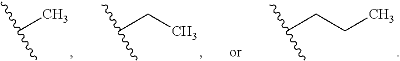

In some embodiments, R^{G6}′ is H, A¹, or

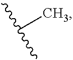

In some embodiments, R^{G6}′ is H or A¹.

In some embodiments, R^{G6}′ is H. In some embodiments, R^{G6}′ is A¹.

In some embodiments, R^{G1}′ is H, A¹, F,

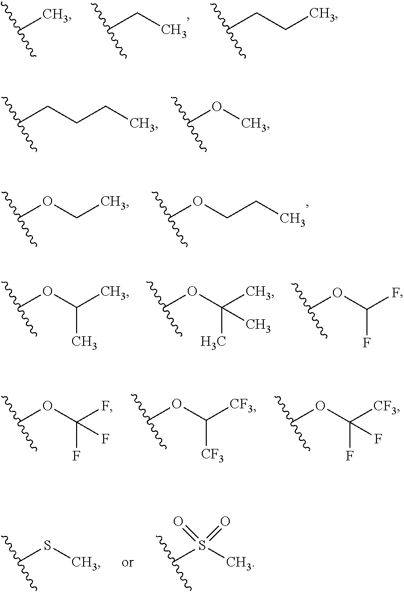

In some embodiments, R^{G1}′ is H.

In some embodiments, R^{G2}′ is H, A¹, F,

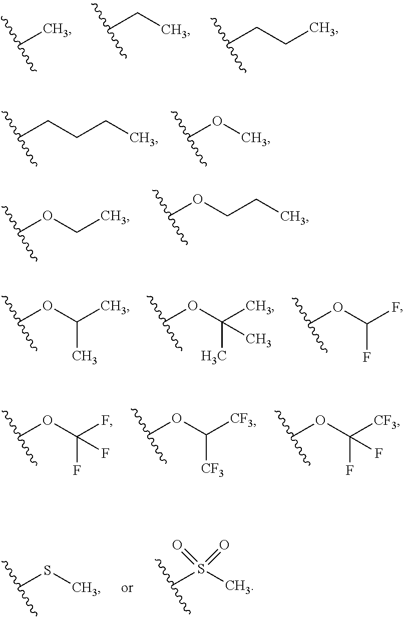

In some embodiments, R^{G2}′ is H.

In some embodiments, R^{G3}′ is H, A¹, F,

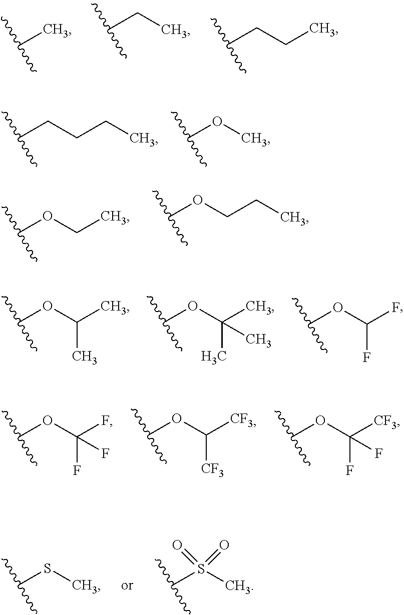

In some embodiments, R^{G3}′ is H.

In some embodiments, R^{G4}′ is H, A¹, F,

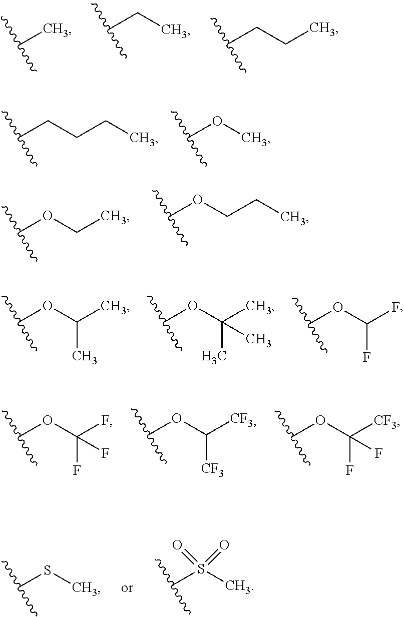

In some embodiments, R^{G4}′ is H.

In some embodiments, R^{G5}′ is H, A¹, F,

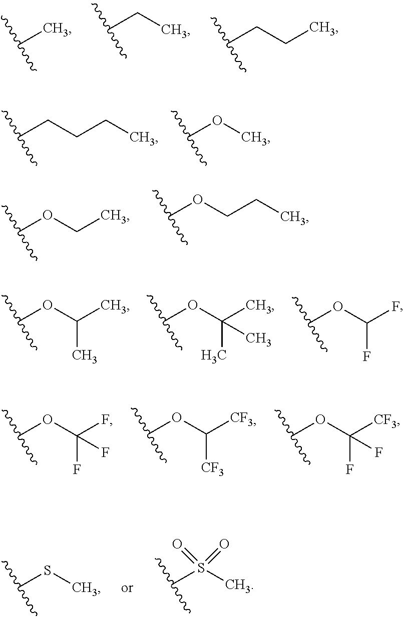

In some embodiments, R^{G5}′ is H.

In some embodiments, one or more of R^{G1}′, R^{G2}, R^{G3}, R^{G4}, and R^{G5}′ is H. In some embodiments, two or more of R^{G1}′, R^{G2}, R^{G3}, R^{G4}, and R^{G5}′ is H. In some embodiments, three or more of R^{G1}′, R^{G2}, R^{G3}, R^{G4}′ and R^{G5}′ is H.

In some embodiments, R^{G1}′ is A¹. In some embodiments, R^{G2}′ is A¹. In some embodiments, R^{G3}′ is A¹. In some embodiments, R^{G4}′ is A¹. In some embodiments, R^{G5}′ is A¹. In some embodiments,

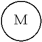

is substituted with A¹.

In some embodiments, G′ is

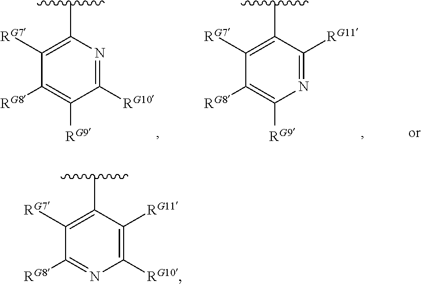

where
each of R^{G7}′, R^{G8}′, R^{G9}′, R^{G10}′, and R^{G11}′ is, independently, H, A¹, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G7}′ and R^{G8}′, R^{G8} and R^{G9}′, R^{G9}′ and R^{G10}′ and/or R^{G10}′ and R^{G11}′ together with the carbon atoms to which each is attached, combine to form

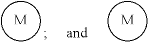

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A¹, where one of R^{G7}′, R^{G8}′, R^{G9}′, R^{G10}′, and R^{G11}′ is A¹; or

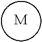

is substituted with A¹.

In some embodiments, each of R^{G7}′, R^{G8}′, R^{G9}′, R^{G10}′, and R^{G11}′ is, independently, H, A¹, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G7}′ and R^{G8}′, R^{G8}′ and R^{G9}′, R^{G9}′ and R^{G10}′ and/or R^{G10}′ and R^{G11}′, together with the carbon atoms to which each is attached, combine to form

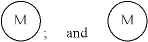

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A¹, where one of R^{G7}′, R^{G8}′, R^{G9}′, R^{G10}′, and R^{G11}′ is A¹; or

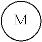

is substituted with A¹.

In some embodiments, each of R^{G7}′, R^{G8}′, R^{G9}′, R^{G10}′, and R^{G11}′ is, independently, H, A¹, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl; or R^{G7}′ and R^{G8}′, R^{G8}′ and R^{G9}′, R^{G9}′ and R^{G10}′, and/or R^{G10}′ and R^{G11}′, together with the carbon atoms to which each is attached, combine to form

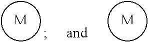

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A¹, where one of R^{G7}′, R^{G8}′, R^{G9}′, R^{G10}′, and R^{G11}′ is A¹; or

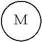

is substituted with A¹.

In some embodiments, each of R^{G7}′, R^{G8}′, R^{G9}′, R^{G10}′, and R^{G11}′ is, independently, H, A¹, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl.

In some embodiments, each of R^{G7}′, R^{G8}′, R^{G9}′, R^{G10}′, and R^{G11}′ is, independently, H, A¹, F, Cl,

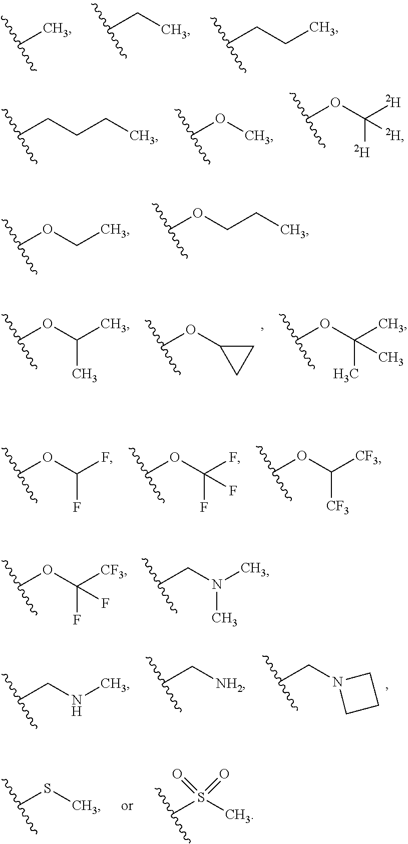

In some embodiments, R^{G8}′ is

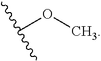 [SMILES: *OC]

In some embodiments, G′ is

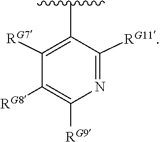

In some embodiments, R^{G7}′ is H; R^{G8}′ is

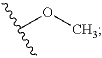 [SMILES: *OC]

R^{G9}′ is A¹; and R^{G11}′ is H.

In some embodiments, G′ is

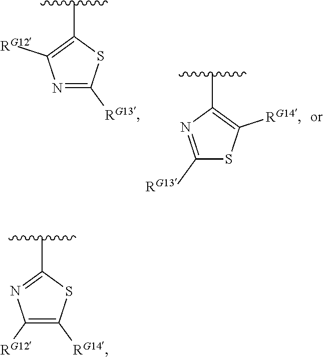

where
each of R^{G12}′, R^{G13}′ and R^{G14}′ is, independently, H, A¹, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G12}′ and R^{G14}′, together with the carbon atoms to which each is attached, combine to form

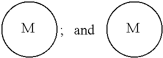

and is optionally substituted C₆-C₁₀ aryl, optionally substituted C₃-C₁₀ carbocyclyl, optionally substituted C₂-C₉ heteroaryl, or optionally substituted C₂-C₉ heterocyclyl, any of which is optionally substituted with A¹, where one of R^{G12}′, R^{G13}′, d R^{G14}′ is A¹; or

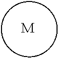

is substituted with A¹.

In some embodiments, each of R^{G12}′, R^{G13}′ and R^{G14}′ is, independently, H, A¹, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G12}′ and R^{G14}′, together with the carbon atoms to which each is attached, combine to form

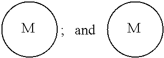

is optionally substituted C₆-C₁₀ aryl, optionally substituted C₃-C₁₀ carbocyclyl, optionally substituted C₂-C₉ heteroaryl, or optionally substituted C₂-C₉ heterocyclyl, any of which is optionally substituted with A¹, where one of R^{G12}′, R^{G13}′, and R^{G14}′ is A¹; or

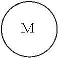

is substituted with A¹.

In some embodiments, R⁷″ is

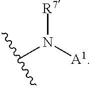

In some embodiments, R^{7′} is H, optionally substituted C₁-C₆ alkyl, or optionally substituted C₃-C₁₀ carbocyclyl. In some embodiments, R^{7′} is H or optionally substituted C₁-C₆ alkyl.

In some embodiments, R⁷′ is H,

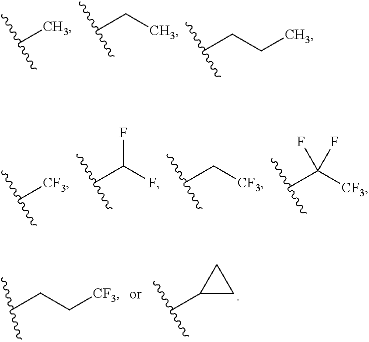

In some embodiments, R⁷′ is H or

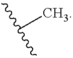

In some embodiments, R^{7′} is H. In some embodiments, R⁷′ is

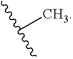

In some embodiments, G″ is optionally substituted C₃-C₁₀ carbocyclyl or optionally substituted C₂-C₉ heterocyclyl. In some embodiments, G″ is optionally substituted C₆-C₁₀ aryl or optionally substituted C₂-C₉ heteroaryl.

In some embodiments, G″ is optionally substituted C₃-C₁₀ carbocyclyl. In some embodiments, G is optionally substituted C₆-C₁₀ aryl. In some embodiments, G is optionally substituted C₂-C₉ heterocyclyl. In some embodiments, G″ is optionally substituted C₂-C₉ heteroaryl.

In some embodiments, G″ is

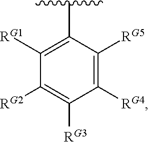

where
each of R^{G1}, R^{G2}, R^{G3}, R^{G4}, and R^{G5} is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G1} and R^{G2}, R^{G2} and R^{G3}, R^{G3} and R^{G4}, and/or R^{G4} and R^{G5}, together with the carbon atoms to which each is attached, combine to form optionally substituted C₆-C₁₀ aryl, optionally substituted C₃-C₁₀ carbocyclyl, optionally substituted C₂-C₉ heteroaryl, or optionally substituted C₂-C₉ heterocyclyl.

In some embodiments, each of R^{G1}, R^{G2}, R^{G3}, R^{G4}, and R^{G5} is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G1} and R^{G2}, R^{G2} and R^{G3}, R^{G3} and R^{G4}, and/or R^{G4} and R^{G5}, together with the carbon atoms to which each is attached, combine to form optionally substituted C₆-C₁₀ aryl, optionally substituted C₃-C₁₀ carbocyclyl, optionally substituted C₂-C₉ heteroaryl, or optionally substituted C₂-C₉ heterocyclyl.

In some embodiments, each of R^{G1}, R^{G2}, R^{G3}, R^{G4}, and R^{G5} is, independently, H, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl; or R^{G1} and R^{G2}, R^{G2} and R^{G3}, R^{G3} and R^{G4}, and/or R^{G4} and R^{G5}, together with the carbon atoms to which each is attached, combine to form optionally substituted C₂-C₉ heteroaryl or optionally substituted C₂-C₉ heterocyclyl.

In some embodiments, each of R^{G1}, R^{G2}, R^{G3}, R^{G4}, and R^{G5} is, independently, H, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl.

In some embodiments, each of R^{G1}, R^{G2}, R^{G3}, R^{G4}, and R^{G5} is, independently, H, F, Cl,

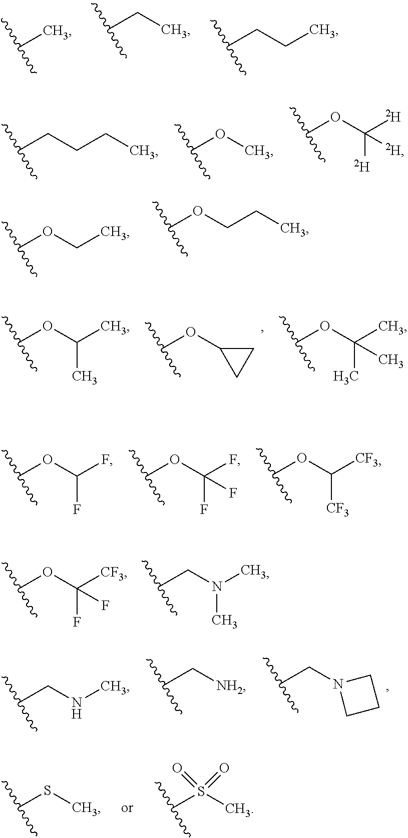

In some embodiments, each of R^{G1}, R^{G2}, R^{G3}, R^{G4}, and R^{G5} is, independently, H, F,

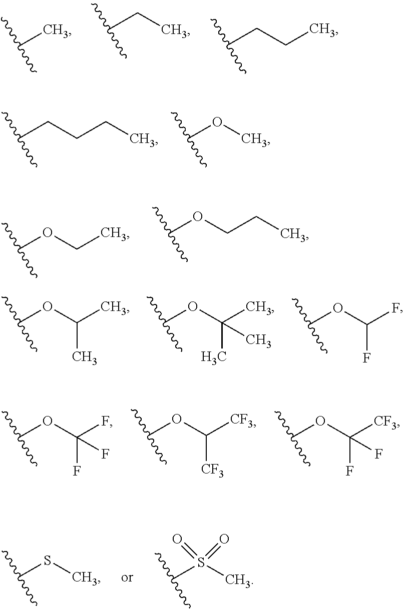

In some embodiments, each of R^{G1}, R^{G2}, R^{G3}, R^{G4}, and R^{G5} is, independently, H, F, Cl,

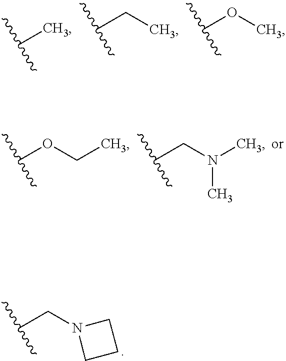

In some embodiments, R^{G1} is H; R^{G2} is

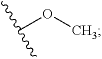 [SMILES: *OC]

### R^{G3} is

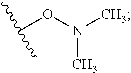 [SMILES: *ON(C)C]

### R^{G4} is

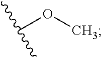 [SMILES: *OC]

and R^{G5} is H. In some embodiments, R^{G1} is H; R^{G2} is

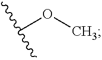 [SMILES: *OC]

### R^{G3} is

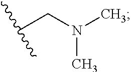 [SMILES: *CN(C)C]

R^{G4} is H; and R^{G5} is

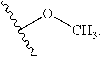 [SMILES: *OC]

In some embodiments, R^{G1} is H; R^{G2} is

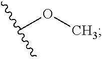 [SMILES: *OC]

### R^{G3} is

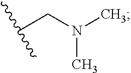 [SMILES: *CN(C)C]

R^{G4} is Cl or F; and R^{G5} is H. In some embodiments, R^{G1} is H; R^{G2} is

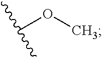 [SMILES: *OC]

### R^{G3} is

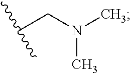 [SMILES: *CN(C)C]

R^{G4} is H; and R^{G5} is H. In some embodiments, R^{G1} is H; R^{G2} is

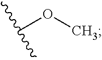 [SMILES: *OC]

### R^{G3} is

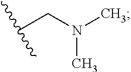 [SMILES: *CN(C)C]

### R^{G4} is

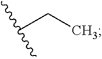 [SMILES: *CC]

### and R^{G5} is H.

In some embodiments, R^{G1} and R^{G2}, R^{G2} and R^{G3}, R^{G3} and R^{G4}; and/or R^{G4} and R^{G5}, together with the carbon atoms to which each is attached, combine to form optionally substituted C₂-C₉ heteroaryl or optionally substituted C₂-C₉ heterocyclyl.

In some embodiments, R^{G1} and R^{G2}, R^{G2} and R^{G3}, R^{G3} and R^{G4}, and/or R^{G4} and R^{G5}, together with the carbon atoms to which each is attached, combine to form optionally substituted C₂-C₉ heterocyclyl. In some embodiments, R^{G1} and R^{G2}, R^{G2} and R^{G3}, R^{G3} and R^{G4}, and/or R^{G4} and R^{G5}, together with the carbon atoms to which each is attached, combine to form optionally substituted C₂-C₉ heteroaryl.

In some embodiments, R^{G1} and R^{G2}, R^{G2} and R^{G3}, R^{G3} and R^{G4}, and/or R^{G4} and R^{G5}, together with the carbon atoms to which each is attached, combine to form optionally substituted C₂-C₉ heterocyclyl. In some embodiments, R^{G1} and R^{G2}, R^{G2} and R^{G3}, R^{G3} and R^{G4}, and/or R^{G4} and R^{G5}, together with the carbon atoms to which each is attached, combine to form optionally substituted C₂-C₉ heteroaryl.

In some embodiments, G″ is

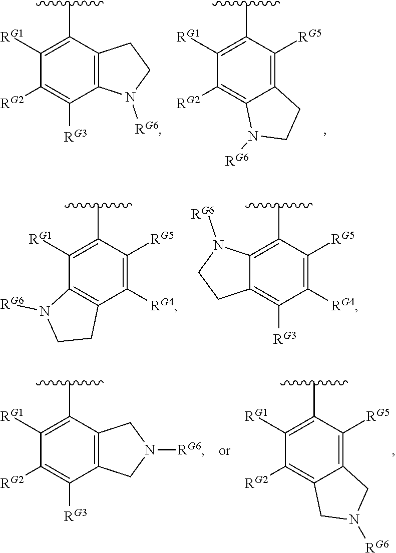

where R^{G6} is H or optionally substituted C₁-C₆ alkyl. In some embodiments, G″ is

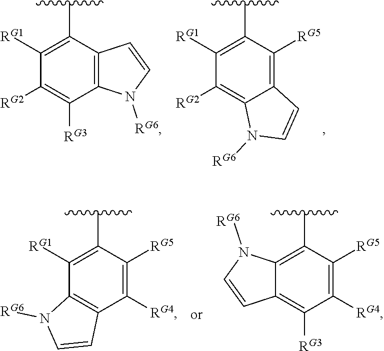

where R^{G6} is H or optionally substituted C₁-C₆ alkyl.

In some embodiments, G″ is

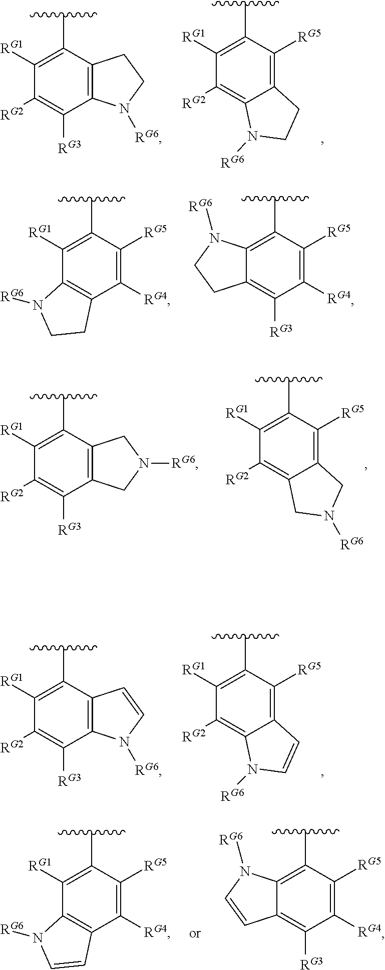

where R^{G6} is H or optionally substituted C₁-C₆ alkyl.

In some embodiments, R^{G6} is H,

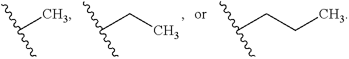

In some embodiments, R^{G6} is H or

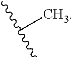

In some embodiments, R^{G6} is H.

In some embodiments, R^{G1} is H, F,

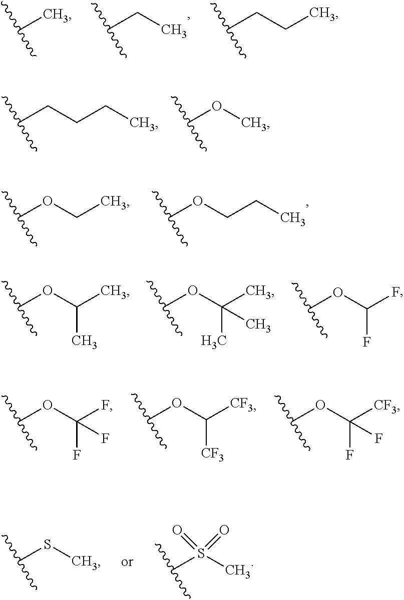

In some embodiments, R^{G1} is H.

In some embodiments, R^{G2} is H, F,

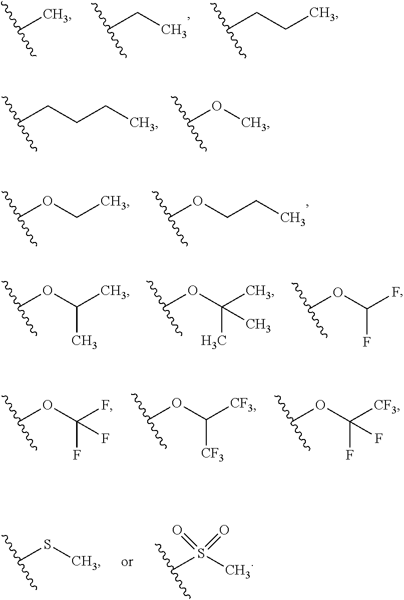

In some embodiments, R^{G2} is H.

In some embodiments, R^{G3} is H, F,

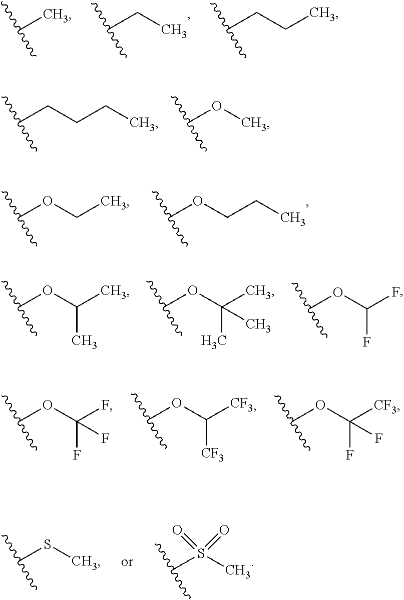

In some embodiments, R^{G3} is H.

In some embodiments, R^{G4} is H, F,

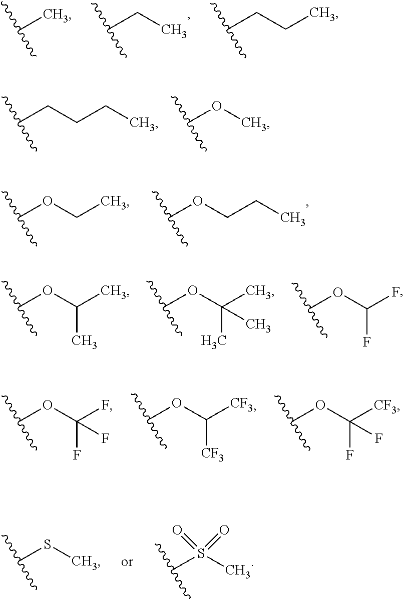

In some embodiments, R^{G4} is H.

In some embodiments, R^{G5} is H, F,

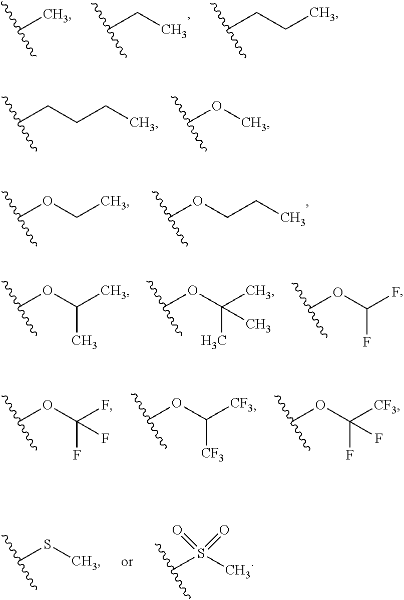

In some embodiments, R^{G5} is H.

In some embodiments, one or more of R^{G1}, R^{G2}, R^{G3}, R^{G4}, and R^{G5} is H. In some embodiments, two or more of R^{G1}, R^{G2}, R^{G3}, R^{G4}, and R^{G5} is H. In some embodiments, three or more of R^{G1}, R^{G2}, R^{G3}, R^{G4}, and R^{G5} is H. In some embodiments, each of R^{G1}, R^{G2}, R^{G3}, R^{G4}, and R^{G5} is H.

In some embodiments, G″ is

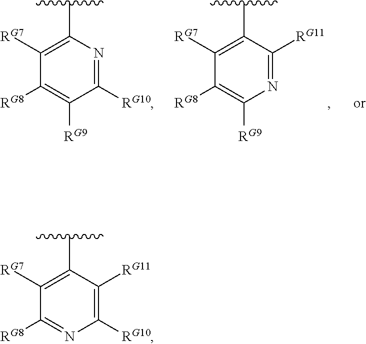

where
each of R^{G7}, R^{G8}, R^{G9}, R^{G10}, and R^{G11} is, independently, H, halogen, optionally substituted C₁-C₉ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G7} and R^{G8}, R^{G8} and R^{G9}, R^{G9} and R^{G10}, and/or R^{G10} and R^{G11} together with the carbon atoms to which each is attached, combine to form optionally substituted C₆-C₁₀ aryl, optionally substituted C₃-C₁₀ carbocyclyl, optionally substituted C₂-C₉ heteroaryl, or C₂-C₉ heterocyclyl.

In some embodiments, each of R^{G7}, R^{G8}, R^{G9}, R^{G10}, and R^{G11} is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{G7} and R^{G8}, R^{G8} and R^{G9}, R^{G9} and R^{G10}, and/or R^{G10} and R^{G11}, together with the carbon atoms to which each is attached, combine to form optionally substituted C₆-C₁₀ aryl, optionally substituted C₃-C₁₀ carbocyclyl, optionally substituted C₂-C₉ heteroaryl, or C₂-C₉ heterocyclyl.

In some embodiments, each of R^{G7}, R^{G8}, R^{G9}, R^{G10}, and R^{G11} is, independently, H, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl; or R^{G7} and R^{G8}, R^{G8} and R^{G9}, R^{G9} and R^{G10}, and/or R^{G10} and R^{G11}, together with the carbon atoms to which each is attached, combine to form optionally substituted C₆-C₁₀ aryl, optionally substituted C₃-C₁₀ carbocyclyl, optionally substituted C₂-C₉ heteroaryl, or C₂-C₉ heterocyclyl.

In some embodiments, each of R^{G7}, R^{G8}, R^{G9}, R^{G10}, and R^{G11} is, independently, H, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl.

In some embodiments, each of R^{G7}, R^{G8}, R^{G9}, R^{G10}, and R^{G11} is, independently, H, F, Cl,

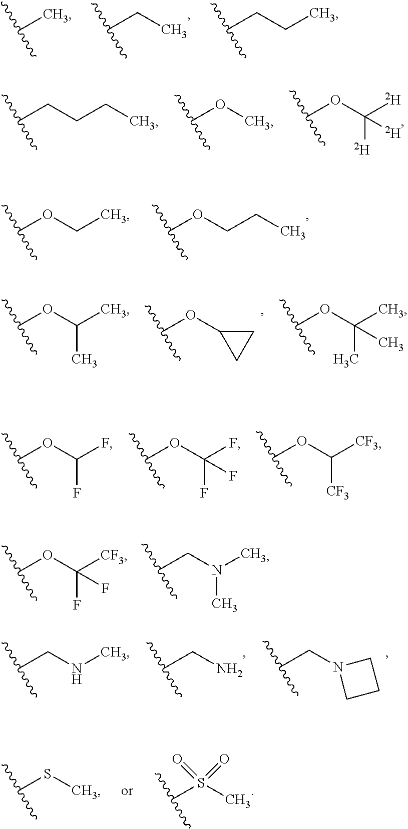

In some embodiments, R^{G8} is

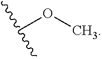 [SMILES: *OC]

In some embodiments, G″ is

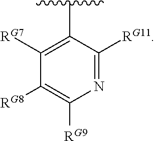

In some embodiments, R^{G7} is H; R^{G8} is

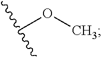 [SMILES: *OC]

R^{G9} is H; and R^{G11} is H.

In some embodiments, G″ is

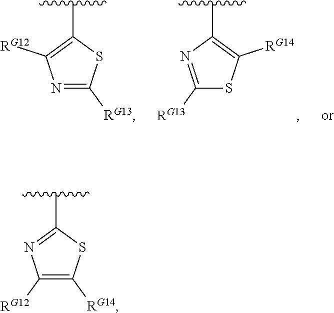

where
each of R^{G12}, R^{G13}, and R^{G14} is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G12} and R^{G14}, together with the carbon atoms to which each is attached, combine to form optionally substituted C₆-C₁₀ aryl, optionally substituted C₃-C₁₀ carbocyclyl, optionally substituted C₂-C₉ heteroaryl, or optionally substituted C₂-C₉ heterocyclyl.

In some embodiments, each of R^{G12}, R^{G13}, and R^{G14} is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{G12} and R^{G14}, together with the carbon atoms to which each is attached combine to form optionally substituted C₆-C₁₀ aryl, optionally substituted C₃-C₁₀ carbocyclyl, optionally substituted C₂-C₉ heteroaryl, or optionally substituted C₂-C₉ heterocyclyl.

In some embodiments, A has the structure of Formula IIIa:

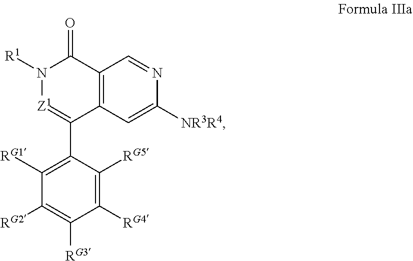

or a pharmaceutically acceptable salt thereof.

In some embodiments, A has the structure of Formula IIIb:

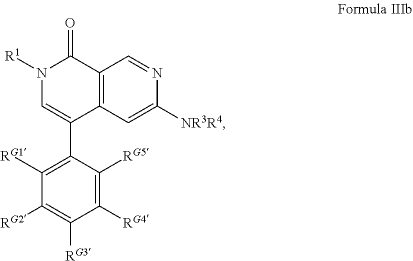

or a pharmaceutically acceptable salt thereof.

In some embodiments, A has the structure of Formula IIIc:

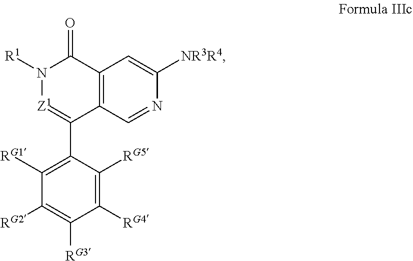

or a pharmaceutically acceptable salt thereof.

In some embodiments, A has the structure of Formula IIId:

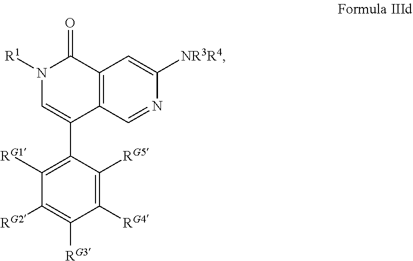

or a pharmaceutically acceptable salt thereof.

In some embodiments, A has the structure of Formula IIIe:

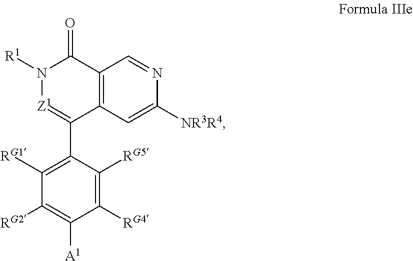

or a pharmaceutically acceptable salt thereof.

In some embodiments, A has the structure of Formula IIIf:

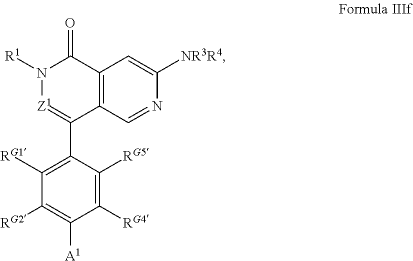

or a pharmaceutically acceptable salt thereof.

In some embodiments, A has the structure of Formula IIIg:

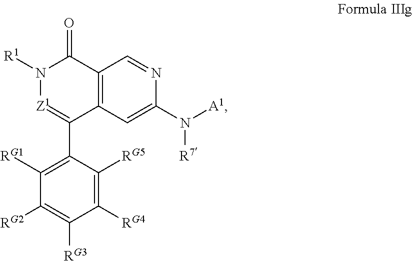

or a pharmaceutically acceptable salt thereof.

In some embodiments, A has the structure of Formula IIIh:

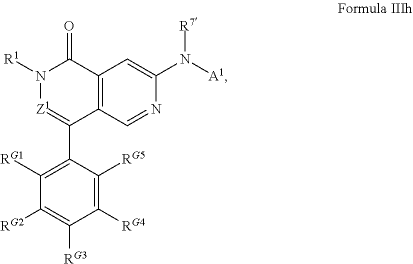

or a pharmaceutically acceptable salt thereof.

In some embodiments, A has the structure of Formula IIIi:

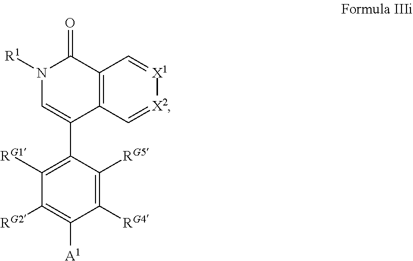

or a pharmaceutically acceptable salt thereof.

In some embodiments, A has the structure of Formula IIIlj:

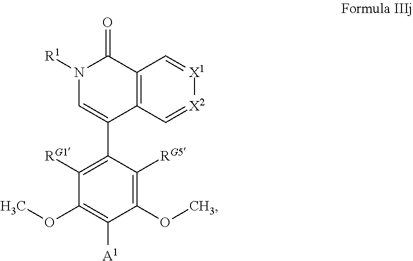

or a pharmaceutically acceptable salt thereof.

In some embodiments, A has the structure of Formula IIIk:

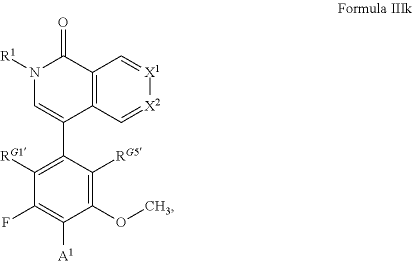

or a pharmaceutically acceptable salt thereof.

In some embodiments, A has the structure of Formula IIIm:

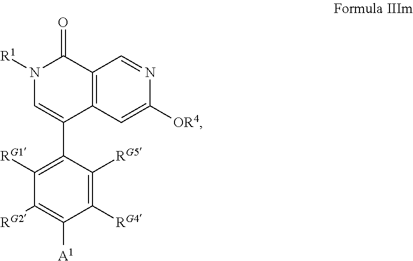

or a pharmaceutically acceptable salt thereof.

In some embodiments, A has the structure of Formula IIIn:

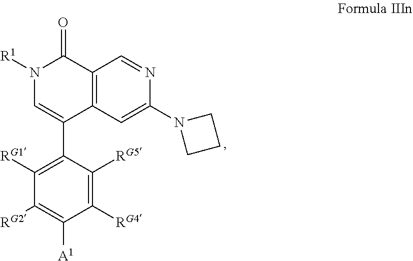

or a pharmaceutically acceptable salt thereof.

In some embodiments, A has the structure of Formula IIIo:

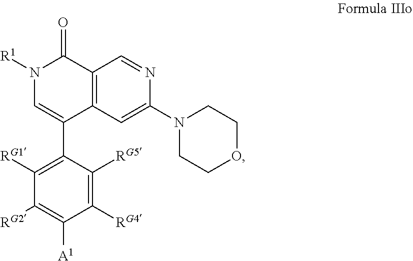

or a pharmaceutically acceptable salt thereof.

In some embodiments, A has the structure of Formula IIIp:

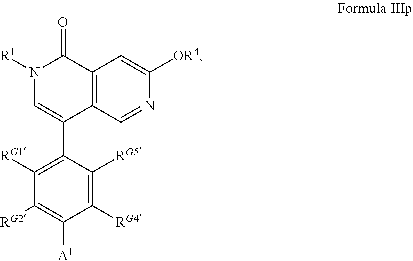

or a pharmaceutically acceptable salt thereof.

In some embodiments, A has the structure of Formula IIIlq:

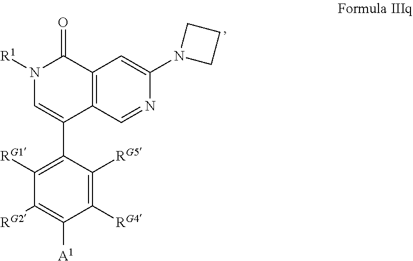

or a pharmaceutically acceptable salt thereof.

In some embodiments, A has the structure of Formula IIIr:

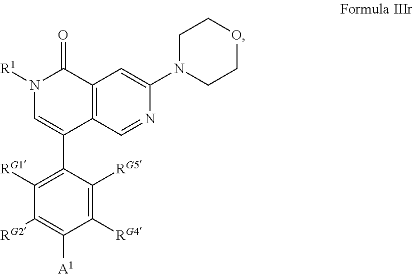

or a pharmaceutically acceptable salt thereof.

In some embodiments, A has the structure of Formula IIIs:

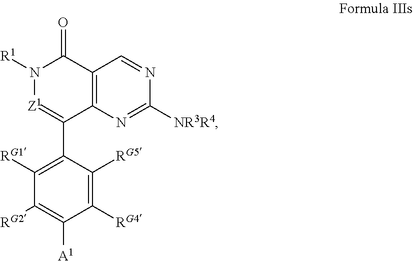

or a pharmaceutically acceptable salt thereof.

In some embodiments, A has the structure of Formula IIIt:

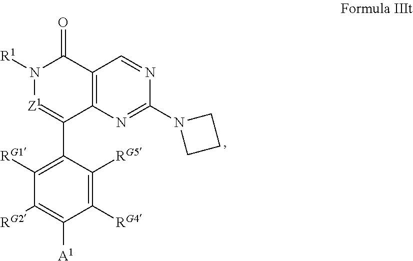

or a pharmaceutically acceptable salt thereof.

In some embodiments, A has the structure of Formula IIIu:

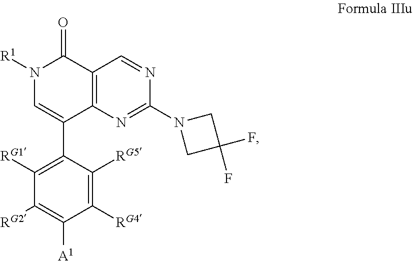

or a pharmaceutically acceptable salt thereof.

In some embodiments, A has the structure of Formula IIIy:

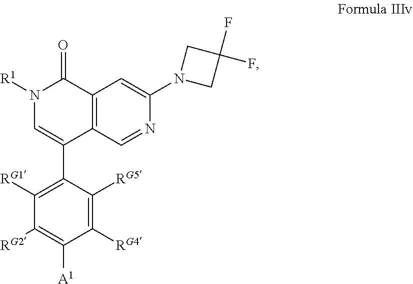

or a pharmaceutically acceptable salt thereof.

In some embodiments, the degradation moiety is a ubiquitin ligase binding moiety.

In some embodiments, the ubiquitin ligase binding moiety comprises Cereblon ligands, IAP (Inhibitors of Apoptosis) ligands, mouse double minute 2 homolog (MDM2), or von Hippel-Lindau (VHL) ligands, or derivatives or analogs thereof.

In some embodiments, the degradation moiety is a ubiquitin ligase binding moiety.

In some embodiments, the ubiquitin ligase binding moiety comprises Cereblon ligands, IAP (Inhibitors of Apoptosis) ligands, mouse double minute 2 homolog (MDM2), or von Hippel-Lindau (VHL) ligands, or derivatives or analogs thereof.

In some embodiments, the degradation moiety has the structure of Formula A:

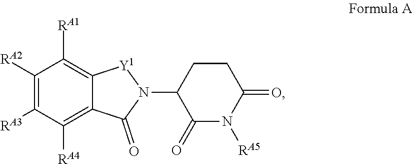

where
Y¹ is

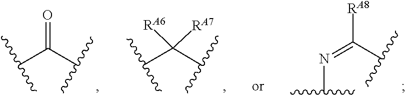

R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3} and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

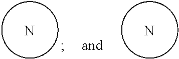

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2}, R^{A3}, and R^{A4} is A², or

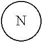

is substituted with A², or a pharmaceutically acceptable salt thereof.

In some embodiments, each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

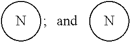

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2}, R^{A3}, and R^{A4} is A², or

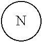

is substituted with A², or a pharmaceutically acceptable salt thereof.

In some embodiments, each of R^{A1}, R^{A2}, R^{A3}, and R^{A4} is, H, A², halogen, optionally substituted C₁-C₆ alkyl, optionally substituted C₁-C₆ heteroalkyl, optionally substituted —O—C₃-C₆ carbocyclyl, hydroxyl, optionally substituted amino; or R^{A1} and R^{A2}, R^{A2} and R^{A3}, or R^{A3} and R^{A4}, together with the carbon atoms to which each is attached, combine to form

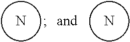

is optionally substituted C₂-C₉ heterocyclyl, which is optionally substituted with A², where one of R^{A1}, R^{A2}, R^{A3} and R^{A4} is A², or

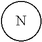

is substituted with A².

In some embodiments, each of R^{A1}, R^{A2}, R^{A3}, and R^{A4} is, independently, H, A², F,

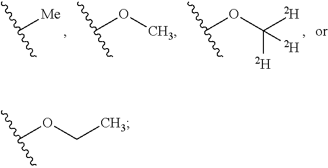

or R^{A1} and R^{A2}, R^{A2} and R^{A3}, or R^{A3} and R^{A4} together with the carbon atoms to which each is attached, combine to form

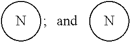

is optionally substituted C₂-C₉ heterocyclyl, which is optionally substituted with A², where one of R^{A1}, R^{A2}, R^{A3}, and R^{A4} is A², or

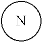

is substituted with A².

In some embodiments, R^{A1} is A². In some embodiments, R^{A2} is A². In some embodiments, R^{A3} is A².

In some embodiments, R^{A4} is A². In some embodiments, R^{A5} is A².

In some embodiments, R^{A5} is H or optionally substituted C₁-C₆ alkyl.

In some embodiments, R^{A5} is H or

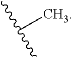

In some embodiments, R^{A5} is H. In some embodiments, R^{A5} is

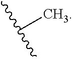

In some embodiments, Y¹ is or

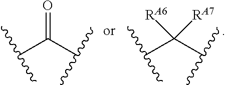

In some embodiments, Y¹ is

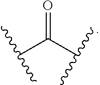 [SMILES: *C(*)=O]

In some embodiments, Y¹ is

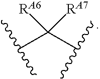

In some embodiments, each of R^{A6} and R^{A7} is, independently, H, F,

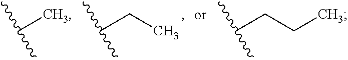

or R^{A6} and R^{A7}, together with the carbon atom to which each is bound, combine to form

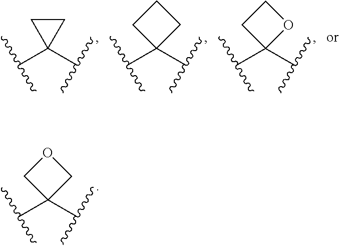 [SMILES: *C1(*)CC1 ; *C1(*)CCC1 ; *C1(*)CCO1 ; *C1(*)COC1]

In some embodiments, R^{A6} is H and R^{A7} is H.

In some embodiments, Y¹ is

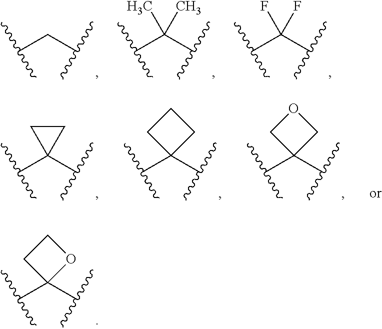

In some embodiments, Y¹ is

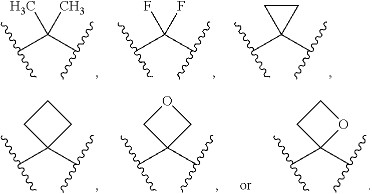

In some embodiments, Y¹ is

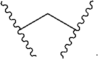 [SMILES: *C*]

In some embodiments, the structure of Formula A has the structure of Formula A1:

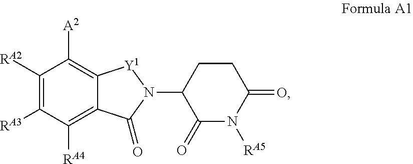

or a pharmaceutically acceptable salt thereof.

In some embodiments, the structure of Formula A has the structure of Formula A2:

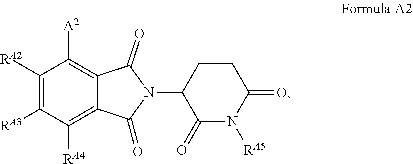

or a pharmaceutically acceptable salt thereof.

In some embodiments, the structure of Formula A has the structure of Formula A3:

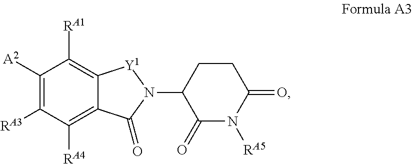

or a pharmaceutically acceptable salt thereof.

In some embodiments, the structure of Formula A has the structure of Formula A4:

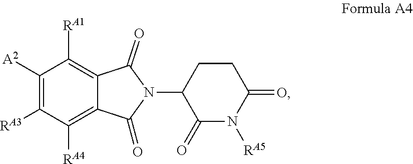

or a pharmaceutically acceptable salt thereof.

In some embodiments, the structure of Formula A has the structure of Formula A5:

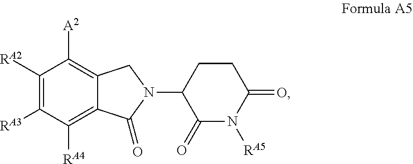

or a pharmaceutically acceptable salt thereof.

In some embodiments, the structure of Formula A has the structure of Formula A6:

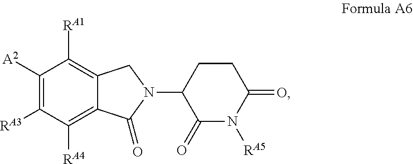

or a pharmaceutically acceptable salt thereof.

In some embodiments, the structure of Formula A has the structure of Formula A7:

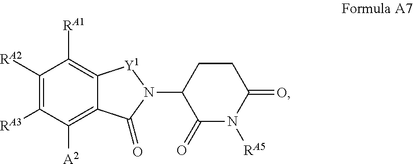

or a pharmaceutically acceptable salt thereof.

In some embodiments, the structure of Formula A has the structure of Formula A8:

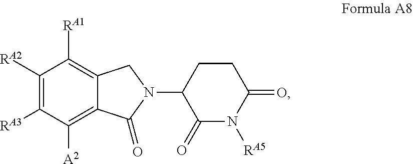

or a pharmaceutically acceptable salt thereof.

In some embodiments, the structure of Formula A has the structure of Formula A9:

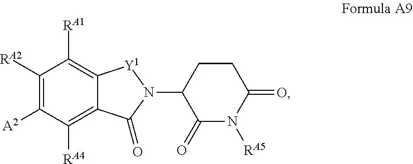

or a pharmaceutically acceptable salt thereof.

In some embodiments, the structure of Formula A has the structure of Formula A10:

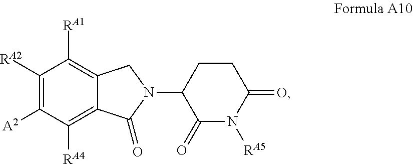

or a pharmaceutically acceptable salt thereof.

In some embodiments, wherein the structure of Formula A is

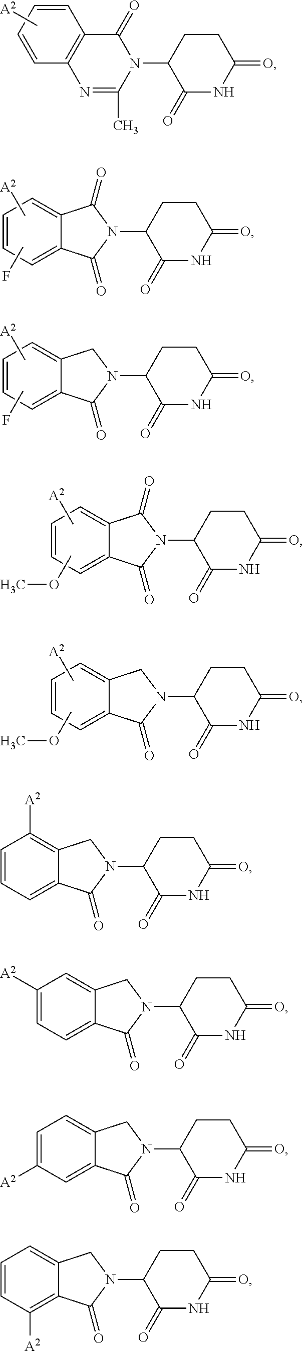

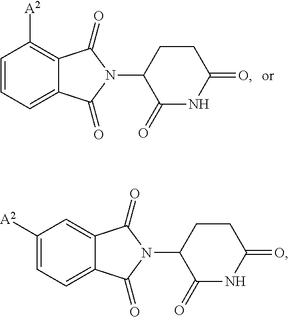

or derivative or analog thereof.

In some embodiments, the structure of Formula A is

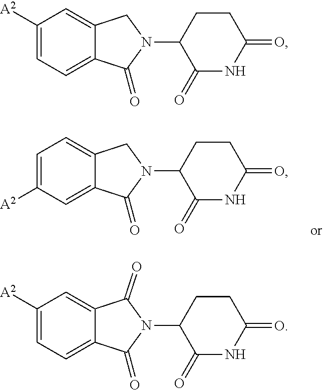

In some embodiments, the structure of Formula A is

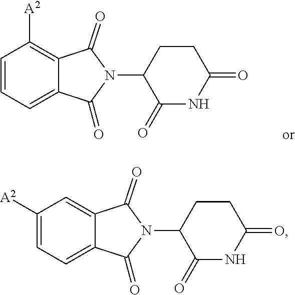

or derivative or analog thereof.

In some embodiments, the linker has the structure of Formula II:

A¹-E¹-F-E²-A², Formula II
A¹ is a bond between the linker and A;
A² is a bond between B and the linker;
each of E¹ and E² is, independently, absent, CH₂, O, or NCH₃; and
F has the structure:

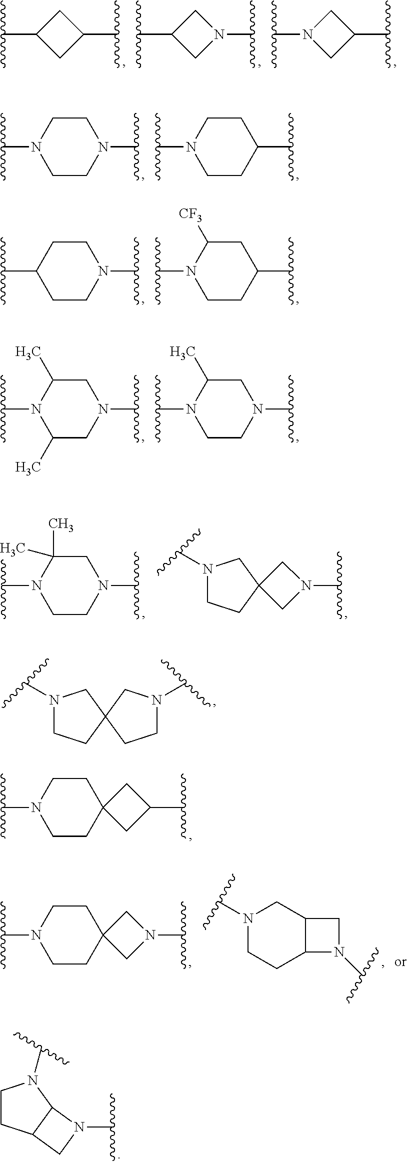

In some embodiments, E¹ is absent. In some embodiments, E¹ is CH₂. In some embodiments, E¹ is O. In some embodiments, E¹ is NCH₃.

In some embodiments, E² is absent. In some embodiments, E² is CH₂. In some embodiments, E² is O. In some embodiments, E² is NCH₃.

In some embodiments, the linker comprises the structure:

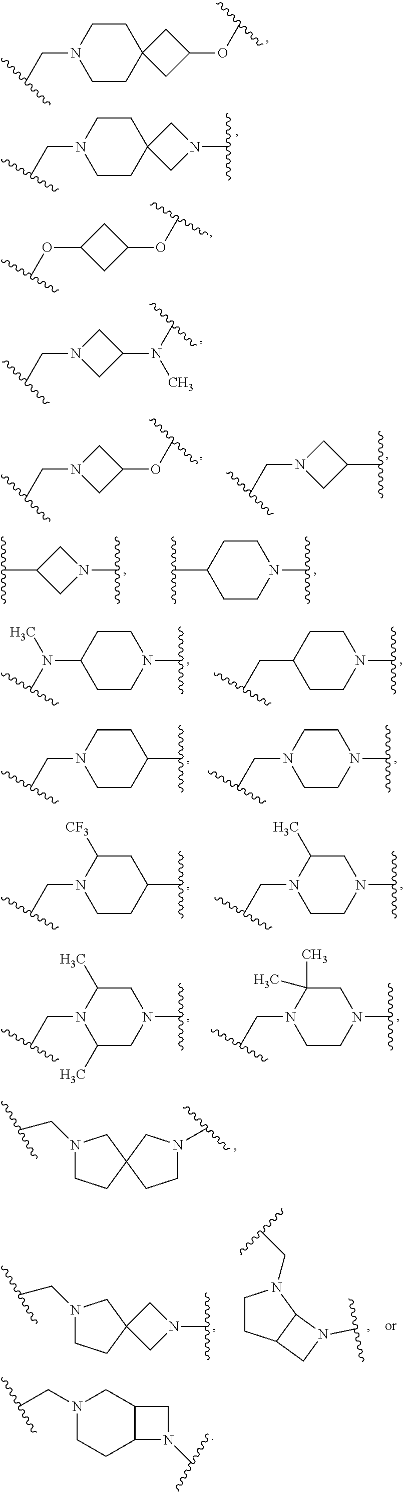

In some embodiments, the compound has the structure of any one of compounds D1-D66 in Table 1, or a pharmaceutically acceptable salt thereof.

TABLE 1
Compounds D1-D184 of the Disclosure

| Compound No. | Structure |
|---|---|
| D1 | 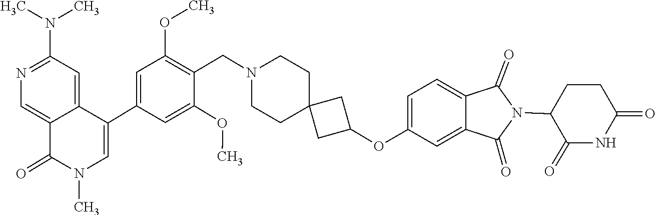 [SMILES: COc1cc(-c2cn(C)c(=O)c3cnc(N(C)C)cc23)cc(OC)c1CN1CCC2(CC1)CC(Oc1ccc3c(c1)C(=O)N(C1CCC(=O)NC1=O)C3=O)C2] |
| D2 | 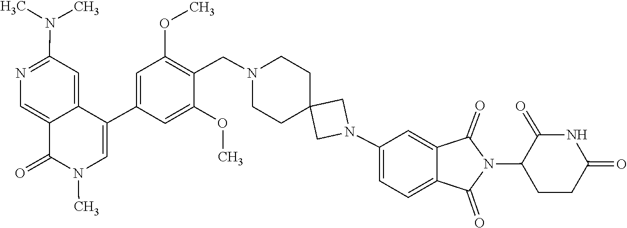 [SMILES: COc1cc(-c2cn(C)c(=O)c3cnc(N(C)C)cc23)cc(OC)c1CN1CCC2(CC1)CN(c1ccc3c(c1)C(=O)N(C1CCC(=O)NC1=O)C3=O)C2] |
| D3 | 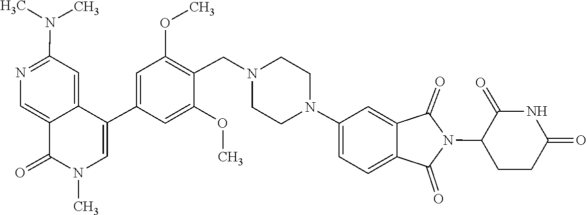 [SMILES: COc1cc(-c2cn(C)c(=O)c3cnc(N(C)C)cc23)cc(OC)c1CN1CCN(c2ccc3c(c2)C(=O)N(C2CCC(=O)NC2=O)C3=O)CC1] |
| D4 | 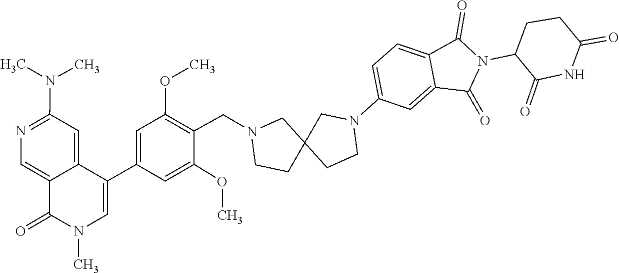 [SMILES: COc1cc(-c2cn(C)c(=O)c3cnc(N(C)C)cc23)cc(OC)c1CN1CCC2(CCN(c3ccc4c(c3)C(=O)N(C3CCC(=O)NC3=O)C4=O)C2)C1] |
| D5 | 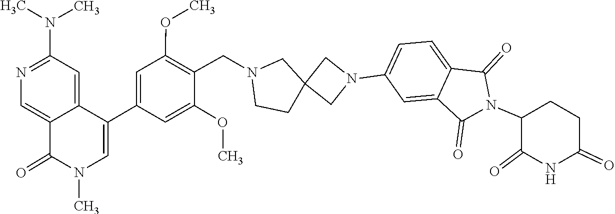 [SMILES: COc1cc(-c2cn(C)c(=O)c3cnc(N(C)C)cc23)cc(OC)c1CN1CCC2(C1)CN(c1ccc3c(c1)C(=O)N(C1CCC(=O)NC1=O)C3=O)C2] |
| D6 | 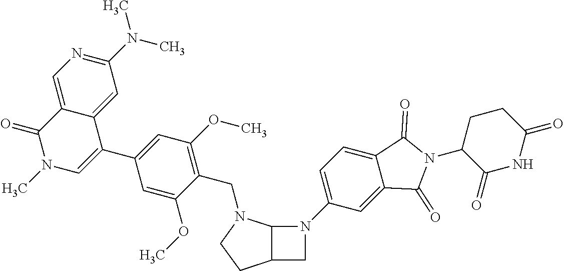 [SMILES: COc1cc(-c2cn(C)c(=O)c3cnc(N(C)C)cc23)cc(OC)c1CN1CCC2CN(c3ccc4c(c3)C(=O)N(C3CCC(=O)NC3=O)C4=O)C21] |
| D7 | 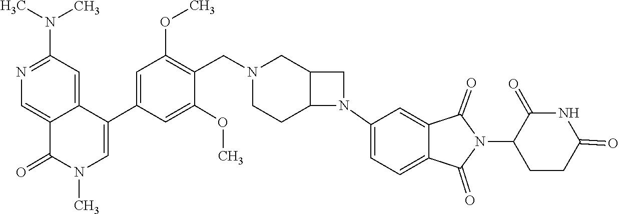 [SMILES: COc1cc(-c2cn(C)c(=O)c3cnc(N(C)C)cc23)cc(OC)c1CN1CCC2C(C1)CN2c1ccc2c(c1)C(=O)N(C1CCC(=O)NC1=O)C2=O] |
| D8 | 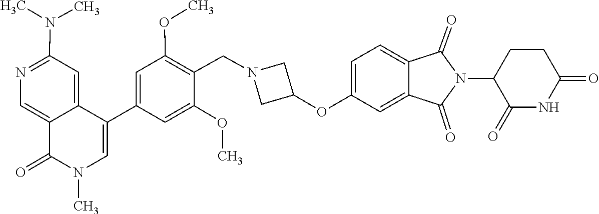 [SMILES: COc1cc(-c2cn(C)c(=O)c3cnc(N(C)C)cc23)cc(OC)c1CN1CC(Oc2ccc3c(c2)C(=O)N(C2CCC(=O)NC2=O)C3=O)C1] |
| D9 | 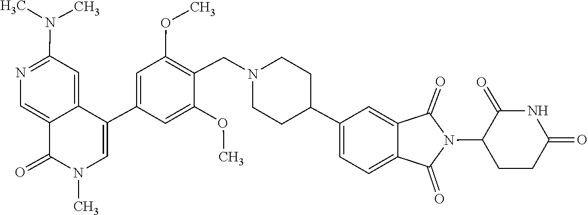 [SMILES: COc1cc(-c2cn(C)c(=O)c3cnc(N(C)C)cc23)cc(OC)c1CN1CCC(c2ccc3c(c2)C(=O)N(C2CCC(=O)NC2=O)C3=O)CC1] |
| D10 | 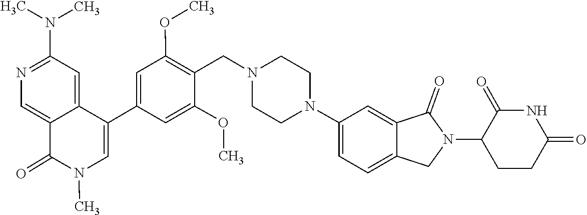 [SMILES: COc1cc(-c2cn(C)c(=O)c3cnc(N(C)C)cc23)cc(OC)c1CN1CCN(c2ccc3c(c2)C(=O)N(C2CCC(=O)NC2=O)C3)CC1] |
| D11 | 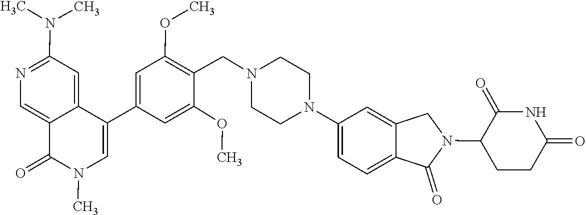 [SMILES: COc1cc(-c2cn(C)c(=O)c3cnc(N(C)C)cc23)cc(OC)c1CN1CCN(c2ccc3c(c2)CN(C2CCC(=O)NC2=O)C3=O)CC1] |
| D12 | 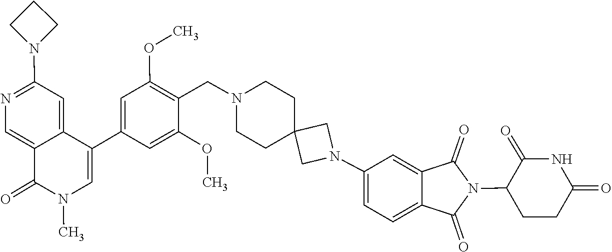 [SMILES: COc1cc(-c2cn(C)c(=O)c3cnc(N4CCC4)cc23)cc(OC)c1CN1CCC2(CC1)CN(c1ccc3c(c1)C(=O)N(C1CCC(=O)NC1=O)C3=O)C2] |
| D13 | 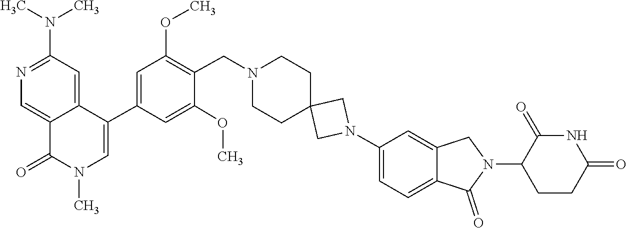 [SMILES: COc1cc(-c2cn(C)c(=O)c3cnc(N(C)C)cc23)cc(OC)c1CN1CCC2(CC1)CN(c1ccc3c(c1)CN(C1CCC(=O)NC1=O)C3=O)C2] |
| D14 | 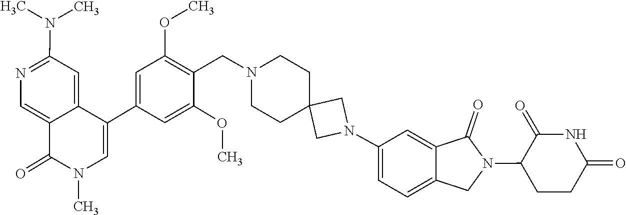 [SMILES: COc1cc(-c2cn(C)c(=O)c3cnc(N(C)C)cc23)cc(OC)c1CN1CCC2(CC1)CN(c1ccc3c(c1)C(=O)N(C1CCC(=O)NC1=O)C3)C2] |
| D15 | 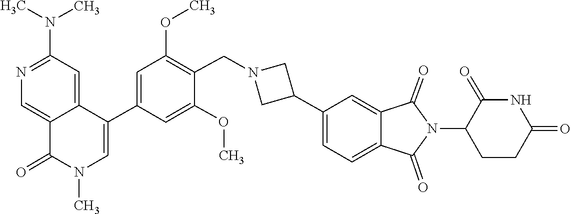 [SMILES: COc1cc(-c2cn(C)c(=O)c3cnc(N(C)C)cc23)cc(OC)c1CN1CC(c2ccc3c(c2)C(=O)N(C2CCC(=O)NC2=O)C3=O)C1] |
| D16 | 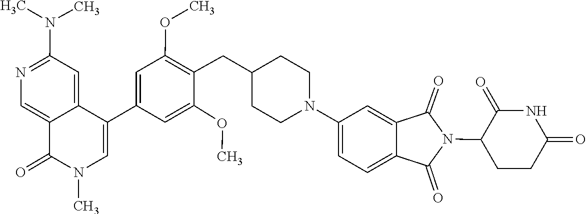 [SMILES: COc1cc(-c2cn(C)c(=O)c3cnc(N(C)C)cc23)cc(OC)c1CC1CCN(c2ccc3c(c2)C(=O)N(C2CCC(=O)NC2=O)C3=O)CC1] |
| D17 | 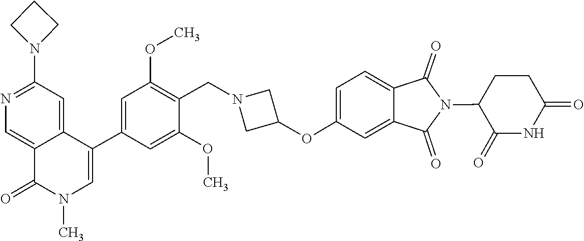 [SMILES: COc1cc(-c2cn(C)c(=O)c3cnc(N4CCC4)cc23)cc(OC)c1CN1CC(Oc2ccc3c(c2)C(=O)N(C2CCC(=O)NC2=O)C3=O)C1] |
| D18 | 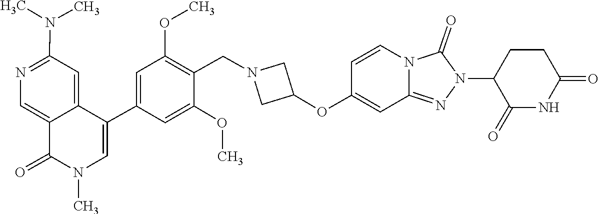 [SMILES: COc1cc(-c2cn(C)c(=O)c3cnc(N(C)C)cc23)cc(OC)c1CN1CC(Oc2ccn3c(=O)n(C4CCC(=O)NC4=O)nc3c2)C1] |
| D19 | 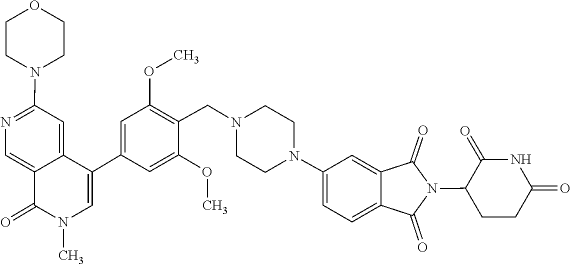 [SMILES: COc1cc(-c2cn(C)c(=O)c3cnc(N4CCOCC4)cc23)cc(OC)c1CN1CCN(c2ccc3c(c2)C(=O)N(C2CCC(=O)NC2=O)C3=O)CC1] |
| D20 | 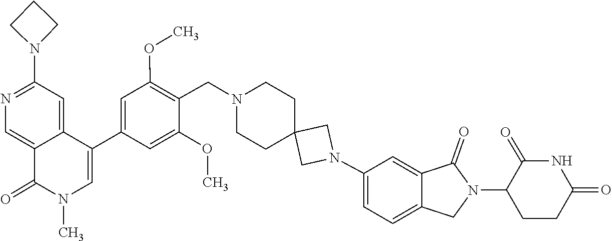 [SMILES: COc1cc(-c2cn(C)c(=O)c3cnc(N4CCC4)cc23)cc(OC)c1CN1CCC2(CC1)CN(c1ccc3c(c1)C(=O)N(C1CCC(=O)NC1=O)C3)C2] |
| D21 | 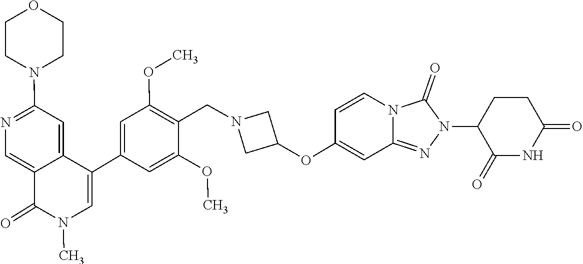 [SMILES: COc1cc(-c2cn(C)c(=O)c3cnc(N4CCOCC4)cc23)cc(OC)c1CN1CC(Oc2ccn3c(=O)n(C4CCC(=O)NC4=O)nc3c2)C1] |
| D22 | 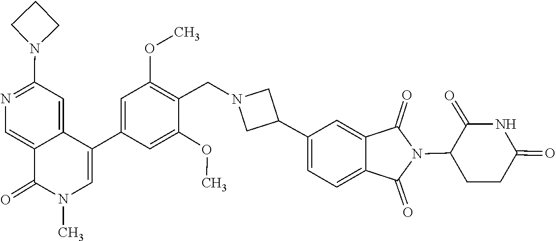 [SMILES: COc1cc(-c2cn(C)c(=O)c3cnc(N4CCC4)cc23)cc(OC)c1CN1CC(c2ccc3c(c2)C(=O)N(C2CCC(=O)NC2=O)C3=O)C1] |
| D23 | 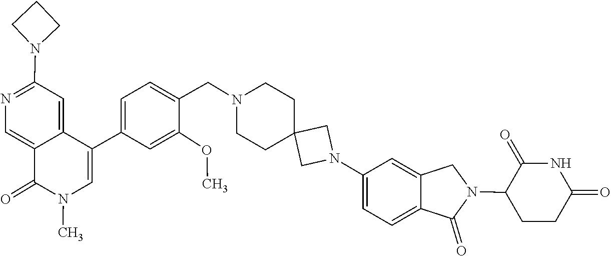 [SMILES: COc1cc(-c2cn(C)c(=O)c3cnc(N4CCC4)cc23)ccc1CN1CCC2(CC1)CN(c1ccc3c(c1)CN(C1CCC(=O)NC1=O)C3=O)C2] |
| D24 | 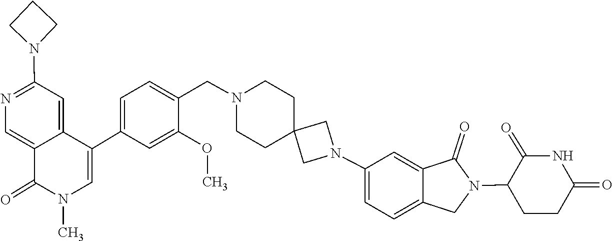 [SMILES: COc1cc(-c2cn(C)c(=O)c3cnc(N4CCC4)cc23)ccc1CN1CCC2(CC1)CN(c1ccc3c(c1)C(=O)N(C1CCC(=O)NC1=O)C3)C2] |
| D25 | 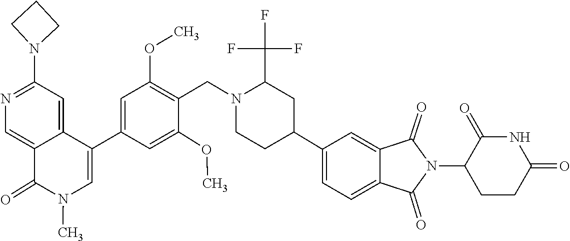 [SMILES: COc1cc(-c2cn(C)c(=O)c3cnc(N4CCC4)cc23)cc(OC)c1CN1CCC(c2ccc3c(c2)C(=O)N(C2CCC(=O)NC2=O)C3=O)CC1C(F)(F)F] |
| D26 | 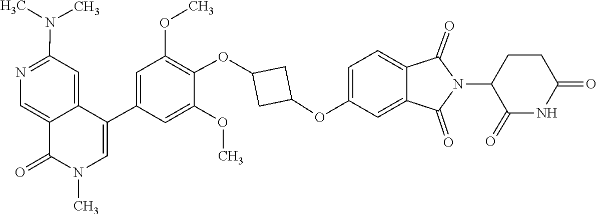 [SMILES: COc1cc(-c2cn(C)c(=O)c3cnc(N(C)C)cc23)cc(OC)c1OC1CC(Oc2ccc3c(c2)C(=O)N(C2CCC(=O)NC2=O)C3=O)C1] |
| D27 | 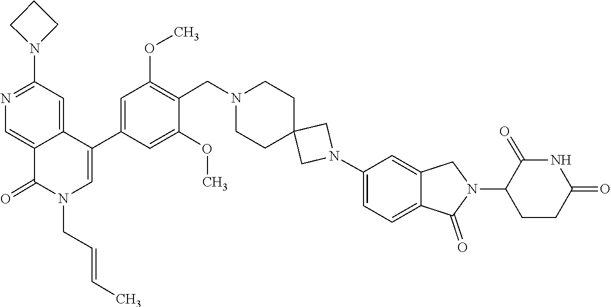 [SMILES: C/C=C/Cn1cc(-c2cc(OC)c(CN3CCC4(CC3)CN(c3ccc5c(c3)CN(C3CCC(=O)NC3=O)C5=O)C4)c(OC)c2)c2cc(N3CCC3)ncc2c1=O] |
| D28 | 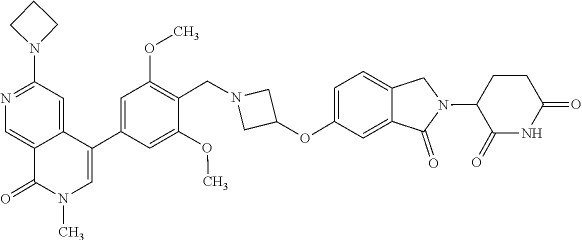 [SMILES: COc1cc(-c2cn(C)c(=O)c3cnc(N4CCC4)cc23)cc(OC)c1CN1CC(Oc2ccc3c(c2)C(=O)N(C2CCC(=O)NC2=O)C3)C1] |
| D29 | 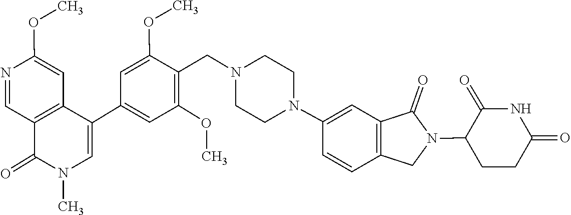 [SMILES: COc1cc2c(-c3cc(OC)c(CN4CCN(c5ccc6c(c5)C(=O)N(C5CCC(=O)NC5=O)C6)CC4)c(OC)c3)cn(C)c(=O)c2cn1] |
| D30 | 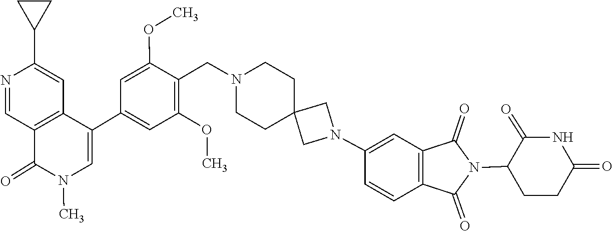 [SMILES: COc1cc(-c2cn(C)c(=O)c3cnc(C4CC4)cc23)cc(OC)c1CN1CCC2(CC1)CN(c1ccc3c(c1)C(=O)N(C1CCC(=O)NC1=O)C3=O)C2] |
| D31 | 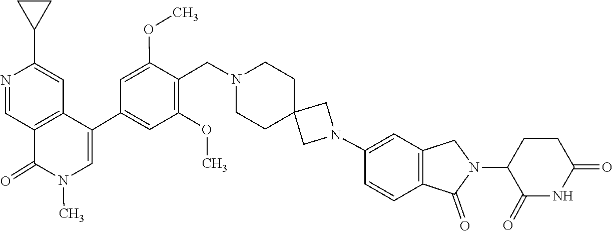 [SMILES: COc1cc(-c2cn(C)c(=O)c3cnc(C4CC4)cc23)cc(OC)c1CN1CCC2(CC1)CN(c1ccc3c(c1)CN(C1CCC(=O)NC1=O)C3=O)C2] |
| D32 | 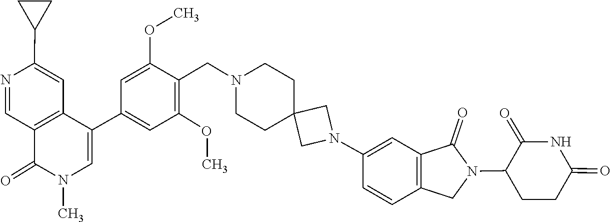 [SMILES: COc1cc(-c2cn(C)c(=O)c3cnc(C4CC4)cc23)cc(OC)c1CN1CCC2(CC1)CN(c1ccc3c(c1)C(=O)N(C1CCC(=O)NC1=O)C3)C2] |
| D33 | 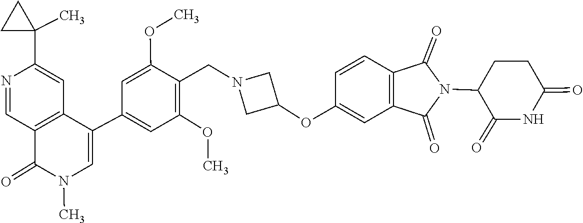 [SMILES: COc1cc(-c2cn(C)c(=O)c3cnc(C4(C)CC4)cc23)cc(OC)c1CN1CC(Oc2ccc3c(c2)C(=O)N(C2CCC(=O)NC2=O)C3=O)C1] |
| D34 | 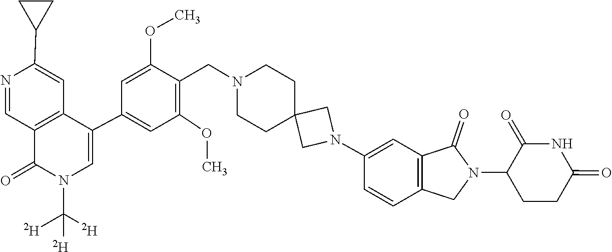 [SMILES: [2H]C([2H])([2H])n1cc(-c2cc(OC)c(CN3CCC4(CC3)CN(c3ccc5c(c3)C(=O)N(C3CCC(=O)NC3=O)C5)C4)c(OC)c2)c2cc(C3CC3)ncc2c1=O] |
| D35 | 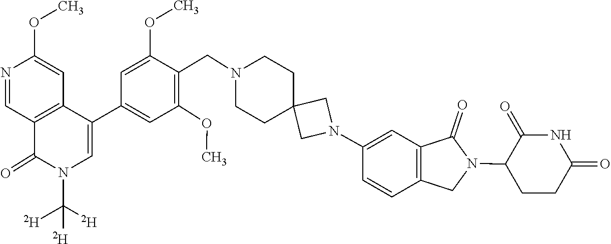 [SMILES: [2H]C([2H])([2H])n1cc(-c2cc(OC)c(CN3CCC4(CC3)CN(c3ccc5c(c3)C(=O)N(C3CCC(=O)NC3=O)C5)C4)c(OC)c2)c2cc(OC)ncc2c1=O] |
| D36 | 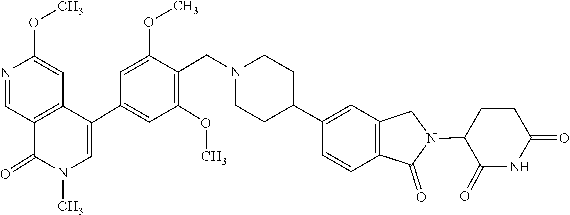 [SMILES: COc1cc2c(-c3cc(OC)c(CN4CCC(c5ccc6c(c5)CN(C5CCC(=O)NC5=O)C6=O)CC4)c(OC)c3)cn(C)c(=O)c2cn1] |
| D37 | 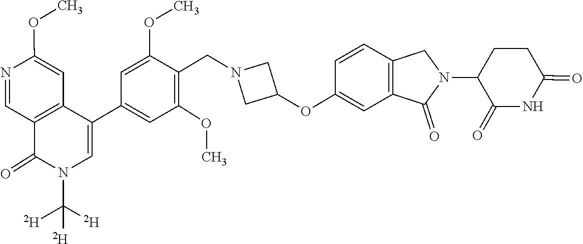 [SMILES: [2H]C([2H])([2H])n1cc(-c2cc(OC)c(CN3CC(Oc4ccc5c(c4)C(=O)N(C4CCC(=O)NC4=O)C5)C3)c(OC)c2)c2cc(OC)ncc2c1=O] |
| D38 | 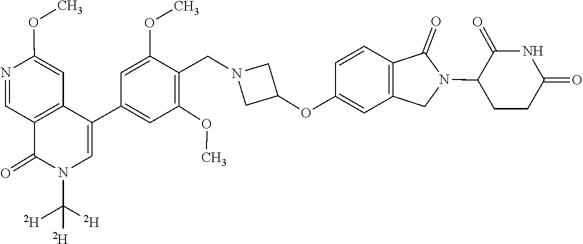 [SMILES: [2H]C([2H])([2H])n1cc(-c2cc(OC)c(CN3CC(Oc4ccc5c(c4)CN(C4CCC(=O)NC4=O)C5=O)C3)c(OC)c2)c2cc(OC)ncc2c1=O] |
| D39 | 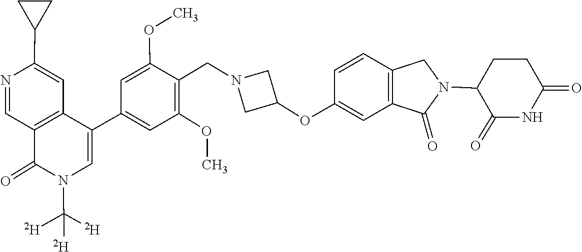 [SMILES: [2H]C([2H])([2H])n1cc(-c2cc(OC)c(CN3CC(Oc4ccc5c(c4)C(=O)N(C4CCC(=O)NC4=O)C5)C3)c(OC)c2)c2cc(C3CC3)ncc2c1=O] |
| D40 | 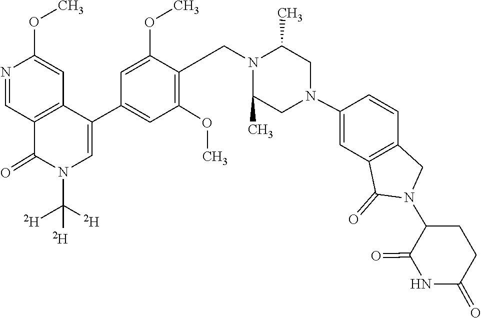 [SMILES: [2H]C([2H])([2H])n1cc(-c2cc(OC)c(CN3[C@H](C)CN(c4ccc5c(c4)C(=O)N(C4CCC(=O)NC4=O)C5)C[C@H]3C)c(OC)c2)c2cc(OC)ncc2c1=O] |
| D41 | 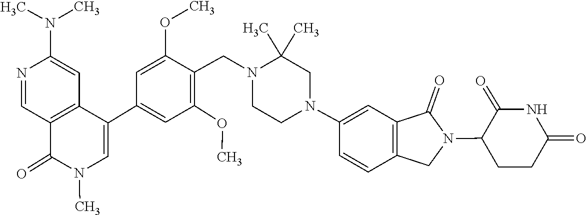 [SMILES: COc1cc(-c2cn(C)c(=O)c3cnc(N(C)C)cc23)cc(OC)c1CN1CCN(c2ccc3c(c2)C(=O)N(C2CCC(=O)NC2=O)C3)CC1(C)C] |
| D42 | 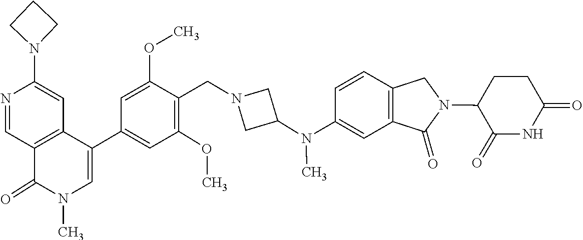 [SMILES: COc1cc(-c2cn(C)c(=O)c3cnc(N4CCC4)cc23)cc(OC)c1CN1CC(N(C)c2ccc3c(c2)C(=O)N(C2CCC(=O)NC2=O)C3)C1] |
| D43 | 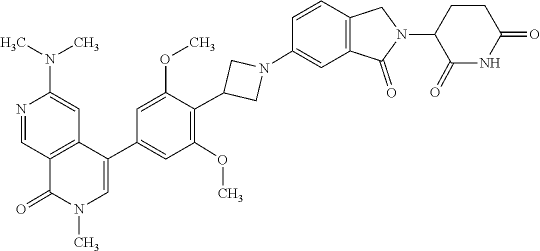 [SMILES: COc1cc(-c2cn(C)c(=O)c3cnc(N(C)C)cc23)cc(OC)c1C1CN(c2ccc3c(c2)C(=O)N(C2CCC(=O)NC2=O)C3)C1] |
| D44 | 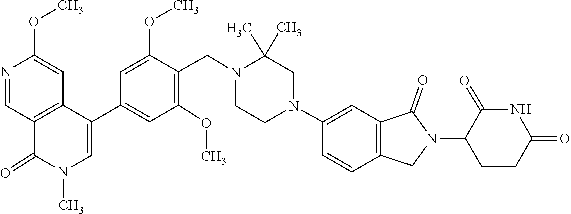 [SMILES: COc1cc2c(-c3cc(OC)c(CN4CCN(c5ccc6c(c5)C(=O)N(C5CCC(=O)NC5=O)C6)CC4(C)C)c(OC)c3)cn(C)c(=O)c2cn1] |
| D45 | 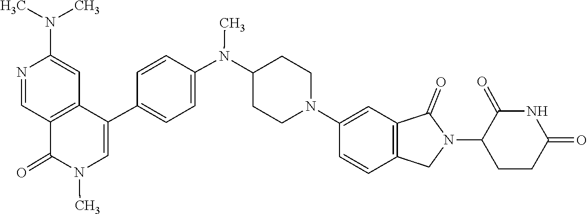 [SMILES: CN(C)c1cc2c(-c3ccc(N(C)C4CCN(c5ccc6c(c5)C(=O)N(C5CCC(=O)NC5=O)C6)CC4)cc3)cn(C)c(=O)c2cn1] |
| D46 | 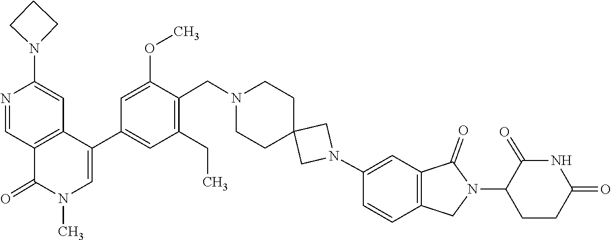 [SMILES: CCc1cc(-c2cn(C)c(=O)c3cnc(N4CCC4)cc23)cc(OC)c1CN1CCC2(CC1)CN(c1ccc3c(c1)C(=O)N(C1CCC(=O)NC1=O)C3)C2] |
| D47 | 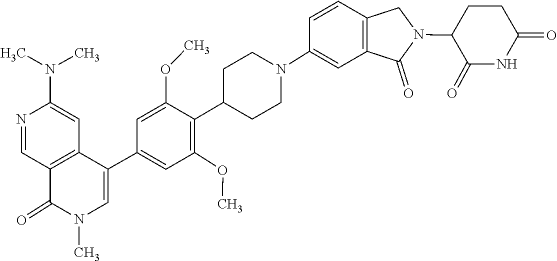 [SMILES: COc1cc(-c2cn(C)c(=O)c3cnc(N(C)C)cc23)cc(OC)c1C1CCN(c2ccc3c(c2)C(=O)N(C2CCC(=O)NC2=O)C3)CC1] |
| D48 | 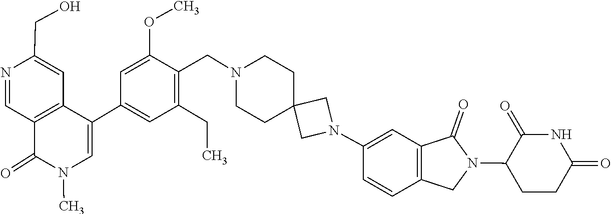 [SMILES: CCc1cc(-c2cn(C)c(=O)c3cnc(CO)cc23)cc(OC)c1CN1CCC2(CC1)CN(c1ccc3c(c1)C(=O)N(C1CCC(=O)NC1=O)C3)C2] |
| D49 | 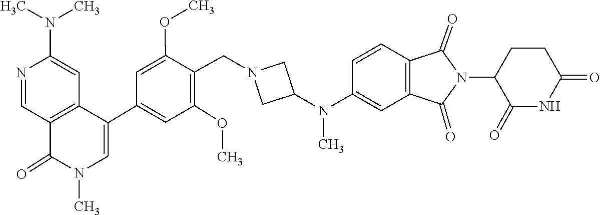 [SMILES: COc1cc(-c2cn(C)c(=O)c3cnc(N(C)C)cc23)cc(OC)c1CN1CC(N(C)c2ccc3c(c2)C(=O)N(C2CCC(=O)NC2=O)C3=O)C1] |
| D50 | 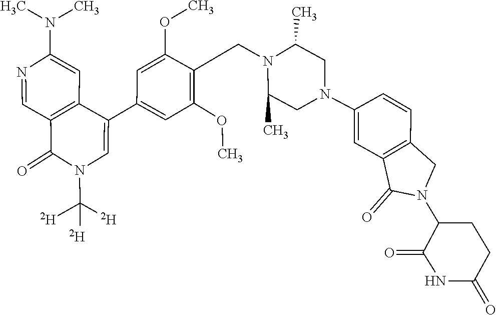 [SMILES: [2H]C([2H])([2H])n1cc(-c2cc(OC)c(CN3[C@H](C)CN(c4ccc5c(c4)C(=O)N(C4CCC(=O)NC4=O)C5)C[C@H]3C)c(OC)c2)c2cc(N(C)C)ncc2c1=O] |
| D51 | 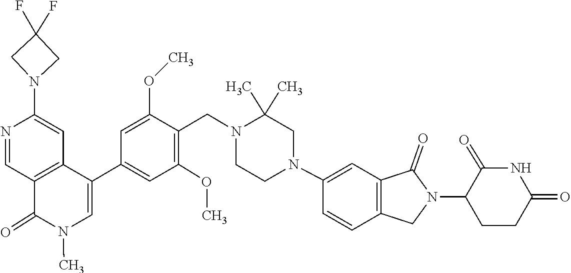 [SMILES: COc1cc(-c2cn(C)c(=O)c3cnc(N4CC(F)(F)C4)cc23)cc(OC)c1CN1CCN(c2ccc3c(c2)C(=O)N(C2CCC(=O)NC2=O)C3)CC1(C)C] |
| D52 | 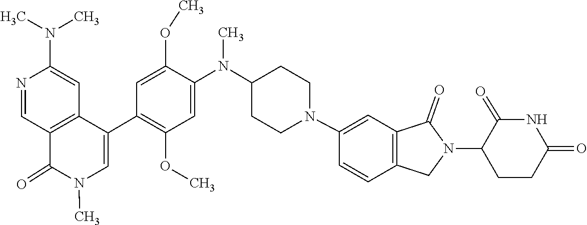 [SMILES: COc1cc(N(C)C2CCN(c3ccc4c(c3)C(=O)N(C3CCC(=O)NC3=O)C4)CC2)c(OC)cc1-c1cn(C)c(=O)c2cnc(N(C)C)cc12] |
| D53 | 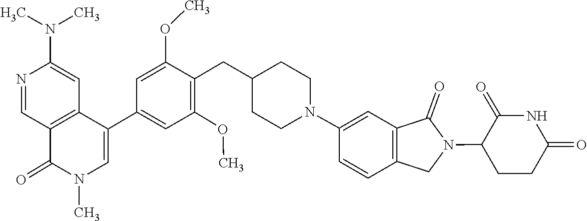 [SMILES: COc1cc(-c2cn(C)c(=O)c3cnc(N(C)C)cc23)cc(OC)c1CC1CCN(c2ccc3c(c2)C(=O)N(C2CCC(=O)NC2=O)C3)CC1] |
| D54 | 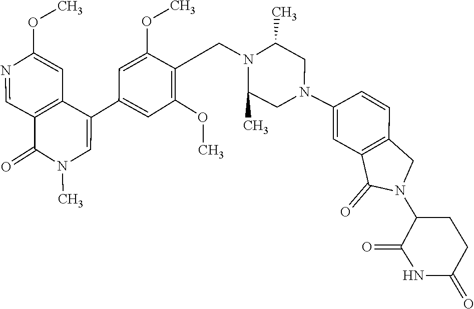 [SMILES: COc1cc2c(-c3cc(OC)c(CN4[C@H](C)CN(c5ccc6c(c5)C(=O)N(C5CCC(=O)NC5=O)C6)C[C@H]4C)c(OC)c3)cn(C)c(=O)c2cn1] |
| D55 | 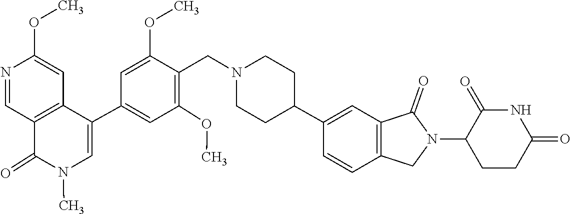 [SMILES: COc1cc2c(-c3cc(OC)c(CN4CCC(c5ccc6c(c5)C(=O)N(C5CCC(=O)NC5=O)C6)CC4)c(OC)c3)cn(C)c(=O)c2cn1] |
| D56 | 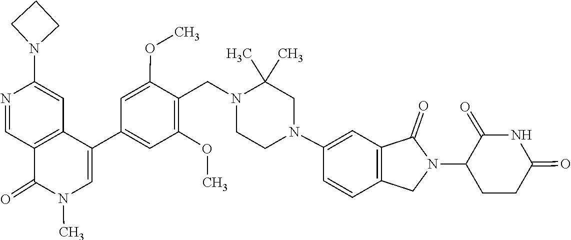 [SMILES: COc1cc(-c2cn(C)c(=O)c3cnc(N4CCC4)cc23)cc(OC)c1CN1CCN(c2ccc3c(c2)C(=O)N(C2CCC(=O)NC2=O)C3)CC1(C)C] |
| D57 | 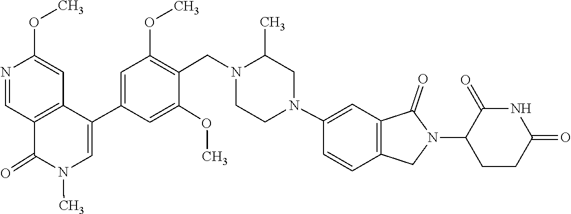 [SMILES: COc1cc2c(-c3cc(OC)c(CN4CCN(c5ccc6c(c5)C(=O)N(C5CCC(=O)NC5=O)C6)CC4C)c(OC)c3)cn(C)c(=O)c2cn1] |
| D58 | 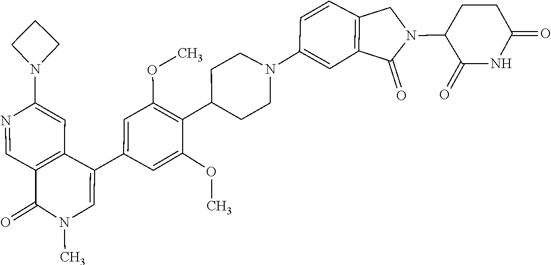 [SMILES: COc1cc(-c2cn(C)c(=O)c3cnc(N4CCC4)cc23)cc(OC)c1C1CCN(c2ccc3c(c2)C(=O)N(C2CCC(=O)NC2=O)C3)CC1] |
| D59 | 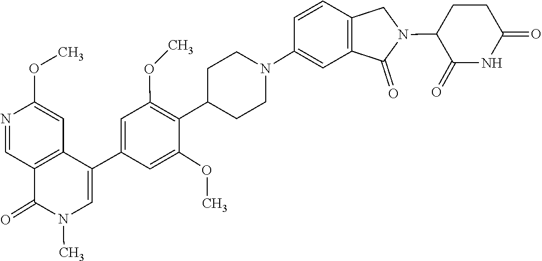 [SMILES: COc1cc2c(-c3cc(OC)c(C4CCN(c5ccc6c(c5)C(=O)N(C5CCC(=O)NC5=O)C6)CC4)c(OC)c3)cn(C)c(=O)c2cn1] |
| D60 | 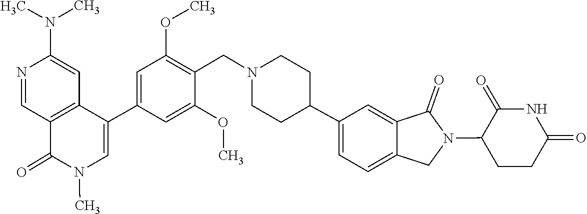 [SMILES: COc1cc(-c2cn(C)c(=O)c3cnc(N(C)C)cc23)cc(OC)c1CN1CCC(c2ccc3c(c2)C(=O)N(C2CCC(=O)NC2=O)C3)CC1] |
| D61 | 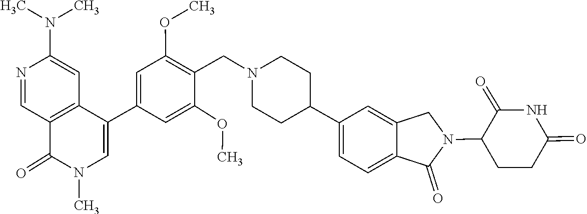 [SMILES: COc1cc(-c2cn(C)c(=O)c3cnc(N(C)C)cc23)cc(OC)c1CN1CCC(c2ccc3c(c2)CN(C2CCC(=O)NC2=O)C3=O)CC1] |
| D62 | 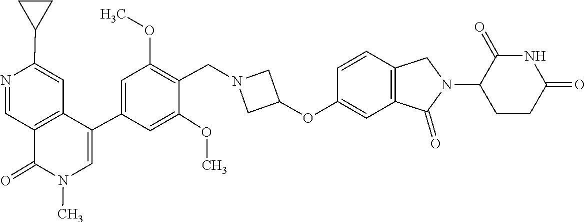 [SMILES: COc1cc(-c2cn(C)c(=O)c3cnc(C4CC4)cc23)cc(OC)c1CN1CC(Oc2ccc3c(c2)C(=O)N(C2CCC(=O)NC2=O)C3)C1] |
| D63 | 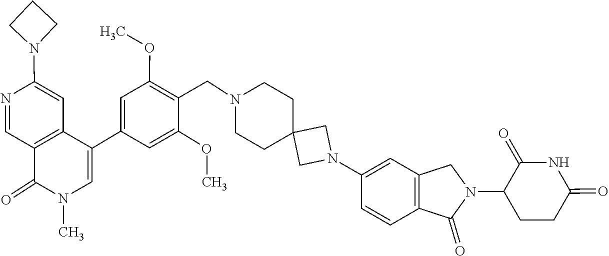 [SMILES: COc1cc(-c2cn(C)c(=O)c3cnc(N4CCC4)cc23)cc(OC)c1CN1CCC2(CC1)CN(c1ccc3c(c1)CN(C1CCC(=O)NC1=O)C3=O)C2] |
| D64 | 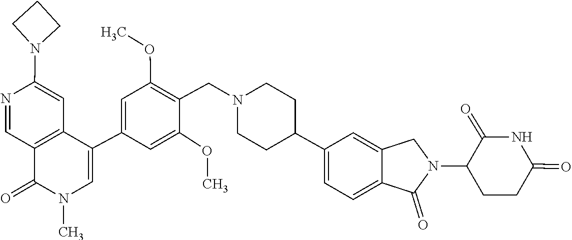 [SMILES: COc1cc(-c2cn(C)c(=O)c3cnc(N4CCC4)cc23)cc(OC)c1CN1CCC(c2ccc3c(c2)CN(C2CCC(=O)NC2=O)C3=O)CC1] |
| D65 | 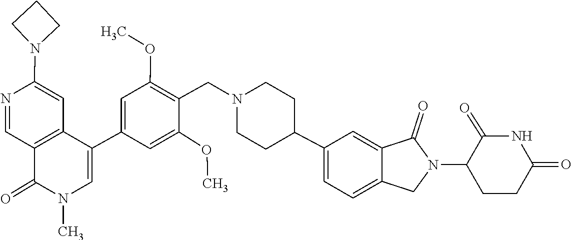 [SMILES: COc1cc(-c2cn(C)c(=O)c3cnc(N4CCC4)cc23)cc(OC)c1CN1CCC(c2ccc3c(c2)C(=O)N(C2CCC(=O)NC2=O)C3)CC1] |
| D66 | 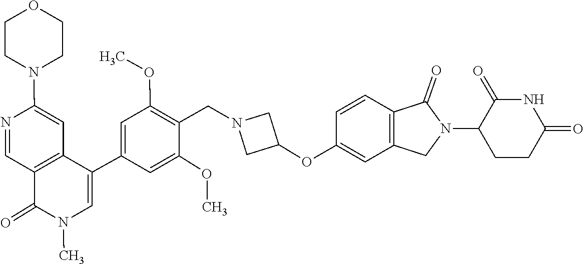 [SMILES: COc1cc(-c2cn(C)c(=O)c3cnc(N4CCOCC4)cc23)cc(OC)c1CN1CC(Oc2ccc3c(c2)CN(C2CCC(=O)NC2=O)C3=O)C1] |

In another aspect, the disclosure features a pharmaceutical composition including any of the foregoing compounds, or pharmaceutically acceptable salts thereof, and a pharmaceutically acceptable excipient.

In an aspect, the disclosure features a method of inhibiting the level and/or activity of BRD9 in a cell, the method involving contacting the cell with an effective amount of any of the foregoing compounds, or pharmaceutically acceptable salts thereof, or a pharmaceutical composition thereof.

In another aspect, the disclosure features a method of reducing the level and/or activity of BRD9 in a cell, the method involving contacting the cell with an effective amount of any of the foregoing compounds, or pharmaceutically acceptable salts thereof, or a pharmaceutical composition thereof.

In some embodiments, the cell is a cancer cell.

In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma (e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, low-grade rhabdomyosarcoma), non-small cell lung cancer (e.g., squamous or adenocarcinoma), stomach cancer, or breast cancer. In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer. In some embodiments, the cancer is a sarcoma (e.g., synovial sarcoma or Ewing's sarcoma), lung cancer (e.g., non-small cell lung cancer (e.g., squamous or adenocarcinoma)), stomach cancer, or breast cancer. In some embodiments, the cancer is sarcoma (e.g., synovial sarcoma or Ewing's sarcoma). In some embodiments, the sarcoma is synovial sarcoma.

In an aspect, the disclosure features a method of treating a BAF complex-related disorder in a subject in need thereof, the method involving administering to the subject an effective amount of any of the foregoing compounds, or pharmaceutically acceptable salts thereof, or a pharmaceutical composition thereof. In some embodiments, the BAF complex-related disorder is cancer. In some embodiments, the BAF complex-related disorder is infection.

In another aspect, the disclosure features a method of treating an SS18-SSX fusion protein-related disorder in a subject in need thereof, the method involving administering to the subject an effective amount of any of the foregoing compounds, or pharmaceutically acceptable salts thereof, or a pharmaceutical composition thereof. In some embodiments, the SS18-SSX fusion protein-related disorder is cancer. In some embodiments, the SS18-SSX fusion protein-related disorder is infection. In some embodiments of any of the foregoing methods, the SS18-SSX fusion protein is a SS18-SSX1 fusion protein, a SS18-SSX2 fusion protein, or a SS18-SSX4 fusion protein.

In yet another aspect, the disclosure features a method of treating a BRD9-related disorder in a subject in need thereof, the method involving administering to the subject an effective amount of any of the foregoing compounds, or pharmaceutically acceptable salts thereof, or a pharmaceutical composition thereof. In some embodiments, the BRD9-related disorder is cancer. In some embodiments, the BRD9-related disorder is infection.

In some embodiments, the cancer is squamous cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma (e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, low-grade rhabdomyosarcoma), non-small cell lung cancer (e.g., squamous or adenocarcinoma), stomach cancer, or breast cancer. In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer. In some embodiments, the cancer is a sarcoma (e.g., synovial sarcoma or Ewing's sarcoma), non-small cell lung cancer (e.g., squamous or adenocarcinoma), stomach cancer, or breast cancer. In some embodiments, the cancer is sarcoma (e.g., synovial sarcoma or Ewing's sarcoma). In some embodiments, the sarcoma is synovial sarcoma.

In some embodiments, the infection is viral infection (e.g., an infection with a virus of the Retroviridae family such as the lentiviruses (e.g. Human immunodeficiency virus (HIV) and deltaretroviruses (e.g., human T cell leukemia virus I (HTLV-I), human T cell leukemia virus II (HTLV-II)); Hepadnaviridae family (e.g. hepatitis B virus (HBV)); Flaviviridae family (e.g. hepatitis C virus (HCV)); Adenoviridae family (e.g. Human Adenovirus); Herpesviridae family (e.g. Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus); Papillomaviridae family (e.g. Human Papillomavirus (HPV, HPV E1)); Parvoviridae family (e.g. Parvovirus B19); Polyomaviridae family (e.g. JC virus and BK virus); Paramyxoviridae family (e.g. Measles virus); or Togaviridae family (e.g. Rubella virus)). In some embodiments, the disorder is Coffin Siris, Neurofibromatosis (e.g., NF-1, NF-2, or Schwannomatosis), or Multiple Meningioma. In an aspect, the disclosure features a method of treating a cancer in a subject in need thereof, the method including administering to the subject an effective amount of any of the foregoing compounds, or pharmaceutically acceptable salts thereof, or any of the foregoing pharmaceutical compositions.

In some embodiments, the cancer is squamous cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma (e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, low-grade rhabdomyosarcoma), non-small cell lung cancer (e.g., squamous or adenocarcinoma), stomach cancer, or breast cancer. In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer. In some embodiments, the cancer is a sarcoma (e.g., synovial sarcoma or Ewing's sarcoma), non-small cell lung cancer (e.g., squamous or adenocarcinoma), stomach cancer, or breast cancer. In some embodiments, the cancer is sarcoma (e.g., synovial sarcoma or Ewing's sarcoma). In some embodiments, the sarcoma is synovial sarcoma.

In another aspect, the disclosure features a method for treating a viral infection in a subject in need thereof. This method includes administering to the subject an effective amount of any of the foregoing compounds, or pharmaceutically acceptable salts thereof, or any of the foregoing pharmaceutical compositions. In some embodiments, the viral infection is an infection with a virus of the Retroviridae family such as the lentiviruses (e.g. Human immunodeficiency virus (HIV) and deltaretroviruses (e.g., human T cell leukemia virus I (HTLV-I), human T cell leukemia virus II (HTLV-II)); Hepadnaviridae family (e.g. hepatitis B virus (HBV)), Flaviviridae family (e.g. hepatitis C virus (HCV)), Adenoviridae family (e.g. Human Adenovirus), Herpesviridae family (e.g. Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g. Human Papillomavirus (HPV, HPV E1)), Parvoviridae family (e.g. Parvovirus B19), Polyomaviridae family (e.g. JC virus and BK virus), Paramyxoviridae family (e.g. Measles virus), Togaviridae family (e.g. Rubella virus).

In another embodiment of any of the foregoing methods, the method further includes administering to the subject an additional anticancer therapy (e.g., chemotherapeutic or cytotoxic agent or radiotherapy).

In particular embodiments, the additional anticancer therapy is: a chemotherapeutic or cytotoxic agent (e.g., doxorubicin or ifosfamide), a differentiation-inducing agent (e.g., retinoic acid, vitamin D, cytokines), a hormonal agent, an immunological agent, or an anti-angiogenic agent. Chemotherapeutic and cytotoxic agents include, but are not limited to, alkylating agents, cytotoxic antibiotics, antimetabolites, vinca alkaloids, etoposides, and others (e.g., paclitaxel, taxol, docetaxel, taxotere, cis-platinum). A list of additional compounds having anticancer activity can be found in L. Brunton, B. Chabner and B. Knollman (eds). Goodman and Gilman's The Pharmacological Basis of Therapeutics, Twelfth Edition, 2011, McGraw Hill Companies, New York, NY.

In particular embodiments, the compound of the invention and the additional anticancer therapy and any of the foregoing compounds or pharmaceutical compositions are administered within 28 days of each other (e.g., within 21, 14, 10, 7, 5, 4, 3, 2, or 1 days) or within 24 hours (e.g., 12, 6, 3, 2, or 1 hours; or concomitantly) each in an amount that together are effective to treat the subject.

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as hydrogen atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The term “aliphatic,” as used herein, refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In one embodiment, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl. Unsaturated aliphatic groups can be in a cis or trans configuration. In one embodiment, the aliphatic group contains from 1 to about 12 carbon atoms, more generally from 1 to about 6 carbon atoms or from 1 to about 4 carbon atoms. In one embodiment, the aliphatic group contains from 1 to about 8 carbon atoms. In certain embodiments, the aliphatic group is C₁-C₂, C₁-C₃, C₁-C₄, C₁-C₅, or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In one embodiment, the aliphatic group is substituted with one or more functional groups that results in the formation of a stable moiety.

The term “heteroaliphatic,” as used herein, refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In one embodiment, the only heteroatom is nitrogen. In one embodiment, the only heteroatom is oxygen. In one embodiment, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In one embodiment, “heteroaliphatic” is used to indicate a heteroaliphatic group (cyclic, acyclic, substituted, unsubstituted, branched or unbranched) having 1-20 carbon atoms. In one embodiment, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, and alkyl-O-haloalkyl.

The term “acyl,” as used herein, represents a hydrogen or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms). An “alkylene” is a divalent alkyl group.

The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms). An “alkenylene” is a divalent alkenyl group.

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms). An “alkynylene” is a divalent alkynyl group.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂).

The term “aryl,” as used herein, refers to an aromatic mono- or polycarbocyclic radical of, e.g., 6 to 12, carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group.

Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged cyclyl,” as used herein, refers to a bridged polycyclic group of 5 to 20 atoms, containing from 1 to 3 bridges. Bridged cyclyl includes bridged carbocyclyl (e.g., norbornyl) and bridged heterocyclyl (e.g., 1,4-diazabicyclo[2.2.2]octane).

The term “cyano,” as used herein, represents a —CN group.

The term “carbocyclyl,” as used herein, refers to a non-aromatic C₃-C₁₂, monocyclic or polycyclic (e.g., bicyclic or tricyclic) structure in which the rings are formed by carbon atoms. Carbocyclyl structures include cycloalkyl groups (e.g., cyclohexyl) and unsaturated carbocyclyl radicals (e.g., cyclohexenyl). Polycyclic carbocyclyl includes spirocyclic carbocyclyl, bridged carbocyclyl, and fused carbocyclyl. A “carbocyclylene” is a divalent carbocyclyl group.

The term “cycloalkyl,” as used herein, refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The terms “halo” or “halogen,” as used herein, mean a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers to alkyl-O— (e.g., methoxy and ethoxy), and an “alkylamino” which, as used herein, refers to —N(alkyl)R^{Na}, where R^{Na} is H or alkyl (e.g., methylamino). A “heteroalkylene” is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers to alkenyl-O—. A “heteroalkenylene” is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers to alkynyl-O—. A “heteroalkynylene” is a divalent heteroalkynyl group.

The term “heteroaryl,” as used herein, refers to an aromatic monocyclic or polycyclic structure of 5 to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl. A “heteroarylene” is a divalent heteroaryl group.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a monocyclic or polycyclic radical (e.g., bicyclic or tricyclic) having 3 to 12 atoms having at least one non-aromatic ring containing 1, 2, 3, or 4 ring atoms selected from N, O, or S, and no aromatic ring containing any N, O, or S atoms. Polycyclic heterocyclyl includes spirocyclic heterocyclyl, bridged heterocyclyl, and fused heterocyclyl. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. A “heterocyclylene” is a divalent heterocyclyl group.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “imine,” as used herein, represents=NRN group, where R^{N} is, e.g., H or alkyl.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “oxo,” as used herein, represents an ═O group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, oxo, sulfonyl, orthiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds described herein (e.g., compounds of the invention) can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein (e.g., the compounds of the invention) may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “adult soft tissue sarcoma” refers to a sarcoma that develops in the soft tissues of the body, typically in adolescent and adult subjects (e.g., subjects who are at least 10 years old, 11 years old, 12 years old, 13 years old, 14 years old, 15 years old, 16 years old, 17 years old, 18 years old, or 19 years old). Non-limiting examples of adult soft tissue sarcoma include, but are not limited to, synovial sarcoma, fibrosarcoma, malignant fibrous histiocytoma, dermatofibrosarcoma, liposarcoma, leiomyosarcoma, hemangiosarcoma, Kaposi's sarcoma, lymphangiosarcoma, malignant peripheral nerve sheath tumor/neurofibrosarcoma, extraskeletal chondrosarcoma, extraskeletal osteosarcoma, extraskeletal myxoid chondrosarcoma, and extraskeletal mesenchymal.

The term “antisense,” as used herein, refers to a nucleic acid comprising a polynucleotide that is sufficiently complementary to all or a portion of a gene, primary transcript, or processed mRNA, so as to interfere with expression of the endogenous gene (e.g., BRD9). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The term “antisense nucleic acid” includes single-stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity (e.g., 80%, 85%, 86%, 87%, 88%, 89%, 90%, 91%, 92%, 93%, 94%, 95%, 96%, 97%, 98%, 99%, 99.9% identity, or more) with the targeted polypeptide sequence (e.g., a BRD9 polypeptide sequence). The antisense nucleic acid can be complementary to an entire coding strand, or to only a portion thereof. In some embodiments, an antisense nucleic acid molecule is antisense to a “coding region” of the coding strand of a nucleotide sequence. The term “coding region” refers to the region of the nucleotide sequence comprising codons that are translated into amino acid residues. In some embodiments, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

As used herein, the term “BAF complex” refers to the BRG1- or HRBM-associated factors complex in a human cell.

As used herein, the term “BAF complex-related disorder” refers to a disorder that is caused or affected by the level and/or activity of a BAF complex.

As used herein, the terms “GBAF complex” and “GBAF” refer to a SWI/SNF ATPase chromatin remodeling complex in a human cell. GBAF complex subunits may include, but are not limited to, ACTB, ACTL6A, ACTL6B, BICRA, BICRAL, BRD9, SMARCA2, SMARCA4, SMARCC1, SMARCD1, SMARCD2, SMARCD3, and SS18. The term “cancer” refers to a condition caused by the proliferation of malignant neoplastic cells, such as tumors, neoplasms, carcinomas, sarcomas, leukemias, and lymphomas.

As used herein, the term “BRD9” refers to bromodomain-containing protein 9, a component of the BAF (BRG1- or BRM-associated factors) complex, a SWI/SNF ATPase chromatin remodeling complex, and belongs to family IV of the bromodomain-containing proteins. BRD9 is encoded by the BRD9 gene, the nucleic acid sequence corresponding to positions 863735-892803 in RefSeq sequence NC_000005.10 of GRCh38.p13 (RefSeq assembly accession No. GCF_000001405.39). The term “BRD9” also refers to natural variants of the wild-type BRD9 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type BRD9, which is set forth in SEQ ID NO: 1.

As used herein, the term “BRD9-related disorder” refers to a disorder that is caused or affected by the level and/or activity of BRD9. The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

A “compound of the present invention” and similar terms as used herein, whether explicitly noted or not, refers to compounds useful for treating BAF-related disorders (e.g., cancer or infection) described herein, including, e.g., compounds of Formula I (e.g., a compound of Table 1), as well as salts (e.g., pharmaceutically acceptable salts), solvates, hydrates, stereoisomers (including atropisomers), and tautomers thereof. Those skilled in the art will appreciate that certain compounds described herein can exist in one or more different isomeric (e.g., stereoisomers, geometric isomers, atropisomers, and tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination. Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

As used herein, the term “degrader” refers to a small molecule compound including a degradation moiety, wherein the compound interacts with a protein (e.g., BRD9) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRD9. In one example, the moiety binds to a protease or a ubiquitin ligase that metabolizes the protein, e.g., BRD9.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

As used herein, the terms “effective amount,” “therapeutically effective amount,” and “a “sufficient amount” of an agent that reduces the level and/or activity of BRD9 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of BRD9 sufficient to achieve a treatment response as compared to the response obtained without administration of the agent that reduces the level and/or activity of BRD9. The amount of a given agent that reduces the level and/or activity of BRD9 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of BRD9 of the present disclosure is an amount which results in a beneficial or desired result in a subject as compared to a control. As defined herein, a therapeutically effective amount of an agent that reduces the level and/or activity of BRD9 of the present disclosure may be readily determined by one of ordinary skill by routine methods known in the art. Dosage regimen may be adjusted to provide the optimum therapeutic response.

As used herein, the term “inhibitor” refers to any agent which reduces the level and/or activity of a protein (e.g., BRD9). Non-limiting examples of inhibitors include small molecule inhibitors, degraders, antibodies, enzymes, or polynucleotides (e.g., siRNA).

The term “inhibitory RNA agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein, or RNA) is down-regulated. Generally, an interfering RNA (“iRNA”) is a double-stranded short-interfering RNA (siRNA), short hairpin RNA (shRNA), or single-stranded micro-RNA (miRNA) that results in catalytic degradation of specific mRNAs, and also can be used to lower or inhibit gene expression.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

The terms “miRNA” and “microRNA” refer to an RNA agent, preferably a single-stranded agent, of about 10-50 nucleotides in length, preferably between about 15-25 nucleotides in length, which is capable of directing or mediating RNA interference. Naturally-occurring miRNAs are generated from stem-loop precursor RNAs (i.e., pre-miRNAs) by Dicer. The term “Dicer” as used herein, includes Dicer as well as any Dicer ortholog or homolog capable of processing dsRNA structures into siRNAs, miRNAs, siRNA-like or miRNA-like molecules. The term microRNA (“miRNA”) is used interchangeably with the term “small temporal RNA” (“stRNA”) based on the fact that naturally-occurring miRNAs have been found to be expressed in a temporal fashion (e.g., during development).

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity.

Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

By “reducing the activity of BRD9,” is meant decreasing the level of an activity related to an BRD9, or a related downstream effect. A non-limiting example of inhibition of an activity of BRD9 is decreasing the level of a BAF complex (e.g., GBAF) in a cell. The activity level of BRD9 may be measured using any method known in the art. In some embodiments, an agent which reduces the activity of BRD9 is a small molecule BRD9 inhibitor. In some embodiments, an agent which reduces the activity of BRD9 is a small molecule BRD9 degrader.

By “reducing the level of BRD9,” is meant decreasing the level of BRD9 in a cell or subject. The level of BRD9 may be measured using any method known in the art.

By a “reference” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

The terms “short interfering RNA” and “siRNA” (also known as “small interfering RNAs”) refer to an RNA agent, preferably a double-stranded agent, of about 10-50 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the term “SS18-SSX fusion protein-related disorder” refers to a disorder that is caused or affected by the level and/or activity of SS18-SSX fusion protein.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a series of graphs illustrating the effect of specific guide RNA (sgRNA) targeting of the BRD9 BAF complex subunit on synovial sarcoma cell growth. The Y-axis indicated the dropout ratio. The X-axis indicates the nucleotide position of the BRD9 gene. The grey box indicates the range of the negative control sgRNAs in the screen. The SYO1 cell line carries SS18-SSX2 fusion protein. The breakpoint joining the N-terminal region of SS18 to the C-terminal region of SSX2 are indicated by the black lines in their respective panel. The linear protein sequence is show with BRD9 PFAM domains annotated from the PFAM database.

FIG. 2 is an image illustrating dose dependent depletion of BRD9 levels in a synovial sarcoma cell line (SYO1) in the presence of a BRD9 degrader.

FIG. 3 is an image illustrating sustained suppression of BRD9 levels in a synovial sarcoma cell line (SYO1) in the presence of a BRD9 degrader over 72 hours.

FIG. 4 is an image illustrating sustained suppression of BRD9 levels in two cell lines (293T and SYO1) in the presence of a BRD9 degrader over 5 days.

FIG. 5 is an image illustrating sustained suppression of BRD9 levels in synovial sarcoma cell lines (SYO1 and Yamato) in the presence of a BRD9 degrader over 7 days compared to the levels in cells treated with CRISPR reagents.

FIG. 6 is an image illustrating the effect on cell growth of six cell lines (SYO1, Yamato, A⁵⁴⁹, HS-SY-II, ASKA, and 293T) in the presence of a BRD9 degrader and a BRD9 inhibitor.

FIG. 7 is an image illustrating the effect on cell growth of two cell lines (SYO1 and G401) in the presence of a BRD9 degrader.

FIG. 8 is an image illustrating the effect on cell growth of three synovial sarcoma cell lines (SYO1, HS-SY-II, and ASKA) in the presence of a BRD9 degrader, BRD9 binder and E3 ligase binder.

FIG. 9 is an image illustrating the effect on cell growth of three non-synovial sarcoma cell lines (RD, HCT116, and Calu6) in the presence of a BRD9 degrader, BRD9 binder and E3 ligase binder.

FIG. 10 is a graph illustrating the percentage of SYO1 in various cell cycle phases following treatment with DMSO, Compound 1 at 200 nM, or Compound 1 at 1 μM for 8 or 13 days.

FIG. 11 is a series of contour plots illustrating the percentage of SYO1 cells in various cell cycle phases following treatment with DMSO, Compound 1 at 200 nM, Compound 1 at 1 μM, or lenalidomide at 200 nM for 8 days. Numerical values corresponding to each contour plot are found in the table below.

FIG. 12 is a series of contour plots illustrating the percentage of SYO1 cells in various cell cycle phases following treatment with DMSO, Compound 1 at 200 nM, Compound 1 at 1 μM, or lenalidomide at 200 nM for 13 days. Numerical values corresponding to each contour plot are found in the table below.

FIG. 13 is a series of contour plots illustrating the percentage of early- and late-apoptotic SYO1 cells following treatment with DMSO, Compound 1 at 200 nM, Compound 1 at 1 μM, or lenalidomide at 200 nM for 8 days. Numerical values corresponding to each contour plot are found in the table below.

FIG. 14 is a graph illustrating the proteins present in BAF complexes including the SS18-SSX fusion protein.

### DETAILED DESCRIPTION

The present disclosure features compositions and methods useful for the treatment of BAF-related disorders (e.g., cancer and infection). The disclosure further features compositions and methods useful for inhibition of the level and/or activity of BRD9, e.g., for the treatment of disorders such as cancer (e.g., sarcoma) and infection (e.g., viral infection), e.g., in a subject in need thereof.

### Compounds

Compounds described herein reduce the level of an activity related to BRD9, or a related downstream effect, or reduce the level of BRD9 in a cell or subject. Exemplary compounds described herein have the structure according to Formula I.

Formula I is:

A-L-B Formula I,
where
L has the structure of Formula II:

A¹-E¹-F-E²-A², Formula II
A¹ is a bond between the linker and A;
A² is a bond between B and the linker;
each of E¹ and E² is, independently, absent, CH₂, O, or NCH₃; and
F is optionally substituted C₃-C₁₀ carbocyclylene or optionally substituted C₂₋₁₀ heterocyclylene;
B is a degradation moiety; and
A has the structure of Formula III:

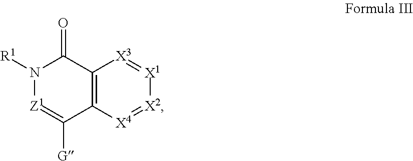

where
R¹ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
Z¹ is CR² or N;
R² is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, or optionally substituted C₂-C₉ heteroaryl;
X¹ is N or CH, and X² is C—R⁷″; or X¹ is C—R⁷″, and X² is N or CH;
R⁷″ is

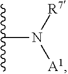

optionally substituted C₁-C₆ alkyl, optionally substituted C₁-C₆ heteroalkyl, optionally substituted C₁-C₆ alkoxy, optionally substituted amino, optionally substituted sulfone, optionally substituted sulfonamide, optionally substituted carbocyclyl having 3 to 6 atoms, or optionally substituted heterocyclyl having 3 to 6 atoms;
R^{7′} is H, optionally substituted C₁-C₆ alkyl, optionally substituted C₁-C₆ heteroalkyl, or optionally substituted C₃-C₁₀ carbocyclyl;
X³ is N or CH;
X⁴ is N or CH;
G″ is

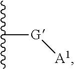

optionally substituted C₃-C₁₀ carbocyclyl, C₂-C₉ heterocyclyl, optionally substituted C₆-C₁₀ aryl, or optionally substituted C₂-C₉ heteroaryl;
G′ is optionally substituted C₃-C₁₀ carbocyclylene, C₂-C₉ heterocyclylene, optionally substituted C₆-C₁₀ arylene, or optionally substituted C₂-C₉ heteroarylene; and
A¹ is a bond between A and the linker
where G″ is

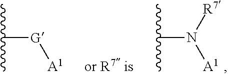

or a pharmaceutically acceptable salt thereof.

In some embodiments, the compound has the structure of any one of compounds D1-D66 in Table 1, or a pharmaceutically acceptable salt thereof.

Other embodiments, as well as exemplary methods for the synthesis of production of these compounds, are described herein.

### Pharmaceutical Uses

The compounds described herein are useful in the methods of the invention and, while not bound by theory, are believed to exert their desirable effects through their ability to modulate the level, status, and/or activity of a BAF complex, e.g., by inhibiting the activity or level of the BRD9 protein in a cell within the BAF complex in a mammal.

An aspect of the present invention relates to methods of treating disorders related to BRD9 such as cancer in a subject in need thereof. In some embodiments, the compound is administered in an amount and for a time effective to result in one of (or more, e.g., two or more, three or more, four or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, and (i) increased progression free survival of a subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound described herein. An increase in average survival time of a population may also be measured, for example, by calculating for a population the average length of survival following completion of a first round of treatment with a pharmaceutically acceptable salt of a compound described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of a compound described herein. A decrease in the mortality rate of a population may also be measured, for example, by calculating for a population the average number of disease-related deaths per unit time following completion of a first round of treatment with a pharmaceutically acceptable salt of a compound described herein.

Preferably, the methods of the inventions include an oral administration of a compound of the invention to a subject in need thereof. In some embodiments, methods of the invention are particularly preferred for subjects suffering from a sarcoma (e.g., synovial sarcoma). In some embodiments, methods of the invention are particularly preferred for subjects suffering from a breast cancer. In some embodiments, methods of the invention are particularly preferred for subjects suffering from a lung cancer (e.g., non-small cell lung cancer). In some embodiments, methods of the invention are particularly preferred for subjects suffering from an ovarian cancer. In some embodiments, methods of the invention are particularly preferred for subjects suffering from acute myeloid leukemia (AML).

### Combination Therapies

A method of the invention can be used alone or in combination with an additional therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; Ionidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the second therapeutic agent is a DNA damaging agent (e.g., a platinum-based antineoplastic agent, topoisomerase inhibitors, PARP inhibitors, alkylating antineoplastic agents, and ionizing radiation).

Examples of platinum-based antineoplastic agent that may be used as a second therapeutic agent in the compositions and methods of the invention are cisplatin, carboplatin, oxaliplatin, dicycloplatin, eptaplatin, lobaplatin, miriplatin, nedaplatin, triplatin tetranitrate, phenanthrilplatin, picoplatin, and satraplatin. In some embodiments, the second therapeutic agent is cisplatin and the treated cancer is a testicular cancer, ovarian cancer, or a bladder cancer (e.g., advanced bladder cancer). In some embodiments, the second therapeutic agent is carboplatin and the treated cancer is an ovarian cancer, lung cancer, head and neck cancer, brain cancer, or neuroblastoma. In some embodiments, the second therapeutic agent is oxaliplatin and the treated cancer is a colorectal cancer. In some embodiments, the second therapeutic agent is dicycloplatin and the treated cancer is a non-small cell ung cancer or prostate cancer. In some embodiments, the second therapeutic agent is eptaplatin and the treated cancer is a gastric cancer. In some embodiments, the second therapeutic agent is lobaplatin and the treated cancer is a breast cancer. In some embodiments, the second therapeutic agent is miriplatin and the treated cancer is a hepatocellular carcinoma. In some embodiments, the second therapeutic agent is nedaplatin and the treated cancer is a nasopharyngeal carcinoma, esophageal cancer, squamous cell carcinoma, or cervical cancer. In some embodiments, the second therapeutic agent is triplatin tetranitrate and the treated cancer is a lung cancer (e.g., small cell lung cancer) or pancreatic cancer. In some embodiments, the second therapeutic agent is picoplatin and the treated cancer is a lung cancer (e.g., small cell lung cancer), prostate cancer, bladder cancer, or colorectal cancer. In some embodiments, the second therapeutic agent is satrapltin and the treated cancer is a prostate cancer, breast cancer, or lung cancer.

Examples of topoisomerase inhibitors that may be used as a second therapeutic agent in the compositions and methods of the invention are etoposide, teniposide, doxorubicin, daunorubicin, mitoxantrone, amsacrine, ellipticine, irinotecan, topotecan, camptothecin, and diflomotecan. In some embodiments, the second therapeutic agent is etoposide and the treated cancer is a lung cancer (e.g., small cell lung cancer) or testicular cancer. In some embodiments, the second therapeutic agent is teniposide and the treated cancer is an acute lymphoblastic leukemia (e.g., childhood acute lymphoblastic leukemia). In some embodiments, the second therapeutic agent is doxorubicin and the treated cancer is an acute lymphoblastic leukemia, acute myeloblastic leukemia, Hodgkin lymphoma, Non-Hodgkin lymphoma, breast cancer, Wilm's tumor, neuroblastoma, soft tissue sarcoma, bone sarcomas, ovarian carcinoma, transitional cell bladder carcinoma, thyroid carcinoma, gastric carcinoma, or bronchogenic carcinoma. In some embodiments, the second therapeutic agent is daunorubicin and the treated cancer is an acute lymphoblastic leukemia or acute myeloid leukemia. In some embodiments, the second therapeutic agent is mitoxantrone and the treated cancer is a prostate cancer or acute nonlymphocytic leukemia. In some embodiments, the second therapeutic agent is amsacrine and the treated cancer is a leukemia (e.g., acute adult leukemia). In some embodiments, the second therapeutic agent is irinotecan and the treated cancer is a colorectal cancer. In some embodiments, the second therapeutic agent is topotecan and the treated cancer is a lung cancer (e.g., small cell lung cancer). In some embodiments, the second therapeutic agent is diflomotecan and the treated cancer is a lung cancer (e.g., small cell lung cancer).

Examples of alkylating antineoplastic agents that may be used as a second therapeutic agent in the compositions and methods of the invention are cyclophosphamide, uramustine, melphalan, chlorambucil, ifosfamide, bendamustine, carmustine, lomustine, chlorozotocin, fotemustine, nimustine, ranimustine, busulfan, improsulfan, piposulfan, chlornaphazine, cholophosphamide, estramustine, mechlorethamine, mechlorethamine oxide hydrochloride, novembichin, phenesterine, prednimustine, trofosfamide, procarbazine, altretamine, dacarbazine, mitozolomide, and temozolomide. In some embodiments, the second therapeutic agent is cyclophosphamide and the treated cancer is a Non-Hodgking lymphoma. In some embodiments, the second therapeutic agent is melphalan and the treated cancer is a multiple myeloma, ovarian cancer, or melanoma. In some embodiments, the second therapeutic agent is chlorambucil and the treated cancer is a chronic lymphatic leukemia, malignant lymphoma (e.g., lymphosarcoma, giant follicular lymphoma, or Hodgkin's lymphoma). In some embodiments, the second therapeutic agent is ifosfamide and the treated cancer is a testicular cancer. In some embodiments, the second therapeutic agent is bendamustine and the treated cancer is a chronic lymphocytic leukemia or non-Hodgkin lymphoma. In some embodiments, the second therapeutic agent is carmustine and the treated cancer is a brain cancer (e.g., glioblastoma, brainstem glioma, medulloblastoma, astrocytoma, ependymoma, or a metastatic brain tumor), multiple myeloma, Hodgkin's disease, or Non-Hodgkin's lymphoma. In some embodiments, the second therapeutic agent is lomustine and the treated cancer is a brain cancer or Hodgkin's lymphoma. In some embodiments, the second therapeutic agent is fotemustine and the treated cancer is a melanoma. In some embodiments, the second therapeutic agent is nimustine and the treated cancer is a brain cancer. In some embodiments, the second therapeutic agent is ranimustine and the treated cancer is a chronic myelogenous leukemia or polycythemia vera. In some embodiments, the second therapeutic agent is busulfan and the treated cancer is a chronic myelogenous leukemia. In some embodiments, the second therapeutic agent is improsulfan and the treated cancer is a sarcoma. In some embodiments, the second therapeutic agent is estramustine and the treated cancer is a prostate cancer (e.g., prostate carcinoma). In some embodiments, the second therapeutic agent is mechlomethamine and the treated cancer is a cutaneous T-cell lymphoma. In some embodiments, the second therapeutic agent is trofosfamide and the treated cancer is a sarcoma (e.g., soft tissue sarcoma). In some embodiments, the second therapeutic agent is procarbazine and the treated cancer is a Hodgkin's disease. In some embodiments, the second therapeutic agent is altretamine and the treated cancer is an ovarian cancer. In some embodiments, the second therapeutic agent is dacarbazine and the treated cancer is a melanoma, Hodgkin's lymphoma, or sarcoma. In some embodiments, the second therapeutic agent is temozolomide and the treated cancer is a brain cancer (e.g., astrocytoma or glioblastoma) or lung cancer (e.g., small cell lung cancer).

Examples of PARP inhibitors that may be used as a second therapeutic agent in the compositions and methods of the invention are niraparib, olaparib, rucaparib, talazoparib, veliparib, pamiparib, CK-102, or E7016. Advantageously, the compounds of the invention and a DNA damaging agent may act synergistically to treat cancer. In some embodiments, the second therapeutic agent is niraparib and the treated cancer is an ovarian cancer (e.g., BRCA mutated ovarian cancer), fallopian tube cancer (e.g., BRCA mutated fallopian tube cancer), or primary peritoneal cancer (e.g., BRCA mutated primary peritoneal cancer). In some embodiments, the second therapeutic agent is olaparib and the treated cancer is a lung cancer (e.g., small cell lung cancer), ovarian cancer (e.g., BRCA mutated ovarian cancer), breast cancer (e.g., BRCA mutated breast cancer), fallopian tube cancer (e.g., BRCA mutated fallopian tube cancer), primary peritoneal cancer (e.g., BRCA mutated primary peritoneal cancer), prostate cancer (e.g., castration-resistant prostate cancer), or pancreatic cancer (e.g., pancreatic adenocarcinoma). In some embodiments, the second therapeutic agent is rucaparib and the treated cancer is an ovarian cancer (e.g., BRCA mutated ovarian cancer), fallopian tube cancer (e.g., BRCA mutated fallopian tube cancer), or primary peritoneal cancer (e.g., BRCA mutated primary peritoneal cancer). In some embodiments, the second therapeutic agent is talazoparib and the treated cancer is a breast cancer (e.g., BRCA mutated breast cancer). In some embodiments, the second therapeutic agent is veliparib and the treated cancer is a lung cancer (e.g., non-small cell lung cancer), malenoma, breast cancer, ovarian cancer, prostate cancer, or brain cancer. In some embodiments, the second therapeutic agent is pamiparib and the treated cancer is an ovarian cancer. In some embodiments, the second therapeutic agent is CK-102 and the treated cancer is a lung cancer (e.g., non-small cell lung cancer). In some embodiments, the second therapeutic agent is E7016 and the treated cancer is a melanoma.

Without wishing to be bound by theory, the synergy between the compounds of the invention and DNA damaging agents may be attributed to the necessity of BRD9 for DNA repair; inhibition of BRD9 may sensitize cancer (e.g., cancer cell or cancer tissue) to DNA damaging agents.

In some embodiments, the second therapeutic agent is a JAK inhibitor (e.g., JAK1 inhibitor). Non-limiting examples of JAK inhibitors that may be used as a second therapeutic agent in the compositions and methods of the invention include tofacitinib, ruxolitinib, oclacitinib, baricitinib, peficitinib, fedratinib, upadacitinib, filgotinib, cerdulatinib, gandotinib, lestaurtinib, momelotinib, pacritinib, abrocitinib, solcitinib, itacitinib, or SHR0302. Without wishing to be bound by theory, the synergy between the compounds of the invention and JAK inhibitors may be inhibitor of SAGA complex to their combined effect of downregulating Foxp3+ Treg cells. In some embodiments, the second therapeutic agent is ruxolitinib and the treated cancer is a myeloproliferative neoplasm (e.g., polycythemia or myelofibrosis), ovarian cancer, breast cancer, pancreatic cancer. In some embodiments, the second therapeutic agent is fedratinib and the treated cancer is a myeloproliferative neoplasm (e.g., myelofibrosis). In some embodiments, the second therapeutic agent is cerdulatinib and the treated cancer is a lymphoma (e.g., peripheral T-cell lymphoma). In some embodiments, the second therapeutic agent is gandotinib and the treated cancer is a myeloproliferative neoplasm (e.g., polycythemia or myelofibrosis). In some embodiments, the second therapeutic agent is lestaurtinib and the treated cancer is a myeloproliferative neoplasm (e.g., polycythemia or myelofibrosis), leukemia (e.g., acute myelogenous leukemia), pancreatic cancer, prostate cancer, or neuroblastoma. In some embodiments, the second therapeutic agent is momelotinib and the treated cancer is a myeloproliferative neoplasm (e.g., polycythemia or myelofibrosis) or pancreatic cancer (e.g., pancreatic ductal adenocarcinoma). In some embodiments, the second therapeutic agent is momelotinib and the treated cancer is a myeloproliferative neoplasm (e.g., polycythemia or myelofibrosis). In some embodiments, the second therapeutic agent is momelotinib and the treated cancer is a myeloproliferative neoplasm (e.g., polycythemia or myelofibrosis) or pancreatic cancer (e.g., pancreatic ductal adenocarcinoma).

In some embodiments, the second therapeutic agent is an inhibitor of SAGA complex or a component thereof. A SAGA complex inhibitor may be, e.g., an inhibitory antibody or small molecule inhibitor, of CCDC101, Tada2B, Tada3, Usp22, Tada1, Taf61, Supt5, Supt20, or a combination thereof. Without wishing to be bound by theory, the synergy between the compounds of the invention and inhibitors of SAGA complex may be attributed to their combined effect of downregulating Foxp3+ Treg cells.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-1-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOLIRIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The second agent may be a therapeutic agent which is a non-drug treatment. For example, the second therapeutic agent is radiation therapy, cryotherapy, hyperthermia, and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or fusion a protein such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MED14736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the second therapeutic agent is ipilimumab and the treated cancer is a melanoma, kidney cancer, lung cancer (e.g., non-small cell lung cancer or small cell lung cancer), or prostate cancer. In some embodiments, the second therapeutic agent is tremelimumab and the treated cancer is a melanoma, mesothelioma, or lung cancer (e.g., non-small cell lung cancer). In some embodiments, the second therapeutic agent is nivolumab and the treated cancer is a melanoma, lung cancer (e.g., non-small cell lung cancer or small cell lung cancer), kidney cancer, Hodgkin lymphoma, head and neck cancer (e.g., squamous cell carcinoma of the head and neck), urothelial carcinoma, hepatocellular carcinoma, or colorectal cancer. In some embodiments, the second therapeutic agent is pembrolizumab and the treated cancer is a melanoma, lung cancer (e.g., non-small cell lung cancer or small cell lung cancer), Hodgkin lymphoma, head and neck cancer (e.g., squamous cell carcinoma of the head and neck), primary mediastinal large B-cell lymphoma, urothelial carcinoma, hepatocellular carcinoma, microsatellite instability-high cancer, gastric cancer, esophageal cancer, cervical cancer, Merkel cell carcinoma, kidney carcinoma, or endometrial carcinoma. In some embodiments, the second therapeutic agent is MPDL3280A and the treated cancer is a lung cancer (e.g., non-small cell lung cancer or small cell lung cancer), urothelial carcinoma, hepatocellular carcinoma, or breast cancer. In some embodiments, the second therapeutic agent is MED14736 and the treated cancer is a lung cancer (e.g., non-small cell lung cancer or small cell lung cancer) or urothelial carcinoma. In some embodiments, the second therapeutic agent is MSB0010718C and the treated cancer is a urothelial carcinoma. In some embodiments, the second therapeutic agent is MSB0010718C and the treated cancer is a melanoma, lung cancer (e.g., non-small cell lung cancer), colorectal cancer, kidney cancer, ovarian cancer, pancreatic cancer, gastric cancer, and breast cancer.

Advantageously, the compounds of the invention and a checkpoint inhibitor may act synergistically to treat cancer. Without wishing to be bound by theory, the synergy between the compounds of the invention and checkpoint inhibitors may be attributed to the checkpoint inhibitor efficacy enhancement associated with the BRD9 inhibition-induced downregulation of Foxp3+ Treg cells.

In some embodiments, the anti-cancer therapy is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The pharmaceutical compositions described herein are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the invention, the described compounds or salts, solvates, or prodrugs thereof may be administered to a patient in a variety of forms depending on the selected route of administration, as will be understood by those skilled in the art. The compounds described herein may be administered, for example, by oral, parenteral, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Solutions of a compound described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response. In general, satisfactory results may be obtained when the compounds described herein are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-100 mg/kg.

### Kits

The invention also features kits including (a) a pharmaceutical composition including an agent that reduces the level and/or activity of BRD9 in a cell or subject described herein, and (b) a package insert with instructions to perform any of the methods described herein. In some embodiments, the kit includes (a) a pharmaceutical composition including an agent that reduces the level and/or activity of BRD9 in a cell or subject described herein, (b) an additional therapeutic agent (e.g., an anti-cancer agent), and (c) a package insert with instructions to perform any of the methods described herein.

### EXAMPLES

### Example 1—High Density Tiling sgRNA Screen Against Human BAF Complex Subunits in Synovial Sarcoma Cell Line SYO1

The following example shows that BRD9 sgRNA inhibits cell growth in synovial sarcoma cells.

Procedure: To perform high density sgRNA tiling screen, an sgRNA library against BAF complex subunits was custom synthesized at Cellecta (Mountain View, CA). Sequences of DNA encoding the BRD9-targeting sgRNAs used in this screen are listed in Table 2. Negative and positive control sgRNA were included in the library. Negative controls consisted of 200 sgRNAs that do not target human genome. The positive controls are sgRNAs targeting essential genes (CDC16, GTF2B, HSPA5, HSPA9, PAFAH1B1, PCNA, POLR2L, RPL9, and SF3A3). DNA sequences encoding all positive and negative control sgRNAs are listed in Table 3. Procedures for virus production, cell infection, and performing the sgRNA screen were previously described (Tsherniak et al, Cell 170:564-576 (2017); Munoz et al, Cancer Discovery 6:900-913 (2016)). For each sgRNA, 50 counts were added to the sequencing counts and for each time point the resulting counts were normalized to the total number of counts. The log 2 of the ratio between the counts (defined as dropout ratio) at day 24 and day 1 post-infection was calculated. For negative control sgRNAs, the 2.5 and 97.5 percentile of the log 2 dropout ratio of all non-targeting sgRNAs was calculated and considered as background (grey box in the graph). Protein domains were obtained from PFAM regions defined for the UNIPROT identifier: Q9H8M2.

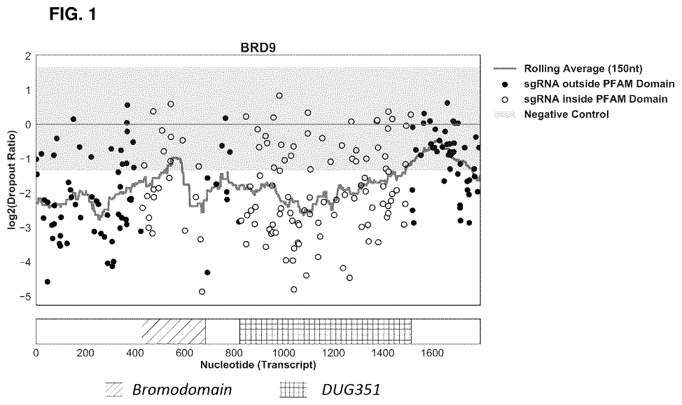

Results: As shown in FIG. 1, targeted inhibition of the GBAF complex component BRD9 by sgRNA resulted in growth inhibition of the SYO1 synovial sarcoma cell line. sgRNAs against other components of the BAF complexes resulted in increased proliferation of cells, inhibition of cell growth, or had no effect on SYO1 cells. These data show that targeting various subunits of the GBAF complex represents a therapeutic strategy for the treatment of synovial sarcoma.

TABLE 2
BRD9 sgRNA Library

| SEQ ID NO | Nucleic Acid Sequence |
|---|---|
| 203 | CAAGAAGCACAAGAAGCACA |
| 204 | CTTGTGCTTCTTGCCCATGG |
| 205 | CTTCTTGTGCTTCTTGCCCA |
| 206 | ACAAGAAGCACAAGGCCGAG |
| 207 | CTCGTAGGACGAGCGCCACT |
| 208 | CGAGTGGCGCTCGTCCTACG |
| 209 | GAGTGGCGCTCGTCCTACGA |
| 210 | AGGCTTCTCCAGGGGCTTGT |
| 211 | AGATTATGCCGACAAGCCCC |
| 212 | ACCTTCAGGACTAGCTTTAG |
| 213 | AGCTTTAGAGGCTTCTCCAG |
| 214 | CTAGCTTTAGAGGCTTCTCC |
| 215 | TAGCTTTAGAGGCTTCTCCA |
| 216 | CTAAAGCTAGTCCTGAAGGT |
| 217 | GCCTCTAAAGCTAGTCCTGA |
| 218 | CTTCACTTCCTCCGACCTTC |
| 219 | AAGCTAGTCCTGAAGGTCGG |
| 220 | AGTGAAGTGACTGAACTCTC |
| 221 | GTGACTGAACTCTCAGGATC |
| 222 | ATAGTAACTGGAGTCGTGGC |
| 223 | CATCATAGTAACTGGAGTCG |
| 224 | TGACCTGTCATCATAGTAAC |
| 225 | ACTCCAGTTACTATGATGAC |
| 226 | CTTTGTGCCTCTCTCGCTCA |
| 227 | GGTCAGACCATGAGCGAGAG |
| 228 | GAAGAAGAAGAAGTCCGAGA |
| 229 | GTCCAGATGCTTCTCCTTCT |
| 230 | GTCCGAGAAGGAGAAGCATC |
| 231 | GGAGAAGCATCTGGACGATG |
| 232 | TGAGGAAAGAAGGAAGCGAA |
| 233 | ATCTGGACGATGAGGAAAGA |
| 234 | AGAAGAAGCGGAAGCGAGAG |
| 235 | GAAGAAGCGGAAGCGAGAGA |
| 236 | CCGCCCAGGAAGAGAAGAAG |
| 237 | AGAGAGGGAGCACTGTGACA |
| 238 | AGGGAGCACTGTGACACGGA |
| 239 | GAGGGAGCACTGTGACACGG |
| 240 | GCACTGTGACACGGAGGGAG |
| 241 | GAGGCTGACGACTTTGATCC |
| 242 | AGGCTGACGACTTTGATCCT |
| 243 | TCCACCTCCACCTTCTTCCC |
| 244 | CGACTTTGATCCTGGGAAGA |
| 245 | CTTTGATCCTGGGAAGAAGG |
| 246 | TGATCCTGGGAAGAAGGTGG |
| 247 | TCCTGGGAAGAAGGTGGAGG |
| 248 | CGGACTGGCCGATCTGGGGG |
| 249 | ACGCTCGGACTGGCCGATCT |
| 250 | AGGTGGAGCCGCCCCCAGAT |
| 251 | CGCTCGGACTGGCCGATCTG |
| 252 | GCTCGGACTGGCCGATCTGG |
| 253 | CACGCTCGGACTGGCCGATC |
| 254 | TGTGTCCGGCACGCTCGGAC |
| 255 | CTGGCTGTGTCCGGCACGCT |
| 256 | ATCGGCCAGTCCGAGCGTGC |
| 257 | CACCCTTGCCTGGCTGTGTC |
| 258 | CGAGCGTGCCGGACACAGCC |
| 259 | TGTTCCAGGAGTTGCTGAAT |
| 260 | CACACCTATTCAGCAACTCC |
| 261 | GCTGGCGGAGGAAGTGTTCC |
| 262 | TTTACCTCTGAAGCTGGCGG |
| 263 | CCCCGGTTTACCTCTGAAGC |
| 264 | ACTTCCTCCGCCAGCTTCAG |
| 265 | CAGGAAAAGCAAAAAATCCA |
| 266 | GCTTTCAGAAAAGATCCCCA |
| 267 | AGGAAAAGCAAAAAATCCAT |
| 268 | GGAAAAGCAAAAAATCCATG |
| 269 | GGAGCAATTGCATCCGTGAC |
| 270 | GTCACGGATGCAATTGCTCC |
| 271 | TTTATTATCATTGAATATCC |
| 272 | AATGATAATAAAACATCCCA |
| 273 | ATAAAACATCCCATGGATTT |
| 274 | TTCATGGTGCCAAAATCCAT |
| 275 | TTTCATGGTGCCAAAATCCA |
| 276 | TAATGAATACAAGTCAGTTA |
| 277 | CAAGTCAGTTACGGAATTTA |
| 278 | ATAATGCAATGACATACAAT |
| 279 | AACTTGTAGTACACGGTATC |
| 280 | CTTCGCCAACTTGTAGTACA |
| 281 | AGATACCGTGTACTACAAGT |
| 282 | GCGAAGAAGATCCTTCACGC |
| 283 | TCATCTTAAAGCCTGCGTGA |
| 284 | TTCTCAGCAGGCAGCTCTTT |
| 285 | CAATGAAGATACAGCTGTTG |
| 286 | ACTGGTACAACTTCAGGGAC |
| 287 | CTTGTACTGGTACAACTTCA |
| 288 | ACTTGTACTGGTACAACTTC |
| 289 | TTGGCAGTTTCTACTTGTAC |
| 290 | TACCTGATAACTTCTCTACT |
| 291 | AGCCGAGTAGAGAAGTTATC |
| 292 | AGCTGCATGTTTGAGCCTGA |
| 293 | GCTGCATGTTTGAGCCTGAA |
| 294 | AAGCTGCAGGCATTCCCTTC |
| 295 | GGTACTGTCCGTCAAGCTGC |
| 296 | AGGGAATGCCTGCAGCTTGA |
| 297 | CTTGACGGACAGTACCGCAG |
| 298 | CGCCAGCACGTGCTCCTCTG |
| 299 | TACCGCAGAGGAGCACGTGC |
| 300 | AGAGGAGCACGTGCTGGCGC |
| 301 | GGAGCACGTGCTGGCGCTGG |
| 302 | AGCACGCAGCTGACGAAGCT |
| 303 | GCACGCAGCTGACGAAGCTC |
| 304 | CAGCTGACGAAGCTCGGGAC |
| 305 | AAGCTCGGGACAGGATCAAC |
| 306 | CCTTGCCGCCTGGGAGGAAC |
| 307 | AGGATCAACCGGTTCCTCCC |
| 308 | ATCAACCGGTTCCTCCCAGG |
| 309 | GCACTACCTTGCCGCCTGGG |
| 310 | AGAGCACTACCTTGCCGCCT |
| 311 | CCGGTTCCTCCCAGGCGGCA |
| 312 | TCCTCTTCAGATAGCCCATC |
| 313 | ATGGGCTATCTGAAGAGGAA |
| 314 | GGGCTATCTGAAGAGGAACG |
| 315 | TGGGCTATCTGAAGAGGAAC |
| 316 | TATCTGAAGAGGAACGGGGA |
| 317 | ATCTGAAGAGGAACGGGGAC |
| 318 | TGTTGACCACGCTGTAGAGC |
| 319 | GCTCTACAGCGTGGTCAACA |
| 320 | CGGGAGCCTGCTCTACAGCG |
| 321 | CGTGGTCAACACGGCCGAGC |
| 322 | CCCACCATCAGCGTCCGGCT |
| 323 | ACGGCCGAGCCGGACGCTGA |
| 324 | GGGCACCCACCATCAGCGTC |
| 325 | GCCGAGCCGGACGCTGATGG |
| 326 | CCATGTCCGTGTTGCAGAGG |
| 327 | CCGAGCCGGACGCTGATGGT |
| 328 | CGAGCTCAAGTCCACCGGGT |
| 329 | GCGAGCTCAAGTCCACCGGG |
| 330 | AGAGCGAGCTCAAGTCCACC |
| 331 | GAGAGCGAGCTCAAGTCCAC |
| 332 | GAAGCCTGGGAGTAGCTTAC |
| 333 | CTCTCCAGTAAGCTACTCCC |
| 334 | AGCCCAGCGTGGTGAAGCCT |
| 335 | AAGCCCAGCGTGGTGAAGCC |
| 336 | ACTCCCAGGCTTCACCACGC |
| 337 | CTCCCAGGCTTCACCACGCT |
| 338 | CTCGTCTTTGAAGCCCAGCG |
| 339 | CACTGGAGAGAAAGGTGACT |
| 340 | GCACTGGAGAGAAAGGTGAC |
| 341 | AGTAGTGGCACTGGAGAGAA |
| 342 | CGAAAGCGCAGTAGTGGCAC |
| 343 | CTGCATCGAAAGCGCAGTAG |
| 344 | ATGCAGAATAATTCAGTATT |
| 345 | AGTATTTGGCGACTTGAAGT |
| 346 | CGACTTGAAGTCGGACGAGA |
| 347 | GAGCTGCTCTACTCAGCCTA |
| 348 | CACGCCTGTCTCATCTCCGT |
| 349 | TCAGCCTACGGAGATGAGAC |
| 350 | CAGGCGTGCAGTGTGCGCTG |
| 351 | CCGCGGCCCCTCTAGCCTGC |
| 352 | CATCCTTCACAAACTCCTGC |
| 353 | TAGCCTGCAGGAGTTTGTGA |
| 354 | CAGGAGTTTGTGAAGGATGC |
| 355 | AGGAGTTTGTGAAGGATGCT |
| 356 | TGGGAGCTACAGCAAGAAAG |
| 357 | GAGCTACAGCAAGAAAGTGG |
| 358 | GAAAGTGGTGGACGACCTCC |
| 359 | CGCCTGTGATCTGGTCCAGG |
| 360 | CTCCGCCTGTGATCTGGTCC |
| 361 | GACCTCCTGGACCAGATCAC |
| 362 | CTCCTGGACCAGATCACAGG |
| 363 | GCTGGAAGAGCGTCCTAGAG |
| 364 | TGCAGCCCACCTGCTTCAGC |
| 365 | GACGCTCTTCCAGCTGAAGC |
| 366 | CTCTTCCAGCTGAAGCAGGT |
| 367 | GCTCTTCCAGCTGAAGCAGG |
| 368 | CCTCCAGATGAAGCCAAGGT |
| 369 | GCTTCATCTGGAGGCTTCAT |
| 370 | GGCTTCATCTGGAGGCTTCA |
| 371 | CTTACCTTGGCTTCATCTGG |
| 372 | AAACTTACCTTGGCTTCATC |
| 373 | GAAGCCTCCAGATGAAGCCA |
| 374 | TCCTAGGGTGTCCCCAACCT |
| 375 | CCTAGGGTGTCCCCAACCTG |
| 376 | GTGTCTGTCTCCACAGGTTG |
| 377 | TGTGTCTGTCTCCACAGGTT |
| 378 | CCACAGGTTGGGGACACCCT |
| 379 | AGAGCTGCTGCTGTCTCCTA |
| 380 | CAGAGCTGCTGCTGTCTCCT |
| 381 | AGACAGCAGCAGCTCTGTTC |
| 382 | ATCCACAGAAACGTCGGGAT |
| 383 | GAGATATCCACAGAAACGTC |
| 384 | GGAGATATCCACAGAAACGT |
| 385 | GTCCTATCCCGACGTTTCTG |
| 386 | TCTCCATGCTCAGCTCTCTG |
| 387 | CTCACCCAGAGAGCTGAGCA |
| 388 | ATCTCCATGCTCAGCTCTCT |
| 389 | TATCTCCATGCTCAGCTCTC |
| 390 | ATGTCCTGTTTACACAGGGA |
| 391 | TTACACAGGGAAGGTGAAGA |
| 392 | AGTTCAAATGGCTGTCGTCA |
| 393 | TGACGACAGCCATTTGAACT |
| 394 | AAGTTCAAATGGCTGTCGTC |
| 395 | TCGTCTCATCCAAGTTCAAA |
| 396 | TGAGACGACGAAGCTCCTGC |
| 397 | GTGCTTCGTGCAGGTCCTGC |
| 398 | GCAGGACCTGCACGAAGCAC |
| 399 | GCTCCGCCTGTGCTTCGTGC |
| 400 | GGACCTGCACGAAGCACAGG |
| 401 | CACGAAGCACAGGCGGAGCG |
| 402 | AGGCGGAGCGCGGCGGCTCT |
| 403 | AGGGAGCTGAGGTTGGACGA |
| 404 | GTTGGACAGGGAGCTGAGGT |
| 405 | AGGCGTTGGACAGGGAGCTG |
| 406 | CCCTCTCGGAGGCGTTGGAC |
| 407 | CCTCTCGGAGGCGTTGGACA |
| 408 | CTGGTCCCTCTCGGAGGCGT |
| 409 | CCCTGTCCAACGCCTCCGAG |
| 410 | CCTGTCCAACGCCTCCGAGA |
| 411 | GTGGTGCTGGTCCCTCTCGG |
| 412 | CAGGTGGTGCTGGTCCCTCT |
| 413 | GCATCTCACCCAGGTGGTGC |
| 414 | CGAGAGGGACCAGCACCACC |
| 415 | GAGAGGGACCAGCACCACCT |
| 416 | GTGGGGGCATCTCACCCAGG |
| 417 | CCCCGACACTCAGGCGAGAA |
| 418 | TCCCCGACACTCAGGCGAGA |
| 419 | AGCCCTTCTCGCCTGAGTGT |
| 420 | CTGGCTGCTCCCCGACACTC |
| 421 | CCCTTCTCGCCTGAGTGTCG |
| 422 | GCCCTTCTCGCCTGAGTGTC |
| 423 | TAGGGGTCGTGGGTGACGTC |
| 424 | AAGAAACTCATAGGGGTCGT |
| 425 | GAAGAAACTCATAGGGGTCG |
| 426 | GAGACTGAAGAAACTCATAG |
| 427 | GGAGACTGAAGAAACTCATA |
| 428 | TGGAGACTGAAGAAACTCAT |
| 429 | TCTTCAGTCTCCAGAGCCTG |
| 430 | TTGGCAGAGGCCGCAGGCTC |
| 431 | TAGGTCTTGGCAGAGGCCGC |
| 432 | CTAGAGTTAGGTCTTGGCAG |
| 433 | GGTGGTCTAGAGTTAGGTCT |

TABLE 3
Control sgRNA Library

| SEQ ID | | | |
|---|---|---|---|
| NO. | gRNA Label | Gene | Nucleic Acid Sequence |
| 434 | 1\|sg_Non_Targeting_Human_0001\| | Non_Targeting_Human | GTAGCGAACGTGTCCGGCGT |
| | Non_Targeting_Human | | |
| 435 | 1\|sg_Non_Targeting_Human_0002\| | Non_Targeting_Human | GACCGGAACGATCTCGCGTA |
| | Non_Targeting_Human | | |
| 436 | 1\|sg_Non_Targeting_Human_0003\| | Non_Targeting_Human | GGCAGTCGTTCGGTTGATAT |
| | Non_Targeting_Human | | |
| 437 | 1\|sg_Non_Targeting_Human_0004\| | Non_Targeting_Human | GCTTGAGCACATACGCGAAT |
| | Non_Targeting_Human | | |
| 438 | 1\|sg_Non_Targeting_Human_0005\| | Non_Targeting_Human | GTGGTAGAATAACGTATTAC |
| | Non_Targeting_Human | | |
| 439 | 1\|sg_Non_Targeting_Human_0006\| | Non_Targeting_Human | GTCATACATGGATAAGGCTA |
| | Non_Targeting_Human | | |
| 440 | 1\|sg_Non_Targeting_Human_0007\| | Non_Targeting_Human | GATACACGAAGCATCACTAG |
| | Non_Targeting_Human | | |
| 441 | 1\|sg_Non_Targeting_Human_0008\| | Non_Targeting_Human | GAACGTTGGCACTACTTCAC |
| | Non_Targeting_Human | | |
| 442 | 1\|sg_Non_Targeting_Human_0009\| | Non_Targeting_Human | GATCCATGTAATGCGTTCGA |
| | Non_Targeting_Human | | |
| 443 | 1\|sg_Non_Targeting_Human_0010\| | Non_Targeting_Human | GTCGTGAAGTGCATTCGATC |
| | Non_Targeting_Human | | |
| 444 | 1\|sg_Non_Targeting_Human_0011\| | Non_Targeting_Human | GTTCGACTCGCGTGACCGTA |
| | Non_Targeting_Human | | |
| 445 | 1\|sg_Non_Targeting_Human_0012\| | Non_Targeting_Human | GAATCTACCGCAGCGGTTCG |
| | Non_Targeting_Human | | |
| 446 | 1\|sg_Non_Targeting_Human_0013\| | Non_Targeting_Human | GAAGTGACGTCGATTCGATA |
| | Non_Targeting_Human | | |
| 447 | 1\|sg_Non_Targeting_Human_0014\| | Non_Targeting_Human | GCGGTGTATGACAACCGCCG |
| | Non_Targeting_Human | | |
| 448 | 1\|sg_Non_Targeting_Human_0015\| | Non_Targeting_Human | GTACCGCGCCTGAAGTTCGC |
| | Non_Targeting_Human | | |
| 449 | 1\|sg_Non_Targeting_Human_0016\| | Non_Targeting_Human | GCAGCTCGTGTGTCGTACTC |
| | Non_Targeting_Human | | |
| 450 | 1\|sg_Non_Targeting_Human_0017\| | Non_Targeting_Human | GCGCCTTAAGAGTACTCATC |
| | Non_Targeting_Human | | |
| 451 | 1\|sg_Non_Targeting_Human_0018\| | Non_Targeting_Human | GAGTGTCGTCGTTGCTCCTA |
| | Non_Targeting_Human | | |
| 452 | 1\|sg_Non_Targeting_Human_0019\| | Non_Targeting_Human | GCAGCTCGACCTCAAGCCGT |
| | Non_Targeting_Human | | |
| 453 | 1\|sg_Non_Targeting_Human_0020\| | Non_Targeting_Human | GTATCCTGACCTACGCGCTG |
| | Non_Targeting_Human | | |
| 454 | 1\|sg_Non_Targeting_Human_0021\| | Non_Targeting_Human | GTGTATCTCAGCACGCTAAC |
| | Non_Targeting_Human | | |
| 455 | 1\|sg_Non_Targeting_Human_0022\| | Non_Targeting_Human | GTCGTCATACAACGGCAACG |
| | Non_Targeting_Human | | |
| 456 | 1\|sg_Non_Targeting_Human_0023\| | Non_Targeting_Human | GTCGTGCGCTTCCGGCGGTA |
| | Non_Targeting_Human | | |
| 457 | 1\|sg_Non_Targeting_Human_0024\| | Non_Targeting_Human | GCGGTCCTCAGTAAGCGCGT |
| | Non_Targeting_Human | | |
| 458 | 1\|sg_Non_Targeting_Human_0025\| | Non_Targeting_Human | GCTCTGCTGCGGAAGGATTC |
| | Non_Targeting_Human | | |
| 459 | 1\|sg_Non_Targeting_Human_0026\| | Non_Targeting_Human | GCATGGAGGAGCGTCGCAGA |
| | Non_Targeting_Human | | |
| 460 | 1\|sg_Non_Targeting_Human_0027\| | Non_Targeting_Human | GTAGCGCGCGTAGGAGTGGC |
| | Non_Targeting_Human | | |
| 461 | 1\|sg_Non_Targeting_Human_0028\| | Non_Targeting_Human | GATCACCTGCATTCGTACAC |
| | Non_Targeting_Human | | |
| 462 | 1\|sg_Non_Targeting_Human_0029\| | Non_Targeting_Human | GCACACCTAGATATCGAATG |
| | Non_Targeting_Human | | |
| 463 | 1\|sg_Non_Targeting_Human_0030\| | Non_Targeting_Human | GTTGATCAACGCGCTTCGCG |
| | Non_Targeting_Human | | |
| 464 | 1\|sg_Non_Targeting_Human_0031\| | Non_Targeting_Human | GCGTCTCACTCACTCCATCG |
| | Non_Targeting_Human | | |
| 465 | 1\|sg_Non_Targeting_Human_0032\| | Non_Targeting_Human | GCCGACCAACGTCAGCGGTA |
| | Non_Targeting_Human | | |
| 466 | 1\|sg_Non_Targeting_Human_0033\| | Non_Targeting_Human | GGATACGGTGCGTCAATCTA |
| | Non_Targeting_Human | | |
| 467 | 1\|sg_Non_Targeting_Human_0034\| | Non_Targeting_Human | GAATCCAGTGGCGGCGACAA |
| | Non_Targeting_Human | | |
| 468 | 1\|sg_Non_Targeting_Human_0035\| | Non_Targeting_Human | GCACTGTCAGTGCAACGATA |
| | Non_Targeting_Human | | |
| 469 | 1\|sg_Non_Targeting_Human_0036\| | Non_Targeting_Human | GCGATCCTCAAGTATGCTCA |
| | Non_Targeting_Human | | |
| 470 | 1\|sg_Non_Targeting_Human_0037\| | Non_Targeting_Human | GCTAATATCGACACGGCCGC |
| | Non_Targeting_Human | | |
| 471 | 1\|sg_Non_Targeting_Human_0038\| | Non_Targeting_Human | GGAGATGCATCGAAGTCGAT |
| | Non_Targeting_Human | | |
| 472 | 1\|sg_Non_Targeting_Human_0039\| | Non_Targeting_Human | GGATGCACTCCATCTCGTCT |
| | Non_Targeting_Human | | |
| 473 | 1\|sg_Non_Targeting_Human_0040\| | Non_Targeting_Human | GTGCCGAGTAATAACGCGAG |
| | Non_Targeting_Human | | |
| 474 | 1\|sg_Non_Targeting_Human_0041\| | Non_Targeting_Human | GAGATTCCGATGTAACGTAC |
| | Non_Targeting_Human | | |
| 475 | 1\|sg_Non_Targeting_Human_0042\| | Non_Targeting_Human | GTCGTCACGAGCAGGATTGC |
| | Non_Targeting_Human | | |
| 476 | 1\|sg_Non_Targeting_Human_0043\| | Non_Targeting_Human | GCGTTAGTCACTTAGCTCGA |
| | Non_Targeting_Human | | |
| 477 | 1\|sg_Non_Targeting_Human_0044\| | Non_Targeting_Human | GTTCACACGGTGTCGGATAG |
| | Non_Targeting_Human | | |
| 478 | 1\|sg_Non_Targeting_Human_0045\| | Non_Targeting_Human | GGATAGGTGACCTTAGTACG |
| | Non_Targeting_Human | | |
| 479 | 1\|sg_Non_Targeting_Human_0046\| | Non_Targeting_Human | GTATGAGTCAAGCTAATGCG |
| | Non_Targeting_Human | | |
| 480 | 1\|sg_Non_Targeting_Human_0047\| | Non_Targeting_Human | GCAACTATTGGAATACGTGA |
| | Non_Targeting_Human | | |
| 481 | 1\|sg_Non_Targeting_Human_0048\| | Non_Targeting_Human | GTTACCTTCGCTCGTCTATA |
| | Non_Targeting_Human | | |
| 482 | 1\|sg_Non_Targeting_Human_0049\| | Non_Targeting_Human | GTACCGAGCACCACAGGCCG |
| | Non_Targeting_Human | | |
| 483 | 1\|sg_Non_Targeting_Human_0050\| | Non_Targeting_Human | GTCAGCCATCGGATAGAGAT |
| | Non_Targeting_Human | | |
| 484 | 1\|sg_Non_Targeting_Human_0051\| | Non_Targeting_Human | GTACGGCACTCCTAGCCGCT |
| | Non_Targeting_Human | | |
| 485 | 1\|sg_Non_Targeting_Human_0052\| | Non_Targeting_Human | GGTCCTGTCGTATGCTTGCA |
| | Non_Targeting_Human | | |
| 486 | 1\|sg_Non_Targeting_Human_0053\| | Non_Targeting_Human | GCCGCAATATATGCGGTAAG |
| | Non_Targeting_Human | | |
| 487 | 1\|sg_Non_Targeting_Human_0054\| | Non_Targeting_Human | GCGCACGTATAATCCTGCGT |
| | Non_Targeting_Human | | |
| 488 | 1\|sg_Non_Targeting_Human_0055\| | Non_Targeting_Human | GTGCACAACACGATCCACGA |
| | Non_Targeting_Human | | |
| 489 | 1\|sg_Non_Targeting_Human_0056\| | Non_Targeting_Human | GCACAATGTTGACGTAAGTG |
| | Non_Targeting_Human | | |
| 490 | 1\|sg_Non_Targeting_Human_0057\| | Non_Targeting_Human | GTAAGATGCTGCTCACCGTG |
| | Non_Targeting_Human | | |
| 491 | 1\|sg_Non_Targeting_Human_0058\| | Non_Targeting_Human | GTCGGTGATCCAACGTATCG |
| | Non_Targeting_Human | | |
| 492 | 1\|sg_Non_Targeting_Human_0059\| | Non_Targeting_Human | GAGCTAGTAGGACGCAAGAC |
| | Non_Targeting_Human | | |
| 493 | 1\|sg_Non_Targeting_Human_0060\| | Non_Targeting_Human | GTACGTGGAAGCTTGTGGCC |
| | Non_Targeting_Human | | |
| 494 | 1\|sg_Non_Targeting_Human_0061\| | Non_Targeting_Human | GAGAACTGCCAGTTCTCGAT |
| | Non_Targeting_Human | | |
| 495 | 1\|sg_Non_Targeting_Human_0062\| | Non_Targeting_Human | GCCATTCGGCGCGGCACTTC |
| | Non_Targeting_Human | | |
| 496 | 1\|sg_Non_Targeting_Human_0063\| | Non_Targeting_Human | GCACACGACCAATCCGCTTC |
| | Non_Targeting_Human | | |
| 497 | 1\|sg_Non_Targeting_Human_0064\| | Non_Targeting_Human | GAGGTGATCGATTAAGTACA |
| | Non_Targeting_Human | | |
| 498 | 1\|sg_Non_Targeting_Human_0065\| | Non_Targeting_Human | GTCACTCGCAGACGCCTAAC |
| | Non_Targeting_Human | | |
| 499 | 1\|sg_Non_Targeting_Human_0066\| | Non_Targeting_Human | GCGCTACGGAATCATACGTT |
| | Non_Targeting_Human | | |
| 500 | 1\|sg_Non_Targeting_Human_0067\| | Non_Targeting_Human | GGTAGGACCTCACGGCGCGC |
| | Non_Targeting_Human | | |
| 501 | 1\|sg_Non_Targeting_Human_0068\| | Non_Targeting_Human | GAACTGCATCTTGTTGTAGT |
| | Non_Targeting_Human | | |
| 502 | 1\|sg_Non_Targeting_Human_0069\| | Non_Targeting_Human | GATCCTGATCCGGCGGCGCG |
| | Non_Targeting_Human | | |
| 503 | 1\|sg_Non_Targeting_Human_0070\| | Non_Targeting_Human | GGTATGCGCGATCCTGAGTT |
| | Non_Targeting_Human | | |
| 504 | 1\|sg_Non_Targeting_Human_0071\| | Non_Targeting_Human | GCGGAGCTAGAGAGCGGTCA |
| | Non_Targeting_Human | | |
| 505 | 1\|sg_Non_Targeting_Human_0072\| | Non_Targeting_Human | GAATGGCAATTACGGCTGAT |
| | Non_Targeting_Human | | |
| 506 | 1\|sg_Non_Targeting_Human_0073\| | Non_Targeting_Human | GTATGGTGAGTAGTCGCTTG |
| | Non_Targeting_Human | | |
| 507 | 1\|sg_Non_Targeting_Human_0074\| | Non_Targeting_Human | GTGTAATTGCGTCTAGTCGG |
| | Non_Targeting_Human | | |
| 508 | 1\|sg_Non_Targeting_Human_0075\| | Non_Targeting_Human | GGTCCTGGCGAGGAGCCTTG |
| | Non_Targeting_Human | | |
| 509 | 1\|sg_Non_Targeting_Human_0076\| | Non_Targeting_Human | GAAGATAAGTCGCTGTCTCG |
| | Non_Targeting_Human | | |
| 510 | 1\|sg_Non_Targeting_Human_0077\| | Non_Targeting_Human | GTCGGCGTTCTGTTGTGACT |
| | Non_Targeting_Human | | |
| 511 | 1\|sg_Non_Targeting_Human_0078\| | Non_Targeting_Human | GAGGCAAGCCGTTAGGTGTA |
| | Non_Targeting_Human | | |
| 512 | 1\|sg_Non_Targeting_Human_0079\| | Non_Targeting_Human | GCGGATCCAGATCTCATTCG |
| | Non_Targeting_Human | | |
| 513 | 1\|sg_Non_Targeting_Human_0080\| | Non_Targeting_Human | GGAACATAGGAGCACGTAGT |
| | Non_Targeting_Human | | |
| 514 | 1\|sg_Non_Targeting_Human_0081\| | Non_Targeting_Human | GTCATCATTATGGCGTAAGG |
| | Non_Targeting_Human | | |
| 515 | 1\|sg_Non_Targeting_Human_0082\| | Non_Targeting_Human | GCGACTAGCGCCATGAGCGG |
| | Non_Targeting_Human | | |
| 516 | 1\|sg_Non_Targeting_Human_0083\| | Non_Targeting_Human | GGCGAAGTTCGACATGACAC |
| | Non_Targeting_Human | | |
| 517 | 1\|sg_Non_Targeting_Human_0084\| | Non_Targeting_Human | GCTGTCGTGTGGAGGCTATG |
| | Non_Targeting_Human | | |
| 518 | 1\|sg_Non_Targeting_Human_0085\| | Non_Targeting_Human | GCGGAGAGCATTGACCTCAT |
| | Non_Targeting_Human | | |
| 519 | 1\|sg_Non_Targeting_Human_0086\| | Non_Targeting_Human | GACTAATGGACCAAGTCAGT |
| | Non_Targeting_Human | | |
| 520 | 1\|sg_Non_Targeting_Human_0087\| | Non_Targeting_Human | GCGGATTAGAGGTAATGCGG |
| | Non_Targeting_Human | | |
| 521 | 1\|sg_Non_Targeting_Human_0088\| | Non_Targeting_Human | GCCGACGGCAATCAGTACGC |
| | Non_Targeting_Human | | |
| 522 | 1\|sg_Non_Targeting_Human_0089\| | Non_Targeting_Human | GTAACCTCTCGAGCGATAGA |
| | Non_Targeting_Human | | |
| 523 | 1\|sg_Non_Targeting_Human_0090\| | Non_Targeting_Human | GACTTGTATGTGGCTTACGG |
| | Non_Targeting_Human | | |
| 524 | 1\|sg_Non_Targeting_Human_0091\| | Non_Targeting_Human | GTCACTGTGGTCGAACATGT |
| | Non_Targeting_Human | | |
| 525 | 1\|sg_Non_Targeting_Human_0092\| | Non_Targeting_Human | GTACTCCAATCCGCGATGAC |
| | Non_Targeting_Human | | |
| 526 | 1\|sg_Non_Targeting_Human_0093\| | Non_Targeting_Human | GCGTTGGCACGATGTTACGG |
| | Non_Targeting_Human | | |
| 527 | 1\|sg_Non_Targeting_Human_0094\| | Non_Targeting_Human | GAACCAGCCGGCTAGTATGA |
| | Non_Targeting_Human | | |
| 528 | 1\|sg_Non_Targeting_Human_0095\| | Non_Targeting_Human | GTATACTAGCTAACCACACG |
| | Non_Targeting_Human | | |
| 529 | 1\|sg_Non_Targeting_Human_0096\| | Non_Targeting_Human | GAATCGGAATAGTTGATTCG |
| | Non_Targeting_Human | | |
| 530 | 1\|sg_Non_Targeting_Human_0097\| | Non_Targeting_Human | GAGCACTTGCATGAGGCGGT |
| | Non_Targeting_Human | | |
| 531 | 1\|sg_Non_Targeting_Human_0098\| | Non_Targeting_Human | GAACGGCGATGAAGCCAGCC |
| | Non_Targeting_Human | | |
| 532 | 1\|sg_Non_Targeting_Human_0099\| | Non_Targeting_Human | GCAACCGAGATGAGAGGTTC |
| | Non_Targeting_Human | | |
| 533 | 1\|sg_Non_Targeting_Human_0100\| | Non_Targeting_Human | GCAAGATCAATATGCGTGAT |
| | Non_Targeting_Human | | |
| 534 | 1\|sg_Non_Targeting_Human_GA_0101\| | Non_Targeting_Human | ACGGAGGCTAAGCGTCGCAA |
| | Non_Targeting_Human | | |
| 535 | 1\|sg_Non_Targeting_Human_GA_0102\| | Non_Targeting_Human | CGCTTCCGCGGCCCGTTCAA |
| | Non_Targeting_Human | | |
| 536 | 1\|sg_Non_Targeting_Human_GA_0103\| | Non_Targeting_Human | ATCGTTTCCGCTTAACGGCG |
| | Non_Targeting_Human | | |
| 537 | 1\|sg_Non_Targeting_Human_GA_0104\| | Non_Targeting_Human | GTAGGCGCGCCGCTCTCTAC |
| | Non_Targeting_Human | | |
| 538 | 1\|sg_Non_Targeting_Human_GA_0105\| | Non_Targeting_Human | CCATATCGGGGCGAGACATG |
| | Non_Targeting_Human | | |
| 539 | 1\|sg_Non_Targeting_Human_GA_0106\| | Non_Targeting_Human | TACTAACGCCGCTCCTACAG |
| | Non_Targeting_Human | | |
| 540 | 1\|sg_Non_Targeting_Human_GA_0107\| | Non_Targeting_Human | TGAGGATCATGTCGAGCGCC |
| | Non_Targeting_Human | | |
| 541 | 1\|sg_Non_Targeting_Human_GA_0108\| | Non_Targeting_Human | GGGCCCGCATAGGATATCGC |
| | Non_Targeting_Human | | |
| 542 | 1\|sg_Non_Targeting_Human_GA_0109\| | Non_Targeting_Human | TAGACAACCGCGGAGAATGC |
| | Non_Targeting_Human | | |
| 543 | 1\|sg_Non_Targeting_Human_GA_0110\| | Non_Targeting_Human | ACGGGCGGCTATCGCTGACT |
| | Non_Targeting_Human | | |
| 544 | 1\|sg_Non_Targeting_Human_GA_0111\| | Non_Targeting_Human | CGCGGAAATTTTACCGACGA |
| | Non_Targeting_Human | | |
| 545 | 1\|sg_Non_Targeting_Human_GA_0112\| | Non_Targeting_Human | CTTACAATCGTCGGTCCAAT |
| | Non_Targeting_Human | | |
| 546 | 1\|sg_Non_Targeting_Human_GA_0113\| | Non_Targeting_Human | GCGTGCGTCCCGGGTTACCC |
| | Non_Targeting_Human | | |
| 547 | 1\|sg_Non_Targeting_Human_GA_0114\| | Non_Targeting_Human | CGGAGTAACAAGCGGACGGA |
| | Non_Targeting_Human | | |
| 548 | 1\|sg_Non_Targeting_Human_GA_0115\| | Non_Targeting_Human | CGAGTGTTATACGCACCGTT |
| | Non_Targeting_Human | | |
| 549 | 1\|sg_Non_Targeting_Human_GA_0116\| | Non_Targeting_Human | CGACTAACCGGAAACTTTTT |
| | Non_Targeting_Human | | |
| 550 | 1\|sg_Non_Targeting_Human_GA_0117\| | Non_Targeting_Human | CAACGGGTTCTCCCGGCTAC |
| | Non_Targeting_Human | | |
| 551 | 1\|sg_Non_Targeting_Human_GA_0118\| | Non_Targeting_Human | CAGGAGTCGCCGATACGCGT |
| | Non_Targeting_Human | | |
| 552 | 1\|sg_Non_Targeting_Human_GA_0119\| | Non_Targeting_Human | TTCACGTCGTCTCGCGACCA |
| | Non_Targeting_Human | | |
| 553 | 1\|sg_Non_Targeting_Human_GA_0120\| | Non_Targeting_Human | GTGTCGGATTCCGCCGCTTA |
| | Non_Targeting_Human | | |
| 554 | 1\|sg_Non_Targeting_Human_GA_0121\| | Non_Targeting_Human | CACGAACTCACACCGCGCGA |
| | Non_Targeting_Human | | |
| 555 | 1\|sg_Non_Targeting_Human_GA_0122\| | Non_Targeting_Human | CGCTAGTACGCTCCTCTATA |
| | Non_Targeting_Human | | |
| 556 | 1\|sg_Non_Targeting_Human_GA_0123\| | Non_Targeting_Human | TCGCGCTTGGGTTATACGCT |
| | Non_Targeting_Human | | |
| 557 | 1\|sg_Non_Targeting_Human_GA_0124\| | Non_Targeting_Human | CTATCTCGAGTGGTAATGCG |
| | Non_Targeting_Human | | |
| 558 | 1\|sg_Non_Targeting_Human_GA_0125\| | Non_Targeting_Human | AATCGACTCGAACTTCGTGT |
| | Non_Targeting_Human | | |
| 559 | 1\|sg_Non_Targeting_Human_GA_0126\| | Non_Targeting_Human | CCCGATGGACTATACCGAAC |
| | Non_Targeting_Human | | |
| 560 | 1\|sg_Non_Targeting_Human_GA_0127\| | Non_Targeting_Human | ACGTTCGAGTACGACCAGCT |
| | Non_Targeting_Human | | |
| 561 | 1\|sg_Non_Targeting_Human_GA_0128\| | Non_Targeting_Human | CGCGACGACTCAACCTAGTC |
| | Non_Targeting_Human | | |
| 562 | 1\|sg_Non_Targeting_Human_GA_0129\| | Non_Targeting_Human | GGTCACCGATCGAGAGCTAG |
| | Non_Targeting_Human | | |
| 563 | 1\|sg_Non_Targeting_Human_GA_0130\| | Non_Targeting_Human | CTCAACCGACCGTATGGTCA |
| | Non_Targeting_Human | | |
| 564 | 1\|sg_Non_Targeting_Human_GA_0131\| | Non_Targeting_Human | CGTATTCGACTCTCAACGCG |
| | Non_Targeting_Human | | |
| 565 | 1\|sg_Non_Targeting_Human_GA_0132\| | Non_Targeting_Human | CTAGCCGCCCAGATCGAGCC |
| | Non_Targeting_Human | | |
| 566 | 1\|sg_Non_Targeting_Human_GA_0133\| | Non_Targeting_Human | GAATCGACCGACACTAATGT |
| | Non_Targeting_Human | | |
| 567 | 1\|sg_Non_Targeting_Human_GA_0134\| | Non_Targeting_Human | ACTTCAGTTCGGCGTAGTCA |
| | Non_Targeting_Human | | |
| 568 | 1\|sg_Non_Targeting_Human_GA_0135\| | Non_Targeting_Human | GTGCGATGTCGCTTCAACGT |
| | Non_Targeting_Human | | |
| 569 | 1\|sg_Non_Targeting_Human_GA_0136\| | Non_Targeting_Human | CGCCTAATTTCCGGATCAAT |
| | Non_Targeting_Human | | |
| 570 | 1\|sg_Non_Targeting_Human_GA_0137\| | Non_Targeting_Human | CGTGGCCGGAACCGTCATAG |
| | Non_Targeting_Human | | |
| 571 | 1\|sg_Non_Targeting_Human_GA_0138\| | Non_Targeting_Human | ACCCTCCGAATCGTAACGGA |
| | Non_Targeting_Human | | |
| 572 | 1\|sg_Non_Targeting_Human_GA_0139\| | Non_Targeting_Human | AAACGGTACGACAGCGTGTG |
| | Non_Targeting_Human | | |
| 573 | 1\|sg_Non_Targeting_Human_GA_0140\| | Non_Targeting_Human | ACATAGTCGACGGCTCGATT |
| | Non_Targeting_Human | | |
| 574 | 1\|sg_Non_Targeting_Human_GA_0141\| | Non_Targeting_Human | GATGGCGCTTCAGTCGTCGG |
| | Non_Targeting_Human | | |
| 575 | 1\|sg_Non_Targeting_Human_GA_0142\| | Non_Targeting_Human | ATAATCCGGAAACGCTCGAC |
| | Non_Targeting_Human | | |
| 576 | 1\|sg_Non_Targeting_Human_GA_0143\| | Non_Targeting_Human | CGCCGGGCTGACAATTAACG |
| | Non_Targeting_Human | | |
| 577 | 1\|sg_Non_Targeting_Human_GA_0144\| | Non_Targeting_Human | CGTCGCCATATGCCGGTGGC |
| | Non_Targeting_Human | | |
| 578 | 1\|sg_Non_Targeting_Human_GA_0145\| | Non_Targeting_Human | CGGGCCTATAACACCATCGA |
| | Non_Targeting_Human | | |
| 579 | 1\|sg_Non_Targeting_Human_GA_0146\| | Non_Targeting_Human | CGCCGTTCCGAGATACTTGA |
| | Non_Targeting_Human | | |
| 580 | 1\|sg_Non_Targeting_Human_GA_0147\| | Non_Targeting_Human | CGGGACGTCGCGAAAATGTA |
| | Non_Targeting_Human | | |
| 581 | 1\|sg_Non_Targeting_Human_GA_0148\| | Non_Targeting_Human | TCGGCATACGGGACACACGC |
| | Non_Targeting_Human | | |
| 582 | 1\|sg_Non_Targeting_Human_GA_0149\| | Non_Targeting_Human | AGCTCCATCGCCGCGATAAT |
| | Non_Targeting_Human | | |
| 583 | 1\|sg_Non_Targeting_Human_GA_0150\| | Non_Targeting_Human | ATCGTATCATCAGCTAGCGC |
| | Non_Targeting_Human | | |
| 584 | 1\|sg_Non_Targeting_Human_GA_0151\| | Non_Targeting_Human | TCGATCGAGGTTGCATTCGG |
| | Non_Targeting_Human | | |
| 585 | 1\|sg_Non_Targeting_Human_GA_0152\| | Non_Targeting_Human | CTCGACAGTTCGTCCCGAGC |
| | Non_Targeting_Human | | |
| 586 | 1\|sg_Non_Targeting_Human_GA_0153\| | Non_Targeting_Human | CGGTAGTATTAATCGCTGAC |
| | Non_Targeting_Human | | |
| 587 | 1\|sg_Non_Targeting_Human_GA_0154\| | Non_Targeting_Human | TGAACGCGTGTTTCCTTGCA |
| | Non_Targeting_Human | | |
| 588 | 1\|sg_Non_Targeting_Human_GA_0155\| | Non_Targeting_Human | CGACGCTAGGTAACGTAGAG |
| | Non_Targeting_Human | | |
| 589 | 1\|sg_Non_Targeting_Human_GA_0156\| | Non_Targeting_Human | CATTGTTGAGCGGGCGCGCT |
| | Non_Targeting_Human | | |
| 590 | 1\|sg_Non_Targeting_Human_GA_0157\| | Non_Targeting_Human | CCGCTATTGAAACCGCCCAC |
| | Non_Targeting_Human | | |
| 591 | 1\|sg_Non_Targeting_Human_GA_0158\| | Non_Targeting_Human | AGACACGTCACCGGTCAAAA |
| | Non_Targeting_Human | | |
| 592 | 1\|sg_Non_Targeting_Human_GA_0159\| | Non_Targeting_Human | TTTACGATCTAGCGGCGTAG |
| | Non_Targeting_Human | | |
| 593 | 1\|sg_Non_Targeting_Human_GA_0160\| | Non_Targeting_Human | TTCGCACGATTGCACCTTGG |
| | Non_Targeting_Human | | |
| 594 | 1\|sg_Non_Targeting_Human_GA_0161\| | Non_Targeting_Human | GGTTAGAGACTAGGCGCGCG |
| | Non_Targeting_Human | | |
| 595 | 1\|sg_Non_Targeting_Human_GA_0162\| | Non_Targeting_Human | CCTCCGTGCTAACGCGGACG |
| | Non_Targeting_Human | | |
| 596 | 1\|sg_Non_Targeting_Human_GA_0163\| | Non_Targeting_Human | TTATCGCGTAGTGCTGACGT |
| | Non_Targeting_Human | | |
| 597 | 1\|sg_Non_Targeting_Human_GA_0164\| | Non_Targeting_Human | TACGCTTGCGTTTAGCGTCC |
| | Non_Targeting_Human | | |
| 598 | 1\|sg_Non_Targeting_Human_GA_0165\| | Non_Targeting_Human | CGCGGCCCACGCGTCATCGC |
| | Non_Targeting_Human | | |
| 599 | 1\|sg_Non_Targeting_Human_GA_0166\| | Non_Targeting_Human | AGCTCGCCATGTCGGTTCTC |
| | Non_Targeting_Human | | |
| 600 | 1\|sg_Non_Targeting_Human_GA_0167\| | Non_Targeting_Human | AACTAGCCCGAGCAGCTTCG |
| | Non_Targeting_Human | | |
| 601 | 1\|sg_Non_Targeting_Human_GA_0168\| | Non_Targeting_Human | CGCAAGGTGTCGGTAACCCT |
| | Non_Targeting_Human | | |
| 602 | 1\|sg_Non_Targeting_Human_GA_0169\| | Non_Targeting_Human | CTTCGACGCCATCGTGCTCA |
| | Non_Targeting_Human | | |
| 603 | 1\|sg_Non_Targeting_Human_GA_0170\| | Non_Targeting_Human | TCCTGGATACCGCGTGGTTA |
| | Non_Targeting_Human | | |
| 604 | 1\|sg_Non_Targeting_Human_GA_0171\| | Non_Targeting_Human | ATAGCCGCCGCTCATTACTT |
| | Non_Targeting_Human | | |
| 605 | 1\|sg_Non_Targeting_Human_GA_0172\| | Non_Targeting_Human | GTCGTCCGGGATTACAAAAT |
| | Non_Targeting_Human | | |
| 606 | 1\|sg_Non_Targeting_Human_GA_0173\| | Non_Targeting_Human | TAATGCTGCACACGCCGAAT |
| | Non_Targeting_Human | | |
| 607 | 1\|sg_Non_Targeting_Human_GA_0174\| | Non_Targeting_Human | TATCGCTTCCGATTAGTCCG |
| | Non_Targeting_Human | | |
| 608 | 1\|sg_Non_Targeting_Human_GA_0175\| | Non_Targeting_Human | GTACCATACCGCGTACCCTT |
| | Non_Targeting_Human | | |
| 609 | 1\|sg_Non_Targeting_Human_GA_0176\| | Non_Targeting_Human | TAAGATCCGCGGGTGGCAAC |
| | Non_Targeting_Human | | |
| 610 | 1\|sg_Non_Targeting_Human_GA_0177\| | Non_Targeting_Human | GTAGACGTCGTGAGCTTCAC |
| | Non_Targeting_Human | | |
| 611 | 1\|sg_Non_Targeting_Human_GA_0178\| | Non_Targeting_Human | TCGCGGACATAGGGCTCTAA |
| | Non_Targeting_Human | | |
| 612 | 1\|sg_Non_Targeting_Human_GA_0179\| | Non_Targeting_Human | AGCGCAGATAGCGCGTATCA |
| | Non_Targeting_Human | | |
| 613 | 1\|sg_Non_Targeting_uman_GA_0180\| | Non_Targeting_Human | GTTCGCTTCGTAACGAGGAA |
| | HNon_Targeting_Human | | |
| 614 | 1\|sg_Non_Targeting_Human_GA_0181\| | Non_Targeting_Human | GACCCCCGATAACTTTTGAC |
| | Non_Targeting_Human | | |
| 615 | 1\|sg_Non_Targeting_Human_GA_0182\| | Non_Targeting_Human | ACGTCCATACTGTCGGCTAC |
| | Non_Targeting_Human | | |
| 616 | 1\|sg_Non_Targeting_Human_GA_0183\| | Non_Targeting_Human | GTACCATTGCCGGCTCCCTA |
| | Non_Targeting_Human | | |
| 617 | 1\|sg_Non_Targeting_Human_GA_0184\| | Non_Targeting_Human | TGGTTCCGTAGGTCGGTATA |
| | Non_Targeting_Human | | |
| 618 | 1\|sg_Non_Targeting_Human_GA_0185\| | Non_Targeting_Human | TCTGGCTTGACACGACCGTT |
| | Non_Targeting_Human | | |
| 619 | 1\|sg_Non_Targeting_Human_GA_0186\| | Non_Targeting_Human | CGCTAGGTCCGGTAAGTGCG |
| | Non_Targeting_Human | | |
| 620 | 1\|sg_Non_Targeting_Human_GA_0187\| | Non_Targeting_Human | AGCACGTAATGTCCGTGGAT |
| | Non_Targeting_Human | | |
| 621 | 1\|sg_Non_Targeting_Human_GA_0188\| | Non_Targeting_Human | AAGGCGCGCGAATGTGGCAG |
| | Non_Targeting_Human | | |
| 622 | 1\|sg_Non_Targeting_Human_GA_0189\| | Non_Targeting_Human | ACTGCGGAGCGCCCAATATC |
| | Non_Targeting_Human | | |
| 623 | 1\|sg_Non_Targeting_Human_GA_0190\| | Non_Targeting_Human | CGTCGAGTGCTCGAACTCCA |
| | Non_Targeting_Human | | |
| 624 | 1\|sg_Non_Targeting_Human_GA_0191\| | Non_Targeting_Human | TCGCAGCGGCGTGGGATCGG |
| | Non_Targeting_Human | | |
| 625 | 1\|sg_Non_Targeting_Human_GA_0192\| | Non_Targeting_Human | ATCTGTCCTAATTCGGATCG |
| | Non_Targeting_Human | | |
| 626 | 1\|sg_Non_Targeting_Human_GA_0193\| | Non_Targeting_Human | TGCGGCGTAATGCTTGAAAG |
| | Non_Targeting_Human | | |
| 627 | 1\|sg_Non_Targeting_Human_GA_0194\| | Non_Targeting_Human | CGAACTTAATCCCGTGGCAA |
| | Non_Targeting_Human | | |
| 628 | 1\|sg_Non_Targeting_Human_GA_0195\| | Non_Targeting_Human | GCCGTGTTGCTGGATACGCC |
| | Non_Targeting_Human | | |
| 629 | 1\|sg_Non_Targeting_Human_GA_0196\| | Non_Targeting_Human | TACCCTCCGGATACGGACTG |
| | Non_Targeting_Human | | |
| 630 | 1\|sg_Non_Targeting_Human_GA_0197\| | Non_Targeting_Human | CCGTTGGACTATGGCGGGTC |
| | Non_Targeting_Human | | |
| 631 | 1\|sg_Non_Targeting_Human_GA_0198\| | Non_Targeting_Human | GTACGGGGCGATCATCCACA |
| | Non_Targeting_Human | | |
| 632 | 1\|sg_Non_Targeting_Human_GA_0199\| | Non_Targeting_Human | AAGAGTAGTAGACGCCCGGG |
| | Non_Targeting_Human | | |
| 633 | 1\|sg_Non_Targeting_Human_GA_0200\| | Non_Targeting_Human | AAGAGCGAATCGATTTCGTG |
| | Non_Targeting_Human | | |
| 634 | 3\|sg_hCDC16_CC_1\|CDC16 | CDC16 | TCAACACCAGTGCCTGACGG |
| 635 | 3\|sg_hCDC16_CC_2\|CDC16 | CDC16 | AAAGTAGCTTCACTCTCTCG |
| 636 | 3\|sg_hCDC16_CC_3\|CDC16 | CDC16 | GAGCCAACCAATAGATGTCC |
| 637 | 3\|sg_hCDC16_CC_4\|CDC16 | CDC16 | GCGCCGCCATGAACCTAGAG |
| 638 | 3\|sg_hGTF2B_CC_1\|GTF2B | GTF2B | ACAAAGGTTGGAACAGAACC |
| 639 | 3\|sg_hGTF2B_CC_2\|GTF2B | GTF2B | GGTGACCGGGTTATTGATGT |
| 640 | 3\|sg_hGTF2B_CC_3\|GTF2B | GTF2B | TTAGTGGAGGACTACAGAGC |
| 641 | 3\|sg_hGTF2B_CC_4\|GTF2B | GTF2B | ACATATAGCCCGTAAAGCTG |
| 642 | 3\|sg_hHSPA5_CC_1\|HSPA5 | HSPA5 | CGTTGGCGATGATCTCCACG |
| 643 | 3\|sg_hHSPA5_CC_2\|HSPA5 | HSPA5 | TGGCCTTTTCTACCTCGCGC |
| 644 | 3\|sg_hHSPA5_CC_3\|HSPA5 | HSPA5 | AATGGAGATACTCATCTGGG |
| 645 | 3\|sg_hHSPA5_CC_4\|HSPA5 | HSPA5 | GAAGCCCGTCCAGAAAGTGT |
| 646 | 3\|sg_hHSPA9_CC_1\|HSPA9 | HSPA9 | CAATCTGAGGAACTCCACGA |
| 647 | 3\|sg_hHSPA9_CC_2\|HSPA9 | HSPA9 | AGGCTGCGGCGCCCACGAGA |
| 648 | 3\|sg_hHSPA9_CC_3\|HSPA9 | HSPA9 | ACTTTGACCAGGCCTTGCTA |
| 649 | 3\|sg_hHSPA9_CC_4\|HSPA9 | HSPA9 | ACCTTCCATAACTGCCACGC |
| 650 | 3\|sg_hPAFAH1B1_CC_1\|PAFAH1B1 | PAFAH1B1 | CGAGGCGTACATACCCAAGG |
| 651 | 3\|sg_hPAFAH1B1_CC_2\|PAFAH1B1 | PAFAH1B1 | ATGGTACGGCCAAATCAAGA |
| 652 | 3\|sg_hPAFAH1B1_CC_3\|PAFAH1B1 | PAFAH1B1 | TCTTGTAATCCCATACGCGT |
| 653 | 3\|sg_hPAFAH1B1_CC_4\|PAFAH1B1 | PAFAH1B1 | ATTCACAGGACACAGAGAAT |
| 654 | 3\|sg_hPCNA_CC_1\|PCNA | PCNA | CCAGGGCTCCATCCTCAAGA |
| 655 | 3\|sg_hPCNA_CC_2\|PCNA | PCNA | TGAGCTGCACCAAAGAGACG |
| 656 | 3\|sg_hPCNA_CC_3\|PCNA | PCNA | ATGTCTGCAGATGTACCCCT |
| 657 | 3\|sg_hPCNA_CC_4\|PCNA | PCNA | CGAAGATAACGCGGATACCT |
| 658 | 3\|sg_hPOLR2L_CC_1\|POLR2L | POLR2L | GCTGCAGGCCGAGTACACCG |
| 659 | 3\|sg_hPOLR2L_CC_2\|POLR2L | POLR2L | ACAAGTGGGAGGCTTACCTG |
| 660 | 3\|sg_hPOLR2L_CC_3\|POLR2L | POLR2L | GCAGCGTACAGGGATGATCA |
| 661 | 3\|sg_hPOLR2L_CC_4\|POLR2L | POLR2L | GCAGTAGCGCTTCAGGCCCA |
| 662 | 3\|sg_hRPL9_CC_1\|RPL9 | RPL9 | CAAATGGTGGGGTAACAGAA |
| 663 | 3\|sg_hRPL9_CC_2\|RPL9 | RPL9 | GAAAGGAACTGGCTACCGTT |
| 664 | 3\|sg_hRPL9_CC_3\|RPL9 | RPL9 | AGGGCTTCCGTTACAAGATG |
| 665 | 3\|sg_hRPL9_CC_4\|RPL9 | RPL9 | GAACAAGCAACACCTAAAAG |
| 666 | 3\|sg_hSF3A3_CC_1\|SF3A3 | SF3A3 | TGAGGAGAAGGAACGGCTCA |
| 667 | 3\|sg_hSF3A3_CC_2\|SF3A3 | SF3A3 | GGAAGAATGCAGAGTATAAG |
| 668 | 3\|sg_hSF3A3_CC_3\|SF3A3 | SF3A3 | GGAATTTGAGGAACTCCTGA |
| 669 | 3\|sg_hSF3A3_CC_4\|SF3A3 | SF3A3 | GCTCACCGGCCATCCAGGAA |
| 670 | 3\|sg_hSF3B3_CC_1\|SF3B3 | SF3B3 | ACTGGCCAGGAACGATGCGA |
| 671 | 3\|sg_hSF3B3_CC_2\|SF3B3 | SF3B3 | GCAGCTCCAAGATCTTCCCA |
| 672 | 3\|sg_hSF3B3_CC_3\|SF3B3 | SF3B3 | GAATGAGTACACAGAACGGA |
| 673 | 3\|sg_hSF3B3_CC_4\|SF3B3 | SF3B3 | GGAGCAGGACAAGGTCGGGG |

### Example 2—BRD9 Degrader Depletes BRD9 Protein

The following example demonstrates the depletion of the BRD9 protein in synovial sarcoma cells treated with a BRD9 degrader.

Procedure: Cells were treated with DMSO or the BRD9 degrader, Compound A (also known as dBRD9, see Remillard et al, Angew. Chem. Int. Ed. Engl. 56(21):5738-5743 (2017); see structure of Compound 1 below), for indicated doses and timepoints.

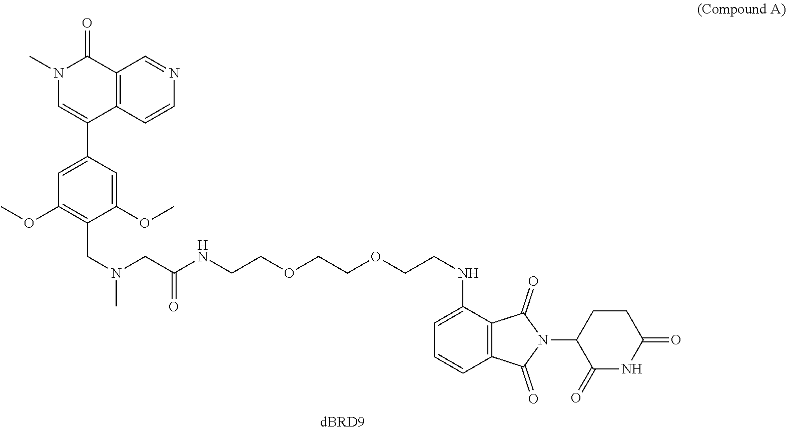 [SMILES: COc1cc(-c2cn(C)c(=O)c3cnccc23)cc(OC)c1CN(C)CC(=O)NCCOCCOCCNc1cccc2c1C(=O)N(C1CCC(=O)NC1=O)C2=O]

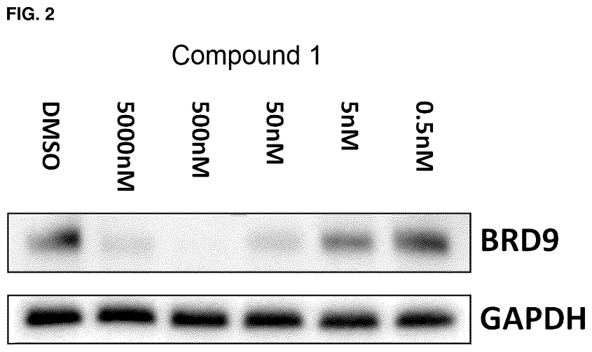

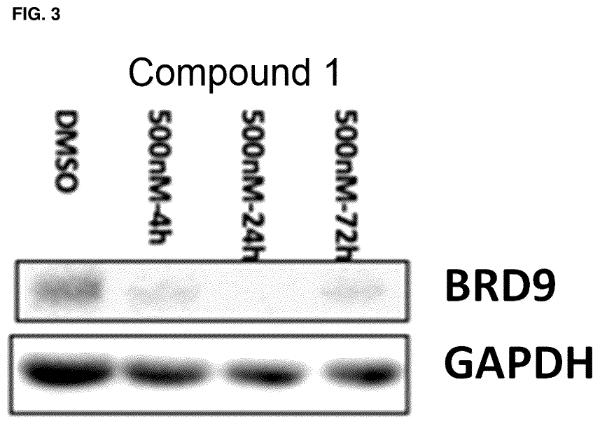

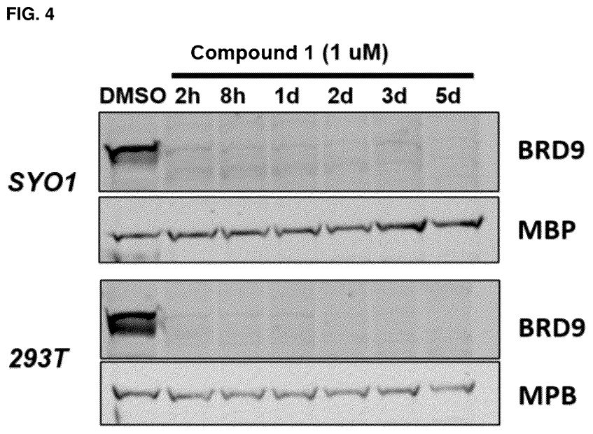

Whole cell extracts were fractionated by SDS-PAGE and transferred to a polyvinylidene difluoride membrane using a transfer apparatus according to the manufacturer's protocols (Bio-Rad). After incubation with 5% nonfat milk in TBST (10 mM Tris, pH 8.0, 150 mM NaCl, 0.5% Tween 20) for 60 minutes, the membrane was incubated with antibodies against BRD9 (1:1,000, Bethyl laboratory A303-781A), GAPDH (1:5,000, Cell Signaling Technology), and/or MBP (1:1,000, BioRad) overnight at 4° C. Membranes were washed three times for 10 min and incubated with anti-mouse or anti-rabbit antibodies conjugated with either horseradish peroxidase (HRP, FIGS. 2-3) or IRDye (FIG. 4, 1:20,000, LI-COR) for at least 1 h. Blots were washed with TBST three times and developed with either the ECL system according to the manufacturer's protocols (FIGS. 2-3) or scanned on an Odyssey CLx Imaging system (FIG. 4).

Results: Treatment of SYO1 synovial sarcoma cells with the BRD9 degrader Compound A results in dose dependent (FIG. 2) and time dependent (FIG. 3) depletion of BRD9 in the cells. Further, as shown in FIG. 4, the depletion of BRD9 by Compound 1 is replicated in a non-synovial sarcoma cell line (293T) and may be sustained for at least 5 days.

### Example 3—Inhibition of Growth of Synovial Cell Lines by BRD9 Inhibitors and BRD9 Degraders

The following example demonstrates that BRD9 degraders and inhibitors selectively inhibit growth of synovial sarcoma cells.

Procedures:

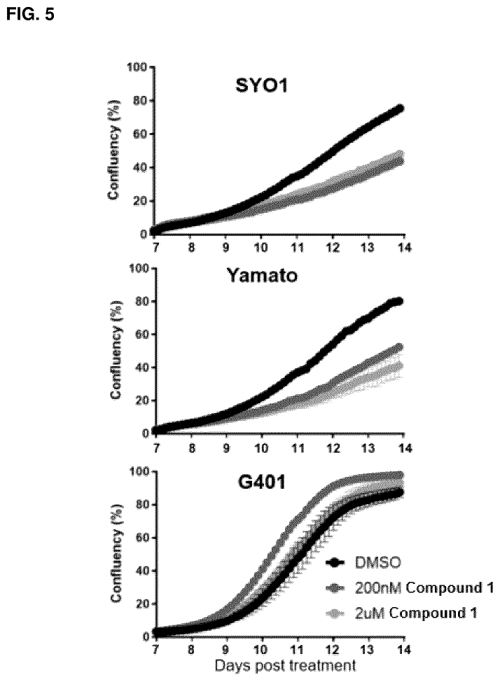

Cells were treated with DMSO or the BRD9 degrader, Compound A, at indicated concentrations, and proliferation was monitored from day 7 to day 14 by measuring confluency overtime using an IncuCyte live cell analysis system (FIG. 5). Growth medium and compounds were refreshed every 3-4 days.

Cells were seeded into 12-well plates and treated with DMSO, 1 μM BRD9 inhibitor, Compound B (also known as BI-7273, see Martin et al, J Med Chem. 59(10):4462-4475 (2016); see structure of Compound B below), or 1 μM BRD9 degrader, Compound A.

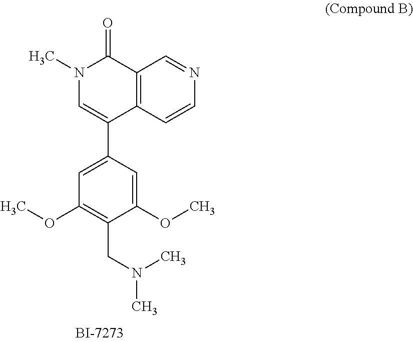

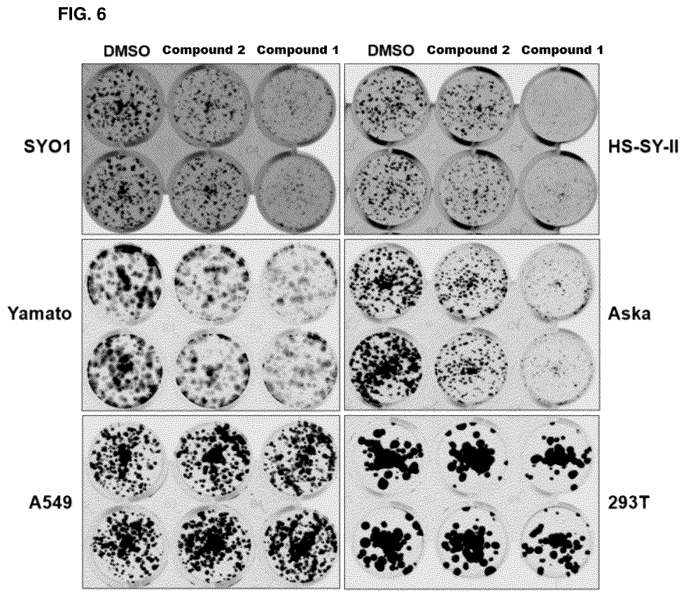

The number of cells was optimized for each cell line. Growth medium and compounds were refreshed every 3-5 days. SYO1, Yamato, A549, 293T and HS-SY-II cells were fixed and stained at day 11. ASKA cells were fixed and stained at day 23. Staining was done by incubation with crystal violet solution (0.5 g Crystal Violet, 27 ml 37% Formaldehyde, 100 mL 10×PBS, 10 mL Methanol, 863 dH20 to 1 L) for 30 min followed by 3× washes with water and drying the plates for at least 24 h at room temperature. Subsequently plates were scanned on an Odyssey CLx Imaging system (FIG. 6).

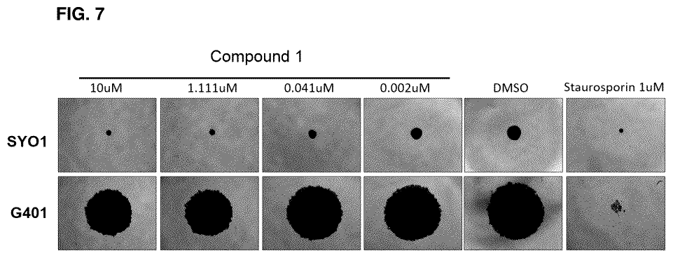

Cells were seeded into 96-well ultra low cluster plate (Costar, #7007) in 200 μL complete media and treated at day 2 with DMSO, Staurosporin, or BRD9 degarder, Compound 1, at indicated doses (FIG. 7). Media and compounds were changed every 5 d and cell colonies were imaged at day 14.

Results: As shown in FIGS. 5, 6, and 7, treatment of synovial sarcoma cell lines (SYO1, Yamato, HS-SY-II, and ASKA) with a BRD9 inhibitor, Compound B, or a BRD9 degrader, Compound A, results in inhibition of the growth of the cells, but does not result in inhibition of the growth of non-synovial control cancer cell lines (293T, A549, G401).

### Example 4—Selective Inhibition of Growth of Synovial Cell Lines by BRD9 Degraders and BRD9 Binders

The following example demonstrates that BRD9 degraders and binders selectively inhibit growth of synovial sarcoma cells.

Procedure: Cells were seeded into 6-well or 12-well plates and were treated daily with a BRD9 degrader (Compound 1), a bromo-domain BRD9 binder (Compound 2), E3 ligase binder (lenalidomide), DMSO, or staurosporin (positive control for cell killing), at indicated concentrations. The number of cells was optimized for each cell line. Growth media was refreshed every 5 days. By day 14, medium was removed, cells were washed with PBS, and stained using 500 μL of 0.005% (w/v) crystal violet solution in 25% (v/v) methanol for at least 1 hour at room temperature. Subsequently plates were scanned on an Odyssey CLx Imaging system.

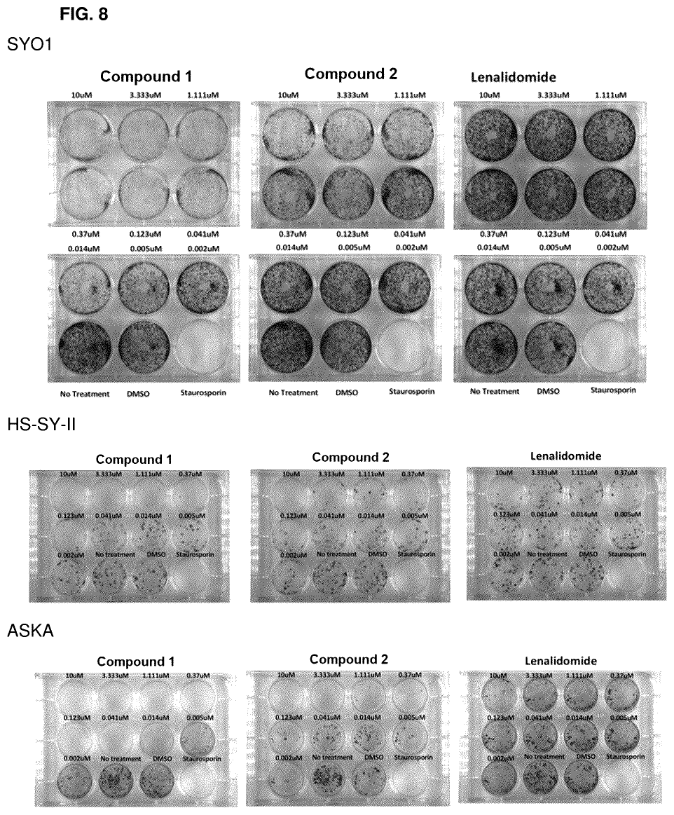

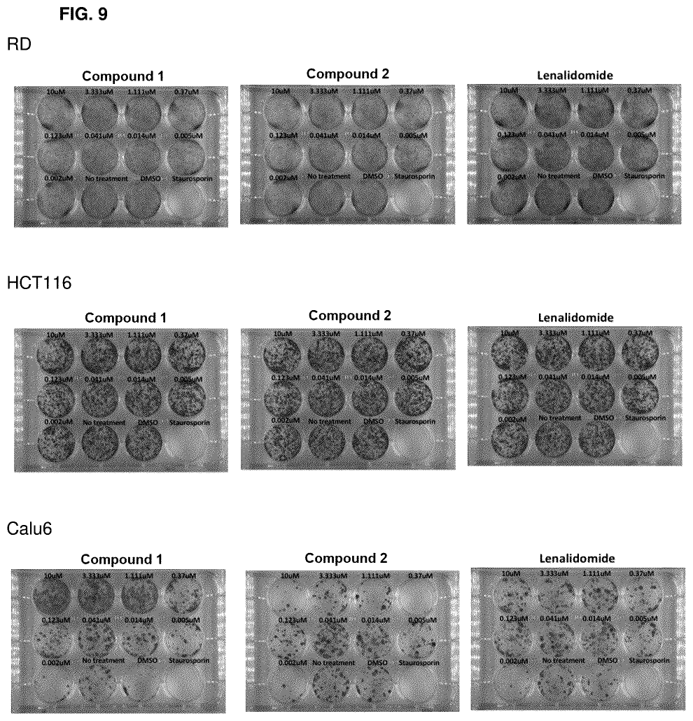

Results: As shown in FIGS. 8 and 9, treatment of synovial sarcoma cell lines (SYO1, HS-SY-II, and ASKA) with Compound A or Compound B resulted in inhibition of the growth of the cells, but did not result in inhibition of the growth of non-synovial control cancer cell lines (RD, HCT116, and Calu6). Overall, Compound A showed most significant growth inhibition in all synovial cell lines.

### Example 5—Inhibition of Cell Growth in Synovial Sarcoma Cells

The following example shows that BRD9 degraders inhibit cell growth and induce apoptosis in synovial sarcoma cells.

Procedure: SYO1 cells were treated for 8 or 13 days with DMSO, a BRD9 degrader (Compound A) at 200 nM or 1 μM, or an E3 ligase binder (lenalidomide) at 200 nM. Compounds were refreshed every 5 days. Cell cycle analysis was performed using the Click-iT™ Plus EdU Flow Cytometry Assay (Invitrogen). The apoptosis assay was performed using the Annexin V-FITC Apoptosis Detection Kit (Sigma A9210). Assays were performed according to the manufacturer's protocol.

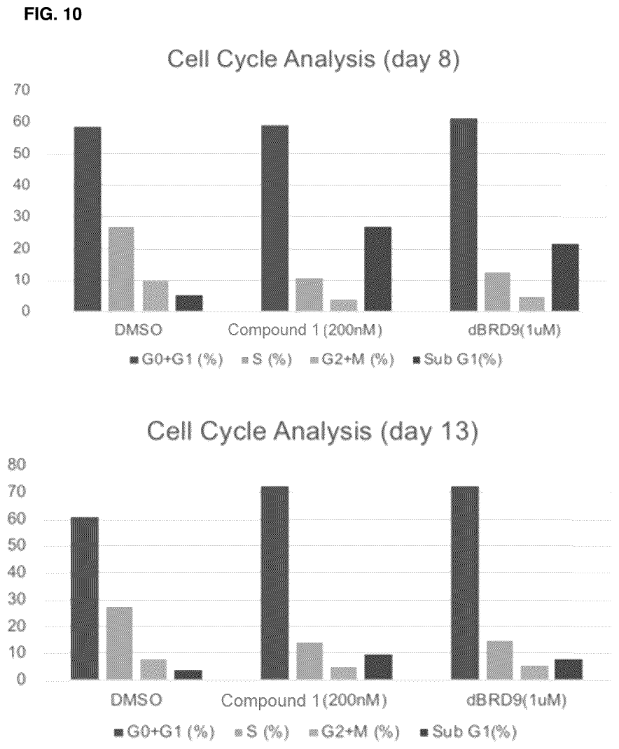

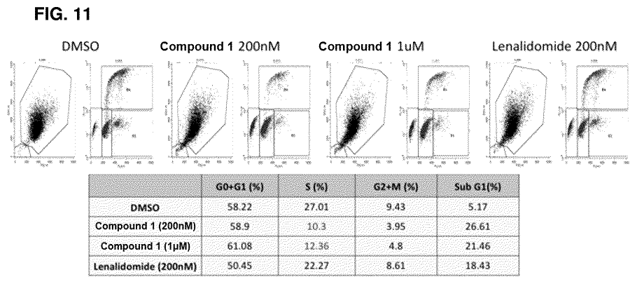

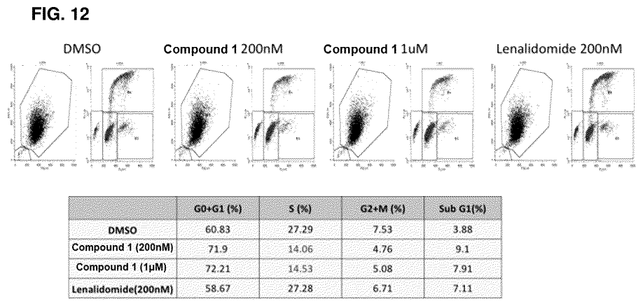

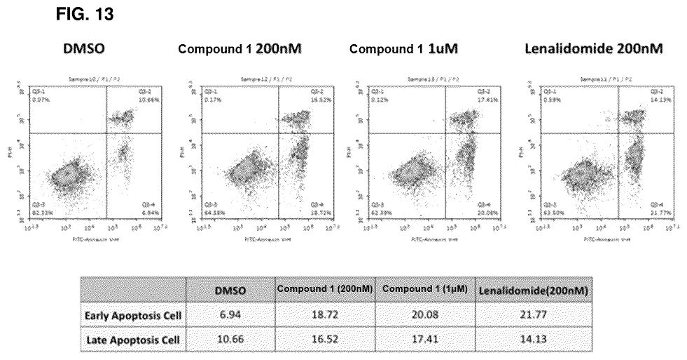

Results: As shown in FIGS. 10-13, treatment with Compound A for 8 or 13 days resulted in reduced numbers of cells in the S-phase of the cell cycle as compared to DMSO and lenalidomide. Treatment with Compound A for 8 days also resulted in increased numbers of early- and late-apoptotic cells as compared to DMSO controls.

### Example 6—Composition for SS18-SSX1-BAF

The following example shows the identification of BRD9 as a component of SS18-SSX containing BAF complexes.

Procedure: A stable 293T cell line expressing HA-SS18SSX1 was generated using lentiviral integration. SS18-SSX1 containing BAF complexes were subject to affinity purification and subsequent mass spectrometry analysis revealed SS18-SSX1 interacting proteins.

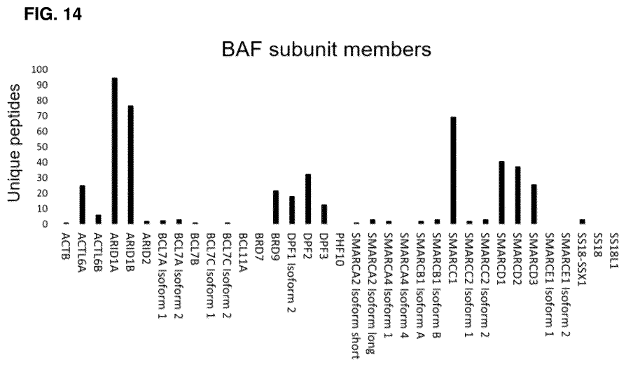

Results: As shown in FIG. 14, BAF complexes including the SS18-SSX fusion protein also included BRD9. More than 5 unique peptides were identified for ARID1A (95 peptides), ARID1B (77 peptides), SMARCC1 (69 peptides), SMARCD1 (41 peptides), SMARCD2 (37 peptides), DPF2 (32 peptides), SMARCD3 (26 peptides), ACTL6A (25 peptides), BRD9 (22 peptides), DPF1 Isoform 2 (18 peptides), DPF3 (13 peptides), and ACTL6B (6 peptides).

### Example 7—Preparation of 4-[10-(5-[4-[(dimethylamino)methyl]-3,5-dimethoxyphenyl]-7-methyl-8-oxo-2,7-naphthyridin-3-yl)-4,7-dioxa-1,10-diazaundecan-1-yl]-2-(2,6-dioxopiperidin-3-yl)isoindole-1,3-dione (Compound C)

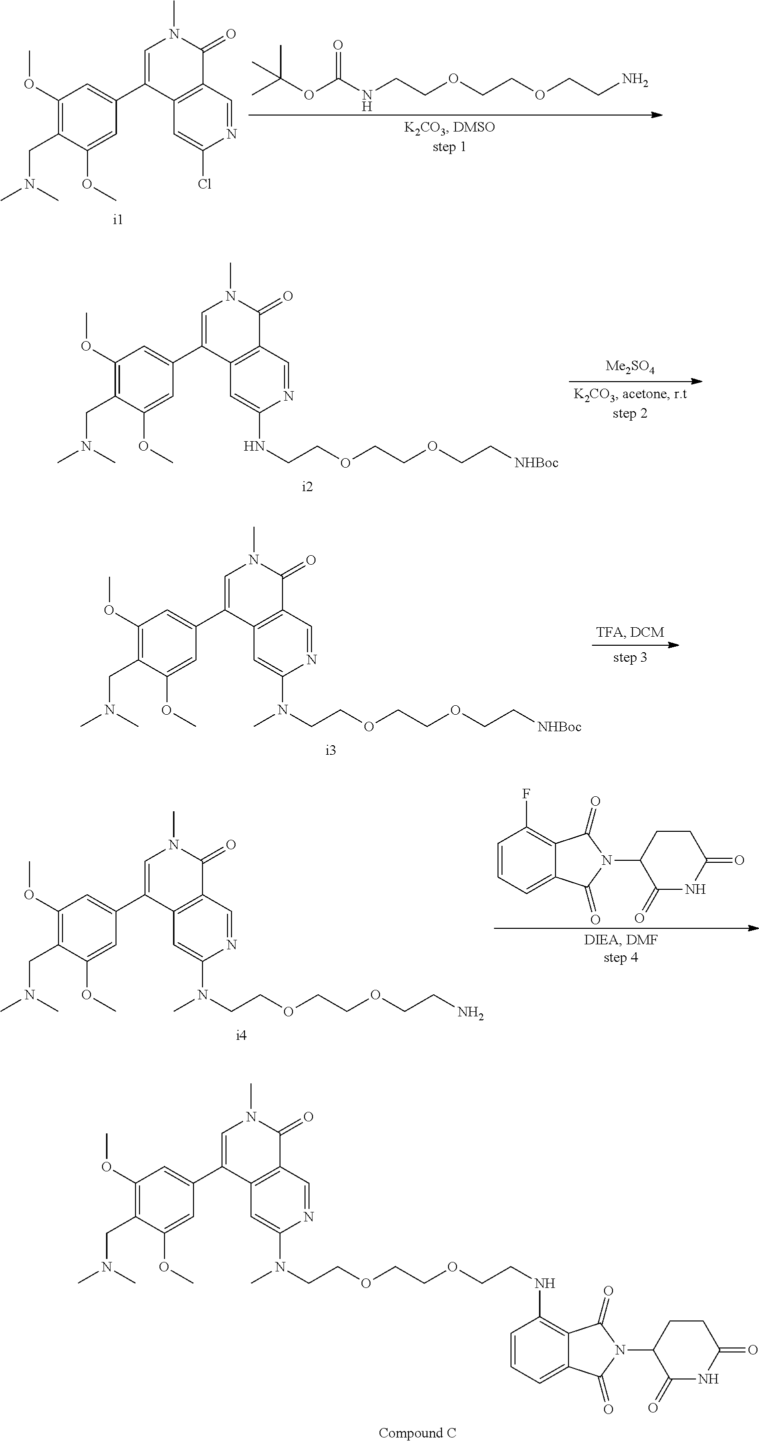

### Step 1: preparation of tert-butyl N-[2-(2-[2-[(5-[4-[(dimethylamino)methyl]-3,5-dimethoxyphenyl]-7-methyl-8-oxo-2,7-naphthyridin-3-yl)amino]ethoxy]ethoxy)ethyl]carbamate (12)

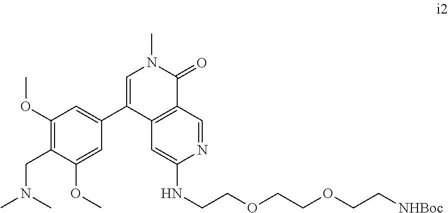 [SMILES: COc1cc(-c2cn(C)c(=O)c3cnc(NCCOCCOCCNC(=O)OC(C)(C)C)cc23)cc(OC)c1CN(C)C]

To a stirred solution of 6-chloro-4-[4-[(dimethylamino)methyl]-3,5-dimethoxyphenyl]-2-methyl-2,7-naphthyridin-1-one (335.0 mg, 0.864 mmol, 1.00 equiv) and tert-butyl N-[2-[2-(2-aminoethoxy)ethoxy]ethyl]carbamate (643.4 mg, 2.591 mmol, 3.00 equiv) in DMSO (2 mL) was added K₂CO₃ (238.7 mg, 1.727 mmol, 2.00 equiv) at room temperature. The resulting mixture was stirred overnight at 130 degrees C. The mixture was allowed to cool down to room temperature. The resulting mixture was filtered, and the filter cake was washed with CH₂Cl₂ (2×3 mL). The filtrate was concentrated under reduced pressure. The residue was purified by reverse flash chromatography (conditions: column, C18 silica gel; Mobile Phase A: Water/0.05% TFA, Mobile Phase B: ACN; Flow rate: 50 mL/min; Gradient: 0% B to 40% B in 15 min; detector, 254 nm) to afford tert-butyl N-[2-(2-[2-[(5-[4-[(dimethylamino)methyl]-3,5-dimethoxyphenyl]-7-methyl-8-oxo-2,7-naphthyridin-3-yl)amino]ethoxy]ethoxy) ethyl]carbamate (380 mg, 73.36%) as a yellow oil. LCMS (ESI) m/z: [M+H]⁺=600.

### Step 2: Preparation of tert-butyl N-[2-(2-[2-[(5-[4-[(dimethylamino)methyl]-3,5-dimethoxyphenyl]-7-methyl-8-oxo-2,7-naphthyridin-3-yl)(methyl)amino]ethoxy]ethoxy)ethyl]carbamate (13)

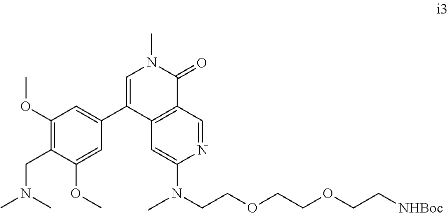 [SMILES: COc1cc(-c2cn(C)c(=O)c3cnc(N(C)CCOCCOCCNC(=O)OC(C)(C)C)cc23)cc(OC)c1CN(C)C]

To a stirred solution/mixture of tert-butyl N-[2-(2-[2-[(5-[4-[(dimethylamino)methyl]-3,5-dimethoxyphenyl]-7-methyl-8-oxo-2,7-naphthyridin-3-yl)amino]ethoxy]ethoxy)ethyl]carbamate (190.0 mg, 0.317 mmol, 1.00 equiv) and K₂CO₃ (87.6 mg, 0.634 mmol, 2 equiv) in acetone (3 mL) was added dimethyl sulfate (44.0 mg, 0.348 mmol, 1.10 equiv) at room temperature. The resulting mixture was stirred overnight at room temperature. The reaction was quenched with water at room temperature. The aqueous layer was extracted with CH₂Cl₂/isopropanol (3×5 mL). The combined organic layers were washed with brine (1×10 mL) and dried over anhydrous Na₂SO₄. After filtration, the filtrate was concentrated under reduced pressure to afford tert-butyl N-[2-(2-[2-[(5-[4-[(dimethylamino)methyl]-3,5-dimethoxyphenyl]-7-methyl-8-oxo-2,7-naphthyridin-3-yl)(methyl)amino]ethoxy]ethoxy)ethyl]carbamate (95.00 mg, 48.86%) as a yellow oil. The crude product was used in the next step directly without further purification. LCMS (ESI) m/z: [M+H]⁺=614.

### Step 3: Preparation of 3,3,3-trifluoropropanoic acid; 6-([2-[2-(2-aminoethoxy)ethoxy]ethyl](methyl)amino)-4-[4-[(dimethylamino)methyl]-3,5-dimethoxyphenyl]-2-methyl-2,7-naphthyridin-1-one (14)

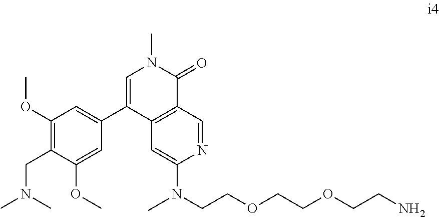 [SMILES: COc1cc(-c2cn(C)c(=O)c3cnc(N(C)CCOCCOCCN)cc23)cc(OC)c1CN(C)C]

To a stirred solution of tert-butyl N-[2-(2-[2-[(5-[4-[(dimethylamino)methyl]-3,5-dimethoxyphenyl]-7-methyl-8-oxo-2,7-naphthyridin-3-yl)(methyl)amino]ethoxy]ethoxy)ethyl]carbamate (75.00 mg, 0.122 mmol, 1.00 equiv) in dichloromethane (3 mL) was added TFA (1 mL) dropwise at room temperature. The resulting mixture was concentrated under vacuum to afford 3,3,3-trifluoropropanoic acid; 6-([2-[2-(2-amino ethoxy)ethoxy]ethyl](methyl)amino)-4-[4-[(dimethylamino)methyl]-3,5-dimethoxyphenyl]-2-methyl-2,7-naphthyridin-1-one (103 mg, crude) as yellow oil. The crude product was used in the next step directly without further purification. LCMS (ESI) m/z: [M+H]⁺=514.

### Step 4: Preparation of 4-[10-(5-[4-[(dimethylamino)methyl]-3,5-dimethoxyphenyl]-7-methyl-8-oxo-2,7-naphthyridin-3-yl)-4,7-dioxa-1,10-diazaundecan-1-yl]-2-(2,6-dioxopiperidin-3-yl)isoindole-1,3-dione (Compound C)

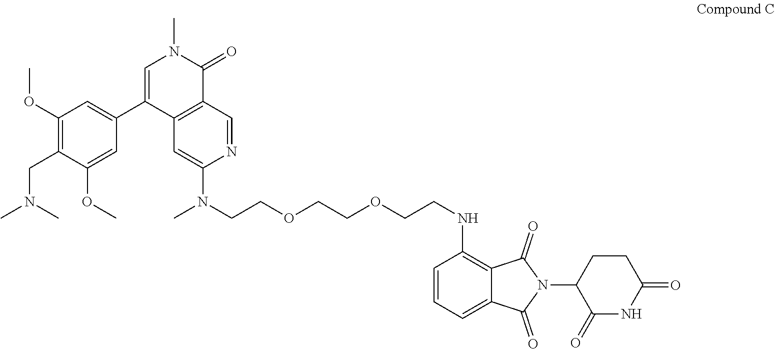 [SMILES: COc1cc(-c2cn(C)c(=O)c3cnc(N(C)CCOCCOCCNc4cccc5c4C(=O)N(C4CCC(=O)NC4=O)C5=O)cc23)cc(OC)c1CN(C)C]

To a stirred solution of 6-([2-[2-(2-aminoethoxy)ethoxy]ethyl](methyl)amino)-4-[4-[(dimethylamino) methyl]-3,5-dimethoxyphenyl]-2-methyl-2,7-naphthyridin-1-one (68.00 mg, 0.132 mmol, 1.00 equiv) in DMF (1 mL) was added 2-(2,6-dioxopiperidin-3-yl)-4-fluoroisoindole-1,3-dione (34.6 mg, 0.125 mmol, 0.95 equiv) and DIEA (85.6 mg, 0.662 mmol, 5.00 equiv) at room temperature. The resulting mixture was stirred for overnight at 80 degrees C. The crude product was purified by Prep-HPLC (conditions: Xselect CSH F-Phenyl OBD Column 19*150 mm Sum; Mobile Phase A: Water (0.1% FA), Mobile Phase B: ACN; Flow rate: 25 mL/min; Gradient: 9 B to 19 B in 12 min; 254 nm; Rt:12.63 minutes) to afford 4-[10-(5-[4-[(dimethylamino)methyl]-3,5-dimethoxyphenyl]-7-methyl-8-oxo-2,7-naphthyridin-3-yl)-4,7-dioxa-1,10-diazaundecan-1-yl]-2-(2,6-dioxopiperidin-3-yl)isoindole-1,3-dione (3.2 mg, 3.14%) as a yellow solid. ¹H NMR (400 MHz, Methanol-d4) δ 8.96 (s, 1H), 7.54-7.46 (m, 2H), 6.99 (dd, J=15.8, 7.7 Hz, 2H), 6.84 (s, 2H), 6.74 (s, 1H), 4.96-4.94 (m, 1H), 4.57 (s, 2H), 3.97 (s, 6H), 3.77-3.69 (m, 8H), 3.59-3.53 (m, 5H), 3.41 (t, J=5.2 Hz, 2H), 3.17-3.11 (m, 9H), 2.83-2.53 (m, 3H), 2.04-1.95 (m, 1H). LCMS (ESI) m/z: [M+H]⁺=770.50.

### Example 8—Preparation of Compounds D32-D184

In analogous procedures to those described for Compound C in the example above, compounds D1-D59 were prepared using the appropriate starting materials.

TABLE 4

| Compound | | |
|---|---|---|
| No. | LCMS | ¹H NMR |
| D1 | 749.74 | |
| D2 | 734.71 | ¹H NMR (400 MHz, DMSO-d6) δ 11.11 (s, 1H), 9.45 (s, 1H), 8.72 |
| | | (d, J = 5.7 Hz, 1H), 7.97 (s, 1H), 7.86 (s, 1H), 7.55 (d, J = 5.7 Hz, |
| | | 1H), 6.84 (s, 2H), 5.14 (d, J = 13.2 Hz, 1H), 4.98 (s, 2H), 4.35 (s, |
| | | 2H), 3.91-3.71 (m, 6H), 3.59 (s, 3H), 3.03-2.78 (m, 1H), 2.73 (s, |
| | | 2H), 2.67-2.49 (m, 1H), 2.05 (s, 2H). |
| D3 | 694.5 | |
| D4 | 734.26 | |
| D5 | 720.54 | |
| D6 | 706.65 | |
| D7 | 720.4 | |
| D8 | 681.35 | ¹H NMR (300 MHz, DMSO-d6) δ 11.13 (s, 1H), 9.04 (s, 1H), 8.17 |
| | | (s, 1H, FA), 7.86-7.80 (m, 1H), 7.59 (s, 1H), 7.29-7.23 (m, 2H), |
| | | 6.77 (s, 2H), 6.47 (s, 1H), 5.12 (dd, J = 12.9, 5.3 Hz, 1H), 4.95 (t, |
| | | J = 5.5 Hz, 1H), 3.81 (s, 6H), 3.75-3.69 (m, 4H), 3.48 (s, 3H), 3.15- |
| | | 3.11 (m, 2H), 3.06 (s, 6H), 2.91-2.84 (m, 1H), 2.65-2.55 (m, 2H), |
| | | 2.07-1.99 (m, 1H). |
| D9 | 693.3 | ¹H NMR (300 MHz, DMSO-d6) δ 11.12 (s, 1H), 9.04 (s, 1H), 8.17 |
| | | (s, 1H, FA), 7.88-7.73 (m, 3H), 7.60 (s, 1H), 6.78 (s, 2H), 6.50 (s, |
| | | 1H), 5.14 (dd, J = 12.9, 5.3 Hz, 1H), 3.84 (s, 6H), 3.65 (s, 2H), 3.49 |
| | | (s, 3H), 3.08 (s, 6H), 3.02 (d, J = 11.3 Hz, 2H), 2.97-2.70 (m, 3H), |
| | | 2.63-2.55 (m, 1H), 2.30-2.20 (m, 2H), 2.10-2.00 (m, 1H), 1.83- |
| | | 1.63 (m, 4H). |
| D10 | 680.2 | ¹H NMR (300 MHz, DMSO-d6) δ 10.97 (s, 1H), 9.04 (s, 1H), 7.60 |
| | | (s, 1H), 7.42 (d, J = 8.5 Hz, 1H), 7.26 (dd, J = 8.5, 2.3 Hz, 1H), 7.15 |
| | | (d, J = 2.3 Hz, 1H), 6.79 (s, 2H), 6.50 (s, 1H), 5.09 (dd, J = 13.2, 5.0 |
| | | Hz, 1H), 4.41-4.14 (m, 2H), 3.84 (s, 6H), 3.67 (s, 2H), 3.48 (s, |
| | | 3H), 3.19 (s, 4H), 3.08 (s, 6H), 2.91 (ddd, J = 17.9, 13.6, 5.5 Hz, |
| | | 1H), 2.65 (s, 4H), 2.48-2.24 (m, 2H), 2.06-1.92 (m, 1H). |
| D11 | 680.3 | ¹H NMR (300 MHz, DMSO-d6) δ 10.94 (s, 1H), 9.04 (s, 1H), 7.60 |
| | | (s, 1H), 7.52 (d, J = 8.7 Hz, 1H), 7.05 (d, J = 7.9 Hz, 2H), 6.79 (s, |
| | | 2H), 6.50 (s, 1H), 5.05 (dd, J = 13.3, 5.1 Hz, 1H), 4.40-4.10 (m, |
| | | 2H), 3.84 (s, 6H), 3.65 (s, 2H), 3.48 (s, 3H), 3.33-3.20 (m, 4H), |
| | | 3.08 (s, 6H), 2.96-2.83 (m, 1H), 2.59 (d, J = 14.6 Hz, 4H), 2.45- |
| | | 2.25 (m, 2H), 1.95 (dd, J = 12.1, 6.5 Hz, 1H). |
| D12 | 746.2 | ¹H NMR (400 MHz, Methanol-d4) δ 8.99-8.94 (m, 1H), 7.67 (d, J = |
| | | 8.3 Hz, 1H), 7.59 (s, 1H), 6.88 (s, 2H), 6.87 (d, J = 2.0 Hz, 1H), 6.75- |
| | | 6.68 (m, 1H), 6.38 (d, J = 1.8 Hz, 1H), 5.12-5.02 (m, 1H), 4.44 |
| | | (s, 2H), 4.23 (t, J = 7.6 Hz, 4H), 3.99 (s, 8H), 3.87 (s, 2H), 3.60 (s, |
| | | 4H), 3.34 (s, 1H), 3.31-3.19 (m, 2H), 2.95-2.81 (m, 1H), 2.81- |
| | | 2.64 (m, 2H), 2.60-2.48 (m, 2H), 2.30 (d, J = 14.4 Hz, 2H), 2.21- |
| | | 2.07 (m, 3H). |
| D13 | 720.45 | ¹H NMR (400 MHz, DMSO-d6) δ 10.90 (s, 1H), 9.01 (s, 1H), 8.17 |
| | | (s, 1H), 7.51 (d, J = 37.4 Hz, 1H), 6.74 (s, 2H), 6.65-6.35 (m, 3H), |
| | | 5.01 (dd, J = 13.3, 5.1 Hz, 1H), 4.37-3.99 (m, 2H), 3.80 (s, 5H), |
| | | 3.59 (s, 3H), 3.54 (s, 2H), 3.46 (s, 2H), 3.15 (s, 1H), 3.05 (s, 5H), |
| | | 2.58 (s, 1H), 2.45-2.39 (m, 5H), 2.39-2.27 (m, 1H), 1.93 (ddq, |
| | | J = 10.4, 5.4, 3.2, 2.6 Hz, 1H), 1.71 (t, J = 5.4 Hz, 4H). |
| D14 | 720.52 | ¹H NMR (400 MHz, DMSO-d6) δ 10.93 (s, 1H), 9.02 (s, 1H), 8.12 |
| | | (s, 1H), 7.57 (s, 1H), 7.36 (d, J = 8.2 Hz, 1H), 6.80 (s, 2H), 6.67 (d, |
| | | J = 7.5 Hz, 2H), 6.47 (s, 1H), 5.05 (dd, J = 13.3, 5.1 Hz, 1H), 4.37- |
| | | 4.07 (m, 2H), 3.84 (s, 7H), 3.60 (s, 4H), 3.47 (s, 3H), 3.06 (s, 6H), |
| | | 2.97-2.84 (m, 1H), 2.81 (d, J = 25.0 Hz, 0H), 2.69-2.52 (m, 1H), |
| | | 2.42-2.26 (m, 1H), 2.05-1.92 (m, 1H), 1.85 (s, 4H). |
| D15 | 665.55 | ¹H NMR (400 MHz, Methanol-d4) δ 8.97 (d, J = 0.8 Hz, 1H), 7.73 (d, |
| | | J = 9.2 Hz, 1H), 7.59 (s, 1H), 7.20 (d, J = 6.5 Hz, 2H), 6.88 (s, 2H), |
| | | 6.39 (s, 1H), 5.14 (dd, J = 13.3, 5.1 Hz, 1H), 4.46 (d, J = 7.1 Hz, |
| | | 4H), 4.23 (t, J = 7.6 Hz, 4H), 3.98 (s, 6H), 3.86-3.63 (m, 6H), 3.60 |
| | | (s, 4H), 3.58-3.46 (m, 3H), 3.31-3.24 (m, 3H), 2.99-2.86 (m, |
| | | 1H), 2.84-2.75 (m, 1H), 2.60-2.42 (m, 5H), 2.18 (d, J = 16.2 Hz, |
| | | 3H). |
| D16 | 693.35 | ¹H NMR (400 MHz, DMSO-d6) δ 11.09 (s, 1H), 9.04 (s, 1H), 7.64 |
| | | (d, J = 9.0 Hz, 1H), 7.58 (d, J = 2.7 Hz, 1H), 7.14 (d, J = 7.2 Hz, |
| | | 2H), 6.76 (s, 2H), 6.49 (s, 1H), 5.06 (dd, J = 12.9, 5.4 Hz, 1H), 3.97 |
| | | (d, J = 13.3 Hz, 1H), 3.82 (s, 6H), 3.76 (d, J = 13.1 Hz, 1H), 3.48 (s, |
| | | 3H), 3.06 (s, 7H), 2.95-2.82 (m, 2H), 2.71-2.54 (m, 4H), 2.01 (d, |
| | | J = 12.7 Hz, 1H), 1.85 (s, 1H), 1.72 (d, J = 11.2 Hz, 2H), 1.35 (tt, J = |
| | | 33.0, 18.0 Hz, 2H). |
| D17 | 693.1 | ¹H NMR (300 MHz, DMSO-d6) δ 11.12 (s, 1H), 9.01 (s, 1H), 7.88- |
| | | 7.79 (m, 1H), 7.60 (s, 1H), 7.27 (d, 2H), 6.74 (s, 2H), 6.18 (s, 1H), |
| | | 5.12-4.96 (m, 2H), 3.99 (t, 4H), 3.82 (s, 6H), 3.78-3.70 (m, |
| | | 3H), 3.48 (s, 4H), 3.24-3.13 (m, 2H), 2.97-2.80 (m, 1H), 2.66- |
| | | 2.62 (m, 1H), 2.61-2.54 (m, 1H), 2.30-2.28 (m, 2H), 2.10-1.92 |
| | | (m, 1H). |
| D18 | 669.15 | ¹H NMR (400 MHz, DMSO-d6) δ 11.09 (s, 1H), 9.04 (s, 1H), 8.16 |
| | | (s, 1H, FA), 7.82 (dd, J = 7.5, 0.9 Hz, 1H), 7.59 (s, 1H), 6.76 (s, 2H), |
| | | 6.46 (s, 1H), 6.38-6.30 (m, 2H), 5.29 (dd, J = 12.5, 5.2 Hz, 1H), |
| | | 4.76 (t, J = 5.6 Hz, 1H), 3.81 (s, 6H), 3.76-3.63 (m, 4H), 3.48 (s, |
| | | 3H), 3.10 (dd, J = 8.2, 4.8 Hz, 2H), 3.06 (s, 6H), 2.97-2.83 (m, |
| | | 1H), 2.68-2.59 (m, 1H), 2.48-2.37 (m, 1H), 2.27-2.07 (m, 1H). |
| D19 | 736.35 | ¹H NMR (400 MHz, DMSO-d6) δ 11.11 (s, 1H), 9.52 (s, 1H, TFA), |
| | | 9.09 (s, 1H), 7.78 (d, J = 8.4 Hz, 1H), 7.65 (s, 1H), 7.49 (d, J = 2.3 |
| | | Hz, 1H), 7.35 (dd, J = 8.6, 2.3 Hz, 1H), 6.90 (s, 2H), 6.67 (s, 1H), |
| | | 5.10 (dd, J = 13.0, 5.3 Hz, 1H), 4.42-4.31 (m, 2H), 4.20 (d, J = |
| | | 12.6 Hz, 2H), 3.92 (s, 6H), 3.70 (t, J = 4.8 Hz, 5H), 3.63-3.55 (m, |
| | | 8H), 3.32 (h, J = 11.6, 10.4 Hz, 4H), 2.90 (ddd, J = 17.4, 14.0, 5.4 |
| | | Hz, 1H), 2.65-2.54 (m, 2H), 2.07-2.00 (m, 1H). |
| D20 | 732.5 | ¹H NMR (300 MHz, DMSO-d6) δ 10.97 (s, 1H), 9.02 (s, 1H), 8.24 |
| | | (s, 1H, FA), 7.61 (s, 1H), 7.37 (d, J = 8.1 Hz, 1H), 6.78-6.63 (m, |
| | | 4H), 6.21 (s, 1H), 5.09 (dd, J = 13.2, 5.1 Hz, 1H), 4.35-4.15 (m, |
| | | 2H), 4.01 (t, J = 7.3 Hz, 4H), 3.82 (s, 6H), 3.58-3.48 (m, 8H), 2.97- |
| | | 2.85 (m, 1H), 2.67-2.55 (m, 2H), 2.42-2.26 (m, 7H), 1.98 (d, |
| | | J = 12.6 Hz, 1H), 1.80-1.62 (m, 4H). |
| D21 | 711.3 | ¹H NMR (300 MHz, DMSO-d6) δ 11.09 (s, 1H), 9.07 (s, 1H), 8.19 |
| | | (s, 0.6H, FA), 7.87-7.78 (m, 1H), 7.63 (s, 1H), 6.74 (s, 2H), 6.63 |
| | | (s, 1H), 6.40-6.29 (m, 2H), 5.29 (dd, J = 12.5, 5.2 Hz, 1H), 4.76 (t, |
| | | J = 5.5 Hz, 1H), 3.81 (s, 6H), 3.68-3.57 (m, 8H), 3.52-3.50 (m, |
| | | 7H), 3.10 (t, J = 6.4 Hz, 2H), 2.99-2.81 (m, 1H), 2.66-2.50 (m, |
| | | 1H), 2.49-2.38 (m, 1H), 2.16-2.08 (m, 1H). |
| D22 | 677.35 | ¹H NMR (400 MHz, DMSO-d6) δ 11.14 (s, 1H), 9.01 (s, 1H), 8.18 |
| | | (s, 1H, FA), 7.90-7.82 (m, 2H), 7.81-7.74 (m, 1H), 7.62 (s, 1H), |
| | | 6.75 (s, 2H), 6.19 (s, 1H), 5.15 (dd, J = 12.8, 5.4 Hz, 1H), 3.99 (t, |
| | | J = 7.4 Hz, 4H), 3.83 (s, 6H), 3.79-3.60 (m, 6H), 3.48 (s, 3H), 3.26 |
| | | (s, 1H), 2.98-2.80 (m, 1H), 2.66-2.52 (m, 2H), 2.33 (m, J = 7.2 |
| | | Hz, 2H), 2.10-2.01 (m, 1H). |
| D23 | 702.46 | |
| D24 | 702.46 | ¹H NMR (400 MHz, DMSO-d6) δ 10.92 (s, 1H), 8.99 (s, 1H), 8.17 |
| | | (s, 1H), 7.59-7.46 (m, 1H), 7.37 (dd, J = 18.7, 7.8 Hz, 2H), 7.17- |
| | | 6.87 (m, 2H), 6.67 (d, J = 8.1 Hz, 2H), 5.05 (dd, J = 13.3, 5.2 Hz, |
| | | 1H), 4.39-4.05 (m, 2H), 4.07-3.89 (m, 5H), 3.82 (d, J = 7.8 Hz, |
| | | 4H), 3.58 (s, 3H), 3.47 (d, J = 16.6 Hz, 5H), 2.97-2.79 (m, 1H), |
| | | 2.67-2.51 (m, 2H), 2.45-2.26 (m, 9H), 2.08-1.88 (m, 2H), 1.77 |
| | | (d, J = 5.4 Hz, 5H). |
| D25 | 773.42 | |
| D26 | 805.4 | ¹H NMR (300 MHz, DMSO-d6) δ 11.07 (s, 1H), 10.20-9.86 (m, |
| | | 1H), 9.30-9.10 (m, 1H), 9.02 (s, 1H), 7.84 (dd, J = 7.8, 4.2 Hz, 1H), |
| | | 7.63 (d, J = 2.1 Hz, 1H), 6.87 (s, 2H), 6.80-6.68 (m, 1H), 6.35 (d, |
| | | J = 5.7 Hz, 1H), 6.23 (d, J = 5.7 Hz, 1H), 5.27 (dd, J = 12.3, 5.1 Hz, |
| | | 1H), 4.22 (d, J = 3.6 Hz, 2H), 4.10-3.96 (m, 6H), 3.90 (s, 6H), 3.65- |
| | | 3.52 (m, 2H), 3.50-3.34 (m, 5H), 3.30-3.10 (m, 6H), 3.08-2.80 |
| | | (m, 2H), 2.75-2.60 (m, 1H), 2.50-2.42 (m, 2H), 2.42-2.28 (m, |
| | | 2H), 2.20-2.08 (m, 1H), 1.96-1.70 (m, 3H), 1.70-1.40 (m, 4H). |
| D27 | 747.25 | ¹H NMR (400 MHz, DMSO-d6) δ 10.96 (s, 1H), 9.01 (s, 1H), 8.19 |
| | | (s, 1H, FA salt), 7.60 (s, 1H), 7.52 (d, J = 9.1 Hz, 1H), 7.08-7.01 (m, |
| | | 2H), 6.74 (s, 2H), 6.19 (s, 1H), 5.05 (dd, J = 13.3, 5.1 Hz, 1H), 4.33 |
| | | (d, J = 16.9 Hz, 1H), 4.20 (d, J = 16.9 Hz, 1H), 4.00 (t, J = 7.4 Hz, |
| | | 4H), 3.82 (s, 6H), 3.68 (s, 2H), 3.48 (s, 3H), 3.30-3.25 (m, 6H), |
| | | 3.05 (t, J = 6.5 Hz, 2H), 2.97-2.80 (m, 2H), 2.63-2.54 (m, 1H), |
| | | 2.44-2.26 (m, 7H), 2.00-1.92 (m, 1H). |
| D28 | 639.2 | ¹H NMR (400 MHz, DMSO-d6) δ 11.08 (s, 1H), 9.02 (s, 1H), 7.63 |
| | | (d, J = 8.6 Hz, 1H), 7.58 (s, 1H), 7.31 (d, J = 2.4 Hz, 1H), 7.04 (dd, |
| | | J = 8.7, 2.4 Hz, 1H), 6.80 (s, 2H), 6.44 (s, 1H), 5.05 (dd, J = 12.9, 5.4 |
| | | Hz, 1H), 4.68 (s, 2H), 3.88 (s, 6H), 3.46 (s, 3H), 3.13 (s, 3H), 3.05 |
| | | (s, 6H), 2.95-2.82 (m, 1H), 2.63-2.56 (m, 1H), 2.55 (s, 1H), 2.06- |
| | | 1.95 (m, 1H). |
| D29 | 667.30 | ¹H NMR (300 MHz, DMSO-d6) δ 10.98 (s, 1H), 9.16 (s, 1H), 7.75 |
| | | (s, 1H), 7.42 (d, J = 8.5 Hz, 1H), 7.26 (d, J = 8.8 Hz, 1H), 7.17- |
| | | 7.11 (m, 1H), 6.78 (s, 1H), 6.74 (s, 2H), 5.10 (dd, J = 13.2, 5.2 Hz, |
| | | 1H), 4.34 (d, J = 16.7 Hz, 1H), 4.20 (d, J = 16.9 Hz, 1H), 3.94 (s, |
| | | 3H), 3.83 (s, 6H), 3.68-3.61 (m, 2H), 3.54 (s, 3H), 3.19-3.12 (m, |
| | | 4H), 2.74 (d, J = 1.9 Hz, 1H), 2.65-2.58 (m, 5H), 2.38 (d, J = 8.0 |
| | | Hz, 1H), 2.30-2.25 (m, 1H). |
| D30 | 731.20 | ¹H NMR (400 MHz, DMSO-d6) δ 11.08 (s, 1H), 9.28 (s, 1H), 8.23 |
| | | (s, 1H, FA), 7.80 (s, 1H), 7.64 (d, J = 8.3 Hz, 1H), 7.44 (s, 1H), 6.81- |
| | | 6.72 (m, 3H), 6.65 (dd, J = 8.3, 2.1 Hz, 1H), 5.06 (dd, J = 12.9, 5.4 |
| | | Hz, 1H), 3.83 (s, 6H), 3.74 (s, 4H), 3.56 (d, J = 5.4 Hz, 5H), 2.95- |
| | | 2.82 (m, 1H), 2.55 (s, 3H), 2.44 (s, 3H), 2.27 (tt, J = 7.8, 3.9 Hz, |
| | | 1H), 2.06-1.97 (m, 1H), 1.74 (t, J = 5.4 Hz, 4H), 1.06-0.94 (m, |
| | | 4H). |
| D31 | 717.25 | ¹H NMR (400 MHz, DMSO-d6) δ 10.95 (s, 1H), 9.28 (s, 1H), 8.21 |
| | | (s, 1H, FA), 7.80 (s, 1H), 7.53-7.42 (m, 2H), 6.75 (s, 2H), 6.54- |
| | | 6.44 (m, 2H), 5.04 (dd, J = 13.2, 5.1 Hz, 1H), 4.36-4.13 (m, 2H), |
| | | 3.83 (s, 6H), 3.63 (s, 4H), 3.56 (s, 5H), 2.90 (ddd, J = 17.0, 13.6, |
| | | 5.4 Hz, 1H), 2.55 (s, 3H), 2.45 (s, 2H), 2.40-2.30 (m, 1H), 2.27 (td, |
| | | J = 7.8, 3.9 Hz, 1H), 2.05-1.85 (m, 1H), 1.74 (t, J = 5.4 Hz, 4H), |
| | | 1.06-0.94 (m, 4H). |
| D32 | 717.25 | ¹H NMR (400 MHz, DMSO-d6) δ 10.98 (s, 1H), 9.29 (s, 1H), 8.15 |
| | | (s, 1H, FA), 7.81 (s, 1H), 7.44 (s, 1H), 7.38 (d, J = 8.1 Hz, 1H), 6.78 |
| | | (s, 2H), 6.70 (d, J = 7.9 Hz, 2H), 5.08 (dd, J = 13.3, 5.1 Hz, 1H), |
| | | 4.36-4.15 (m, 2H), 3.86 (s, 6H), 3.76 (s, 2H), 3.61 (s, 4H), 3.57 (s, |
| | | 4H), 2.98-2.84 (m, 1H), 2.71-2.65 (m, 2H), 2.60 (d, J = 16.6 Hz, |
| | | 2H), 2.38 (dd, J = 13.3, 4.5 Hz, 1H), 2.30-2.21 (m, 1H), 1.98 (d, |
| | | J = 13.1 Hz, 1H), 1.83 (s, 4H), 1.05-0.96 (m, 4H). |
| D33 | 692.15 | ¹H NMR (300 MHz, DMSO-d6) δ 11.13 (s, 1H), 9.30 (s, 1H), 7.85 |
| | | (d, J = 9.0 Hz, 2H), 7.52 (s, 1H), 7.28 (d, J = 7.9 Hz, 2H), 6.82 (s, |
| | | 2H), 5.24-5.05 (m, 1H), 5.00 (s, 1H), 4.00-3.67 (m, 10H), 3.58 |
| | | (s, 3H), 3.32-3.27 (m, 2H), 3.02-2.78 (m, 1H), 2.67-2.54 (m, |
| | | 2H), 2.14-1.97 (m, 1H), 1.40 (s, 3H), 1.33-1.20 (m, 2H), 0.96- |
| | | 0.80 (m, 2H). |
| D34 | 720.35 | ¹H NMR (300 MHz, DMSO-d6) δ 10.98 (s, 1H), 9.29 (s, 1H), 8.14 |
| | | (s, 1H, FA), 7.80 (s, 1H), 7.44 (s, 1H), 7.38 (d, J = 7.9 Hz, 1H), 6.75 |
| | | (s, 2H), 6.69 (d, J = 8.1 Hz, 2H), 5.09 (dd, J = 13.2, 5.2 Hz, 1H), |
| | | 4.41-4.06 (m, 2H), 3.84 (s, 6H), 3.59 (s, 6H), 2.95-2.84 (m, 1H), |
| | | 2.64-2.61 (m, 2H), 2.42-2.34 (m, 4H), 2.05-1.92 (m, 2H), 1.76 (s, |
| | | 4H), 1.01 (s, 4H). |
| D35 | 710.35 | ¹H NMR (300 MHz, Methanol-d4) δ 9.26 (s, 1H), 7.58 (s, 1H), 7.42 |
| | | (d, J = 8.2 Hz, 1H), 6.89 (d, J = 4.7 Hz, 3H), 6.85-6.79 (m, 2H), |
| | | 5.15 (dd, J = 13.2, 5.1 Hz, 1H), 4.49-4.32 (m, 4H), 4.00 (d, J = 7.0 |
| | | Hz, 9H), 3.87 (s, 2H), 3.74 (s, 2H), 3.64-3.52 (m, 2H), 3.29-3.19 |
| | | (m, 2H), 3.01-2.86 (m, 1H), 2.85-2.74 (m, 1H), 2.60-2.41 (m, |
| | | 1H), 2.36-2.25 (m, 2H), 2.24-2.04 (m, 3H). |
| D36 | 666.30 | ¹H NMR (300 MHz, Methanol-d4) δ 9.25 (s, 1H), 8.56 (d, 1H, FA), |
| | | 7.79 (d, J = 7.9 Hz, 1H), 7.58 (s, 1H), 7.54 (s, 1H), 7.48 (d, J = 8.1 |
| | | Hz, 1H), 6.94-6.78 (m, 3H), 5.17 (dd, J = 13.3, 5.1 Hz, 1H), 4.51 |
| | | (d, J = 5.0 Hz, 2H), 4.37-4.24 (m, 2H), 4.01 (s, 3H), 3.97 (s, 6H), |
| | | 3.65 (s, 3H), 3.57 (d, J = 12.0 Hz, 2H), 3.16-2.97 (m, 3H), 2.97- |
| | | 2.86 (m, 1H), 2.86-2.75 (m, 1H), 2.51 (qd, J = 13.1, 4.7 Hz, 1H), |
| | | 2.27-2.15 (m, 1H), 2.15-2.03 (m, 4H). |
| D37 | 657.35 | ¹H NMR (300 MHz, Methanol-d4) δ 7.70 (s, 1H), 6.04 (d, J = 9.4 Hz, |
| | | 2H), 5.76-5.64 (m, 2H), 5.31 (s, 2H), 5.25 (s, 1H), 3.75-3.57 (m, |
| | | 2H), 3.25-3.19 (m, 2H), 3.15-3.05 (m, 2H), 3.00-2.89 (m, 2H), |
| | | 2.89-2.75 (m, 2H), 2.50-2.34 (m, 9H), 1.45-1.20 (m, 2H), 1.08- |
| | | 0.89 (m, 1H), 0.73-0.59 (m, 1H). |
| D38 | 657.30 | ¹H NMR (300 MHz, DMSO-d6) δ 11.00 (s, 1H), 10.29 (s, 1H, TFA), |
| | | 9.17 (s, 1H), 7.77-7.66 (m, 2H), 7.24-6.99 (m, 2H), 6.80 (d, J = |
| | | 30.9 Hz, 3H), 5.33-5.02 (m, 2H), 4.81-4.55 (m, 2H), 4.55-4.13 |
| | | (m, 6H), 4.00-3.82 (m, 9H), 3.02-2.85 (m, 1H), 2.63 (s, 1H), 2.44- |
| | | 2.31 (m, 1H), 2.08-1.93 (m, 1H). |
| D39 | 667.35 | ¹H NMR (300 MHz, Methanol-d4) δ 9.39 (s, 1H), 7.72 (s, 1H), 7.63- |
| | | 7.57 (m, 1H), 7.38 (d, J = 4.4 Hz, 1H), 7.32-7.19 (m, 2H), 6.89 (s, |
| | | 2H), 5.17 (dd, J = 13.4, 5.2 Hz, 2H), 4.83-4.74 (m, 1H), 4.67 (d, J = |
| | | 15.1 Hz, 2H), 4.51-4.30 (m, 4H), 3.98 (d, J = 16.9 Hz, 6H), 3.79- |
| | | 3.54 (m, 1H), 3.01-2.77 (m, 2H), 2.60-2.45 (m, 1H), 2.25-2.13 (m, |
| | | 2H), 1.11 (d, J = 8.9 Hz, 4H). |
| D40 | 698.35 | ¹H NMR (400 MHz, Methanol-d4) δ 9.25 (s, 1H), 8.55 (s, 1H, FA), |
| | | 7.58 (s, 1H), 7.52 (d, J = 8.2 Hz, 1H), 7.39 (s, 1H), 7.37 (s, 1H), |
| | | 6.88 (s, 2H), 6.83 (s, 1H), 5.16 (dd, J = 13.4, 5.1 Hz, 1H), 4.60 (d, |
| | | J = 13.5 Hz, 1H), 4.52-4.37 (m, 3H), 4.00 (d, J = 7.0 Hz, 9H), 3.89- |
| | | 3.85 (m, 2H), 3.64-3.59 (m, 2H), 3.48-3.33 (m, 2H), 2.95-2.86 |
| | | (m, 1H), 2.81 (d, J = 17.2 Hz, 1H), 2.59-2.44 (m, 1H), 2.23-2.16 |
| | | (m, 1H), 1.62 (d, J = 6.4 Hz, 6H). |
| D41 | 708.45 | ¹H NMR (300 MHz, Methanol-d4) δ 9.16 (s, 1H), 8.56 (s, 1H, FA), |
| | | 7.51 (d, J = 9.0 Hz, 1H), 7.44 (s, 1H), 7.35 (d, J = 7.1 Hz, 2H), 6.88 |
| | | (s, 2H), 6.52 (s, 1H), 5.16 (dd, J = 13.2, 5.1 Hz, 1H), 4.64 (s, 2H), |
| | | 4.52-4.35 (m, 2H), 4.25 (br s, 2H), 3.97 (s, 6H), 3.68-3.54 (m, |
| | | 4H), 3.45-3.37 (m, 2H), 3.14 (s, 7H), 3.03-2.73 (m, 2H), 2.59- |
| | | 2.43 (m, 1H), 2.27-2.14 (m, 1H), 1.63 (s, 6H). |
| D42 | 692.20 | ¹H NMR (300 MHz, Methanol-d4) δ 9.09 (d, J = 3.5 Hz, 1H), 8.56 (s, |
| | | 1H), 7.67-7.37 (m, 2H), 7.21 (dd, J = 8.4, 2.3 Hz, 1H), 7.11 (s, |
| | | 1H), 6.85 (s, 2H), 6.18 (s, 1H), 5.15 (dd, J = 13.3, 5.1 Hz, 1H), |
| | | 4.55-4.26 (m, 7H), 4.15-4.00 (m, 6H), 3.94 (s, 6H), 3.58 (d, J = |
| | | 1.3 Hz, 3H), 2.96 (s, 3H), 2.95-2.87 (m, 1H), 2.85-2.73 (m, 1H), |
| | | 2.55-2.29 (m, 3H), 2.25-2.12 (m, 1H). |
| D43 | 637.15 | ¹H NMR (300 MHz, Methanol-d4) δ 9.14 (d, J = 0.7 Hz, 1H), 7.46- |
| | | 7.36 (m, 2H), 6.94 (d, J = 2.1 Hz, 1H), 6.86 (dd, J = 8.2, 2.2 Hz, |
| | | 1H), 6.73 (s, 2H), 6.52 (d, J = 0.8 Hz, 1H), 5.15 (dd, J = 13.2, 5.2 |
| | | Hz, 1H), 4.55-4.33 (m, 5H), 4.14-4.00 (m, 2H), 3.80 (s, 6H), 3.58 |
| | | (s, 3H), 3.12 (s, 6H), 2.98-2.86 (m, 1H), 2.85-2.76 (m, 1H), 2.57- |
| | | 2.44 (m, 1H), 2.24-2.13 (m, 1H). |
| D44 | 695.35 | ¹H NMR (400 MHz, Methanol-d4) δ 9.26 (s, 1H), 8.56 (s, 0.49H, |
| | | FA), 7.57 (s, 1H), 7.50 (d, J = 8.2 Hz, 1H), 7.35 (d, J = 8.3 Hz, 2H), |
| | | 6.83 (d, J = 5.9 Hz, 3H), 5.16 (dd, J = 13.4, 5.2 Hz, 1H), 4.46-4.39 |
| | | (m, 2H), 4.28-4.11 (m, 2H), 4.01 (s, 3H), 3.96 (s, 6H), 3.65 (s, 4H), |
| | | 3.42-3.36 (m, 2H), 3.30-3.18 (m, 3H), 2.95-2.89 (m, 1H), 2.83- |
| | | 2.77 (m, 1H), 2.54-2.47 (m, 1H), 2.22-2.16 (m, 1H), 1.62 (s, |
| | | 6H). |
| D45 | 634.30 | ¹H NMR (400 MHz, DMSO-d6) δ 10.98 (s, 1H), 9.02 (s, 1H), 7.45 |
| | | (d, J = 8.4 Hz, 1H), 7.36 (s, 1H), 7.34-7.25 (m, 3H), 7.21 (d, J = |
| | | 2.4 Hz, 1H), 7.00-6.92 (m, 2H), 6.39 (s, 1H), 5.11 (dd, J = 13.3, |
| | | 5.1 Hz, 1H), 4.40-4.15 (m, 2H), 4.03-3.81 (m, 3H), 3.46 (s, 3H), |
| | | 3.06 (s, 6H), 2.99-2.85 (m, 3H), 2.78 (s, 3H), 2.70-2.58 (m, 1H), |
| | | 2.44-2.32 (m, 1H), 2.05-1.95 (m, 1H), 1.97-1.80 (m, 2H), 1.75 |
| | | (d, J = 12.0 Hz, 2H). |
| D46 | 730.45 | ¹H NMR (300 MHz, DMSO-d6) δ 10.98 (s, 1H), 9.03 (s, 1H), 8.70 |
| | | (s, 1H, TFA salt), 7.59 (s, 1H), 7.42 (d, J = 8.8 Hz, 1H), 7.08 (s, 2H), |
| | | 6.75-6.67 (m, 2H), 6.17 (s, 1H), 5.08 (dd, J = 13.2, 5.0 Hz, 1H), |
| | | 4.34 (s, 2H), 4.31 (s, 1H), 4.20 (d, J = 16.7 Hz, 1H), 4.01 (t, J = 7.4 |
| | | Hz, 4H), 3.93 (s, 3H), 3.79 (s, 2H), 3.65 (s, 2H), 3.50 (s, 3H), 3.45- |
| | | 3.34 (m, 2H), 3.33-3.15 (m, 2H), 2.88-2.75 (m, 3H), 2.66-2.54 (m, |
| | | 1H), 2.44-2.30 (m, 3H), 2.20-2.09 (m, 2H), 2.08-1.94 (m, 3H), 1.22 |
| | | (t, J = 7.4 Hz, 3H). |
| D47 | 665.30 | ¹H NMR (400 MHz, DMSO-d6) δ 10.98 (s, 1H), 9.04 (s, 1H), 7.57 |
| | | (s, 1H), 7.45 (d, J = 8.4 Hz, 1H), 7.36-7.29 (m, 1H), 7.20 (d, J = |
| | | 2.3 Hz, 1H), 6.75 (s, 2H), 6.55-6.49 (m, 1H), 5.11 (dd, J = 13.3, |
| | | 5.1 Hz, 1H), 4.39-4.19 (m, 2H), 3.89-3.83 (m, 2H), 3.81 (s, 6H), |
| | | 3.48 (s, 3H), 3.45-3.37 (m, 2H), 3.08 (s, 6H), 2.99-2.86 (m, 1H), |
| | | 2.82-2.70 (m, 2H), 2.65-2.56 (m, 1H), 2.47-2.35 (m, 2H), 2.06- |
| | | 1.96 (m, 1H), 1.61-1.53 (m, 2H). |
| D48 | 707.20 | ¹H NMR (300 MHz, Methanol-d4) δ 9.48 (s, 1H), 8.55 (s, 1H, FA), |
| | | 7.85-7.69 (m, 2H), 7.34 (d, J = 8.6 Hz, 1H), 7.08-6.93 (m, 2H), |
| | | 6.86 (s, 2H), 5.24-5.06 (m, 1H), 4.82 (s, 2H), 4.63 (d, J = 8.0 Hz, |
| | | 2H), 4.46-4.26 (m, 2H), 3.92-3.83 (m, 6H), 3.76-3.69 (m, 4H), |
| | | 3.65 (d, J = 20.3 Hz, 3H), 3.56-3.46 (m, 2H), 3.29-3.17 (m, 2H), |
| | | 2.97-2.73 (m, 2H), 2.60-2.41 (m, 1H), 2.39-2.12 (m, 3H), 2.03- |
| | | 1.85 (m, 2H). |
| D49 | 694.35 | ¹H NMR (400 MHz, Methanol-d4) δ 9.15 (d, J = 0.8 Hz, 1H), 8.48 (s, |
| | | 0.2H, FA), 7.73 (d, J = 8.5 Hz, 1H), 7.42 (s, 1H), 7.19 (d, J = 2.3 Hz, |
| | | 1H), 7.06 (dd, J = 8.5, 2.4 Hz, 1H), 6.90-6.83 (m, 2H), 6.48 (s, |
| | | 1H), 5.10 (dd, J = 12.5, 5.4 Hz, 1H), 4.38-4.17 (m, 4H), 4.08- |
| | | 3.77 (m, 8H), 3.67-3.54 (m, 3H), 3.22-2.96 (m, 9H), 2.95-2.67 |
| | | (m, 4H), 2.16-2.07 (m, 1H). |
| D50 | 711.20 | ¹H NMR (400 MHz, DMSO-d6) δ 10.96 (s, 1H), 9.03 (s, 1H), 7.59 |
| | | (s, 1H), 7.40 (d, J = 8.4 Hz, 1H), 7.22 (dd, J = 8.5, 2.4 Hz, 1H), 7.13 |
| | | (d, J = 2.3 Hz, 1H), 6.75 (s, 2H), 6.49 (s, 1H), 5.09 (dd, J = 13.3, 5.1 |
| | | Hz, 1H), 4.37-4.15 (m, 2H), 3.91 (d, J = 12.1 Hz, 1H), 3.83 (s, 6H), |
| | | 3.53 (d, J = 12.9 Hz, 1H), 3.15 (d, J = 10.9 Hz, 2H), 3.07 (s, 6H), |
| | | 3.04-2.98 (m, 2H), 2.96-2.84 (m, 3H), 2.69-2.54 (m, 1H), 2.45- |
| | | 2.30 (m, 1H), 2.04-1.92 (m, 1H), 1.22 (d, J = 6.1 Hz, 6H). |
| D51 | 756.35 | ¹H NMR (300 MHz, DMSO-d6) δ 10.97 (s, 1H), 9.10 (s, 1H), 7.69 |
| | | (s, 1H), 7.41 (d, J = 8.4 Hz, 1H), 7.31-7.18 (m, 1H), 7.14 (d, J = |
| | | 2.3 Hz, 1H), 6.75 (s, 2H), 6.49 (s, 1H), 5.10 (dd, J = 13.3, 5.1 Hz, |
| | | 1H), 4.49 (t, J = 12.3 Hz, 4H), 4.41-4.12 (m, 2H), 3.83 (s, 6H), |
| | | 3.58 (s, 2H), 3.51 (s, 3H), 3.02 (d, J = 23.7 Hz, 5H), 2.63 (s, 3H), |
| | | 2.46-2.24 (m, 1H), 2.10-1.91 (m, 1H), 1.25 (s, 6H). |
| D52 | 694.40 | ¹H NMR (400 MHz, DMSO-d6) δ 10.97 (s, 1H), 8.99 (s, 1H), 8.13 |
| | | (s, 0.2H, FA), 7.45-7.38 (m, 2H), 7.28 (dd, J = 8.6, 2.5 Hz, 1H), |
| | | 7.18 (d, J = 2.4 Hz, 1H), 6.83 (s, 1H), 6.72 (s, 1H), 5.97 (s, 1H), |
| | | 5.20-5.03 (m, 1H), 4.41-4.15 (m, 2H), 3.85 (d, J = 11.8 Hz, 2H), |
| | | 3.76 (s, 3H), 3.66 (s, 3H), 3.51 (s, 1H), 3.44 (s, 3H), 3.01 (s, 6H), |
| | | 5.23-4.94 (m, 1H), 2.82-2.68 (m, 5H), 2.65-2.55 (m, 1H), 2.45- |
| | | 2.30 (m, 1H), 7.31-7.25 (m, 1H), 1.81 (s, 4H). |
| D53 | 679.30 | ¹H NMR (400 MHz, DMSO-d6) δ 10.98 (s, 1H), 9.02 (s, 1H), 7.58 |
| | | (s, 1H), 7.55-7.51 (m, 1H), 7.47 (s, 2H), 6.76 (s, 2H), 6.51 (s, 1H), |
| | | 5.10 (dd, J = 13.3, 5.1 Hz, 1H), 4.39 (d, J = 17.1 Hz, 1H), 4.26 (d, |
| | | J = 17.1 Hz, 1H), 3.83 (s, 6H), 3.70 (d, J = 11.9 Hz, 2H), 3.48 (s, 3H), |
| | | 3.08 (s, 6H), 3.03-2.83 (m, 3H), 2.65-2.56 (m, 3H), 2.47-2.32 |
| | | (m, 1H), 2.06-1.94 (m, 1H), 1.82 (s, 1H), 1.72 (d, J = 12.8 Hz, 2H), |
| | | 1.54-1.47 (m, 2H). |
| D54 | 695.50 | ¹H NMR (400 MHz, Methanol-d4) δ 9.27 (s, 1H), 7.59 (s, 1H), 7.53 |
| | | (d, J = 8.3 Hz, 1H), 7.39 (d, J = 9.7 Hz, 2H), 6.89 (s, 2H), 6.83 (s, |
| | | 1H), 5.17 (dd, J = 13.3, 5.2 Hz, 1H), 4.63 (d, J = 20.8 Hz, 1H), 4.57- |
| | | 4.38 (m, 3H), 4.01 (d, J = 5.1 Hz, 10H), 3.96-3.85 (m, 3H), 3.65 |
| | | (s, 3H), 3.60-3.44 (m, 1H), 2.99-2.87 (m, 1H), 2.86-2.75 (m, |
| | | 1H), 2.59-2.45 (m, 1H), 2.25-2.13 (m, 1H), 1.74-1.51 (m, 7H). |
| D55 | 666.25 | ¹H NMR (300 MHz, Methanol-d4) δ 9.25 (s, 1H), 8.56 (d, 1H), 7.79 |
| | | (d, J = 7.9 Hz, 1H), 7.58 (s, 1H), 7.54 (s, 1H), 7.48 (d, J = 8.1 Hz, |
| | | 1H), 6.94-6.78 (m, 3H), 5.17 (dd, J = 13.3, 5.1 Hz, 1H), 4.51 (d, |
| | | J = 5.0 Hz, 2H), 4.37-4.24 (m, 2H), 4.01 (s, 3H), 3.97 (s, 6H), 3.65 |
| | | (s, 3H), 3.57 (d, J = 12.0 Hz, 2H), 3.16-2.97 (m, 3H), 2.97-2.86 |
| | | (m, 1H), 2.86-2.75 (m, 1H), 2.51 (qd, J = 13.1, 4.7 Hz, 1H), 2.27- |
| | | 2.15 (m, 1H), 2.15-2.03 (m, 4H). |
| D56 | 720.40 | ¹H NMR (300 MHz, Methanol-d4) δ 9.10 (s, 1H), 8.51 (s, 0.2H, FA), |
| | | 7.46 (d, J = 11.0 Hz, 2H), 7.31 (d, J = 9.3 Hz, 2H), 6.80 (s, 2H), 6.23 |
| | | (s, 1H), 5.21-5.09 (m, 1H), 4.51-4.33 (m, 2H), 4.14-4.03 (m, |
| | | 4H), 3.93 (s, 8H), 3.59 (s, 3H), 3.20-3.14 (m, 5H), 2.96-2.70 (m, |
| | | 3H), 2.56-2.37 (m, 3H), 2.24-2.13 (m, 1H), 1.49 (s, 6H). |
| D57 | 681.40 | ¹H NMR (300 MHz, DMSO-d6) δ 10.97 (s, 1H), 9.16 (s, 1H), 7.74 |
| | | (s, 1H), 7.42 (d, J = 8.4 Hz, 1H), 7.32-7.21 (m, 1H), 7.16 (d, J = |
| | | 2.3 Hz, 1H), 6.78 (s, 1H), 6.74 (s, 2H), 5.10 (dd, J = 13.2, 5.0 Hz, |
| | | 1H), 4.40-4.13 (m, 2H), 4.09-3.98 (m, 1H), 3.94 (s, 3H), 3.83 (s, |
| | | 6H), 3.64-3.46 (m, 5H), 3.00-2.68 (m, 4H), 2.67-2.53 (m, 3H), |
| | | 2.46-2.25 (m, 2H), 2.07-1.90 (m, 1H), 1.30 (d, J = 5.0 Hz, 3H). |
| D58 | 677.45 | ¹H NMR (400 MHz, Methanol-d4) δ 8.91 (s, 1H), 7.95 (d, J = 2.2 Hz, |
| | | 1H), 7.85 (dd, J = 8.3, 2.3 Hz, 1H), 7.76 (d, J = 8.3 Hz, 1H), 7.54 (s, |
| | | 1H), 6.75 (s, 2H), 6.43 (s, 1H), 5.18 (dd, J = 13.3, 5.1 Hz, 1H), 4.63- |
| | | 4.47 (m, 2H), 4.24 (t, J = 7.6 Hz, 4H), 3.89 (s, 6H), 3.87-3.73 (m, |
| | | 3H), 3.63 (t, J = 12.1 Hz, 2H), 3.57 (s, 3H), 2.99-2.74 (m, 4H), |
| | | 2.60-2.44 (m, 3H), 2.21 (ddd, J = 9.7, 5.3, 2.7 Hz, 1H), 1.86 (d, J = |
| | | 13.8 Hz, 2H). |
| D59 | 652.40 | ¹H NMR (400 MHz, Methanol-d4) δ 9.23 (s, 1H), 8.09 (d, J = 2.2 Hz, |
| | | 1H), 7.97 (dd, J = 8.3, 2.3 Hz, 1H), 7.84 (d, J = 8.3 Hz, 1H), 7.51 (s, |
| | | 1H), 6.80 (s, 1H), 6.74 (s, 2H), 5.19 (dd, J = 13.3, 5.1 Hz, 1H), 4.67- |
| | | 4.49 (m, 2H), 3.99 (s, 3H), 3.90 (s, 6H), 3.88-3.76 (m, 5H), 3.62 |
| | | (s, 3H), 3.03-2.86 (m, 3H), 2.80 (ddd, J = 17.5, 4.8, 2.4 Hz, 1H), |
| | | 2.53 (qd, J = 13.2, 4.7 Hz, 1H), 2.21 (ddd, J = 10.9, 5.4, 3.0 Hz, |
| | | 1H), 1.95 (d, J = 13.5 Hz, 2H). |

### Example 9—Preparation of 4-(6-(dimethylamino)-2-methyl-1-oxo-1,2-dihydro-2,7-naphthyridin-4-yl)-2,6-dimethoxybenzaldehyde

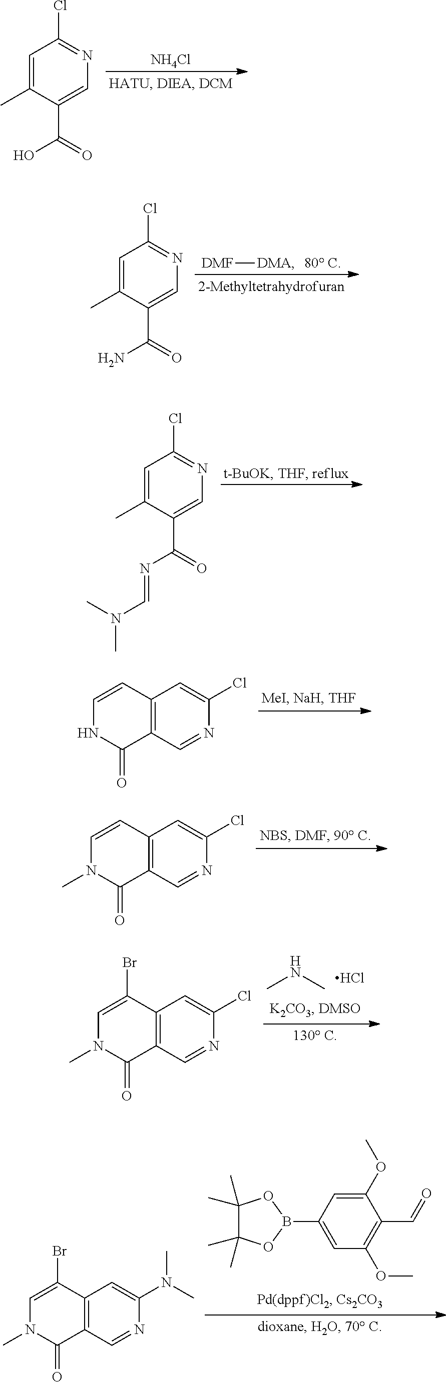

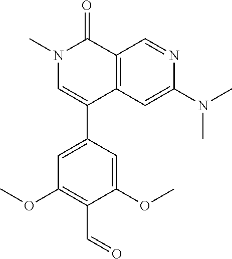

### Step 1: Preparation of 6-chloro-4-methylpyridine-3-carboxamide

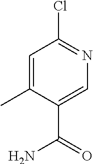

To a stirred mixture of 6-chloro-4-methylpyridine-3-carboxylic acid (20.00 g, 116.564 mmol, 1.00 equivalent) and NH₄Cl (62.35 g, 1.17 mol, 10.00 equivalent) in DCM (400 mL) was added DIEA (22.60 g, 174.846 mmol, 3.00 equivalent). After stirring for 5 min, HATU (66.48 g, 174.846 mmol, 1.50 equivalent) was added in portions. The resulting mixture was stirred for 3 hours at room temperature. The resulting mixture was concentrated under reduced pressure, and the residue was purified by silica gel column chromatography, eluted with PE/EtOAc from 1/1 to 3/2 to afford 6-chloro-4-methylpyridine-3-carboxamide (18.30 g, 61.3%) as a yellow solid. LCMS (ESI) m/z: [M+H]⁺=171.

### Step 2: Preparation of 6-chloro-N-[(1E)-(dimethylamino)methylidene]-4-methylpyridine-3-carboxamide

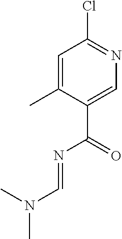

To a stirred mixture of 6-chloro-4-methylpyridine-3-carboxamide (18.30 g, 107.268 mmol, 1.00 equivalent) and in 2-methyltetrahydrofuran (100 mL) was added DMF-DMA (19.17 g, 160.903 mmol, 1.50 equivalent) at 80° C. under nitrogen atmosphere and stirred for additional 1 hour. Then the mixture was cooled and concentrated to afford 6-chloro-N-[(1E)-(dimethylamino)methylidene]-4-methylpyridine-3-carboxamide (26.3 g, 91.3%) as a yellow crude solid, that was used directly without further purification. LCMS (ESI) m/z: [M+H]⁺=226.

### Step 3: Preparation of 6-chloro-2H-2,7-naphthyridin-1-one

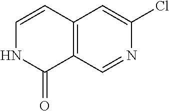 [SMILES: O=c1[nH]ccc2cc(Cl)ncc12]

To a stirred mixture of 6-chloro-N-[(1E)-(dimethylamino)methylidene]-4-methylpyridine-3-carboxamide (26.30 g) in THE (170.00 mL) was added t-BuOK (174.00 mL, 1 mol/L in THF), the resulting solution was stirred at 60° C. under nitrogen atmosphere for 30 min. Then the mixture was cooled and concentrated under reduced pressure, the crude solid was washed with saturated NaHCO₃ solution (100 mL) and collected to give 6-chloro-2H-2,7-naphthyridin-1-one (14.1 g, 67.0%) as a pink solid, that was used directly without further purification. LCMS (ESI) m/z: [M+H]⁺=181.

### Step 4: Preparation of 6-chloro-2-methyl-2,7-naphthyridin-1-one

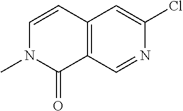

To a stirred mixture of 6-chloro-2H-2, 7-naphthyridin-1-one (14.10 g, 78.077 mmol, 1.00 equivalent) in anhydrous THE (280.00 mL) was added NaH (9.37 g, 234.232 mmol, 3.00 equivalent, 60%) in portions at 0° C. After 10 min, to above mixture was added Mel (33.25 g, 234.232 mmol, 3.00 equivalent) at 0° C., the mixture was allowed to stir for 10 min at 0 degrees. Then the mixture was allowed to stir for 12 h at room temperature. The resulting mixture was concentrated under reduced pressure. The crude solid was slurried with water (100 mL), and the solid was filtered and collected to give the 6-chloro-2-methyl-2,7-naphthyridin-1-one (14.6 g, 94.1%) as a yellow solid, that was used directly without further purification. LCMS (ESI) m/z: [M+H]⁺=195.

### Step 5: Preparation of 4-bromo-6-chloro-2-methyl-2,7-naphthyridin-1-one

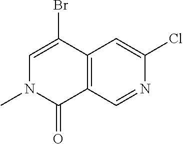

To a stirred mixture of 6-chloro-2-methyl-2,7-naphthyridin-1-one (8.00 g, 41.106 mmol, 1.00 equivalent) in DMF (160.00 mL) was added NBS (8.78 g, 49.327 mmol, 1.20 equivalent), the resulting mixture was stirred for 2 h at 90° C. The reaction mixture was cooled and diluted with DCM (150 mL), and washed with water (3×100 mL), the organic layers were dried and concentrated. Then the residue was slurried with EtOAc (20 mL), the slurry was filtered, the filter cake was washed with EtOAc (20 mL) to give 4-bromo-6-chloro-2-methyl-2,7-naphthyridin-1-one (6.32 g, 55.7%) as a white solid, that was used directly without further purification. LCMS (ESI) m/z: [M+H]⁺=273.

### Step 6: Preparation of 4-bromo-6-(dimethylamino)-2-methyl-2,7-naphthyridin-1-one

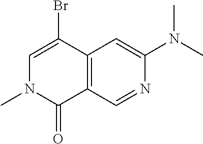

A stirred mixture of 4-bromo-6-chloro-2-methyl-2,7-naphthyridin-1-one (6.00 g, 21.937 mmol, 1.00 equivalent), dimethylamine hydrochloride (5.37 g, 65.811 mmol, 3.00 equivalent) and K₂CO₃ (15.16 g, 109.685 mmol, 5.00 equivalent) in DMSO (60.00 mL) was heated at 130° C. under nitrogen atmosphere. After 3 h, the resulting mixture was cooled and diluted with water (100 mL), and then extracted with EtOAc (3×100 mL). The combined organic layers were washed with saturated NaCl solution (3×50 mL), dried over anhydrous Na₂SO₄, concentrated under reduced pressure to afford 4-bromo-6-(dimethylamino)-2-methyl-2,7-naphthyridin-1-one (5.91 g, 93.6%) as a yellow solid, that was used directly without further purification. LCMS (ESI) m/z: [M+H]⁺=282.

### Step 7: Preparation of (4-[6-(dimethylamino)-2-methyl-1-oxo-2,7-naphthyridin-4-yl]-2,6-dimethoxy benzaldehyde

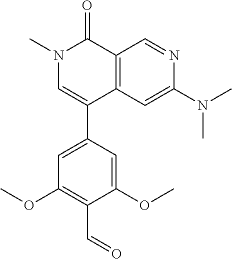

To a stirred mixture of 4-bromo-6-(dimethylamino)-2-methyl-2,7-naphthyridin-1-one (5.70 g, 20.203 mmol, 1.00 equivalent) and 2,6-dimethoxy-4-(4,4,5,5-tetramethyl-1,3,2-dioxaborolan-2-yl)benzaldehyde (8.26 g, 28.284 mmol, 1.40 equivalent) in dioxane (100.00 mL) and H₂O (10.00 mL) was added Pd(dppf)Cl₂·CH₂Cl₂ (1.65 g, 2.020 mmol, 0.10 equivalent) and Cs₂CO₃ (13.16 g, 40.405 mmol, 2.00 equivalent), then the mixture was allowed to stir for 4 h at 70° C. under nitrogen atmosphere. The resulting mixture was cooled and concentrated under reduced pressure, the residue was slurried with water (100 mL) and filtered, the filter cake was collected. And this solid was further slurried with MeOH (100 mL) and filtered, the solid was collected to afford product to afford 4-[6-(dimethylamino)-2-methyl-1-oxo-2,7-naphthyridin-4-yl]-2,6-dimethoxybenzaldehyde (6.10 g, 77.6%) as a brown solid. LCMS (ESI) m/z: [M+H]⁺=368.

### Example 10—Preparation of 3-(6-(1-(4-(6-(dimethylamino)-2-methyl-1-oxo-1,2-dihydro-2,7-naphthyridin-4-yl)-2,6-dimethoxybenzyl)piperidin-4-yl)-1-oxoisoindolin-2-yl)piperidine-2,6-dione formic acid; and 3-(5-(1-(4-(6-(dimethylamino)-2-methyl-1-oxo-1,2-dihydro-2,7-naphthyridin-4-yl)-2,6-dimethoxybenzyl)piperidin-4-yl)-1-oxoisoindolin-2-yl)piperidine-2,6-dione formic acid

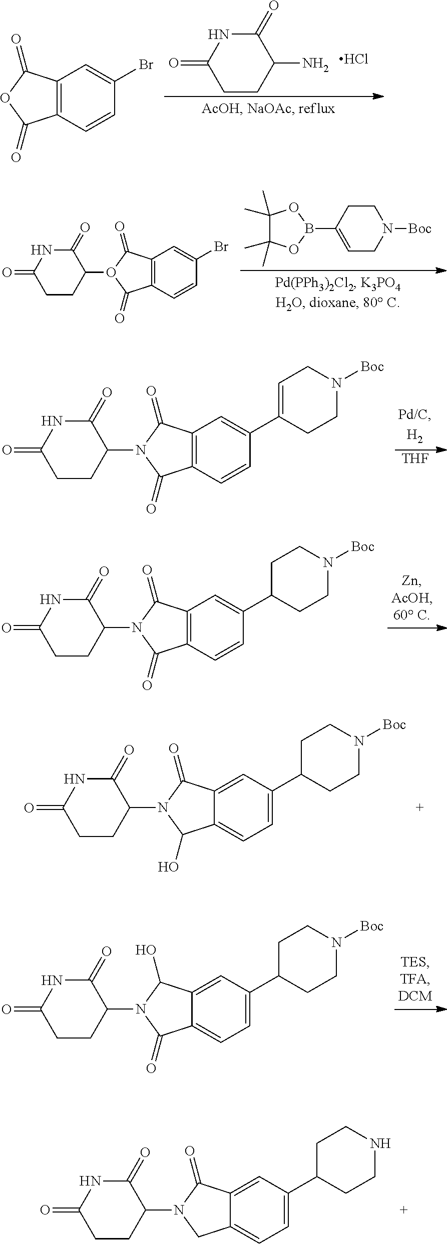

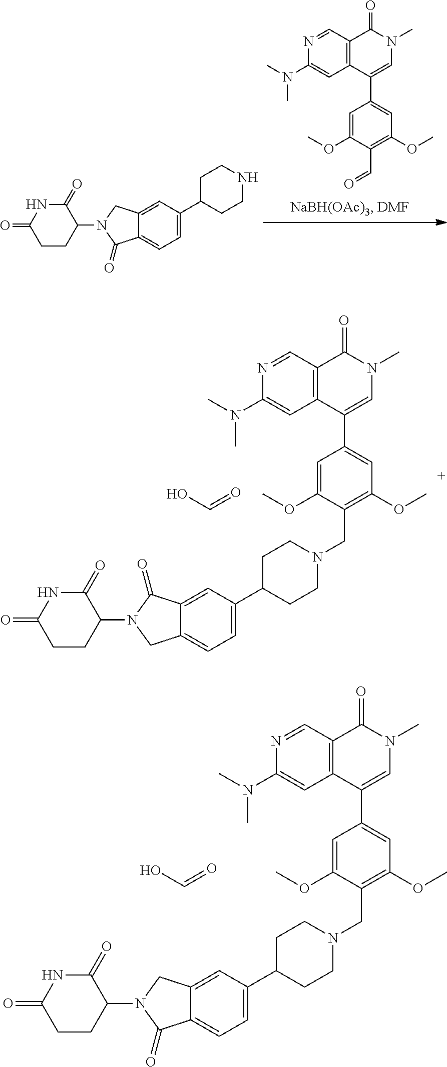

### Step 1: Preparation of 5-bromo-2-(2,6-dioxopiperidin-3-yl)isoindole-1,3-dione

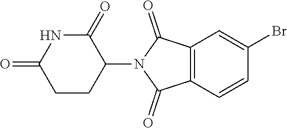 [SMILES: O=C1CCC(N2C(=O)c3ccc(Br)cc3C2=O)C(=O)N1]

To a stirred solution of 5-bromo-2-benzofuran-1,3-dione (10.00 g, 44.050 mmol, 1.00 equivalent), NaOAc (7.23 mg, 88.134 mmol, 2.00 equivalent) and 3-aminopiperidine-2,6-dione (11.29 g, 88.113 mmol, 2.00 equivalent) in AcOH (80.00 mL) at room temperature. The resulting mixture was stirred for 16 h at 115° C. The resulting mixture was concentrated under reduced pressure. The residue was purified by silica gel column chromatography, eluted with petroleum ether/EtOAc (10:1) to afford 5-bromo-2-(2,6-dioxopiperidin-3-yl)isoindole-1,3-dione (13.6 g, 91.6%) as a dark brown solid. LCMS (ESI) m/z: [M+H]⁺=337.

### Step 2: Preparation of tert-butyl 4-[2-(2,6-dioxopiperidin-3-yl)-1,3-dioxoisoindol-5-yl]-3,6-dihydro-2H-pyridine-1-carboxylate

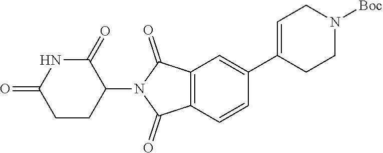 [SMILES: CC(C)(C)OC(=O)N1CC=C(c2ccc3c(c2)C(=O)N(C2CCC(=O)NC2=O)C3=O)CC1]

To a stirred solution of 5-bromo-2-(2,6-dioxopiperidin-3-yl)isoindole-1,3-dione (3.00 g, 8.899 mmol, 1.00 equivalent), tert-butyl4-(4,4,5,5-tetramethyl-1,3,2-dioxaborolan-2-yl)-3,6-dihydro-2H-pyridine-1-carboxylate (3.30 g, 10.672 mmol, 1.20 equivalent), K₃PO₄ (5.67 g, 26.712 mmol, 3.00 equivalent) in dioxane (20.00 mL) and H₂O (4.00 mL) was added Pd(PPh₃)₂Cl₂ (0.62 g, 0.883 mmol, 0.10 equivalent) at room temperature under nitrogen atmosphere. The resulting mixture was stirred for 16 h at 80° C. under nitrogen atmosphere. The resulting mixture was concentrated under reduced pressure. The residue was purified by silica gel column chromatography, eluted with PE/EtOAc (8/1) to afford tert-butyl 4-[2-(2,6-dioxopiperidin-3-yl)-1,3-dioxoisoindol-5-yl]-3,6-dihydro-2H-pyridine-1-carboxylate (0.8 g, 20.5%) as a colorless oil. LCMS (ESI) m/z: [M+H]⁺=440.

### Step 3: Preparation of tert-butyl 4-[2-(2,6-dioxopiperidin-3-yl)-1,3-dioxoisoindol-5-yl]piperidine-1-carboxylate

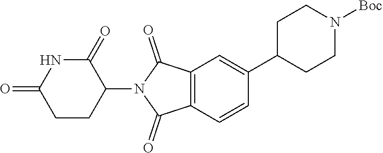 [SMILES: CC(C)(C)OC(=O)N1CCC(c2ccc3c(c2)C(=O)N(C2CCC(=O)NC2=O)C3=O)CC1]

To a stirred solution of tert-butyl 4-[2-(2,6-dioxopiperidin-3-yl)-1,3-dioxoisoindol-5-yl]-3,6-dihydro-2H-pyridine-1-carboxylate (0.80 g) in THE (20.00 mL) was added 10% Pd/C (500.0 mg) under nitrogen atmosphere in a 100 mL round-bottom flask. The mixture was hydrogenated at room temperature for 12 h under hydrogen atmosphere using a hydrogen balloon, filtered through a Celite pad and concentrated under reduced pressure. This resulted in tert-butyl 4-[2-(2,6-dioxopiperidin-3-yl)-1,3-dioxoisoindol-5-yl]piperidine-1-carboxylate (0.73 g, crude) as a white solid that was used in the next step directly without further purification. LCMS (ESI) m/z: [M+H]⁺=442.

### Step 4: Preparation of tert-butyl 4-(2-(2,6-dioxopiperidin-3-yl)-1-hydroxy-3-oxoisoindolin-5-yl)piperidine-1-carboxylate; tert-butyl 4-(2-(2,6-dioxopiperidin-3-yl)-3-hydroxy-1-oxoisoindolin-5-yl)piperidine-1-carboxylate

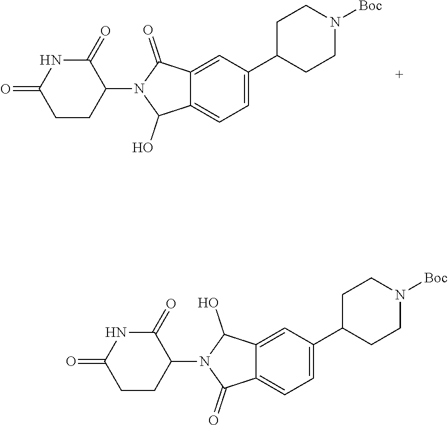 [SMILES: CC(C)(C)OC(=O)N1CCC(c2ccc3c(c2)C(=O)N(C2CCC(=O)NC2=O)C3O)CC1 ; CC(C)(C)OC(=O)N1CCC(c2ccc3c(c2)C(O)N(C2CCC(=O)NC2=O)C3=O)CC1]

To a stirred solution of tert-butyl 4-[2-(2,6-dioxopiperidin-3-yl)-1,3-dioxoisoindol-5-yl]piperidine-1-carboxylate (0.73 g, 16.55 mmol, 1.00 equivalent) and Zn (1.08 g, 1.65 mmol, 10.00 equivalent) in AcOH (10.00 mL) at room temperature. The resulting mixture was stirred for 2 h at 60° C. The resulting mixture was concentrated under reduced pressure. The residue was purified by silica gel column chromatography, eluted with PE/EtOAc (2:1) to afford tert-butyl 4-(2-(2,6-dioxopiperidin-3-yl)-1-hydroxy-3-oxoisoindolin-5-yl)piperidine-1-carboxylate; tert-butyl 4-(2-(2,6-dioxopiperidin-3-yl)-3-hydroxy-1-oxoisoindolin-5-yl)piperidine-1-carboxylate (0.546 g, 74.8%, mixture of two regio-isomers) as a colorless solid. LCMS (ESI) m/z: [M+H]⁺=444.

### Step 5: Preparation of 3-(1-oxo-6-(piperidin-4-yl)isoindolin-2-yl)piperidine-2,6-dione; 3-(1-oxo-5-(piperidin-4-yl)isoindolin-2-yl)piperidine-2,6-dione

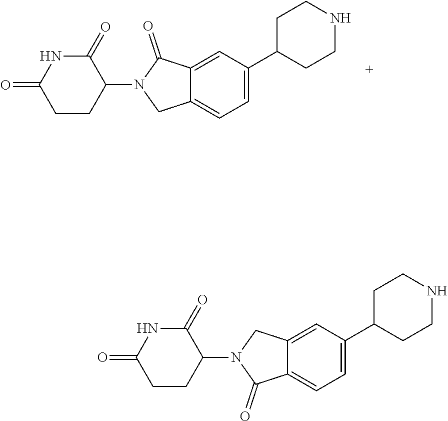 [SMILES: O=C1CCC(N2Cc3ccc(C4CCNCC4)cc3C2=O)C(=O)N1 ; O=C1CCC(N2Cc3cc(C4CCNCC4)ccc3C2=O)C(=O)N1]

To a stirred solution of tert-butyl 4-(2-(2,6-dioxopiperidin-3-yl)-1-hydroxy-3-oxoisoindolin-5-yl)piperidine-1-carboxylate; tert-butyl 4-(2-(2,6-dioxopiperidin-3-yl)-3-hydroxy-1-oxoisoindolin-5-yl)piperidine-1-carboxylate (mixture of two regio-isomers, 573.00 mg, 1.00 equivalent) and TFA (3.00 mL) in DCM (9.00 mL) was added TES (450.7 mg, 3.876 mmol, 3.00 equivalent) at room temperature. The resulting mixture was stirred for 2 h at room temperature. The resulting mixture was concentrated under reduced pressure, This was used directly without further purification, to afford 3-(1-oxo-6-(piperidin-4-yl)isoindolin-2-yl)piperidine-2,6-dione; 3-(1-oxo-5-(piperidin-4-yl)isoindolin-2-yl)piperidine-2,6-dione (200 mg 36.6% mixture of two regio-isomers) as an off-white oil. LCMS (ESI) m/z: [M+H]⁺=328.

### Step 6: Preparation of 3-(6-(1-(4-(6-(dimethylamino)-2-methyl-1-oxo-1,2-dihydro-2,7-naphthyridin-4-yl)-2,6-dimethoxybenzyl)piperidin-4-yl)-1-oxoisoindolin-2-yl)piperidine-2,6-dione formic acid; and 3-(5-(1-(4-(6-(dimethylamino)-2-methyl-1-oxo-1,2-dihydro-2,7-naphthyridin-4-yl)-2,6-dimethoxybenzyl)piperidin-4-yl)-1-oxoisoindolin-2-yl)piperidine-2,6-dione formic acid

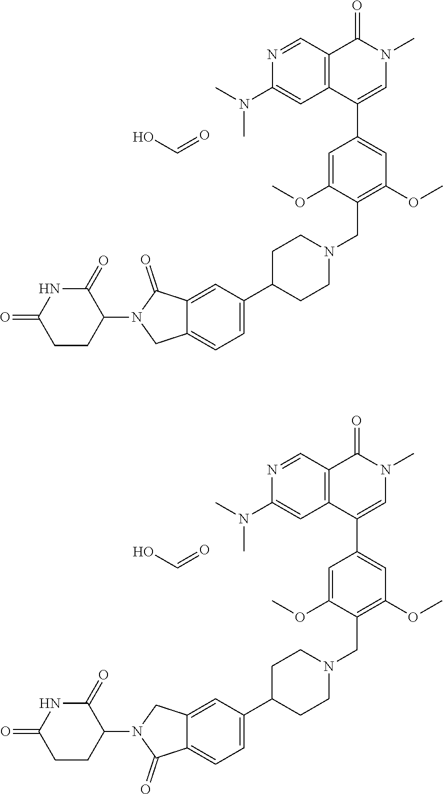

To a stirred solution of 3-[1-oxo-6-(piperidin-4-yl)-3H-isoindol-2-yl]piperidine-2,6-dione (165.0 mg, 0.504 mmol, 1.00 equivalent), and 3-(1-oxo-6-(piperidin-4-yl)isoindolin-2-yl)piperidine-2,6-dione; 3-(1-oxo-5-(piperidin-4-yl)isoindolin-2-yl)piperidine-2,6-dione (mixture of two regio-isomers, 222.2 mg, 0.605 mmol, 1.20 equivalent) in DMF (4.00 mL) was added NaBH(OAc)₃ (427.3 mg, 2.016 mmol, 4.00 equivalent) at room temperature. The resulting mixture was stirred for 16 h at room temperature. The residue was purified by reverse flash chromatography with the following conditions: column, C18 silica gel; mobile phase, CH₃CN in water (0.05% FA), 0% to 50% gradient in 30 min; detector, UV 254 nm. The crude product was purified by Prep-HPLC with the following conditions: Column, Sunfire Prep C18 OBD Column, 10 μm, 19*250 mm; mobile phase, water (0.05% FA) and CH₃CN (15% to 22% CH₃CN in 15 min); Detector, UV 254 nm. This resulted in 3-[6-[1-([4-[6-(dimethylamino)-2-methyl-1-oxo-2,7-naphthyridin-4-yl]-2,6-dimethoxyphenyl]methyl)piperidin-4-yl]-1-oxo-3H-isoindol-2-yl]piperidine-2,6-dione; formic acid (52.5 mg, 26.3%) as a white solid and 3-[5-[1-([4-[6-(dimethylamino)-2-methyl-1-oxo-2,7-naphthyridin-4-yl]-2,6-dimethoxyphenyl]methyl)piperidin-4-yl]-1-oxo-3H-isoindol-2-yl]piperidine-2,6-dione; formic acid (68.4 mg, 34.2%) as a yellow solid.

For 3-[6-[1-([4-[6-(dimethylamino)-2-methyl-1-oxo-2,7-naphthyridin-4-yl]-2,6-dimethoxyphenyl]methyl)piperidin-4-yl]-1-oxo-3H-isoindol-2-yl]piperidine-2,6-dione; formic acid: ¹H NMR (400 MHz, DMSO-d6) δ 10.97 (s, 1H), 9.04 (s, 1H), 8.20 (s, 1H, FA), 7.58 (d, J=14.5 Hz, 2H), 7.52 (s, 2H), 6.79 (s, 2H), 6.50 (s, 1H), 5.10 (dd, J=13.4, 5.1 Hz, 1H), 4.41 (d, J=17.1 Hz, 1H), 4.28 (d, J=17.0 Hz, 1H), 3.84 (s, 6H), 3.68 (s, 2H), 3.49 (s, 3H), 3.08-3.05 (m, 8H), 2.91-2.89 (m, 1H), 2.66-2.56 (m, 2H), 2.40-2.35 (m, 1H), 2.30 (t, J=11.3 Hz, 2H), 2.03-1.95 (m, 1H), 1.88-1.57 (m, 4H). LCMS (ESI) m/z: [M+H]⁺=679.32.

For 3-[5-[1-([4-[6-(dimethylamino)-2-methyl-1-oxo-2,7-naphthyridin-4-yl]-2,6-dimethoxyphenyl]methyl)piperidin-4-yl]-1-oxo-3H-isoindol-2-yl]piperidine-2,6-dione; formic acid: 1H NMR (400 MHz, DMSO-d6) δ 10.98 (s, 1H), 9.05 (s, 1H), 8.15 (s, 1H, FA), 7.69 (d, J=7.8 Hz, 1H), 7.60 (s, 1H), 7.48 (s, 1H), 7.40 (d, J=7.9 Hz, 1H), 6.87 (s, 2H), 6.51 (s, 1H), 5.11 (dd, J=13.3, 5.1 Hz, 1H), 4.44 (d, J=17.3 Hz, 1H), 4.31 (d, J=17.3 Hz, 1H), 4.05 (s, 2H), 3.90 (s, 6H), 3.49 (s, 3H), 3.31 (d, J=11.7 Hz, 2H), 3.09 (s, 6H), 2.99-2.71 (m, 4H), 2.65-2.56 (m, 1H), 2.47-2.33 (m, 1H), 2.04-1.96 (m, 1H), 1.92 (m, 4H). LCMS (ESI) m/z: [M+H]⁺=679.32.

### Example 11—Preparation 4-(6-cyclopropyl-2-methyl-1-oxo-1,2-dihydro-2,7-naphthyridin-4-yl)-2,6-dimethoxybenzaldehyde

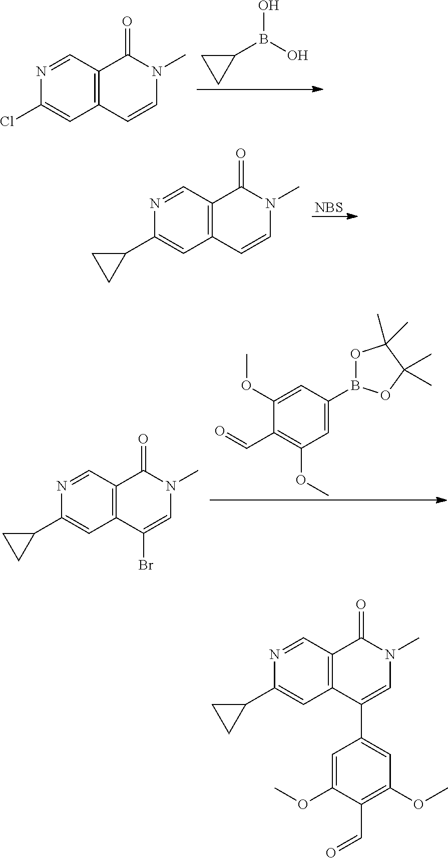

### Step 1: Preparation of 6-cyclopropyl-2-methyl-2,7-naphthyridin-1-one

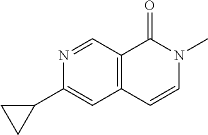

To a stirred solution of 6-chloro-2-methyl-2,7-naphthyridin-1-one (500.00 mg, 2.569 mmol, 1.00 equivalent) and cyclopropylboronic acid (441.37 mg, 5.138 mmol, 2 equivalent) in toluene (20.00 mL) and water (1.00 mL) was added tricyclohexylphosphane (144.09 mg, 0.514 mmol, 0.20 equivalent), Pd(AcO)₂ (57.68 mg, 0.257 mmol, 0.10 equivalent) and K₃PO₄ (1636.01 mg, 7.707 mmol, 3.00 equivalent) at room temperature under nitrogen atmosphere. The resulting mixture was stirred for 1 h at 110° C. The mixture was concentrated under reduced pressure. The residue was purified by silica gel column chromatography, eluted with CH₂Cl₂/MeOH (50:1) to afford 6-cyclopropyl-2-methyl-2,7-naphthyridin-1-one (340 mg, 59.48%) as a brown solid. LCMS (ESI) m/z: [M+H]+=201.

### Step 2: Preparation of 4-bromo-6-cyclopropyl-2-methyl-2,7-naphthyridin-1-one

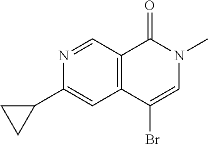 [SMILES: Cn1cc(Br)c2cc(C3CC3)ncc2c1=O]

To a stirred solution of 6-cyclopropyl-2-methyl-2,7-naphthyridin-1-one (100.00 mg, 0.499 mmol, 1.00 equivalent) in DMF (4.00 mL) was added NBS (106.66 mg, 0.599 mmol, 1.20 equivalent) at room temperature under nitrogen atmosphere. The resulting mixture was stirred for 2 h at 90° C. The resulting mixture was diluted with water (12 mL), extracted with EtOAc (3×100 mL). The combined organic layers were washed with brine (2×50 mL), dried over anhydrous Na₂SO₄. After filtration, the filtrate was concentrated under reduced pressure to afford 4-bromo-6-cyclopropyl-2-methyl-2,7-naphthyridin-1-one (400 mg, 75.96%) as a brown solid. That was used directly without further purification. LCMS (ESI) m/z: [M+H]+=279.

### Step 3: Preparation of 4-(6-cyclopropyl-2-methyl-1-oxo-2,7-naphthyridin-4-yl)-2,6-dimethoxybenzaldehyde

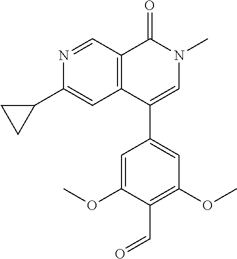 [SMILES: COc1cc(-c2cn(C)c(=O)c3cnc(C4CC4)cc23)cc(OC)c1C=O]

To a stirred solution of 4-bromo-6-cyclopropyl-2-methyl-2,7-naphthyridin-1-one (420.00 mg, 1.505 mmol, 1.00 equivalent) and 2,6-dimethoxy-4-(4,4,5,5-tetramethyl-1,3,2-dioxaborolan-2-yl)benzaldehyde (527.48 mg, 1.806 mmol, 1.2 equivalent) in dioxane (10.00 mL) and water (2.00 mL) was added Pd(dppf)Cl₂ (110.09 mg, 0.150 mmol, 0.10 equivalent) and K₂CO₃ (415.90 mg, 3.009 mmol, 2.00 equivalent) at room temperature under nitrogen atmosphere. The resulting mixture was stirred for overnight at 80° C. The mixture was concentrated under reduced pressure. The residue was purified by Prep-TLC (CH₂Cl₂/MeOH 50:1) to afford 4-(6-cyclopropyl-2-methyl-1-oxo-2,7-naphthyridin-4-yl)-2,6-dimethoxybenzaldehyde (440 mg, 72.22%) as a yellow solid. LCMS (ESI) m/z: [M+H]+=365.

### Example 12—Preparation of 3-[6-[(1-[[4-(6-cyclopropyl-2-methyl-1-oxo-2,7-naphthyridin-4-yl)-2,6-dimethoxyphenyl]methyl]135zetidine-3-yl)oxy]-1-oxo-3H-isoindol-2-yl]piperidine-2,6-dione

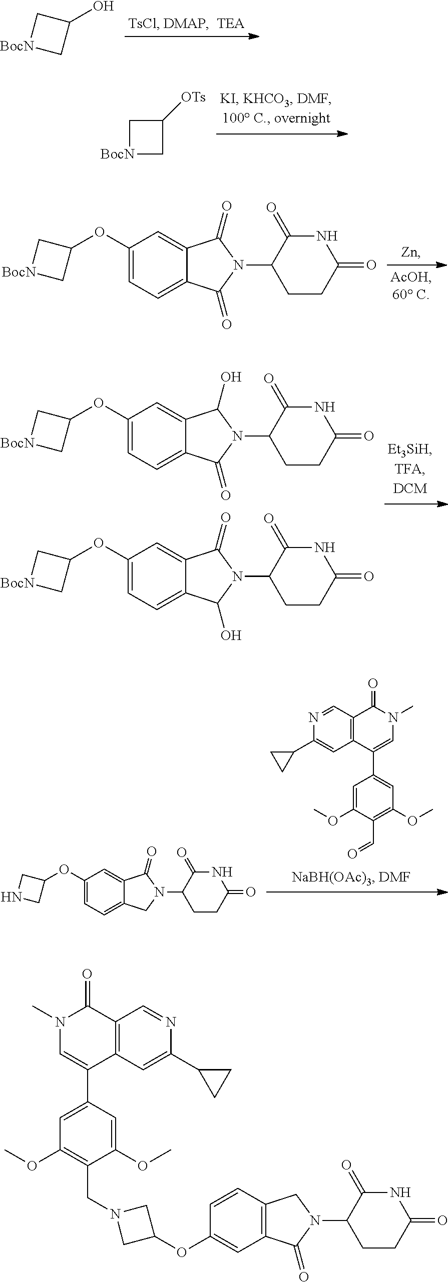

### Step 1: Preparation of tert-butyl 3-[(4-methylbenzenesulfonyl)oxy]azetidine-1-carboxylate (25)

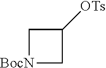 [SMILES: Cc1ccc(S(=O)(=O)OC2CN(C(=O)OC(C)(C)C)C2)cc1]

To a stirred solution of tert-butyl 3-hydroxyazetidine-1-carboxylate (2.50 g, 14.433 mmol, 1.00 equivalent) and TsCl (4.13 g, 21.650 mmol, 1.50 equivalent) in DCM were added DMAP (264.49 mg, 2.165 mmol, 0.15 equivalent) and TEA (4.38 g, 43.300 mmol, 3.00 equivalent) in portions at 0° C. under air atmosphere. The resulting mixture was concentrated under reduced pressure. The residue was purified by silica gel column chromatography, eluted with Petroleum ether/EtOAc (1:1) to afford tert-butyl 3-[(4-methylbenzenesulfonyl)oxy]azetidine-1-carboxylate (4.4 g, 93.11%) as a brown oil. LCMS (ESI) m/z: [M+H]⁺=328.

### Step 2: Preparation of tert-butyl 3-[[2-(2,6-dioxopiperidin-3-yl)-1,3-dioxoisoindol-5-yl]oxy] azetidine-1-carboxylate

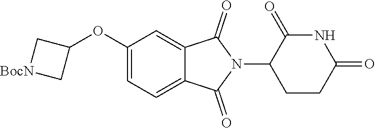 [SMILES: CC(C)(C)OC(=O)N1CC(Oc2ccc3c(c2)C(=O)N(C2CCC(=O)NC2=O)C3=O)C1]

To a solution of tert-butyl 3-[(4-methylbenzenesulfonyl)oxy]azetidine-1-carboxylate (4.40 g, 13.439 mmol, 1.00 equivalent) and KI (0.22 g, 1.344 mmol, 0.10 equivalent) in DMF was added KHCO₃ (4.04 g, 40.318 mmol, 3.00 equivalent) in portions at 100° C. under air atmosphere. The resulting mixture was washed with 3×150 mL of EtOAc. The resulting mixture was concentrated under reduced pressure. The residue was purified by reverse flash chromatography with the following conditions: column, C18 silica gel; mobile phase, MeCN in water, 0% to 100% gradient in 40 min; detector, UV 254 nm. This resulted in tert-butyl 3-[[2-(2,6-dioxopiperidin-3-yl)-1,3-dioxoisoindol-5-yl]oxy]azetidine-1-carboxylate (1.73 g, 29.98%) as an off-white solid. LCMS (ESI) m/z: [M+H]⁺=430.

### Step 3: Preparation of tert-butyl 3-[[2-(2,6-dioxopiperidin-3-yl)-1-hydroxy-3-oxo-1H-isoindol-5-yl]oxy]azetidine-1-carboxylate, and tert-butyl 3-[[2-(2,6-dioxopiperidin-3-yl)-3-hydroxy-1-oxo-3H-isoindol-5-yl]oxy]azetidine-1-carboxylate

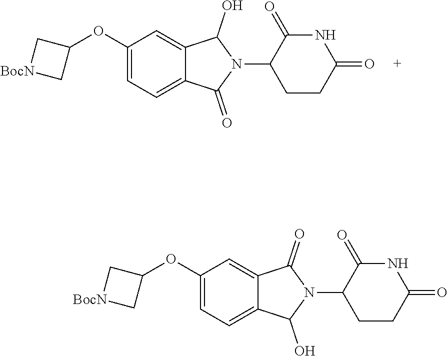

A solution of tert-butyl 3-[[2-(2,6-dioxopiperidin-3-yl)-1,3-dioxoisoindol-5-yl]oxy] azetidine-1-carboxylate (1.73 g, 4.029 mmol, 1.00 equivalent) and Zn (2.64 g, 40.286 mmol, 10.00 equivalent) in AcOH was stirred for 2 h at 60° C. under air atmosphere. The resulting mixture was washed with 3×100 mL of ethyl acetate. The resulting mixture was concentrated under reduced pressure. The crude product was used in the next step directly without further purification to afford tert-butyl 3-[[2-(2,6-dioxopiperidin-3-yl)-1-hydroxy-3-oxo-1H-isoindol-5-yl] oxy]azetidine-1-carboxylate and tert-butyl 3-[[2-(2,6-dioxopiperidin-3-yl)-3-hydroxy-1-oxo-3H-isoindol-5-yl]oxy]azetidine-1-carboxylate (2.73 g, 78.53%) as an off-white solid. LCMS (ESI) m/z: [M+H]⁺=432.

### Step 4: Preparation of 3-[6-(137zetidine-3-yloxy)-1-oxo-3H-isoindol-2-yl]piperidine-2,6-dione

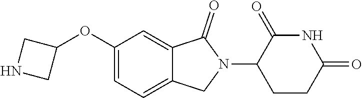 [SMILES: O=C1CCC(N2Cc3ccc(OC4CNC4)cc3C2=O)C(=O)N1]

To a solution of tert-butyl 3-[[2-(2,6-dioxopiperidin-3-yl)-1-hydroxy-3-oxo-1H-isoindol-5-yl]oxy]azetidine-1-carboxylate and tert-butyl 3-[[2-(2,6-dioxopiperidin-3-yl)-3-hydroxy-1-oxo-3H-isoindol-5-yl]oxy]azetidine-1-carboxylate (2.73 g, 3.164 mmol, 1.00 equivalent) and TFA (1.50 mL, 20.195 mmol, 6.38 equivalent) in DCM was added Et₃SiH (3.68 g, 31.638 mmol, 10.00 equivalent) in portions at room temperature under air atmosphere. The resulting mixture was concentrated under reduced pressure. The crude product (mg) was purified by Prep-HPLC with the following conditions (Column: Xcelect CSH F-pheny OBD Column, 19*250 mm, 5 μm; Mobile Phase A:Water (0.05% TFA), Mobile Phase B: can; Flow rate: 30 mL/min; Gradient:5 B to 21 B in 10 min; 254/220 nm; RT1: 7.20/8.67 min) to afford 3-[6-(azetidin-3-yloxy)-1-oxo-3H-isoindol-2-yl]piperidine-2,6-dione (165 mg, 8.27%) as an off-white solid. LCMS (ESI) m/z: [M+H]⁺=316.

### Step 5: Preparation of 3-[6-[(1-[[4-(6-cyclopropyl-2-methyl-1-oxo-2,7-naphthyridin-4-yl)-2,6-dimethoxyphenyl]methyl]azetidin-3-yl)oxy]-1-oxo-3H-isoindol-2-yl]piperidine-2,6-dione

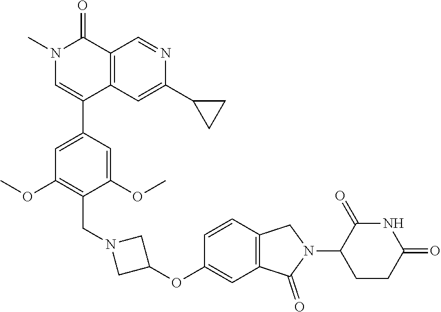 [SMILES: COc1cc(-c2cn(C)c(=O)c3cnc(C4CC4)cc23)cc(OC)c1CN1CC(Oc2ccc3c(c2)C(=O)N(C2CCC(=O)NC2=O)C3)C1]

To a stirred solution of 3-[6-(azetidin-3-yloxy)-1-oxo-3H-isoindol-2-yl piperidine-2,6-dione (75.00 mg, 0.238 mmol, 1.00 equivalent) and 4-(6-cyclopropyl-2-methyl-1-oxo-2,7-naphthyridin-4-yl)-2,6-dimethoxybenzaldehyde (86.67 mg, 0.238 mmol, 1.00 equivalent) in DMF was added NaBH(OAc)₃ (100.82 mg, 0.476 mmol, 2.00 equivalent) dropwise at room temperature under air atmosphere for 2 hours. The crude product (mg) was purified by Prep-HPLC with the following conditions (Column: XSelect CSH Prep C18 OBD Column, 19*250 mm, 5 μm; Mobile Phase A:Water (0.05% TFA), Mobile Phase B:ACN; Flow rate: 25 mL/min; Gradient:15 B to 23 B in 12 min; 254/220 nm; RT1: 10.38 min) to afford 3-[6-[(1-[[4-(6-cyclopropyl-2-methyl-1-oxo-2,7-naphthyridin-4-yl)-2,6-dimethoxyphenyl] methyl]azetidin-3-yl)oxy]-1-oxo-3H-isoindol-2-yl]piperidine-2,6-dione (18.9 mg, 11.69%) as an off-white solid. ¹H NMR (400 MHz, Methanol-d4) δ 9.39 (d, J=0.8 Hz, 1H), 7.80 (d, J=4.5 Hz, 1H), 7.60 (t, J=7.2 Hz, 1H), 7.42 (d, J=5.4 Hz, 1H), 7.32-7.24 (m, 1H), 7.22 (d, J=3.2 Hz, 1H), 6.89 (s, 2H), 5.35-5.19 (m, 1H), 5.16 (dd, J=13.3, 5.2 Hz, 1H), 4.84-4.69 (m, 2H), 4.65 (s, 2H), 4.48 (d, J=10.6 Hz, 2H), 4.42 (s, 2H), 3.98 (d, J=22.6 Hz, 6H), 3.69 (s, 3H), 2.93 (ddd, J=17.6, 13.5, 5.4 Hz, 1H), 2.80 (ddd, J=17.6, 4.7, 2.4 Hz, 1H), 2.52 (qd, J=13.2, 4.7 Hz, 1H), 2.21 (dddd, J=14.5, 10.7, 6.9, 3.9 Hz, 2H), 1.23-1.12 (m, 2H), 1.09 (d, J=4.4 Hz, 2H). LCMS (ESI) m/z: [M+H]⁺=664.

### Example 13—Preparation of 4-(6-cyclopropyl-2-(methyl-d3)-1-oxo-1,2-dihydro-2,7-naphthyridin-4-yl)-2,6-dimethoxybenzaldehyde

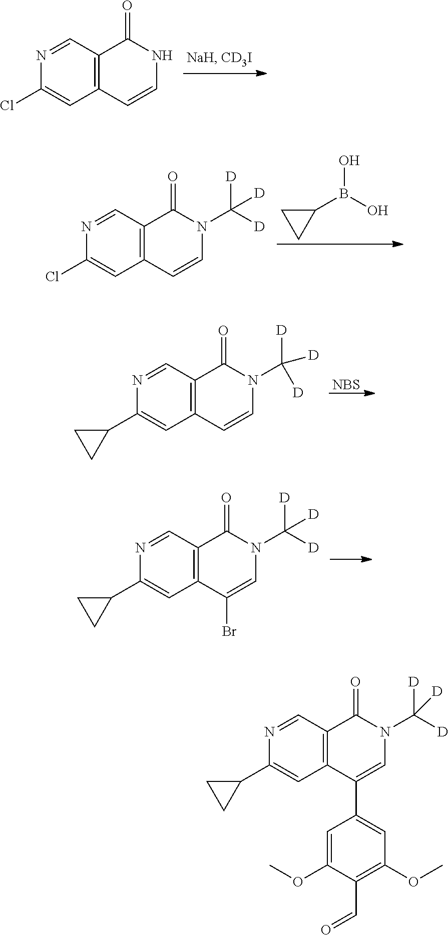

### Step 1: Preparation of 6-chloro-2-(2H3)methyl-2,7-naphthyridin-1-one

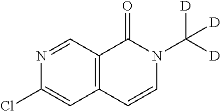 [SMILES: [2H]C([2H])([2H])n1ccc2cc(Cl)ncc2c1=O]

A solution of 6-chloro-2H-2,7-naphthyridin-1-one (500.00 mg, 2.769 mmol, 1.00 equivalent) in THE (5.00 mL) was treated with NaH (132.89 mg, 5.537 mmol, 2.00 equivalent) for 5 min at 0° C. followed by the addition of CD₃I (802.69 mg, 5.537 mmol, 2.00 equivalent) in portions at 0° C. After stirring at 0° C. for 1 h, the reaction mixture was poured into ice-water (50 mL), the precipitated solids were collected by filtration and washed with water (3×50 mL), then the solid was dried under vacuum to afford 6-chloro-2-(2H3)methyl-2,7-naphthyridin-1-one (500 mg, 91.37%) as a light yellow solid that was used directly without further purification. LCMS (ESI) m/z: [M+H]⁺=198.

### Step 2: Preparation of 6-cyclopropyl-2-(2H3)methyl-2,7-naphthyridin-1-one

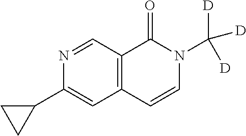 [SMILES: [2H]C([2H])([2H])n1ccc2cc(C3CC3)ncc2c1=O]

A mixture of 6-chloro-2-(2H3)methyl-2,7-naphthyridin-1-one (400.00 mg, 2.024 mmol, 1.00 equivalent), cyclopropylboronic acid (260.78 mg, 3.036 mmol, 1.50 equivalent), K₃PO₄ (1288.81 mg, 6.072 mmol, 3.00 equivalent), PCy₃ (113.51 mg, 0.405 mmol, 0.20 equivalent) and Pd(AcO)₂ (45.44 mg, 0.202 mmol, 0.10 equivalent) in Toluene (20.00 mL) and H₂O (1.00 mL) was stirred for 2 h at 110° C. under nitrogen atmosphere. The resulting mixture was concentrated under reduced pressure. The residue was purified by silica gel column chromatography, eluted with CH₂Cl₂/MeOH (10:1) to afford 6-cyclopropyl-2-(2H3)methyl-2,7-naphthyridin-1-one (350 mg, 85.08%) as a white solid. LCMS (ESI) m/z: [M+H]⁺=204

### Step 3: Preparation of 4-bromo-6-cyclopropyl-2-(2H3)methyl-2,7-naphthyridin-1-one

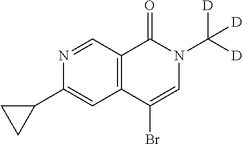 [SMILES: [2H]C([2H])([2H])n1cc(Br)c2cc(C3CC3)ncc2c1=O]

A mixture of 6-cyclopropyl-2-(2H3)methyl-2,7-naphthyridin-1-one (300.00 mg, 1.476 mmol, 1.00 equivalent) and NBS (315.23 mg, 1.771 mmol, 1.20 equivalent) in ACN (3.00 mL) was stirred for 2 h at 90° C. The resulting mixture was diluted with 1×50 mL of water. The resulting mixture was extracted with EtOAc (3×50 mL). The combined organic layers were washed with water (3×50 mL), dried over anhydrous Na₂SO₄. After filtration, the filtrate was concentrated under reduced pressure. The resulting mixture was concentrated under reduced pressure. to afford 4-bromo-6-cyclopropyl-2-(2H3)methyl-2,7-naphthyridin-1-one (350 mg, 84.04%) as a yellow solid that was used directly without further purification. LCMS (ESI) m/z: [M+H]⁺=282.

### Step 4: Preparation of 4-[6-cyclopropyl-2-(2H3)methyl-1-oxo-2,7-naphthyridin-4-yl]-2,6-dimethoxybenzaldehyde

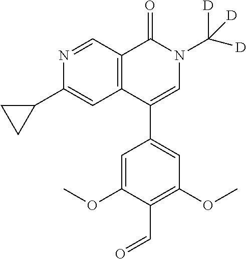 [SMILES: [2H]C([2H])([2H])n1cc(-c2cc(OC)c(C=O)c(OC)c2)c2cc(C3CC3)ncc2c1=O]

A mixture of 4-bromo-6-cyclopropyl-2-(2H3)methyl-2,7-naphthyridin-1-one (350.00 mg, 1.240 mmol, 1.00 equivalent), 2,6-dimethoxy-4-(4,4,5,5-tetramethyl-1,3,2-dioxaborolan-2-yl)benzaldehyde (434.86 mg, 1.489 mmol, 1.20 equivalent), Cs₂CO₃ (808.33 mg, 2.481 mmol, 2.00 equivalent) and Pd(dppf)Cl₂ (90.76 mg, 0.124 mmol, 0.10 equivalent) in dioxane (3.00 mL) and H₂O (1.00 mL) was stirred for 3 hours at 90° C. under nitrogen atmosphere. The resulting mixture was concentrated under reduced pressure. The residue was purified by silica gel column chromatography, eluted with CH₂Cl₂/MeOH (10:1) to afford 4-[6-cyclopropyl-2 (2H3) methyl-1-oxo-2,7-naphthyridin-4-yl]-2,6-dimethoxybenzaldehyde (200 mg, 43.88%) as an orange solid. LCMS (ESI) m/z: [M+H]⁺=368.

### Example 14—Preparation of 3-[5-[7-([4-[6-(azetidin-1-yl)-2-methyl-1-oxo-2,7-naphthyridin-4-yl]-2,6-dimethoxyphenyl]methyl)-2,7-diazaspiro[3.5]nonan-2-yl]-1-oxo-3H-isoindol-2-yl]piperidine-2,6-dione formic acid

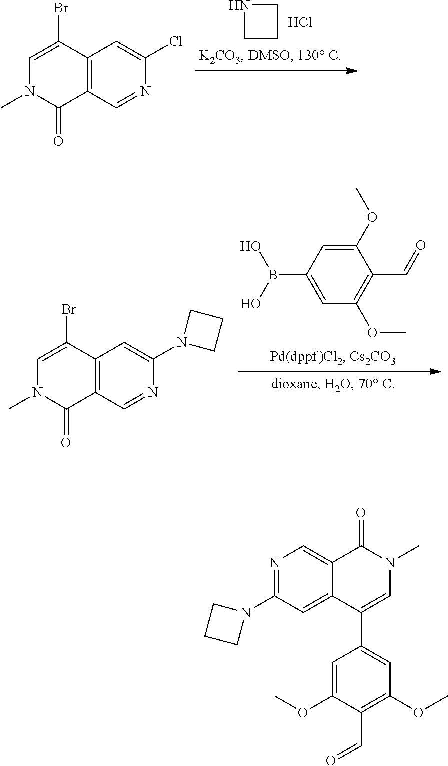

### Step 1: Preparation of 6-(azetidin-1-yl)-4-bromo-2-methyl-2, 7-naphthyridin-1-one

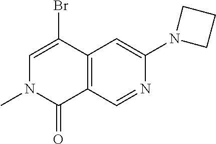 [SMILES: Cn1cc(Br)c2cc(N3CCC3)ncc2c1=O]

To a solution of 4-bromo-6-chloro-2-methyl-2,7-naphthyridin-1-one (5.00 g, 18.281 mmol, 1.00 equivalent) and azetidine hydrochloride (3.2 g, 54.843 mmol, 3 equivalent) in DMSO (50.00 mL) was added K₂CO₃ (12.6 g, 91.404 mmol, 5 equivalent). The resulting solution was stirred at 130° C. for 2 hours. The resulting mixture was cooled and diluted with water (100 mL), and then extracted with EtOAc (3×100 mL). The combined organic layers were washed with saturated NaCl solution (3×50 mL), dried over anhydrous Na₂SO₄, concentrated under reduced pressure to afford 6-(azetidin-1-yl)-4-bromo-2-methyl-2,7-naphthyridin-1-one (3.7 g, 68.8%) as a grey solid, that was used directly without further purification. LCMS (ESI) m/z: [M+H]⁺=294.

### Step 2: Preparation of 4-[6-(azetidin-1-yl)-2-methyl-1-oxo-2,7-naphthyridin-4-yl]-2,6-dimethoxybenzaldehyde

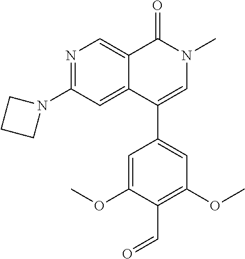 [SMILES: COc1cc(-c2cn(C)c(=O)c3cnc(N4CCC4)cc23)cc(OC)c1C=O]

To a solution of 6-(azetidin-1-yl)-4-bromo-2-methyl-2,7-naphthyridin-1-one (1.42 g, 4.827 mmol, 1.00 equivalent) and 4-formyl-3,5-dimethoxyphenylboronic acid (1.52 g, 7.241 mmol, 1.5 equivalent) in dioxane (16.00 mL) and H₂O (4.00 mL) were added Pd(dppf)Cl₂ (353.2 mg, 0.483 mmol, 0.1 equivalent) and Cs₂CO₃ (3.15 g, 9.655 mmol, 2 equivalent), and the resulting solution was stirred at 70° C. for 2 hours. The resulting mixture was cooled and concentrated under reduced pressure. The residue was slurried with water (30 mL) and filtered, the filter cake was collected. And this solid was further slurried with MeOH (30 mL) and filtered. The solid was collected to afford product to afford 4-[6-(azetidin-1-yl)-2-methyl-1-oxo-2,7-naphthyridin-4-yl]-2,6-dimethoxybenzaldehyde (1.42 g, 77.5%) as a grey and solid. LCMS (ESI) m/z: [M+H]⁺=380.

### Example 15—Preparation of 3-[5-[7-([4-[6-(azetidin-1-yl)-2-methyl-1-oxo-2,7-naphthyridin-4-yl]-2,6-dimethoxyphenyl]methyl)-2,7-diazaspiro[3.5]nonan-2-yl]-1-oxo-3H-isoindol-2-yl]piperidine-2,6-dione formic acid

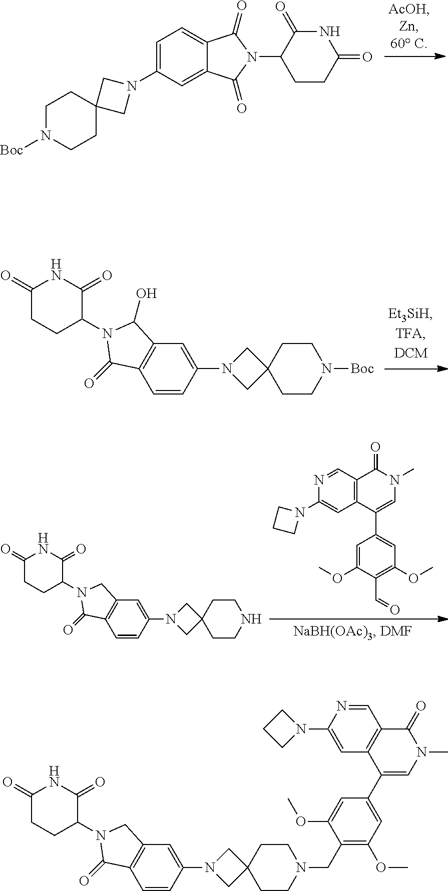 [SMILES: CC(C)(C)OC(=O)N1CCC2(CC1)CN(c1ccc3c(c1)C(=O)N(C1CCC(=O)NC1=O)C3=O)C2 ; CC(C)(C)OC(=O)N1CCC2(CC1)CN(c1ccc3c(c1)C(O)N(C1CCC(=O)NC1=O)C3=O)C2 ; O=C1CCC(N2Cc3cc(N4CC5(CCNCC5)C4)ccc3C2=O)C(=O)N1 ; COc1cc(-c2cn(C)c(=O)c3cnc(N4CCC4)cc23)cc(OC)c1C=O ; COc1cc(-c2cn(C)c(=O)c3cnc(N4CCC4)cc23)cc(OC)c1CN1CCC2(CC1)CN(c1ccc3c(c1)CN(C1CCC(=O)NC1=O)C3=O)C2]

### Step 1: Preparation of tert-butyl 2-[2-(2,6-dioxopiperidin-3-yl)-3-hydroxy-1-oxo-3H-isoindol-5-yl]-2,7-diaza spiro[3.5]nonane-7-carboxylate

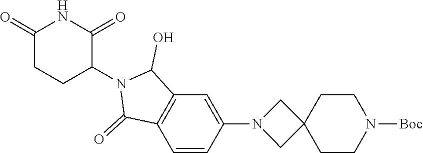 [SMILES: CC(C)(C)OC(=O)N1CCC2(CC1)CN(c1ccc3c(c1)C(O)N(C1CCC(=O)NC1=O)C3=O)C2]

To a stirred solution of tert-butyl 2-[2-(2,6-dioxopiperidin-3-yl)-1,3-dioxoisoindol-5-yl]-2,7-diazaspiro[3.5]nonane-7-carboxylate (500.0 mg, 1.036 mmol, 1.00 equivalent) in AcOH (4.00 mL) was added Zn (677.7 mg, 10.362 mmol, 10.00 equivalent). The resulting mixture was stirred at 60° C. for 2 h. The reaction mixture was filtered, and the filtrate was evaporated to afford crude product. The crude product was purified by reverse phase column, elution gradient 0 to 30% MeCN in water (containing 0.1% formic acid). Pure fractions were evaporated to dryness to afford tert-butyl 2-[2-(2,6-dioxopiperidin-3-yl)-3-hydroxy-1-oxo-3H-isoindol-5-yl]-2,7-diaza spiro[3.5]nonane-7-carboxylate (277.3 mg, 55.2%) as a yellow solid. LCMS (ESI) m/z: [M+H]⁺=485.

### Step 2: Preparation of 3-(5-[2,7-diazaspiro[3.5]nonan-2-yl]-1-oxo-3H-isoindol-2-yl)piperidine-2,6-dione

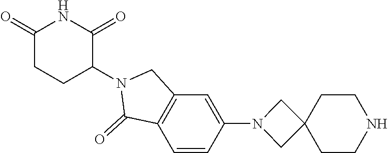 [SMILES: O=C1CCC(N2Cc3cc(N4CC5(CCNCC5)C4)ccc3C2=O)C(=O)N1]

To a stirred solution of tert-butyl 2-[2-(2,6-dioxopiperidin-3-yl)-3-hydroxy-1-oxo-3H-isoindol-5-yl]-2,7-diazaspiro[3.5]nonane-7-carboxylate (250.0 mg, 0.516 mmol, 1.00 equivalent) in DCM (2.00 mL) were added TFA (0.50 mL) and Et₃SiH (0.20 mL). The resulting mixture was stirred at room temperature for 1 hour. The resulting mixture was concentrated under reduced pressure. This resulted in 3-(5-[2,7-diazaspiro[3.5]nonan-2-yl]-1-oxo-3H-isoindol-2-yl)piperidine-2,6-dione (267.5 mg, crude) as a yellow gum. The crude product was used in the next step directly without further purification. LCMS (ESI) m/z: [M+H]⁺=369.

### Step 3: Preparation of 3-[5-[7-([4-[6-(azetidin-1-yl)-2-methyl-1-oxo-2,7-naphthyridin-4-yl]-2,6-dimethoxyphenyl]methyl)-2,7-diazaspiro[3.5]nonan-2-yl]-1-oxo-3H-isoindol-2-yl]piperidine-2,6-dione; formic acid

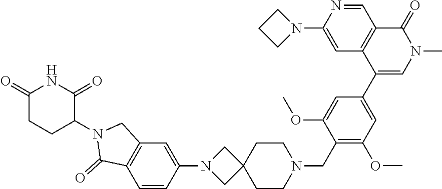 [SMILES: COc1cc(-c2cn(C)c(=O)c3cnc(N4CCC4)cc23)cc(OC)c1CN1CCC2(CC1)CN(c1ccc3c(c1)CN(C1CCC(=O)NC1=O)C3=O)C2]

To a stirred solution of 3-(5-[2,7-diazaspiro[3.5]nonan-2-yl]-1-oxo-3H-isoindol-2-yl)piperidine-2,6-dione (400.0 mg, 1.086 mmol, 1.00 equivalent) and 4-[6-(azetidin-1-yl)-2-methyl-1-oxo-2,7-naphthyridin-4-yl]-2,6-dimethoxybenzaldehyde (494.3 mg, 1.303 mmol, 1.20 equivalent) in DMF (3.00 mL) was added NaBH(OAc)₃ (920.4 mg, 4.343 mmol, 4.00 equivalent) at room temperature. The resulting mixture was stirred at room temperature for 2 hours. The crude reaction solution was directly purified by Prep-HPLC with the following conditions (Column: XSelect CSH Prep C18 OBD Column, 5 μm, 19*150 mm; Mobile Phase A: Water (0.05% FA), Mobile Phase B: ACN; Flow rate:25 mL/min; Gradient:14 B to 22 B in 15 min; 254/220 nm; RT1: 11.72 min) to afford 3-[6-[7-([4-[6-(azetidin-1-yl)-2-methyl-1-oxo-2,7-naphthyridin-4-yl]-2,6-dimethoxyphenyl]methyl)-2,7-diazaspiro[3.5]nonan-2-yl]-1-oxo-3H-isoindol-2-yl]piperidine-2,6-dione; formic acid (99.2 mg, 12.5%) as a white solid. ¹H NMR (400 MHz, DMSO-d6) δ 10.94 (s, 1H), 9.02 (s, 1H), 8.15 (s, 1H, FA), 7.61 (s, 1H), 7.48 (d, J=8.2 Hz, 1H), 6.75 (s, 2H), 6.53-6.44 (m, 2H), 6.21 (s, 1H), 5.04 (dd, J=13.3, 5.2 Hz, 1H), 4.30 (d, J=17.0 Hz, 1H), 4.17 (d, J=16.9 Hz, 1H), 4.01 (t, J=7.4 Hz, 4H), 3.83 (s, 6H), 3.61 (d, J=13.2 Hz, 6H), 3.48 (s, 3H), 2.96-2.84 (m, 1H), 2.63-2.54 (m, 3H), 2.51-2.45 (m, 2H), 2.35 (q, J=6.6 Hz, 3H), 1.95 (d, J=12.9 Hz, 1H), 1.75 (s, 4H). LCMS (ESI) m/z: [M+H]⁺=732.45.

### Example 16—Preparation of 3-[5-[1-([4-[6-(azetidin-1-yl)-2-methyl-1-oxo-2,7-naphthyridin-4-yl]-2,6-dimethoxy phenyl]methyl)piperidin-4-yl]-1-oxo-3H-isoindol-2-yl]piperidine-2,6-dione formic acid; and 3-[6-[1-([4-[6-(azetidin-1-yl)-2-methyl-1-oxo-2,7-naphthyridin-4-yl]-2,6-dimethoxyphenyl] methyl)piperidin-4-yl]-1-oxo-3H-isoindol-2-yl]piperidine-2,6-dione formic acid

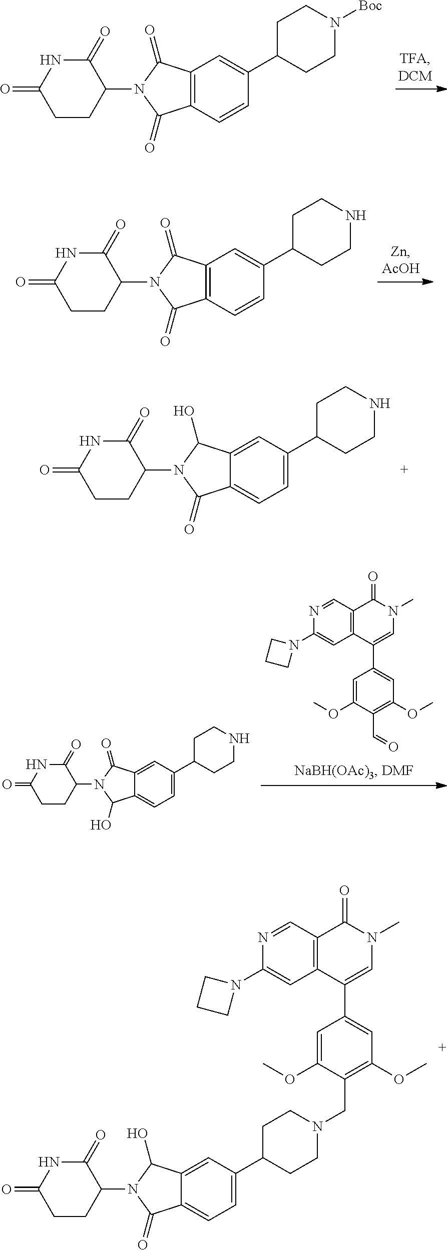 [SMILES: CC(C)(C)OC(=O)N1CCC(c2ccc3c(c2)C(=O)N(C2CCC(=O)NC2=O)C3=O)CC1 ; O=C1CCC(N2C(=O)c3ccc(C4CCNCC4)cc3C2=O)C(=O)N1 ; O=C1CCC(N2C(=O)c3ccc(C4CCNCC4)cc3C2O)C(=O)N1 ; O=C1CCC(N2C(=O)c3cc(C4CCNCC4)ccc3C2O)C(=O)N1 ; COc1cc(-c2cn(C)c(=O)c3cnc(N4CCC4)cc23)cc(OC)c1C=O ; COc1cc(-c2cn(C)c(=O)c3cnc(N4CCC4)cc23)cc(OC)c1CN1CCC(c2ccc3c(c2)C(O)N(C2CCC(=O)NC2=O)C3=O)CC1]

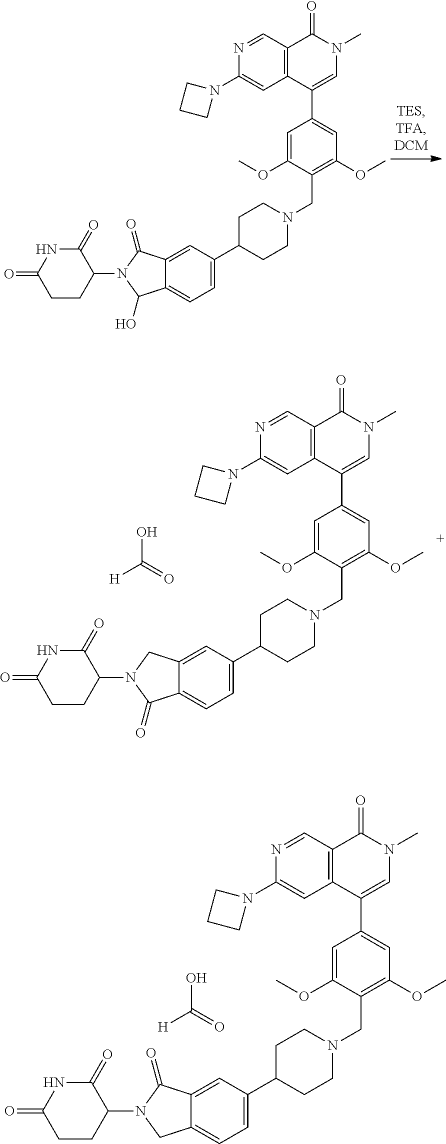

### Step 1: Preparation of 2-(2,6-dioxopiperidin-3-yl)-5-(piperidin-4-yl)isoindole-1,3-dione

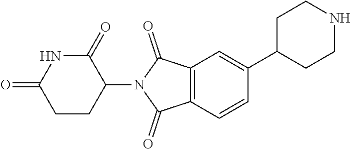 [SMILES: O=C1CCC(N2C(=O)c3ccc(C4CCNCC4)cc3C2=O)C(=O)N1]

To a stirred solution of tert-butyl 4-[2-(2, 6-dioxopiperidin-3-yl)-1,3-dioxoisoindol-5-yl]piperidine-1-carboxylate (1.00 g, 2.265 mmol, 1.00 equivalent) in DCM (8 mL) was added TFA (2.00 mL) at room temperature. The resulting mixture was stirred for 2 h at room temperature. The resulting mixture was concentrated under reduced pressure. This resulted in 2-(2,6-dioxopiperidin-3-yl)-5-(piperidin-4-yl)isoindole-1,3-dione (1.23 g, crude) as a white solid that was used in the next step directly without further purification. LCMS (ESI) m/z: [M+H]⁺=342.

### Step 2: Preparation of 3-[3-hydroxy-1-oxo-5-(piperidin-4-yl)-3H-isoindol-2-yl]piperidine-2,6-dione and 3-[1-hydroxy-3-oxo-5-(piperidin-4-yl)-1H-isoindol-2-yl]piperidine-2,6-dione

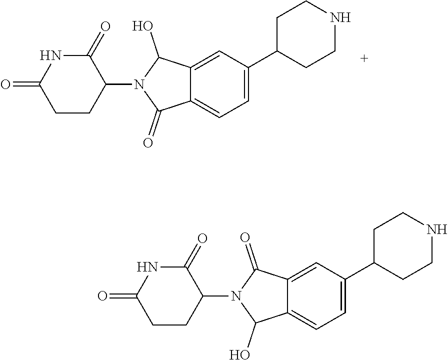 [SMILES: O=C1CCC(N2C(=O)c3ccc(C4CCNCC4)cc3C2O)C(=O)N1 ; O=C1CCC(N2C(=O)c3cc(C4CCNCC4)ccc3C2O)C(=O)N1]

To a solution of 2-(2,6-dioxopiperidin-3-yl)-5-(piperidin-4-yl)isoindole-1,3-dione (300.0 mg, 0.879 mmol, 1.00 equivalent) in AcOH (5.00 mL) was added Zn (574.9 mg, 8.788 mmol, 10 equivalent), and the resulting solution was stirred at 25° C. for 2 hours. The mixture was diluted with EtOAc (30 mL) and washed with water (30 mL×3). The organic layers were combined and dried over anhydrous sodium sulfate, filtered and concentrated to give a crude product. The crude product was purified by flash C18 chromatography (elution gradient 0 to 11% ACN in H₂O) to give 3-[3-hydroxy-1-oxo-5-(piperidin-4-yl)-3H-isoindol-2-yl]piperidine-2,6-dione and 3-[1-hydroxy-3-oxo-5-(piperidin-4-yl)-1H-isoindol-2-yl]piperidine-2,6-dione (280 mg, mixture of two regio-isomers, 92.8%) as a white solid. LCMS (ESI) m/z: [M+H]⁺=344.

### Step 3: Preparation of 3-[5-[1-([4-[6-(azetidin-1-yl)-2-methyl-1-oxo-2,7-naphthyridin-4-yl]-2,6-dimethoxyphenyl]methyl)piperidin-4-yl]-3-hydroxy-1-oxo-3H-isoindol-2-yl]piperidine-2,6-dione and 3-[5-[1-([4-[6-(azetidin-1-yl)-2-methyl-1-oxo-2,7-naphthyridin-4-yl]-2,6-dimethoxyphenyl]methyl)piperidin-4-yl]-1-hydroxy-3-oxo-1H-isoindol-2-yl]piperidine-2,6-dione

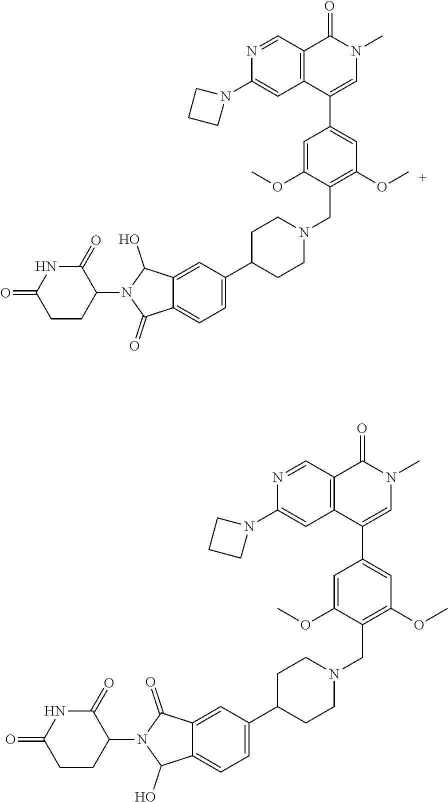

To a solution of 3-[3-hydroxy-1-oxo-5-(piperidin-4-yl)-3H-isoindol-2-yl]piperidine-2,6-dione and 3-[1-hydroxy-3-oxo-5-(piperidin-4-yl)-1H-isoindol-2-yl]piperidine-2,6-dione (mixture of two regio-isomers, 260.0 mg, 0.757 mmol, 1.00 equivalent), 4-[6-(azetidin-1-yl)-2-methyl-1-oxo-2,7-naphthyridin-4-yl]-2,6-dimethoxybenzaldehyde (287.3 mg, 0.757 mmol, 1 equivalent) in DMF (3 mL) was added NaBH(OAc)₃ (321.0 mg, 1.514 mmol, 2 equivalent), and the resulting solution was stirred at 25° C. for 4 hours. The mixture was diluted with EtOAc (20 mL) and washed with water (20 mL×3). The organic layers were combined and dried over anhydrous sodium sulfate, filtered and concentrated to give a crude product. The crude product was purified by Prep-TLC (CH₂Cl₂/MeOH 10:1) to give 3-[5-[1-([4-[6-(azetidin-1-yl)-2-methyl-1-oxo-2,7-naphthyridin-4-yl]-2,6-dimethoxyphenyl]methyl)piperidin-4-yl]-3-hydroxy-1-oxo-3H-isoindol-2-yl]piperidine-2,6-dione and 3-[5-[1-([4-[6-(azetidin-1-yl)-2-methyl-1-oxo-2,7-naphthyridin-4-yl]-2,6-dimethoxyphenyl]methyl)piperidin-4-yl]-1-hydroxy-3-oxo-1H-isoindol-2-yl]piperidine-2,6-dione (208 mg, mixture of two regio-isomers, 38.9%) as a white solid. LCMS (ESI) m/z: [M+H]⁺=707.

### Step 4: Preparation of 3-[5-[1-([4-[6-(azetidin-1-yl)-2-methyl-1-oxo-2,7-naphthyridin-4-yl]-2,6-dimethoxy phenyl]methyl) piperidin-4-yl]-1-oxo-3H-isoindol-2-yl]piperidine-2,6-dione formic acid; and 3-[6-[1-([4-[6-(azetidin-1-yl)-2-methyl-1-oxo-2,7-naphthyridin-4-yl]-2,6-dimethoxyphenyl]methyl)piperidin-4-yl]-1-oxo-3H-isoindol-2-yl]piperidine-2,6-dione formic acid

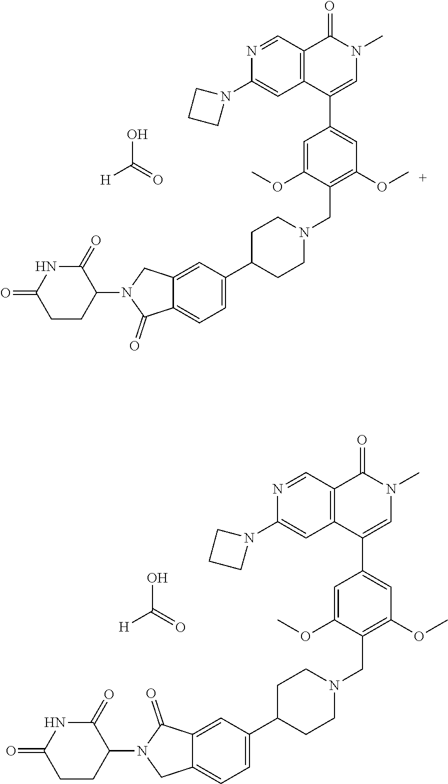

To a solution of 3-[5-[1-([4-[6-(azetidin-1-yl)-2-methyl-1-oxo-2,7-naphthyridin-4-yl]-2,6-dimethoxyphenyl]methyl)piperidin-4-yl]-3-hydroxy-1-oxo-3H-isoindol-2-yl]piperidine-2,6-dione and 3-[5-[1-([4-[6-(azetidin-1-yl)-2-methyl-1-oxo-2,7-naphthyridin-4-yl]-2,6-dimethoxyphenyl]methyl)piperidin-4-yl]-1-hydroxy-3-oxo-1H-isoindol-2-yl]piperidine-2,6-dione (mixture of two regio-isomers, 200.0 mg, 0.141 mmol, 1.00 equivalent) in DCM (3.00 mL) was added TFA (2.00 mL, 26.926 mmol, 95.16 equivalent) and triethylsilane (1.00 mL, 6.192 mmol, 21.88 equivalent), and the resulting solution was stirred at 25° C. for 1 hour. The crude product was purified by Prep-HPLC (Column: XSelect CSH Prep C18 OBD Column, 5 μm, 19*150 mm; Mobile Phase A: Water (0.05% FA), Mobile Phase B: ACN; Flow rate:25 mL/min; Gradient: 3 B to 26 B in 14 minutes; 254 nm; RT1: 13.32 min) to afford 3-[5-[1-([4-[6-(azetidin-1-yl)-2-methyl-1-oxo-2,7-naphthyridin-4-yl]-2,6-dimethoxyphenyl]methyl) piperidin-4-yl]-1-oxo-3H-isoindol-2-yl]piperidine-2,6-dione (39.5 mg, 39.1%) and 3-[6-[1-([4-[6-(azetidin-1-yl)-2-methyl-1-oxo-2,7-naphthyridin-4-yl]-2,6-dimethoxyphenyl] methyl)piperidin-4-yl]-1-oxo-3H-isoindol-2-yl]piperidine-2,6-dione; formic acid (24.8 mg, 22.7%) both as a white solid.

For 3-[5-[1-([4-[6-(azetidin-1-yl)-2-methyl-1-oxo-2,7-naphthyridin-4-yl]-2,6-dimethoxyphenyl]methyl) piperidin-4-yl]-1-oxo-3H-isoindol-2-yl]piperidine-2,6-dione: ¹H NMR (400 MHz, DMSO-d6) δ 10.99 (s, 1H), 9.02 (s, 1H), 8.16 (s, 1H, FA), 7.68-7.60 (m, 2H), 7.49 (s, 1H), 7.39 (dd, J=7.8, 1.4 Hz, 1H), 6.76 (s, 2H), 6.22 (s, 1H), 5.10 (dd, J=13.3, 5.1 Hz, 1H), 4.42 (d, J=17.3 Hz, 1H), 4.28 (d, J=17.3 Hz, 1H), 4.01 (t, J=7.4 Hz, 4H), 3.84 (s, 6H), 3.69 (s, 2H), 3.49 (s, 3H), 3.05 (d, J=11.2 Hz, 2H), 2.92 (ddd, J=17.3, 13.6, 5.4 Hz, 1H), 2.66-2.60 (m, 1H), 2.60-2.55 (m, 1H), 2.46-2.38 (m, 1H), 2.37-2.28 (m, 4H), 2.04-1.95 (m, 1H), 1.78-1.65 (m, 4H). LCMS (ESI) m/z: [M+H]⁺=691.35.

For 3-[6-[1-([4-[6-(azetidin-1-yl)-2-methyl-1-oxo-2,7-naphthyridin-4-yl]-2,6-dimethoxyphenyl]methyl)piperidin-4-yl]-1-oxo-3H-isoindol-2-yl]piperidine-2,6-dione; formic acid: ¹H NMR (400 MHz, DMSO-d6) δ 10.99 (s, 1H), 9.02 (s, 1H), 8.18 (s, FA), 7.62 (s, 1H), 7.58-7.48 (m, 3H), 6.75 (s, 2H), 6.22 (s, 1H), 5.10 (dd, J=13.3, 5.1 Hz, 1H), 4.41 (d, J=17.1 Hz, 1H), 4.27 (d, J=17.1 Hz, 1H), 4.01 (t, J=7.4 Hz, 4H), 3.84 (s, 6H), 3.63 (s, 2H), 3.48 (s, 3H), 3.00 (d, J=11.0 Hz, 2H), 2.97-2.85 (m, 1H), 2.65-2.60 (m, 1H), 2.60-2.56 (m, 1H), 2.45-2.37 (m, 1H), 2.37-2.30 (m, 1H), 2.24 (t, J=11.3 Hz, 2H), 2.03-1.96 (m, 1H), 1.80-1.73 (m, 2H), 1.73-1.62 (m, 2H). LCMS (ESI) m/z: [M+H]⁺=691.55.

### Example 17—Preparation of 3-(5-[[1-([2,6-dimethoxy-4-[2-methyl-6-(morpholin-4-yl)-1-oxo-2,7-naphthyridin-4-yl]phenyl]methyl)azetidin-3-yl]oxy]-1-oxo-3H-isoindol-2-yl)piperidine-2,6-dione

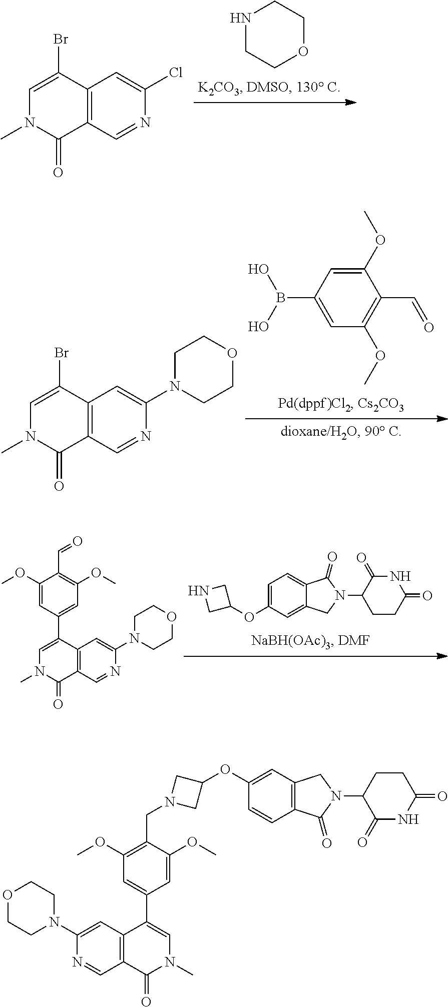

### Step 1: preparation of 4-bromo-2-methyl-6-(morpholin-4-yl)-2,7-naphthyridin-1-one

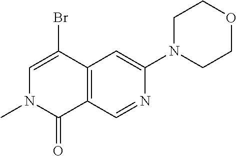 [SMILES: Cn1cc(Br)c2cc(N3CCOCC3)ncc2c1=O]

To a stirred solution of 4-bromo-6-chloro-2-methyl-2,7-naphthyridin-1-one (547.00 mg, 2.000 mmol, 1.00 equivalent) and morpholine (522.71 mg, 6.000 mmol, 3.00 equivalent) in DMSO (6.00 mL) was added K₂CO₃ (1382.00 mg, 10.000 mmol, 5.00 equivalent). The resulting mixture was stirred for 1 h at 130° C. under nitrogen atmosphere. The reaction mixture was diluted with EA (100 mL).

The resulting mixture was washed with 3×100 mL of water and 1×100 mL saturated brine. The organic layer was dried over Na₂SO₄, filtered and evaporated to afford crude product. The residue was purified by silica gel column chromatography, elution gradient 0 to 10% MeOH in DCM. Pure fractions were evaporated to dryness to afford 4-bromo-2-methyl-6-(morpholin-4-yl)-2,7-naphthyridin-1-one (541 mg, 83.44%) as a light yellow solid. LCMS (ESI) m/z: [M+H]⁺=324.

### Step 2: Preparation of 2,6-dimethoxy-4-[2-methyl-6-(morpholin-4-yl)-1-oxo-2,7-naphthyridin-4-yl]benzaldehyde

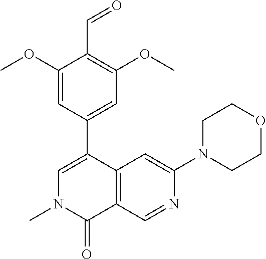 [SMILES: COc1cc(-c2cn(C)c(=O)c3cnc(N4CCOCC4)cc23)cc(OC)c1C=O]

To a solution of 4-bromo-2-methyl-6-(morpholin-4-yl)-2,7-naphthyridin-1-one (540.00 mg, 1.666 mmol, 1.00 equivalent) and 4-formyl-3,5-dimethoxyphenylboronic acid (454.73 mg, 2.165 mmol, 1.30 equivalent), Cs₂CO₃ (1628.20 mg, 4.997 mmol, 3.00 equivalent) in H₂O (1.00 mL) and dioxane (5.00 mL) was added Pd(dppf)Cl₂CH₂Cl₂ (136.03 mg, 0.167 mmol, 0.10 equivalent) under nitrogen. After stirring for 1 h at 90° C. under a nitrogen atmosphere, the resulting mixture was concentrated under reduced pressure. The residue was purified by silica gel column chromatography, elution gradient 0 to 10% MeOH in DCM. Pure fractions were evaporated to dryness to afford 2,6-dimethoxy-4-[2-methyl-6-(morpholin-4-yl)-1-oxo-2,7 naphthyridin-4-yl] benzaldehyde (356 mg, 52.20%) as a yellow solid. LCMS (ESI) m/z: [M+H]⁺=410.

### Step 3: Preparation of 3-(5-[[1-([2,6-dimethoxy-4-[2-methyl-6-(morpholin-4-yl)-1-oxo-2,7-naphthyridin-4-yl]phenyl]methyl)azetidin-3-yl]oxy]-1-oxo-3H-isoindol-2-yl)piperidine-2,6-dione

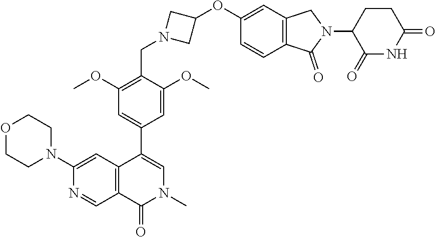 [SMILES: COc1cc(-c2cn(C)c(=O)c3cnc(N4CCOCC4)cc23)cc(OC)c1CN1CC(Oc2ccc3c(c2)CN(C2CCC(=O)NC2=O)C3=O)C1]

To a stirred solution of 3-[5-(azetidin-3-yloxy)-1-oxo-3H-isoindol-2-yl]piperidine-2,6-dione (100.00 mg, 0.317 mmol, 1.00 equivalent) and 2,6-dimethoxy-4-[2-methyl-6-(morpholin-4-yl)-1-oxo-2,7-naphthyridin-4-yl]benzaldehyde (129.85 mg, 0.317 mmol, 1.00 equivalent) in DMF was added NaBH(OAc)₃ (134.43 mg, 0.634 mmol, 2.00 equivalent) dropwise at room temperature under air atmosphere for 2 hours. The residue was purified by reverse flash chromatography with the following conditions: column, C18 silica gel; mobile phase, MeCN in water, 0% to 100% gradient in 45 min; detector, UV 254 nm. The crude product was purified by Prep-HPLC with the following conditions (Column: Xcelect CSH F-pheny OBD Column, 19*250 mm, 5 μm; Mobile Phase A: Water (0.05% FA); Mobile Phase B: ACN; Flow rate: 30 mL/min; Gradient: 13 B to 33 B in 14 min; 254/220 nm; RT1: 12.85 min) to afford 3-(5-[[1-([2,6-dimethoxy-4-[2-methyl-6-(morpholin-4-yl)-1-oxo-2,7-naphthyridin-4-yl]phenyl]methyl)azetidin-3-yl]oxy]-1-oxo-3H-isoindol-2-yl)piperidine-2,6-dione (100 mg, 44.15%) as a yellow solid. ¹H NMR (400 MHz, Methanol-d4) δ 9.18 (s, 1H), 7.80 (t, J=6.7 Hz, 1H), 7.49 (s, 1H), 7.09 (t, J=7.3 Hz, 2H), 6.88 (s, 2H), 6.63 (d, J=4.9 Hz, 1H), 5.40-5.20 (m, 1H), 5.15 (dd, J=13.3, 5.2 Hz, 1H), 4.77 (ddd, J=24.3, 12.5, 6.8 Hz, 2H), 4.65 (d, J=22.0 Hz, 2H), 4.48 (d, J=6.3 Hz, 2H), 4.44-4.28 (m, 2H), 3.96 (d, J=23.6 Hz, 6H), 3.78 (t, J=4.8 Hz, 4H), 3.61 (s, 3H), 3.56 (d, J=4.7 Hz, 4H), 2.93 (ddd, J=18.5, 13.5, 5.3 Hz, 1H), 2.80 (ddd, J=17.5, 4.6, 2.3 Hz, 1H), 2.49 (qd, J=13.2, 4.7 Hz, 1H), 2.23-2.14 (m, 1H). LCMS (ESI) m/z: [M+H]⁺=709.

### Example 18—SYO1 BRD9 NanoLuc Degradation Assay

This example demonstrates the ability of the compounds of the disclosure to degrade a Nanoluciferase-BRD9 fusion protein in a cell-based degradation assay.

Procedure: A stable SYO-1 cell line expressing 3×FLAG-NLuc-BRD9 was generated. On day 0 cells were seeded in 30 μL media into each well of 384-well cell culture plates. The seeding density was 8000 cells/well. On day 1, cells were treated with 30 nL DMSO or 30 nL of 3-fold serially DMSO-diluted compounds (10 points in duplicates with 1 μM as final top dose). Subsequently plates were incubated for 6 hours in a standard tissue culture incubator and equilibrated at room temperature for 15 minutes.

Nanoluciferase activity was measured by adding 15 μL of freshly prepared Nano-Glo Luciferase Assay Reagent (Promega N1130), shaking the plates for 10 minutes and reading the bioluminescence using an EnVision reader.

Results: The Inhibition % was calculated using the following formula: % Inhibition=100×(Lum_{HC}-Lum_{Sample})/(Lum_{HC}−Lum_{LC}). DMSO treated cells are employed as High Control (HC) and 1 μM of a known BRD9 degrader standard treated cells are employed as Low Control (LC). The data was fit to a four parameter, non-linear curve fit to calculate IC₅₀ (μM) values as shown in Table 4. As shown by the results in Table 4, a number of compounds of the present disclosure exhibit an IC₅₀ value of <1 μM for the degradation of BRD9, indicating their use as compounds for reducing the levels and/or activity of BRD9 and their potential for treating BRD9-related disorders.

TABLE 4
SYO1 BRD9-NanoLuc Degradation

| Compound No. | SYO1 BRD9-NanoLuc degradation IC₅₀ (nM) |
|---|---|
| D1 | ++++ |
| D2 | ++++ |
| D3 | ++++ |
| D4 | ++++ |
| D5 | ++++ |
| D6 | ++++ |
| D7 | ++++ |
| D8 | ++++ |
| D9 | ++++ |
| D10 | ++++ |
| D11 | ++++ |
| D12 | ++++ |
| D13 | ++++ |
| D14 | ++++ |
| D15 | ++++ |
| D16 | ++++ |
| D17 | ++++ |
| D18 | ++++ |
| D19 | ++++ |
| D20 | ++++ |
| D21 | ++++ |
| D22 | ++++ |
| D23 | ++++ |
| D24 | ++++ |
| D25 | ++++ |
| D26 | ++++ |
| D27 | ++++ |
| D28 | ++++ |
| D29 | ++++ |
| D30 | ++++ |
| D31 | ++++ |
| D32 | ++++ |
| D33 | ++++ |
| D34 | ++++ |
| D35 | ++++ |
| D36 | ++++ |
| D37 | ++++ |
| D38 | ++++ |
| D39 | ++++ |
| D40 | ++++ |
| D41 | ++++ |
| D42 | ++++ |
| D43 | ++++ |
| D44 | ++++ |
| D45 | ++++ |
| D46 | ++++ |
| D47 | ++++ |
| D48 | ++ |
| D49 | ++++ |
| D50 | ++++ |
| D51 | ++++ |
| D52 | ++++ |
| D53 | ++++ |
| D54 | ++++ |
| D55 | ++++ |
| D56 | ++++ |
| D57 | ++++ |
| D58 | ++++ |
| D59 | ++++ |
| D60 | ++++ |
| D61 | ++++ |
| D62 | ++++ |
| D63 | ++++ |
| D64 | ++++ |
| D65 | ++++ |
| D66 | ++++ |

“+” indicates inhibitory effect of ≥1000 nM;
“++” indicates inhibitory effect of ≥100 nM;
“+++” indicates inhibitory effect of ≥10 nM;
“++++” indicates inhibitory effect of <10 nM;
“NT” indicates not tested

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A compound having the structure of Formula I:
A-L-B Formula I,
where
L has the structure of Formula II:
A¹-E¹-F-E²-A², Formula II
A¹ is a bond between the linker and A;
A² is a bond between B and the linker;
each of E¹ and E² is, independently, absent, CH₂, O, or NCH₃; and
F is optionally substituted C₃-C₁₀ carbocyclylene or optionally substituted C₂₋₁₀ heterocyclylene;
B is a degradation moiety; and
A has the structure of Formula III: 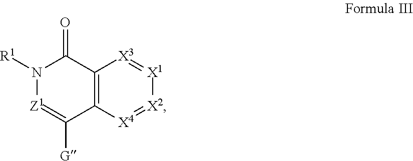
wherein
R¹ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
Z¹ is CR² or N;
R² is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, or optionally substituted C₂-C₉ heteroaryl;
X¹ is N or CH, and X² is C—R⁷″; or X¹ is C—R⁷″, and X² is N or CH;
R⁷″ is 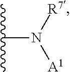
optionally substituted C₁-C₆ alkyl, optionally substituted C₁-C₆ heteroalkyl, optionally substituted C₁-C₆ alkoxy, optionally substituted amino, optionally substituted sulfone, optionally substituted sulfonamide, optionally substituted carbocyclyl having 3 to 6 atoms, or optionally substituted heterocyclyl having 3 to 6 atoms;
R^{7′} is H, optionally substituted C₁-C₆ alkyl, optionally substituted C₁-C₆ heteroalkyl, or optionally substituted C₃-C₁₀ carbocyclyl;
X³ is N or CH;
X⁴ is N or CH;
G″ is 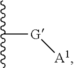
optionally substituted C₃-C₁₀ carbocyclyl, C₂-C₉ heterocyclyl, optionally substituted C₆-C₁₀ aryl, or optionally substituted C₂-C₉ heteroaryl;
G′ is optionally substituted C₃-C₁₀ carbocyclylene, C₂-C₉ heterocyclylene, optionally substituted C₆-C₁₀ arylene, or optionally substituted C₂-C₉ heteroarylene; and
A¹ is a bond between A and the linker,
where G″ is 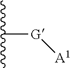
or R⁷″ is 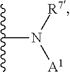
or a pharmaceutically acceptable salt thereof.

2. The compound of claim 1, wherein R¹ is optionally substituted C₁-C₆ alkyl.

3. The compound of claim 2, wherein R¹ is

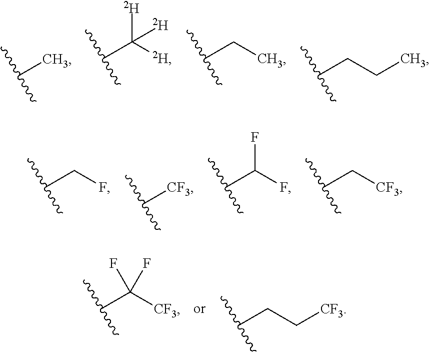

4. The compound of claim 3, wherein R¹ is

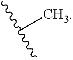

5. The compound of claim 1, wherein Z¹ is CR².

6. The compound of claim 5, wherein R² is H, F, or

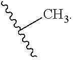

7. The compound of claim 1, wherein X¹ is N and X² is C—R⁷″.

8. The compound of claim 7, wherein R⁷″ is optionally substituted C₁-C₆ alkyl, optionally substituted C₁-C₆ heteroalkyl, optionally substituted carbocyclyl having 3 to 6 atoms, or optionally substituted heterocyclyl having 3 to 6 atoms.

9. The compound of claim 8, wherein R⁷″ is optionally substituted C₁-C₆ heteroalkyl.

10. The compound of claim 9, wherein R⁷″ is —NR³R⁴.

11. The compound of claim 8, wherein R⁷″ is optionally substituted heterocyclyl having 3 to 6 atoms.

12. The compound of claim 11, wherein R⁷″ is

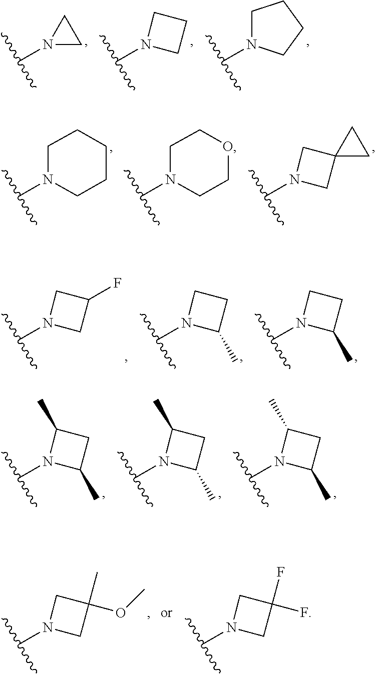

13. The compound of claim 1, wherein G″ is optionally substituted C₆-C₁₀ aryl.

14. The compound of claim 13, wherein G″ is 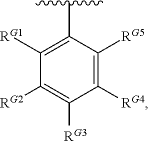
wherein
each of R^{G1}, R^{G2}, R^{G3}, R^{G4}, and R^{G5} is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, optionally substituted —C₁-C₃ alkyl-C₃-C₆ carbocyclyl, optionally substituted —C₁-C₃ alkyl-C₂-C₅ heterocyclyl, hydroxyl, thiol, or optionally substituted amino; or R^{G1} and R^{G2}, R^{G2} and R^{G3}, R^{G3} and R^{G4}, and/or R^{G4} and R^{G5}, together with the carbon atoms to which each is attached, combine to form optionally substituted C₆-C₁₀ aryl, optionally substituted C₃-C₁₀ carbocyclyl, optionally substituted C₂-C₉ heteroaryl, or optionally substituted C₂-C₉ heterocyclyl.

15. The compound of claim 14, wherein each of R^{G1}, R^{G2}, R^{G3}, R^{G4}, and R^{G5} is, independently, H, halogen, optionally substituted C₁-C₆ alkyl, optionally substituted C₁-C₆ heteroalkyl, optionally substituted —O—C₃-C₆ carbocyclyl, or optionally substituted —C₁-C₃ alkyl-C₂-C₅ heterocyclyl.

16. The compound of claim 15, wherein each of R^{G1}, R^{G2}, R^{G3}, R^{G4}, and R^{G5} is, independently, H, F, Cl,

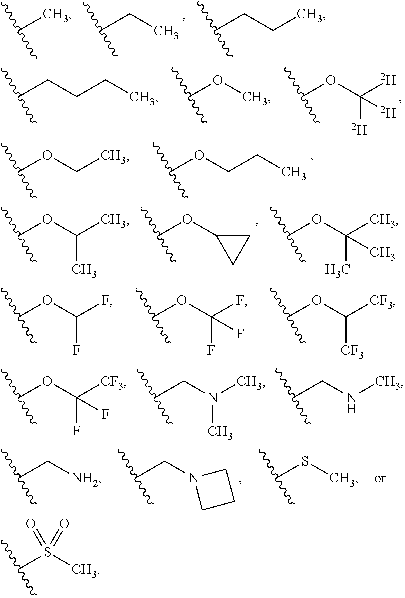

17. The compound of claim 1, wherein A has the structure of Formula IIIa: 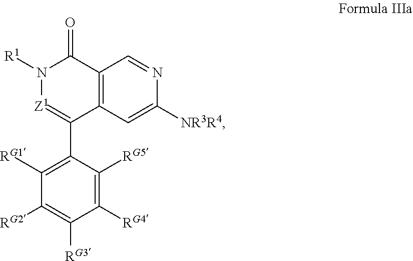
or a pharmaceutically acceptable salt thereof.

18. The compound of claim 1, wherein A has the structure of Formula IIIlu: 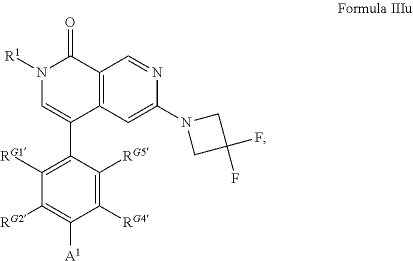
or a pharmaceutically acceptable salt thereof.

19. The compound of claim 1, wherein the degradation moiety is a ubiquitin ligase binding moiety.

20. The compound of claim 19, wherein the structure of Formula A has the structure of Formula A6: 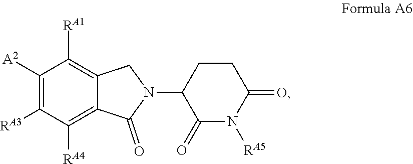
or a pharmaceutically acceptable salt thereof.

21. The compound of claim 20, wherein the structure of Formula A is 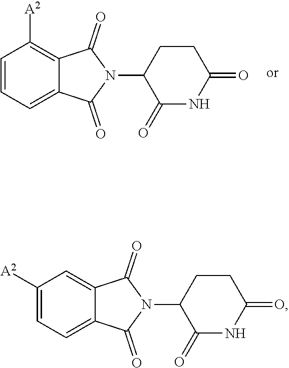
or derivative or analog thereof.

22. The compound of claim 1, wherein the linker has the structure of Formula II:
A¹-E¹-F-E²-A², Formula II
A¹ is a bond between the linker and A;
A² is a bond between B and the linker;
each of E¹ and E² is, independently, absent, CH₂, O, or NCH₃; and
F has the structure:

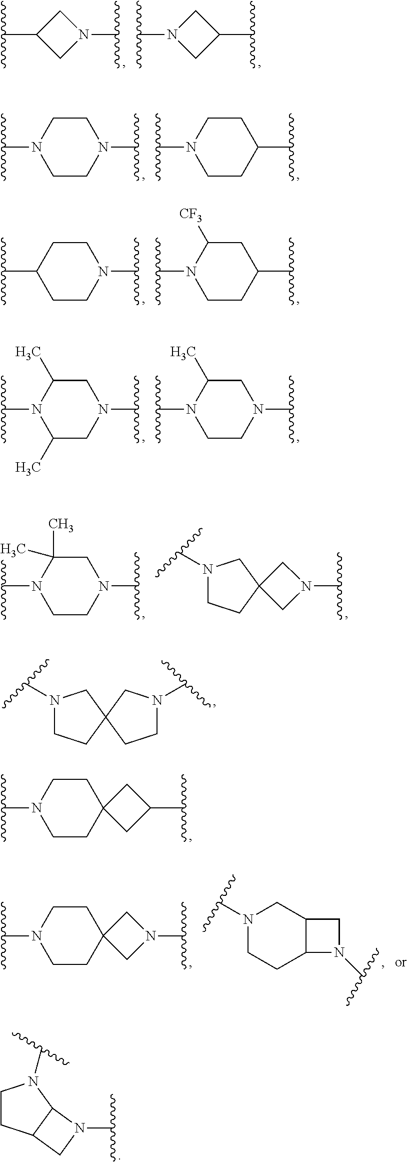

23. A pharmaceutical composition comprising the compound of claim 1 and a pharmaceutically acceptable excipient.

24. A method of treating a cancer in a subject in need thereof, the method including administering to the subject an effective amount of a compound of claim 1, or a pharmaceutically acceptable salt thereof.

25. The method of claim 24, wherein the compound, or a pharmaceutically acceptable salt thereof, or the pharmaceutical composition is administered orally.


SEQUENCE LISTING (673 sequences)

SEQ ID NO: 1 - protein, 141 aa, synthetic construct
MGSSHHHHHHENLYFQGDYKDDDDKGSLEVLFQGPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSK

SEQ ID NO: 2-202 - skipped (201 entries)

SEQ ID NO: 203 - DNA, 20 bp, synthetic construct
  source 1..20: mol_type = other DNA
caagaagcacaagaagcaca

SEQ ID NO: 204 - DNA, 20 bp, synthetic construct
  source 1..20: mol_type = other DNA
cttgtgcttcttgcccatgg

SEQ ID NO: 205 - DNA, 20 bp, synthetic construct
  source 1..20: mol_type = other DNA
cttcttgtgcttcttgccca

SEQ ID NO: 206 - DNA, 20 bp, synthetic construct
  source 1..20: mol_type = other DNA
acaagaagcacaaggccgag

SEQ ID NO: 207 - DNA, 20 bp, synthetic construct
  source 1..20: mol_type = other DNA
ctcgtaggacgagcgccact

SEQ ID NO: 208 - DNA, 20 bp, synthetic construct
  source 1..20: mol_type = other DNA
cgagtggcgctcgtcctacg

SEQ ID NO: 209 - DNA, 20 bp, synthetic construct
  source 1..20: mol_type = other DNA
gagtggcgctcgtcctacga

SEQ ID NO: 210 - DNA, 20 bp, synthetic construct
  source 1..20: mol_type = other DNA
aggcttctccaggggcttgt

SEQ ID NO: 211 - DNA, 20 bp, synthetic construct
  source 1..20: mol_type = other DNA
agattatgccgacaagcccc

SEQ ID NO: 212 - DNA, 20 bp, synthetic construct
  source 1..20: mol_type = other DNA
accttcaggactagctttag

SEQ ID NO: 213 - DNA, 20 bp, synthetic construct
  source 1..20: mol_type = other DNA
agctttagaggcttctccag

SEQ ID NO: 214 - DNA, 20 bp, synthetic construct
  source 1..20: mol_type = other DNA
ctagctttagaggcttctcc

SEQ ID NO: 215 - DNA, 20 bp, synthetic construct
  source 1..20: mol_type = other DNA
tagctttagaggcttctcca

SEQ ID NO: 216 - DNA, 20 bp, synthetic construct
  source 1..20: mol_type = other DNA
ctaaagctagtcctgaaggt

SEQ ID NO: 217 - DNA, 20 bp, synthetic construct
  source 1..20: mol_type = other DNA
gcctctaaagctagtcctga

SEQ ID NO: 218 - DNA, 20 bp, synthetic construct
  source 1..20: mol_type = other DNA
cttcacttcctccgaccttc

SEQ ID NO: 219 - DNA, 20 bp, synthetic construct
  source 1..20: mol_type = other DNA
aagctagtcctgaaggtcgg

SEQ ID NO: 220 - DNA, 20 bp, synthetic construct
  source 1..20: mol_type = other DNA
agtgaagtgactgaactctc

SEQ ID NO: 221 - DNA, 20 bp, synthetic construct
  source 1..20: mol_type = other DNA
gtgactgaactctcaggatc

SEQ ID NO: 222 - DNA, 20 bp, synthetic construct
  source 1..20: mol_type = other DNA
atagtaactggagtcgtggc

SEQ ID NO: 223 - DNA, 20 bp, synthetic construct
  source 1..20: mol_type = other DNA
catcatagtaactggagtcg

SEQ ID NO: 224 - DNA, 20 bp, synthetic construct
  source 1..20: mol_type = other DNA
tgacctgtcatcatagtaac

SEQ ID NO: 225 - DNA, 20 bp, synthetic construct
  source 1..20: mol_type = other DNA
actccagttactatgatgac

SEQ ID NO: 226 - DNA, 20 bp, synthetic construct
  source 1..20: mol_type = other DNA
ctttgtgcctctctcgctca

SEQ ID NO: 227 - DNA, 20 bp, synthetic construct
  source 1..20: mol_type = other DNA
ggtcagaccatgagcgagag

SEQ ID NO: 228 - DNA, 20 bp, synthetic construct
  source 1..20: mol_type = other DNA
gaagaagaagaagtccgaga

SEQ ID NO: 229 - DNA, 20 bp, synthetic construct
  source 1..20: mol_type = other DNA
gtccagatgcttctccttct

SEQ ID NO: 230 - DNA, 20 bp, synthetic construct
  source 1..20: mol_type = other DNA
gtccgagaaggagaagcatc

SEQ ID NO: 231 - DNA, 20 bp, synthetic construct
  source 1..20: mol_type = other DNA
ggagaagcatctggacgatg

SEQ ID NO: 232 - DNA, 20 bp, synthetic construct
  source 1..20: mol_type = other DNA
tgaggaaagaaggaagcgaa

SEQ ID NO: 233 - DNA, 20 bp, synthetic construct
  source 1..20: mol_type = other DNA
atctggacgatgaggaaaga

SEQ ID NO: 234 - DNA, 20 bp, synthetic construct
  source 1..20: mol_type = other DNA
agaagaagcggaagcgagag

SEQ ID NO: 235 - DNA, 20 bp, synthetic construct
  source 1..20: mol_type = other DNA
gaagaagcggaagcgagaga

SEQ ID NO: 236 - DNA, 20 bp, synthetic construct
  source 1..20: mol_type = other DNA
ccgcccaggaagagaagaag

SEQ ID NO: 237 - DNA, 20 bp, synthetic construct
  source 1..20: mol_type = other DNA
agagagggagcactgtgaca

SEQ ID NO: 238 - DNA, 20 bp, synthetic construct
  source 1..20: mol_type = other DNA
agggagcactgtgacacgga

SEQ ID NO: 239 - DNA, 20 bp, synthetic construct
  source 1..20: mol_type = other DNA
gagggagcactgtgacacgg

SEQ ID NO: 240 - DNA, 20 bp, synthetic construct
  source 1..20: mol_type = other DNA
gcactgtgacacggagggag

SEQ ID NO: 241 - DNA, 20 bp, synthetic construct
  source 1..20: mol_type = other DNA
gaggctgacgactttgatcc

SEQ ID NO: 242 - DNA, 20 bp, synthetic construct
  source 1..20: mol_type = other DNA
aggctgacgactttgatcct

SEQ ID NO: 243 - DNA, 20 bp, synthetic construct
  source 1..20: mol_type = other DNA
tccacctccaccttcttccc

SEQ ID NO: 244 - DNA, 20 bp, synthetic construct
  source 1..20: mol_type = other DNA
cgactttgatcctgggaaga

SEQ ID NO: 245 - DNA, 20 bp, synthetic construct
  source 1..20: mol_type = other DNA
ctttgatcctgggaagaagg

SEQ ID NO: 246 - DNA, 20 bp, synthetic construct
  source 1..20: mol_type = other DNA
tgatcctgggaagaaggtgg

SEQ ID NO: 247 - DNA, 20 bp, synthetic construct
  source 1..20: mol_type = other DNA
tcctgggaagaaggtggagg

SEQ ID NO: 248 - DNA, 20 bp, synthetic construct
  source 1..20: mol_type = other DNA
cggactggccgatctggggg

SEQ ID NO: 249 - DNA, 20 bp, synthetic construct
  source 1..20: mol_type = other DNA
acgctcggactggccgatct

SEQ ID NO: 250 - DNA, 20 bp, synthetic construct
  source 1..20: mol_type = other DNA
aggtggagccgcccccagat

SEQ ID NO: 251 - DNA, 20 bp, synthetic construct
  source 1..20: mol_type = other DNA
cgctcggactggccgatctg

SEQ ID NO: 252 - DNA, 20 bp, synthetic construct
  source 1..20: mol_type = other DNA
gctcggactggccgatctgg

SEQ ID NO: 253 - DNA, 20 bp, synthetic construct
  source 1..20: mol_type = other DNA
cacgctcggactggccgatc

SEQ ID NO: 254 - DNA, 20 bp, synthetic construct
  source 1..20: mol_type = other DNA
tgtgtccggcacgctcggac

SEQ ID NO: 255 - DNA, 20 bp, synthetic construct
  source 1..20: mol_type = other DNA
ctggctgtgtccggcacgct

SEQ ID NO: 256 - DNA, 20 bp, synthetic construct
  source 1..20: mol_type = other DNA
atcggccagtccgagcgtgc

SEQ ID NO: 257 - DNA, 20 bp, synthetic construct
  source 1..20: mol_type = other DNA
cacccttgcctggctgtgtc

SEQ ID NO: 258 - DNA, 20 bp, synthetic construct
  source 1..20: mol_type = other DNA
cgagcgtgccggacacagcc

SEQ ID NO: 259 - DNA, 20 bp, synthetic construct
  source 1..20: mol_type = other DNA
tgttccaggagttgctgaat

SEQ ID NO: 260 - DNA, 20 bp, synthetic construct
  source 1..20: mol_type = other DNA
cacacctattcagcaactcc

SEQ ID NO: 261 - DNA, 20 bp, synthetic construct
  source 1..20: mol_type = other DNA
gctggcggaggaagtgttcc

SEQ ID NO: 262 - DNA, 20 bp, synthetic construct
  source 1..20: mol_type = other DNA
tttacctctgaagctggcgg

SEQ ID NO: 263 - DNA, 20 bp, synthetic construct
  source 1..20: mol_type = other DNA
ccccggtttacctctgaagc

SEQ ID NO: 264 - DNA, 20 bp, synthetic construct
  source 1..20: mol_type = other DNA
acttcctccgccagcttcag

SEQ ID NO: 265 - DNA, 20 bp, synthetic construct
  source 1..20: mol_type = other DNA
caggaaaagcaaaaaatcca

SEQ ID NO: 266 - DNA, 20 bp, synthetic construct
  source 1..20: mol_type = other DNA
gctttcagaaaagatcccca

SEQ ID NO: 267 - DNA, 20 bp, synthetic construct
  source 1..20: mol_type = other DNA
aggaaaagcaaaaaatccat

SEQ ID NO: 268 - DNA, 20 bp, synthetic construct
  source 1..20: mol_type = other DNA
ggaaaagcaaaaaatccatg

SEQ ID NO: 269 - DNA, 20 bp, synthetic construct
  source 1..20: mol_type = other DNA
ggagcaattgcatccgtgac

SEQ ID NO: 270 - DNA, 20 bp, synthetic construct
  source 1..20: mol_type = other DNA
gtcacggatgcaattgctcc

SEQ ID NO: 271 - DNA, 20 bp, synthetic construct
  source 1..20: mol_type = other DNA
tttattatcattgaatatcc

SEQ ID NO: 272 - DNA, 20 bp, synthetic construct
  source 1..20: mol_type = other DNA
aatgataataaaacatccca

SEQ ID NO: 273 - DNA, 20 bp, synthetic construct
  source 1..20: mol_type = other DNA
ataaaacatcccatggattt

SEQ ID NO: 274 - DNA, 20 bp, synthetic construct
  source 1..20: mol_type = other DNA
ttcatggtgccaaaatccat

SEQ ID NO: 275 - DNA, 20 bp, synthetic construct
  source 1..20: mol_type = other DNA
tttcatggtgccaaaatcca

SEQ ID NO: 276 - DNA, 20 bp, synthetic construct
  source 1..20: mol_type = other DNA
taatgaatacaagtcagtta

SEQ ID NO: 277 - DNA, 20 bp, synthetic construct
  source 1..20: mol_type = other DNA
caagtcagttacggaattta

SEQ ID NO: 278 - DNA, 20 bp, synthetic construct
  source 1..20: mol_type = other DNA
ataatgcaatgacatacaat

SEQ ID NO: 279 - DNA, 20 bp, synthetic construct
  source 1..20: mol_type = other DNA
aacttgtagtacacggtatc

SEQ ID NO: 280 - DNA, 20 bp, synthetic construct
  source 1..20: mol_type = other DNA
cttcgccaacttgtagtaca

SEQ ID NO: 281 - DNA, 20 bp, synthetic construct
  source 1..20: mol_type = other DNA
agataccgtgtactacaagt

SEQ ID NO: 282 - DNA, 20 bp, synthetic construct
  source 1..20: mol_type = other DNA
gcgaagaagatccttcacgc

SEQ ID NO: 283 - DNA, 20 bp, synthetic construct
  source 1..20: mol_type = other DNA
tcatcttaaagcctgcgtga

SEQ ID NO: 284 - DNA, 20 bp, synthetic construct
  source 1..20: mol_type = other DNA
ttctcagcaggcagctcttt

SEQ ID NO: 285 - DNA, 20 bp, synthetic construct
  source 1..20: mol_type = other DNA
caatgaagatacagctgttg

SEQ ID NO: 286 - DNA, 20 bp, synthetic construct
  source 1..20: mol_type = other DNA
actggtacaacttcagggac

SEQ ID NO: 287 - DNA, 20 bp, synthetic construct
  source 1..20: mol_type = other DNA
cttgtactggtacaacttca

SEQ ID NO: 288 - DNA, 20 bp, synthetic construct
  source 1..20: mol_type = other DNA
acttgtactggtacaacttc

SEQ ID NO: 289 - DNA, 20 bp, synthetic construct
  source 1..20: mol_type = other DNA
ttggcagtttctacttgtac

SEQ ID NO: 290 - DNA, 20 bp, synthetic construct
  source 1..20: mol_type = other DNA
tacctgataacttctctact

SEQ ID NO: 291 - DNA, 20 bp, synthetic construct
  source 1..20: mol_type = other DNA
agccgagtagagaagttatc

SEQ ID NO: 292 - DNA, 20 bp, synthetic construct
  source 1..20: mol_type = other DNA
agctgcatgtttgagcctga

SEQ ID NO: 293 - DNA, 20 bp, synthetic construct
  source 1..20: mol_type = other DNA
gctgcatgtttgagcctgaa

SEQ ID NO: 294 - DNA, 20 bp, synthetic construct
  source 1..20: mol_type = other DNA
aagctgcaggcattcccttc

SEQ ID NO: 295 - DNA, 20 bp, synthetic construct
  source 1..20: mol_type = other DNA
ggtactgtccgtcaagctgc

SEQ ID NO: 296 - DNA, 20 bp, synthetic construct
  source 1..20: mol_type = other DNA
agggaatgcctgcagcttga

SEQ ID NO: 297 - DNA, 20 bp, synthetic construct
  source 1..20: mol_type = other DNA
cttgacggacagtaccgcag

SEQ ID NO: 298 - DNA, 20 bp, synthetic construct
  source 1..20: mol_type = other DNA
cgccagcacgtgctcctctg

SEQ ID NO: 299 - DNA, 20 bp, synthetic construct
  source 1..20: mol_type = other DNA
taccgcagaggagcacgtgc

SEQ ID NO: 300 - DNA, 20 bp, synthetic construct
  source 1..20: mol_type = other DNA
agaggagcacgtgctggcgc

SEQ ID NO: 301 - DNA, 20 bp, synthetic construct
  source 1..20: mol_type = other DNA
ggagcacgtgctggcgctgg

SEQ ID NO: 302 - DNA, 20 bp, synthetic construct
  source 1..20: mol_type = other DNA
agcacgcagctgacgaagct

SEQ ID NO: 303 - DNA, 20 bp, synthetic construct
  source 1..20: mol_type = other DNA
gcacgcagctgacgaagctc

SEQ ID NO: 304 - DNA, 20 bp, synthetic construct
  source 1..20: mol_type = other DNA
cagctgacgaagctcgggac

SEQ ID NO: 305 - DNA, 20 bp, synthetic construct
  source 1..20: mol_type = other DNA
aagctcgggacaggatcaac

SEQ ID NO: 306 - DNA, 20 bp, synthetic construct
  source 1..20: mol_type = other DNA
ccttgccgcctgggaggaac

SEQ ID NO: 307 - DNA, 20 bp, synthetic construct
  source 1..20: mol_type = other DNA
aggatcaaccggttcctccc

SEQ ID NO: 308 - DNA, 20 bp, synthetic construct
  source 1..20: mol_type = other DNA
atcaaccggttcctcccagg

SEQ ID NO: 309 - DNA, 20 bp, synthetic construct
  source 1..20: mol_type = other DNA
gcactaccttgccgcctggg

SEQ ID NO: 310 - DNA, 20 bp, synthetic construct
  source 1..20: mol_type = other DNA
agagcactaccttgccgcct

SEQ ID NO: 311 - DNA, 20 bp, synthetic construct
  source 1..20: mol_type = other DNA
ccggttcctcccaggcggca

SEQ ID NO: 312 - DNA, 20 bp, synthetic construct
  source 1..20: mol_type = other DNA
tcctcttcagatagcccatc

SEQ ID NO: 313 - DNA, 20 bp, synthetic construct
  source 1..20: mol_type = other DNA
atgggctatctgaagaggaa

SEQ ID NO: 314 - DNA, 20 bp, synthetic construct
  source 1..20: mol_type = other DNA
gggctatctgaagaggaacg

SEQ ID NO: 315 - DNA, 20 bp, synthetic construct
  source 1..20: mol_type = other DNA
tgggctatctgaagaggaac

SEQ ID NO: 316 - DNA, 20 bp, synthetic construct
  source 1..20: mol_type = other DNA
tatctgaagaggaacgggga

SEQ ID NO: 317 - DNA, 20 bp, synthetic construct
  source 1..20: mol_type = other DNA
atctgaagaggaacggggac

SEQ ID NO: 318 - DNA, 20 bp, synthetic construct
  source 1..20: mol_type = other DNA
tgttgaccacgctgtagagc

SEQ ID NO: 319 - DNA, 20 bp, synthetic construct
  source 1..20: mol_type = other DNA
gctctacagcgtggtcaaca

SEQ ID NO: 320 - DNA, 20 bp, synthetic construct
  source 1..20: mol_type = other DNA
cgggagcctgctctacagcg

SEQ ID NO: 321 - DNA, 20 bp, synthetic construct
  source 1..20: mol_type = other DNA
cgtggtcaacacggccgagc

SEQ ID NO: 322 - DNA, 20 bp, synthetic construct
  source 1..20: mol_type = other DNA
cccaccatcagcgtccggct

SEQ ID NO: 323 - DNA, 20 bp, synthetic construct
  source 1..20: mol_type = other DNA
acggccgagccggacgctga

SEQ ID NO: 324 - DNA, 20 bp, synthetic construct
  source 1..20: mol_type = other DNA
gggcacccaccatcagcgtc

SEQ ID NO: 325 - DNA, 20 bp, synthetic construct
  source 1..20: mol_type = other DNA
gccgagccggacgctgatgg

SEQ ID NO: 326 - DNA, 20 bp, synthetic construct
  source 1..20: mol_type = other DNA
ccatgtccgtgttgcagagg

SEQ ID NO: 327 - DNA, 20 bp, synthetic construct
  source 1..20: mol_type = other DNA
ccgagccggacgctgatggt

SEQ ID NO: 328 - DNA, 20 bp, synthetic construct
  source 1..20: mol_type = other DNA
cgagctcaagtccaccgggt

SEQ ID NO: 329 - DNA, 20 bp, synthetic construct
  source 1..20: mol_type = other DNA
gcgagctcaagtccaccggg

SEQ ID NO: 330 - DNA, 20 bp, synthetic construct
  source 1..20: mol_type = other DNA
agagcgagctcaagtccacc

SEQ ID NO: 331 - DNA, 20 bp, synthetic construct
  source 1..20: mol_type = other DNA
gagagcgagctcaagtccac

SEQ ID NO: 332 - DNA, 20 bp, synthetic construct
  source 1..20: mol_type = other DNA
gaagcctgggagtagcttac

SEQ ID NO: 333 - DNA, 20 bp, synthetic construct
  source 1..20: mol_type = other DNA
ctctccagtaagctactccc

SEQ ID NO: 334 - DNA, 20 bp, synthetic construct
  source 1..20: mol_type = other DNA
agcccagcgtggtgaagcct

SEQ ID NO: 335 - DNA, 20 bp, synthetic construct
  source 1..20: mol_type = other DNA
aagcccagcgtggtgaagcc

SEQ ID NO: 336 - DNA, 20 bp, synthetic construct
  source 1..20: mol_type = other DNA
actcccaggcttcaccacgc

SEQ ID NO: 337 - DNA, 20 bp, synthetic construct
  source 1..20: mol_type = other DNA
ctcccaggcttcaccacgct

SEQ ID NO: 338 - DNA, 20 bp, synthetic construct
  source 1..20: mol_type = other DNA
ctcgtctttgaagcccagcg

SEQ ID NO: 339 - DNA, 20 bp, synthetic construct
  source 1..20: mol_type = other DNA
cactggagagaaaggtgact

SEQ ID NO: 340 - DNA, 20 bp, synthetic construct
  source 1..20: mol_type = other DNA
gcactggagagaaaggtgac

SEQ ID NO: 341 - DNA, 20 bp, synthetic construct
  source 1..20: mol_type = other DNA
agtagtggcactggagagaa

SEQ ID NO: 342 - DNA, 20 bp, synthetic construct
  source 1..20: mol_type = other DNA
cgaaagcgcagtagtggcac

SEQ ID NO: 343 - DNA, 20 bp, synthetic construct
  source 1..20: mol_type = other DNA
ctgcatcgaaagcgcagtag

SEQ ID NO: 344 - DNA, 20 bp, synthetic construct
  source 1..20: mol_type = other DNA
atgcagaataattcagtatt

SEQ ID NO: 345 - DNA, 20 bp, synthetic construct
  source 1..20: mol_type = other DNA
agtatttggcgacttgaagt

SEQ ID NO: 346 - DNA, 20 bp, synthetic construct
  source 1..20: mol_type = other DNA
cgacttgaagtcggacgaga

SEQ ID NO: 347 - DNA, 20 bp, synthetic construct
  source 1..20: mol_type = other DNA
gagctgctctactcagccta

SEQ ID NO: 348 - DNA, 20 bp, synthetic construct
  source 1..20: mol_type = other DNA
cacgcctgtctcatctccgt

SEQ ID NO: 349 - DNA, 20 bp, synthetic construct
  source 1..20: mol_type = other DNA
tcagcctacggagatgagac

SEQ ID NO: 350 - DNA, 20 bp, synthetic construct
  source 1..20: mol_type = other DNA
caggcgtgcagtgtgcgctg

SEQ ID NO: 351 - DNA, 20 bp, synthetic construct
  source 1..20: mol_type = other DNA
ccgcggcccctctagcctgc

SEQ ID NO: 352 - DNA, 20 bp, synthetic construct
  source 1..20: mol_type = other DNA
catccttcacaaactcctgc

SEQ ID NO: 353 - DNA, 20 bp, synthetic construct
  source 1..20: mol_type = other DNA
tagcctgcaggagtttgtga

SEQ ID NO: 354 - DNA, 20 bp, synthetic construct
  source 1..20: mol_type = other DNA
caggagtttgtgaaggatgc

SEQ ID NO: 355 - DNA, 20 bp, synthetic construct
  source 1..20: mol_type = other DNA
aggagtttgtgaaggatgct

SEQ ID NO: 356 - DNA, 20 bp, synthetic construct
  source 1..20: mol_type = other DNA
tgggagctacagcaagaaag

SEQ ID NO: 357 - DNA, 20 bp, synthetic construct
  source 1..20: mol_type = other DNA
gagctacagcaagaaagtgg

SEQ ID NO: 358 - DNA, 20 bp, synthetic construct
  source 1..20: mol_type = other DNA
gaaagtggtggacgacctcc

SEQ ID NO: 359 - DNA, 20 bp, synthetic construct
  source 1..20: mol_type = other DNA
cgcctgtgatctggtccagg

SEQ ID NO: 360 - DNA, 20 bp, synthetic construct
  source 1..20: mol_type = other DNA
ctccgcctgtgatctggtcc

SEQ ID NO: 361 - DNA, 20 bp, synthetic construct
  source 1..20: mol_type = other DNA
gacctcctggaccagatcac

SEQ ID NO: 362 - DNA, 20 bp, synthetic construct
  source 1..20: mol_type = other DNA
ctcctggaccagatcacagg

SEQ ID NO: 363 - DNA, 20 bp, synthetic construct
  source 1..20: mol_type = other DNA
gctggaagagcgtcctagag

SEQ ID NO: 364 - DNA, 20 bp, synthetic construct
  source 1..20: mol_type = other DNA
tgcagcccacctgcttcagc

SEQ ID NO: 365 - DNA, 20 bp, synthetic construct
  source 1..20: mol_type = other DNA
gacgctcttccagctgaagc

SEQ ID NO: 366 - DNA, 20 bp, synthetic construct
  source 1..20: mol_type = other DNA
ctcttccagctgaagcaggt

SEQ ID NO: 367 - DNA, 20 bp, synthetic construct
  source 1..20: mol_type = other DNA
gctcttccagctgaagcagg

SEQ ID NO: 368 - DNA, 20 bp, synthetic construct
  source 1..20: mol_type = other DNA
cctccagatgaagccaaggt

SEQ ID NO: 369 - DNA, 20 bp, synthetic construct
  source 1..20: mol_type = other DNA
gcttcatctggaggcttcat

SEQ ID NO: 370 - DNA, 20 bp, synthetic construct
  source 1..20: mol_type = other DNA
ggcttcatctggaggcttca

SEQ ID NO: 371 - DNA, 20 bp, synthetic construct
  source 1..20: mol_type = other DNA
cttaccttggcttcatctgg

SEQ ID NO: 372 - DNA, 20 bp, synthetic construct
  source 1..20: mol_type = other DNA
aaacttaccttggcttcatc

SEQ ID NO: 373 - DNA, 20 bp, synthetic construct
  source 1..20: mol_type = other DNA
gaagcctccagatgaagcca

SEQ ID NO: 374 - DNA, 20 bp, synthetic construct
  source 1..20: mol_type = other DNA
tcctagggtgtccccaacct

SEQ ID NO: 375 - DNA, 20 bp, synthetic construct
  source 1..20: mol_type = other DNA
cctagggtgtccccaacctg

SEQ ID NO: 376 - DNA, 20 bp, synthetic construct
  source 1..20: mol_type = other DNA
gtgtctgtctccacaggttg

SEQ ID NO: 377 - DNA, 20 bp, synthetic construct
  source 1..20: mol_type = other DNA
tgtgtctgtctccacaggtt

SEQ ID NO: 378 - DNA, 20 bp, synthetic construct
  source 1..20: mol_type = other DNA
ccacaggttggggacaccct

SEQ ID NO: 379 - DNA, 20 bp, synthetic construct
  source 1..20: mol_type = other DNA
agagctgctgctgtctccta

SEQ ID NO: 380 - DNA, 20 bp, synthetic construct
  source 1..20: mol_type = other DNA
cagagctgctgctgtctcct

SEQ ID NO: 381 - DNA, 20 bp, synthetic construct
  source 1..20: mol_type = other DNA
agacagcagcagctctgttc

SEQ ID NO: 382 - DNA, 20 bp, synthetic construct
  source 1..20: mol_type = other DNA
atccacagaaacgtcgggat

SEQ ID NO: 383 - DNA, 20 bp, synthetic construct
  source 1..20: mol_type = other DNA
gagatatccacagaaacgtc

SEQ ID NO: 384 - DNA, 20 bp, synthetic construct
  source 1..20: mol_type = other DNA
ggagatatccacagaaacgt

SEQ ID NO: 385 - DNA, 20 bp, synthetic construct
  source 1..20: mol_type = other DNA
gtcctatcccgacgtttctg

SEQ ID NO: 386 - DNA, 20 bp, synthetic construct
  source 1..20: mol_type = other DNA
tctccatgctcagctctctg

SEQ ID NO: 387 - DNA, 20 bp, synthetic construct
  source 1..20: mol_type = other DNA
ctcacccagagagctgagca

SEQ ID NO: 388 - DNA, 20 bp, synthetic construct
  source 1..20: mol_type = other DNA
atctccatgctcagctctct

SEQ ID NO: 389 - DNA, 20 bp, synthetic construct
  source 1..20: mol_type = other DNA
tatctccatgctcagctctc

SEQ ID NO: 390 - DNA, 20 bp, synthetic construct
  source 1..20: mol_type = other DNA
atgtcctgtttacacaggga

SEQ ID NO: 391 - DNA, 20 bp, synthetic construct
  source 1..20: mol_type = other DNA
ttacacagggaaggtgaaga

SEQ ID NO: 392 - DNA, 20 bp, synthetic construct
  source 1..20: mol_type = other DNA
agttcaaatggctgtcgtca

SEQ ID NO: 393 - DNA, 20 bp, synthetic construct
  source 1..20: mol_type = other DNA
tgacgacagccatttgaact

SEQ ID NO: 394 - DNA, 20 bp, synthetic construct
  source 1..20: mol_type = other DNA
aagttcaaatggctgtcgtc

SEQ ID NO: 395 - DNA, 20 bp, synthetic construct
  source 1..20: mol_type = other DNA
tcgtctcatccaagttcaaa

SEQ ID NO: 396 - DNA, 20 bp, synthetic construct
  source 1..20: mol_type = other DNA
tgagacgacgaagctcctgc

SEQ ID NO: 397 - DNA, 20 bp, synthetic construct
  source 1..20: mol_type = other DNA
gtgcttcgtgcaggtcctgc

SEQ ID NO: 398 - DNA, 20 bp, synthetic construct
  source 1..20: mol_type = other DNA
gcaggacctgcacgaagcac

SEQ ID NO: 399 - DNA, 20 bp, synthetic construct
  source 1..20: mol_type = other DNA
gctccgcctgtgcttcgtgc

SEQ ID NO: 400 - DNA, 20 bp, synthetic construct
  source 1..20: mol_type = other DNA
ggacctgcacgaagcacagg

SEQ ID NO: 401 - DNA, 20 bp, synthetic construct
  source 1..20: mol_type = other DNA
cacgaagcacaggcggagcg

SEQ ID NO: 402 - DNA, 20 bp, synthetic construct
  source 1..20: mol_type = other DNA
aggcggagcgcggcggctct

SEQ ID NO: 403 - DNA, 20 bp, synthetic construct
  source 1..20: mol_type = other DNA
agggagctgaggttggacga

SEQ ID NO: 404 - DNA, 20 bp, synthetic construct
  source 1..20: mol_type = other DNA
gttggacagggagctgaggt

SEQ ID NO: 405 - DNA, 20 bp, synthetic construct
  source 1..20: mol_type = other DNA
aggcgttggacagggagctg

SEQ ID NO: 406 - DNA, 20 bp, synthetic construct
  source 1..20: mol_type = other DNA
ccctctcggaggcgttggac

SEQ ID NO: 407 - DNA, 20 bp, synthetic construct
  source 1..20: mol_type = other DNA
cctctcggaggcgttggaca

SEQ ID NO: 408 - DNA, 20 bp, synthetic construct
  source 1..20: mol_type = other DNA
ctggtccctctcggaggcgt

SEQ ID NO: 409 - DNA, 20 bp, synthetic construct
  source 1..20: mol_type = other DNA
ccctgtccaacgcctccgag

SEQ ID NO: 410 - DNA, 20 bp, synthetic construct
  source 1..20: mol_type = other DNA
cctgtccaacgcctccgaga

SEQ ID NO: 411 - DNA, 20 bp, synthetic construct
  source 1..20: mol_type = other DNA
gtggtgctggtccctctcgg

SEQ ID NO: 412 - DNA, 20 bp, synthetic construct
  source 1..20: mol_type = other DNA
caggtggtgctggtccctct

SEQ ID NO: 413 - DNA, 20 bp, synthetic construct
  source 1..20: mol_type = other DNA
gcatctcacccaggtggtgc

SEQ ID NO: 414 - DNA, 20 bp, synthetic construct
  source 1..20: mol_type = other DNA
cgagagggaccagcaccacc

SEQ ID NO: 415 - DNA, 20 bp, synthetic construct
  source 1..20: mol_type = other DNA
gagagggaccagcaccacct

SEQ ID NO: 416 - DNA, 20 bp, synthetic construct
  source 1..20: mol_type = other DNA
gtgggggcatctcacccagg

SEQ ID NO: 417 - DNA, 20 bp, synthetic construct
  source 1..20: mol_type = other DNA
ccccgacactcaggcgagaa

SEQ ID NO: 418 - DNA, 20 bp, synthetic construct
  source 1..20: mol_type = other DNA
tccccgacactcaggcgaga

SEQ ID NO: 419 - DNA, 20 bp, synthetic construct
  source 1..20: mol_type = other DNA
agcccttctcgcctgagtgt

SEQ ID NO: 420 - DNA, 20 bp, synthetic construct
  source 1..20: mol_type = other DNA
ctggctgctccccgacactc

SEQ ID NO: 421 - DNA, 20 bp, synthetic construct
  source 1..20: mol_type = other DNA
cccttctcgcctgagtgtcg

SEQ ID NO: 422 - DNA, 20 bp, synthetic construct
  source 1..20: mol_type = other DNA
gcccttctcgcctgagtgtc

SEQ ID NO: 423 - DNA, 20 bp, synthetic construct
  source 1..20: mol_type = other DNA
taggggtcgtgggtgacgtc

SEQ ID NO: 424 - DNA, 20 bp, synthetic construct
  source 1..20: mol_type = other DNA
aagaaactcataggggtcgt

SEQ ID NO: 425 - DNA, 20 bp, synthetic construct
  source 1..20: mol_type = other DNA
gaagaaactcataggggtcg

SEQ ID NO: 426 - DNA, 20 bp, synthetic construct
  source 1..20: mol_type = other DNA
gagactgaagaaactcatag

SEQ ID NO: 427 - DNA, 20 bp, synthetic construct
  source 1..20: mol_type = other DNA
ggagactgaagaaactcata

SEQ ID NO: 428 - DNA, 20 bp, synthetic construct
  source 1..20: mol_type = other DNA
tggagactgaagaaactcat

SEQ ID NO: 429 - DNA, 20 bp, synthetic construct
  source 1..20: mol_type = other DNA
tcttcagtctccagagcctg

SEQ ID NO: 430 - DNA, 20 bp, synthetic construct
  source 1..20: mol_type = other DNA
ttggcagaggccgcaggctc

SEQ ID NO: 431 - DNA, 20 bp, synthetic construct
  source 1..20: mol_type = other DNA
taggtcttggcagaggccgc

SEQ ID NO: 432 - DNA, 20 bp, synthetic construct
  source 1..20: mol_type = other DNA
ctagagttaggtcttggcag

SEQ ID NO: 433 - DNA, 20 bp, synthetic construct
  source 1..20: mol_type = other DNA
ggtggtctagagttaggtct

SEQ ID NO: 434 - DNA, 20 bp, synthetic construct
  source 1..20: mol_type = other DNA
gtagcgaacgtgtccggcgt

SEQ ID NO: 435 - DNA, 20 bp, synthetic construct
  source 1..20: mol_type = other DNA
gaccggaacgatctcgcgta

SEQ ID NO: 436 - DNA, 20 bp, synthetic construct
  source 1..20: mol_type = other DNA
ggcagtcgttcggttgatat

SEQ ID NO: 437 - DNA, 20 bp, synthetic construct
  source 1..20: mol_type = other DNA
gcttgagcacatacgcgaat

SEQ ID NO: 438 - DNA, 20 bp, synthetic construct
  source 1..20: mol_type = other DNA
gtggtagaataacgtattac

SEQ ID NO: 439 - DNA, 20 bp, synthetic construct
  source 1..20: mol_type = other DNA
gtcatacatggataaggcta

SEQ ID NO: 440 - DNA, 20 bp, synthetic construct
  source 1..20: mol_type = other DNA
gatacacgaagcatcactag

SEQ ID NO: 441 - DNA, 20 bp, synthetic construct
  source 1..20: mol_type = other DNA
gaacgttggcactacttcac

SEQ ID NO: 442 - DNA, 20 bp, synthetic construct
  source 1..20: mol_type = other DNA
gatccatgtaatgcgttcga

SEQ ID NO: 443 - DNA, 20 bp, synthetic construct
  source 1..20: mol_type = other DNA
gtcgtgaagtgcattcgatc

SEQ ID NO: 444 - DNA, 20 bp, synthetic construct
  source 1..20: mol_type = other DNA
gttcgactcgcgtgaccgta

SEQ ID NO: 445 - DNA, 20 bp, synthetic construct
  source 1..20: mol_type = other DNA
gaatctaccgcagcggttcg

SEQ ID NO: 446 - DNA, 20 bp, synthetic construct
  source 1..20: mol_type = other DNA
gaagtgacgtcgattcgata

SEQ ID NO: 447 - DNA, 20 bp, synthetic construct
  source 1..20: mol_type = other DNA
gcggtgtatgacaaccgccg

SEQ ID NO: 448 - DNA, 20 bp, synthetic construct
  source 1..20: mol_type = other DNA
gtaccgcgcctgaagttcgc

SEQ ID NO: 449 - DNA, 20 bp, synthetic construct
  source 1..20: mol_type = other DNA
gcagctcgtgtgtcgtactc

SEQ ID NO: 450 - DNA, 20 bp, synthetic construct
  source 1..20: mol_type = other DNA
gcgccttaagagtactcatc

SEQ ID NO: 451 - DNA, 20 bp, synthetic construct
  source 1..20: mol_type = other DNA
gagtgtcgtcgttgctccta

SEQ ID NO: 452 - DNA, 20 bp, synthetic construct
  source 1..20: mol_type = other DNA
gcagctcgacctcaagccgt

SEQ ID NO: 453 - DNA, 20 bp, synthetic construct
  source 1..20: mol_type = other DNA
gtatcctgacctacgcgctg

SEQ ID NO: 454 - DNA, 20 bp, synthetic construct
  source 1..20: mol_type = other DNA
gtgtatctcagcacgctaac

SEQ ID NO: 455 - DNA, 20 bp, synthetic construct
  source 1..20: mol_type = other DNA
gtcgtcatacaacggcaacg

SEQ ID NO: 456 - DNA, 20 bp, synthetic construct
  source 1..20: mol_type = other DNA
gtcgtgcgcttccggcggta

SEQ ID NO: 457 - DNA, 20 bp, synthetic construct
  source 1..20: mol_type = other DNA
gcggtcctcagtaagcgcgt

SEQ ID NO: 458 - DNA, 20 bp, synthetic construct
  source 1..20: mol_type = other DNA
gctctgctgcggaaggattc

SEQ ID NO: 459 - DNA, 20 bp, synthetic construct
  source 1..20: mol_type = other DNA
gcatggaggagcgtcgcaga

SEQ ID NO: 460 - DNA, 20 bp, synthetic construct
  source 1..20: mol_type = other DNA
gtagcgcgcgtaggagtggc

SEQ ID NO: 461 - DNA, 20 bp, synthetic construct
  source 1..20: mol_type = other DNA
gatcacctgcattcgtacac

SEQ ID NO: 462 - DNA, 20 bp, synthetic construct
  source 1..20: mol_type = other DNA
gcacacctagatatcgaatg

SEQ ID NO: 463 - DNA, 20 bp, synthetic construct
  source 1..20: mol_type = other DNA
gttgatcaacgcgcttcgcg

SEQ ID NO: 464 - DNA, 20 bp, synthetic construct
  source 1..20: mol_type = other DNA
gcgtctcactcactccatcg

SEQ ID NO: 465 - DNA, 20 bp, synthetic construct
  source 1..20: mol_type = other DNA
gccgaccaacgtcagcggta

SEQ ID NO: 466 - DNA, 20 bp, synthetic construct
  source 1..20: mol_type = other DNA
ggatacggtgcgtcaatcta

SEQ ID NO: 467 - DNA, 20 bp, synthetic construct
  source 1..20: mol_type = other DNA
gaatccagtggcggcgacaa

SEQ ID NO: 468 - DNA, 20 bp, synthetic construct
  source 1..20: mol_type = other DNA
gcactgtcagtgcaacgata

SEQ ID NO: 469 - DNA, 20 bp, synthetic construct
  source 1..20: mol_type = other DNA
gcgatcctcaagtatgctca

SEQ ID NO: 470 - DNA, 20 bp, synthetic construct
  source 1..20: mol_type = other DNA
gctaatatcgacacggccgc

SEQ ID NO: 471 - DNA, 20 bp, synthetic construct
  source 1..20: mol_type = other DNA
ggagatgcatcgaagtcgat

SEQ ID NO: 472 - DNA, 20 bp, synthetic construct
  source 1..20: mol_type = other DNA
ggatgcactccatctcgtct

SEQ ID NO: 473 - DNA, 20 bp, synthetic construct
  source 1..20: mol_type = other DNA
gtgccgagtaataacgcgag

SEQ ID NO: 474 - DNA, 20 bp, synthetic construct
  source 1..20: mol_type = other DNA
gagattccgatgtaacgtac

SEQ ID NO: 475 - DNA, 20 bp, synthetic construct
  source 1..20: mol_type = other DNA
gtcgtcacgagcaggattgc

SEQ ID NO: 476 - DNA, 20 bp, synthetic construct
  source 1..20: mol_type = other DNA
gcgttagtcacttagctcga

SEQ ID NO: 477 - DNA, 20 bp, synthetic construct
  source 1..20: mol_type = other DNA
gttcacacggtgtcggatag

SEQ ID NO: 478 - DNA, 20 bp, synthetic construct
  source 1..20: mol_type = other DNA
ggataggtgaccttagtacg

SEQ ID NO: 479 - DNA, 20 bp, synthetic construct
  source 1..20: mol_type = other DNA
gtatgagtcaagctaatgcg

SEQ ID NO: 480 - DNA, 20 bp, synthetic construct
  source 1..20: mol_type = other DNA
gcaactattggaatacgtga

SEQ ID NO: 481 - DNA, 20 bp, synthetic construct
  source 1..20: mol_type = other DNA
gttaccttcgctcgtctata

SEQ ID NO: 482 - DNA, 20 bp, synthetic construct
  source 1..20: mol_type = other DNA
gtaccgagcaccacaggccg

SEQ ID NO: 483 - DNA, 20 bp, synthetic construct
  source 1..20: mol_type = other DNA
gtcagccatcggatagagat

SEQ ID NO: 484 - DNA, 20 bp, synthetic construct
  source 1..20: mol_type = other DNA
gtacggcactcctagccgct

SEQ ID NO: 485 - DNA, 20 bp, synthetic construct
  source 1..20: mol_type = other DNA
ggtcctgtcgtatgcttgca

SEQ ID NO: 486 - DNA, 20 bp, synthetic construct
  source 1..20: mol_type = other DNA
gccgcaatatatgcggtaag

SEQ ID NO: 487 - DNA, 20 bp, synthetic construct
  source 1..20: mol_type = other DNA
gcgcacgtataatcctgcgt

SEQ ID NO: 488 - DNA, 20 bp, synthetic construct
  source 1..20: mol_type = other DNA
gtgcacaacacgatccacga

SEQ ID NO: 489 - DNA, 20 bp, synthetic construct
  source 1..20: mol_type = other DNA
gcacaatgttgacgtaagtg

SEQ ID NO: 490 - DNA, 20 bp, synthetic construct
  source 1..20: mol_type = other DNA
gtaagatgctgctcaccgtg

SEQ ID NO: 491 - DNA, 20 bp, synthetic construct
  source 1..20: mol_type = other DNA
gtcggtgatccaacgtatcg

SEQ ID NO: 492 - DNA, 20 bp, synthetic construct
  source 1..20: mol_type = other DNA
gagctagtaggacgcaagac

SEQ ID NO: 493 - DNA, 20 bp, synthetic construct
  source 1..20: mol_type = other DNA
gtacgtggaagcttgtggcc

SEQ ID NO: 494 - DNA, 20 bp, synthetic construct
  source 1..20: mol_type = other DNA
gagaactgccagttctcgat

SEQ ID NO: 495 - DNA, 20 bp, synthetic construct
  source 1..20: mol_type = other DNA
gccattcggcgcggcacttc

SEQ ID NO: 496 - DNA, 20 bp, synthetic construct
  source 1..20: mol_type = other DNA
gcacacgaccaatccgcttc

SEQ ID NO: 497 - DNA, 20 bp, synthetic construct
  source 1..20: mol_type = other DNA
gaggtgatcgattaagtaca

SEQ ID NO: 498 - DNA, 20 bp, synthetic construct
  source 1..20: mol_type = other DNA
gtcactcgcagacgcctaac

SEQ ID NO: 499 - DNA, 20 bp, synthetic construct
  source 1..20: mol_type = other DNA
gcgctacggaatcatacgtt

SEQ ID NO: 500 - DNA, 20 bp, synthetic construct
  source 1..20: mol_type = other DNA
ggtaggacctcacggcgcgc

SEQ ID NO: 501 - DNA, 20 bp, synthetic construct
  source 1..20: mol_type = other DNA
gaactgcatcttgttgtagt

SEQ ID NO: 502 - DNA, 20 bp, synthetic construct
  source 1..20: mol_type = other DNA
gatcctgatccggcggcgcg

SEQ ID NO: 503 - DNA, 20 bp, synthetic construct
  source 1..20: mol_type = other DNA
ggtatgcgcgatcctgagtt

SEQ ID NO: 504 - DNA, 20 bp, synthetic construct
  source 1..20: mol_type = other DNA
gcggagctagagagcggtca

SEQ ID NO: 505 - DNA, 20 bp, synthetic construct
  source 1..20: mol_type = other DNA
gaatggcaattacggctgat

SEQ ID NO: 506 - DNA, 20 bp, synthetic construct
  source 1..20: mol_type = other DNA
gtatggtgagtagtcgcttg

SEQ ID NO: 507 - DNA, 20 bp, synthetic construct
  source 1..20: mol_type = other DNA
gtgtaattgcgtctagtcgg

SEQ ID NO: 508 - DNA, 20 bp, synthetic construct
  source 1..20: mol_type = other DNA
ggtcctggcgaggagccttg

SEQ ID NO: 509 - DNA, 20 bp, synthetic construct
  source 1..20: mol_type = other DNA
gaagataagtcgctgtctcg

SEQ ID NO: 510 - DNA, 20 bp, synthetic construct
  source 1..20: mol_type = other DNA
gtcggcgttctgttgtgact

SEQ ID NO: 511 - DNA, 20 bp, synthetic construct
  source 1..20: mol_type = other DNA
gaggcaagccgttaggtgta

SEQ ID NO: 512 - DNA, 20 bp, synthetic construct
  source 1..20: mol_type = other DNA
gcggatccagatctcattcg

SEQ ID NO: 513 - DNA, 20 bp, synthetic construct
  source 1..20: mol_type = other DNA
ggaacataggagcacgtagt

SEQ ID NO: 514 - DNA, 20 bp, synthetic construct
  source 1..20: mol_type = other DNA
gtcatcattatggcgtaagg

SEQ ID NO: 515 - DNA, 20 bp, synthetic construct
  source 1..20: mol_type = other DNA
gcgactagcgccatgagcgg

SEQ ID NO: 516 - DNA, 20 bp, synthetic construct
  source 1..20: mol_type = other DNA
ggcgaagttcgacatgacac

SEQ ID NO: 517 - DNA, 20 bp, synthetic construct
  source 1..20: mol_type = other DNA
gctgtcgtgtggaggctatg

SEQ ID NO: 518 - DNA, 20 bp, synthetic construct
  source 1..20: mol_type = other DNA
gcggagagcattgacctcat

SEQ ID NO: 519 - DNA, 20 bp, synthetic construct
  source 1..20: mol_type = other DNA
gactaatggaccaagtcagt

SEQ ID NO: 520 - DNA, 20 bp, synthetic construct
  source 1..20: mol_type = other DNA
gcggattagaggtaatgcgg

SEQ ID NO: 521 - DNA, 20 bp, synthetic construct
  source 1..20: mol_type = other DNA
gccgacggcaatcagtacgc

SEQ ID NO: 522 - DNA, 20 bp, synthetic construct
  source 1..20: mol_type = other DNA
gtaacctctcgagcgataga

SEQ ID NO: 523 - DNA, 20 bp, synthetic construct
  source 1..20: mol_type = other DNA
gacttgtatgtggcttacgg

SEQ ID NO: 524 - DNA, 20 bp, synthetic construct
  source 1..20: mol_type = other DNA
gtcactgtggtcgaacatgt

SEQ ID NO: 525 - DNA, 20 bp, synthetic construct
  source 1..20: mol_type = other DNA
gtactccaatccgcgatgac

SEQ ID NO: 526 - DNA, 20 bp, synthetic construct
  source 1..20: mol_type = other DNA
gcgttggcacgatgttacgg

SEQ ID NO: 527 - DNA, 20 bp, synthetic construct
  source 1..20: mol_type = other DNA
gaaccagccggctagtatga

SEQ ID NO: 528 - DNA, 20 bp, synthetic construct
  source 1..20: mol_type = other DNA
gtatactagctaaccacacg

SEQ ID NO: 529 - DNA, 20 bp, synthetic construct
  source 1..20: mol_type = other DNA
gaatcggaatagttgattcg

SEQ ID NO: 530 - DNA, 20 bp, synthetic construct
  source 1..20: mol_type = other DNA
gagcacttgcatgaggcggt

SEQ ID NO: 531 - DNA, 20 bp, synthetic construct
  source 1..20: mol_type = other DNA
gaacggcgatgaagccagcc

SEQ ID NO: 532 - DNA, 20 bp, synthetic construct
  source 1..20: mol_type = other DNA
gcaaccgagatgagaggttc

SEQ ID NO: 533 - DNA, 20 bp, synthetic construct
  source 1..20: mol_type = other DNA
gcaagatcaatatgcgtgat

SEQ ID NO: 534 - DNA, 20 bp, synthetic construct
  source 1..20: mol_type = other DNA
acggaggctaagcgtcgcaa

SEQ ID NO: 535 - DNA, 20 bp, synthetic construct
  source 1..20: mol_type = other DNA
cgcttccgcggcccgttcaa

SEQ ID NO: 536 - DNA, 20 bp, synthetic construct
  source 1..20: mol_type = other DNA
atcgtttccgcttaacggcg

SEQ ID NO: 537 - DNA, 20 bp, synthetic construct
  source 1..20: mol_type = other DNA
gtaggcgcgccgctctctac

SEQ ID NO: 538 - DNA, 20 bp, synthetic construct
  source 1..20: mol_type = other DNA
ccatatcggggcgagacatg

SEQ ID NO: 539 - DNA, 20 bp, synthetic construct
  source 1..20: mol_type = other DNA
tactaacgccgctcctacag

SEQ ID NO: 540 - DNA, 20 bp, synthetic construct
  source 1..20: mol_type = other DNA
tgaggatcatgtcgagcgcc

SEQ ID NO: 541 - DNA, 20 bp, synthetic construct
  source 1..20: mol_type = other DNA
gggcccgcataggatatcgc

SEQ ID NO: 542 - DNA, 20 bp, synthetic construct
  source 1..20: mol_type = other DNA
tagacaaccgcggagaatgc

SEQ ID NO: 543 - DNA, 20 bp, synthetic construct
  source 1..20: mol_type = other DNA
acgggcggctatcgctgact

SEQ ID NO: 544 - DNA, 20 bp, synthetic construct
  source 1..20: mol_type = other DNA
cgcggaaattttaccgacga

SEQ ID NO: 545 - DNA, 20 bp, synthetic construct
  source 1..20: mol_type = other DNA
cttacaatcgtcggtccaat

SEQ ID NO: 546 - DNA, 20 bp, synthetic construct
  source 1..20: mol_type = other DNA
gcgtgcgtcccgggttaccc

SEQ ID NO: 547 - DNA, 20 bp, synthetic construct
  source 1..20: mol_type = other DNA
cggagtaacaagcggacgga

SEQ ID NO: 548 - DNA, 20 bp, synthetic construct
  source 1..20: mol_type = other DNA
cgagtgttatacgcaccgtt

SEQ ID NO: 549 - DNA, 20 bp, synthetic construct
  source 1..20: mol_type = other DNA
cgactaaccggaaacttttt

SEQ ID NO: 550 - DNA, 20 bp, synthetic construct
  source 1..20: mol_type = other DNA
caacgggttctcccggctac

SEQ ID NO: 551 - DNA, 20 bp, synthetic construct
  source 1..20: mol_type = other DNA
caggagtcgccgatacgcgt

SEQ ID NO: 552 - DNA, 20 bp, synthetic construct
  source 1..20: mol_type = other DNA
ttcacgtcgtctcgcgacca

SEQ ID NO: 553 - DNA, 20 bp, synthetic construct
  source 1..20: mol_type = other DNA
gtgtcggattccgccgctta

SEQ ID NO: 554 - DNA, 20 bp, synthetic construct
  source 1..20: mol_type = other DNA
cacgaactcacaccgcgcga

SEQ ID NO: 555 - DNA, 20 bp, synthetic construct
  source 1..20: mol_type = other DNA
cgctagtacgctcctctata

SEQ ID NO: 556 - DNA, 20 bp, synthetic construct
  source 1..20: mol_type = other DNA
tcgcgcttgggttatacgct

SEQ ID NO: 557 - DNA, 20 bp, synthetic construct
  source 1..20: mol_type = other DNA
ctatctcgagtggtaatgcg

SEQ ID NO: 558 - DNA, 20 bp, synthetic construct
  source 1..20: mol_type = other DNA
aatcgactcgaacttcgtgt

SEQ ID NO: 559 - DNA, 20 bp, synthetic construct
  source 1..20: mol_type = other DNA
cccgatggactataccgaac

SEQ ID NO: 560 - DNA, 20 bp, synthetic construct
  source 1..20: mol_type = other DNA
acgttcgagtacgaccagct

SEQ ID NO: 561 - DNA, 20 bp, synthetic construct
  source 1..20: mol_type = other DNA
cgcgacgactcaacctagtc

SEQ ID NO: 562 - DNA, 20 bp, synthetic construct
  source 1..20: mol_type = other DNA
ggtcaccgatcgagagctag

SEQ ID NO: 563 - DNA, 20 bp, synthetic construct
  source 1..20: mol_type = other DNA
ctcaaccgaccgtatggtca

SEQ ID NO: 564 - DNA, 20 bp, synthetic construct
  source 1..20: mol_type = other DNA
cgtattcgactctcaacgcg

SEQ ID NO: 565 - DNA, 20 bp, synthetic construct
  source 1..20: mol_type = other DNA
ctagccgcccagatcgagcc

SEQ ID NO: 566 - DNA, 20 bp, synthetic construct
  source 1..20: mol_type = other DNA
gaatcgaccgacactaatgt

SEQ ID NO: 567 - DNA, 20 bp, synthetic construct
  source 1..20: mol_type = other DNA
acttcagttcggcgtagtca

SEQ ID NO: 568 - DNA, 20 bp, synthetic construct
  source 1..20: mol_type = other DNA
gtgcgatgtcgcttcaacgt

SEQ ID NO: 569 - DNA, 20 bp, synthetic construct
  source 1..20: mol_type = other DNA
cgcctaatttccggatcaat

SEQ ID NO: 570 - DNA, 20 bp, synthetic construct
  source 1..20: mol_type = other DNA
cgtggccggaaccgtcatag

SEQ ID NO: 571 - DNA, 20 bp, synthetic construct
  source 1..20: mol_type = other DNA
accctccgaatcgtaacgga

SEQ ID NO: 572 - DNA, 20 bp, synthetic construct
  source 1..20: mol_type = other DNA
aaacggtacgacagcgtgtg

SEQ ID NO: 573 - DNA, 20 bp, synthetic construct
  source 1..20: mol_type = other DNA
acatagtcgacggctcgatt

SEQ ID NO: 574 - DNA, 20 bp, synthetic construct
  source 1..20: mol_type = other DNA
gatggcgcttcagtcgtcgg

SEQ ID NO: 575 - DNA, 20 bp, synthetic construct
  source 1..20: mol_type = other DNA
ataatccggaaacgctcgac

SEQ ID NO: 576 - DNA, 20 bp, synthetic construct
  source 1..20: mol_type = other DNA
cgccgggctgacaattaacg

SEQ ID NO: 577 - DNA, 20 bp, synthetic construct
  source 1..20: mol_type = other DNA
cgtcgccatatgccggtggc

SEQ ID NO: 578 - DNA, 20 bp, synthetic construct
  source 1..20: mol_type = other DNA
cgggcctataacaccatcga

SEQ ID NO: 579 - DNA, 20 bp, synthetic construct
  source 1..20: mol_type = other DNA
cgccgttccgagatacttga

SEQ ID NO: 580 - DNA, 20 bp, synthetic construct
  source 1..20: mol_type = other DNA
cgggacgtcgcgaaaatgta

SEQ ID NO: 581 - DNA, 20 bp, synthetic construct
  source 1..20: mol_type = other DNA
tcggcatacgggacacacgc

SEQ ID NO: 582 - DNA, 20 bp, synthetic construct
  source 1..20: mol_type = other DNA
agctccatcgccgcgataat

SEQ ID NO: 583 - DNA, 20 bp, synthetic construct
  source 1..20: mol_type = other DNA
atcgtatcatcagctagcgc

SEQ ID NO: 584 - DNA, 20 bp, synthetic construct
  source 1..20: mol_type = other DNA
tcgatcgaggttgcattcgg

SEQ ID NO: 585 - DNA, 20 bp, synthetic construct
  source 1..20: mol_type = other DNA
ctcgacagttcgtcccgagc

SEQ ID NO: 586 - DNA, 20 bp, synthetic construct
  source 1..20: mol_type = other DNA
cggtagtattaatcgctgac

SEQ ID NO: 587 - DNA, 20 bp, synthetic construct
  source 1..20: mol_type = other DNA
tgaacgcgtgtttccttgca

SEQ ID NO: 588 - DNA, 20 bp, synthetic construct
  source 1..20: mol_type = other DNA
cgacgctaggtaacgtagag

SEQ ID NO: 589 - DNA, 20 bp, synthetic construct
  source 1..20: mol_type = other DNA
cattgttgagcgggcgcgct

SEQ ID NO: 590 - DNA, 20 bp, synthetic construct
  source 1..20: mol_type = other DNA
ccgctattgaaaccgcccac

SEQ ID NO: 591 - DNA, 20 bp, synthetic construct
  source 1..20: mol_type = other DNA
agacacgtcaccggtcaaaa

SEQ ID NO: 592 - DNA, 20 bp, synthetic construct
  source 1..20: mol_type = other DNA
tttacgatctagcggcgtag

SEQ ID NO: 593 - DNA, 20 bp, synthetic construct
  source 1..20: mol_type = other DNA
ttcgcacgattgcaccttgg

SEQ ID NO: 594 - DNA, 20 bp, synthetic construct
  source 1..20: mol_type = other DNA
ggttagagactaggcgcgcg

SEQ ID NO: 595 - DNA, 20 bp, synthetic construct
  source 1..20: mol_type = other DNA
cctccgtgctaacgcggacg

SEQ ID NO: 596 - DNA, 20 bp, synthetic construct
  source 1..20: mol_type = other DNA
ttatcgcgtagtgctgacgt

SEQ ID NO: 597 - DNA, 20 bp, synthetic construct
  source 1..20: mol_type = other DNA
tacgcttgcgtttagcgtcc

SEQ ID NO: 598 - DNA, 20 bp, synthetic construct
  source 1..20: mol_type = other DNA
cgcggcccacgcgtcatcgc

SEQ ID NO: 599 - DNA, 20 bp, synthetic construct
  source 1..20: mol_type = other DNA
agctcgccatgtcggttctc

SEQ ID NO: 600 - DNA, 20 bp, synthetic construct
  source 1..20: mol_type = other DNA
aactagcccgagcagcttcg

SEQ ID NO: 601 - DNA, 20 bp, synthetic construct
  source 1..20: mol_type = other DNA
cgcaaggtgtcggtaaccct

SEQ ID NO: 602 - DNA, 20 bp, synthetic construct
  source 1..20: mol_type = other DNA
cttcgacgccatcgtgctca

SEQ ID NO: 603 - DNA, 20 bp, synthetic construct
  source 1..20: mol_type = other DNA
tcctggataccgcgtggtta

SEQ ID NO: 604 - DNA, 20 bp, synthetic construct
  source 1..20: mol_type = other DNA
atagccgccgctcattactt

SEQ ID NO: 605 - DNA, 20 bp, synthetic construct
  source 1..20: mol_type = other DNA
gtcgtccgggattacaaaat

SEQ ID NO: 606 - DNA, 20 bp, synthetic construct
  source 1..20: mol_type = other DNA
taatgctgcacacgccgaat

SEQ ID NO: 607 - DNA, 20 bp, synthetic construct
  source 1..20: mol_type = other DNA
tatcgcttccgattagtccg

SEQ ID NO: 608 - DNA, 20 bp, synthetic construct
  source 1..20: mol_type = other DNA
gtaccataccgcgtaccctt

SEQ ID NO: 609 - DNA, 20 bp, synthetic construct
  source 1..20: mol_type = other DNA
taagatccgcgggtggcaac

SEQ ID NO: 610 - DNA, 20 bp, synthetic construct
  source 1..20: mol_type = other DNA
gtagacgtcgtgagcttcac

SEQ ID NO: 611 - DNA, 20 bp, synthetic construct
  source 1..20: mol_type = other DNA
tcgcggacatagggctctaa

SEQ ID NO: 612 - DNA, 20 bp, synthetic construct
  source 1..20: mol_type = other DNA
agcgcagatagcgcgtatca

SEQ ID NO: 613 - DNA, 20 bp, synthetic construct
  source 1..20: mol_type = other DNA
gttcgcttcgtaacgaggaa

SEQ ID NO: 614 - DNA, 20 bp, synthetic construct
  source 1..20: mol_type = other DNA
gacccccgataacttttgac

SEQ ID NO: 615 - DNA, 20 bp, synthetic construct
  source 1..20: mol_type = other DNA
acgtccatactgtcggctac

SEQ ID NO: 616 - DNA, 20 bp, synthetic construct
  source 1..20: mol_type = other DNA
gtaccattgccggctcccta

SEQ ID NO: 617 - DNA, 20 bp, synthetic construct
  source 1..20: mol_type = other DNA
tggttccgtaggtcggtata

SEQ ID NO: 618 - DNA, 20 bp, synthetic construct
  source 1..20: mol_type = other DNA
tctggcttgacacgaccgtt

SEQ ID NO: 619 - DNA, 20 bp, synthetic construct
  source 1..20: mol_type = other DNA
cgctaggtccggtaagtgcg

SEQ ID NO: 620 - DNA, 20 bp, synthetic construct
  source 1..20: mol_type = other DNA
agcacgtaatgtccgtggat

SEQ ID NO: 621 - DNA, 20 bp, synthetic construct
  source 1..20: mol_type = other DNA
aaggcgcgcgaatgtggcag

SEQ ID NO: 622 - DNA, 20 bp, synthetic construct
  source 1..20: mol_type = other DNA
actgcggagcgcccaatatc

SEQ ID NO: 623 - DNA, 20 bp, synthetic construct
  source 1..20: mol_type = other DNA
cgtcgagtgctcgaactcca

SEQ ID NO: 624 - DNA, 20 bp, synthetic construct
  source 1..20: mol_type = other DNA
tcgcagcggcgtgggatcgg

SEQ ID NO: 625 - DNA, 20 bp, synthetic construct
  source 1..20: mol_type = other DNA
atctgtcctaattcggatcg

SEQ ID NO: 626 - DNA, 20 bp, synthetic construct
  source 1..20: mol_type = other DNA
tgcggcgtaatgcttgaaag

SEQ ID NO: 627 - DNA, 20 bp, synthetic construct
  source 1..20: mol_type = other DNA
cgaacttaatcccgtggcaa

SEQ ID NO: 628 - DNA, 20 bp, synthetic construct
  source 1..20: mol_type = other DNA
gccgtgttgctggatacgcc

SEQ ID NO: 629 - DNA, 20 bp, synthetic construct
  source 1..20: mol_type = other DNA
taccctccggatacggactg

SEQ ID NO: 630 - DNA, 20 bp, synthetic construct
  source 1..20: mol_type = other DNA
ccgttggactatggcgggtc

SEQ ID NO: 631 - DNA, 20 bp, synthetic construct
  source 1..20: mol_type = other DNA
gtacggggcgatcatccaca

SEQ ID NO: 632 - DNA, 20 bp, synthetic construct
  source 1..20: mol_type = other DNA
aagagtagtagacgcccggg

SEQ ID NO: 633 - DNA, 20 bp, synthetic construct
  source 1..20: mol_type = other DNA
aagagcgaatcgatttcgtg

SEQ ID NO: 634 - DNA, 20 bp, synthetic construct
  source 1..20: mol_type = other DNA
tcaacaccagtgcctgacgg

SEQ ID NO: 635 - DNA, 20 bp, synthetic construct
  source 1..20: mol_type = other DNA
aaagtagcttcactctctcg

SEQ ID NO: 636 - DNA, 20 bp, synthetic construct
  source 1..20: mol_type = other DNA
gagccaaccaatagatgtcc

SEQ ID NO: 637 - DNA, 20 bp, synthetic construct
  source 1..20: mol_type = other DNA
gcgccgccatgaacctagag

SEQ ID NO: 638 - DNA, 20 bp, synthetic construct
  source 1..20: mol_type = other DNA
acaaaggttggaacagaacc

SEQ ID NO: 639 - DNA, 20 bp, synthetic construct
  source 1..20: mol_type = other DNA
ggtgaccgggttattgatgt

SEQ ID NO: 640 - DNA, 20 bp, synthetic construct
  source 1..20: mol_type = other DNA
ttagtggaggactacagagc

SEQ ID NO: 641 - DNA, 20 bp, synthetic construct
  source 1..20: mol_type = other DNA
acatatagcccgtaaagctg

SEQ ID NO: 642 - DNA, 20 bp, synthetic construct
  source 1..20: mol_type = other DNA
cgttggcgatgatctccacg

SEQ ID NO: 643 - DNA, 20 bp, synthetic construct
  source 1..20: mol_type = other DNA
tggccttttctacctcgcgc

SEQ ID NO: 644 - DNA, 20 bp, synthetic construct
  source 1..20: mol_type = other DNA
aatggagatactcatctggg

SEQ ID NO: 645 - DNA, 20 bp, synthetic construct
  source 1..20: mol_type = other DNA
gaagcccgtccagaaagtgt

SEQ ID NO: 646 - DNA, 20 bp, synthetic construct
  source 1..20: mol_type = other DNA
caatctgaggaactccacga

SEQ ID NO: 647 - DNA, 20 bp, synthetic construct
  source 1..20: mol_type = other DNA
aggctgcggcgcccacgaga

SEQ ID NO: 648 - DNA, 20 bp, synthetic construct
  source 1..20: mol_type = other DNA
actttgaccaggccttgcta

SEQ ID NO: 649 - DNA, 20 bp, synthetic construct
  source 1..20: mol_type = other DNA
accttccataactgccacgc

SEQ ID NO: 650 - DNA, 20 bp, synthetic construct
  source 1..20: mol_type = other DNA
cgaggcgtacatacccaagg

SEQ ID NO: 651 - DNA, 20 bp, synthetic construct
  source 1..20: mol_type = other DNA
atggtacggccaaatcaaga

SEQ ID NO: 652 - DNA, 20 bp, synthetic construct
  source 1..20: mol_type = other DNA
tcttgtaatcccatacgcgt

SEQ ID NO: 653 - DNA, 20 bp, synthetic construct
  source 1..20: mol_type = other DNA
attcacaggacacagagaat

SEQ ID NO: 654 - DNA, 20 bp, synthetic construct
  source 1..20: mol_type = other DNA
ccagggctccatcctcaaga

SEQ ID NO: 655 - DNA, 20 bp, synthetic construct
  source 1..20: mol_type = other DNA
tgagctgcaccaaagagacg

SEQ ID NO: 656 - DNA, 20 bp, synthetic construct
  source 1..20: mol_type = other DNA
atgtctgcagatgtacccct

SEQ ID NO: 657 - DNA, 20 bp, synthetic construct
  source 1..20: mol_type = other DNA
cgaagataacgcggatacct

SEQ ID NO: 658 - DNA, 20 bp, synthetic construct
  source 1..20: mol_type = other DNA
gctgcaggccgagtacaccg

SEQ ID NO: 659 - DNA, 20 bp, synthetic construct
  source 1..20: mol_type = other DNA
acaagtgggaggcttacctg

SEQ ID NO: 660 - DNA, 20 bp, synthetic construct
  source 1..20: mol_type = other DNA
gcagcgtacagggatgatca

SEQ ID NO: 661 - DNA, 20 bp, synthetic construct
  source 1..20: mol_type = other DNA
gcagtagcgcttcaggccca

SEQ ID NO: 662 - DNA, 20 bp, synthetic construct
  source 1..20: mol_type = other DNA
caaatggtggggtaacagaa

SEQ ID NO: 663 - DNA, 20 bp, synthetic construct
  source 1..20: mol_type = other DNA
gaaaggaactggctaccgtt

SEQ ID NO: 664 - DNA, 20 bp, synthetic construct
  source 1..20: mol_type = other DNA
agggcttccgttacaagatg

SEQ ID NO: 665 - DNA, 20 bp, synthetic construct
  source 1..20: mol_type = other DNA
gaacaagcaacacctaaaag

SEQ ID NO: 666 - DNA, 20 bp, synthetic construct
  source 1..20: mol_type = other DNA
tgaggagaaggaacggctca

SEQ ID NO: 667 - DNA, 20 bp, synthetic construct
  source 1..20: mol_type = other DNA
ggaagaatgcagagtataag

SEQ ID NO: 668 - DNA, 20 bp, synthetic construct
  source 1..20: mol_type = other DNA
ggaatttgaggaactcctga

SEQ ID NO: 669 - DNA, 20 bp, synthetic construct
  source 1..20: mol_type = other DNA
gctcaccggccatccaggaa

SEQ ID NO: 670 - DNA, 20 bp, synthetic construct
  source 1..20: mol_type = other DNA
actggccaggaacgatgcga

SEQ ID NO: 671 - DNA, 20 bp, synthetic construct
  source 1..20: mol_type = other DNA
gcagctccaagatcttccca

SEQ ID NO: 672 - DNA, 20 bp, synthetic construct
  source 1..20: mol_type = other DNA
gaatgagtacacagaacgga

SEQ ID NO: 673 - DNA, 20 bp, synthetic construct
  source 1..20: mol_type = other DNA
ggagcaggacaaggtcgggg